SEQUENCE LISTING (56 sequences)

SEQ ID NO: 1 - DNA, 2821 bp, Homo sapiens
  source 1..2821: mol_type = other DNA
agtttgaggctgcactgcacacctgcacaacctgccttctacttagttcttctgagacatttctgaaagtctgaattcctaggactgctcaatgacctttgtccctgttgggcacacgcagtgtctcatcgctggtattgcacctttaatgagaccaggagttccgcaaaagtaaaacaaaggggtcacagactggttctggttctaccacttcctcagtatgtgctctgggacaaaacagcatatttggcaacatctcggtgtccttatctgcagcttgaagaggcgccacagcaacatcctcagagtctgagcgaactgcgcccagcgcgggcacggagcctcccaccgccagcaacctgcggccccggagaaggcagcgagcgcagtgacagcgcctcaccgccaccagctcctggaccaccatggccaagaaccgcagggacagaaacagttggggtggattttcggaaaagacatatgaatggagctcagaagaggaggagccagtgaaaaaggcaggaccagtccaagtcctcattgtcaaagatgaccattcctttgagttagatgaaactgcattaaatcggatccttctctcggaggctgtcagagacaaggaggttgttgctgtatctgttgctggagcatttagaaaaggaaaatcattcctgatggacttcatgttgagatacatgtacaaccaggaatcagttgattgggttggagactacaatgaaccattgactggtttttcatggagaggtggatctgagcgagagaccacaggaattcagatatggagtgaaatcttccttatcaataaacctgatggtaaaaaggttgcagtgttattgatggatactcagggaacctttgatagtcagtcaactttgagagattcagccacagtatttgcccttagcacaatgatcagctcaatacaggtatataacttatcccaaaatgtccaggaggatgatcttcagcacctccagcttttcactgagtatggcagactggcaatggaggaaacattcctgaagccatttcagagtctgatatttcttgttcgagactggagtttcccatacgaattttcatatggagccgatggtggtgccaaattcttggaaaaacgcctcaaggtctcagggaaccagcatgaagaactacagaacgtcagaaaacacatccattcctgtttcaccaacatttcctgttttctgctacctcatcctggcttaaaagtagctaccaatccaaactttgatggaaaattgaaagaaatagatgatgaattcatcaaaaacttgaaaatactgattccttggctacttagtcccgagagcctagatattaaagagatcaatgggaataaaatcacctgccggggtctggtggagtacttcaaggcttatataaagatctatcaaggtgaagaattaccacatcccaaatccatgttacaggccacagcagaagctaacaatttagcagccgtggcaactgccaaggacacatacaacaaaaaaatggaagagatttgtggtggtgacaaaccatttctggccccaaatgacttgcagaccaaacacctgcaacttaaggaagaatctgtgaagctattccgaggggtgaagaagatgggtggggaagaatttagccggcgttacctgcagcagttggagagtgaaatagatgaactttacatccaatatatcaagcacaatgatagcaaaaatatcttccatgcagctcgtaccccagccacactgtttgtagtcatctttatcacatatgtgattgctggtgtgactggattcattggtttggacatcatagctagcctatgcaatatgataatgggactgacccttatcaccctgtgcacttgggcatatatccggtactctggagaataccgagagctgggagctgtaatagaccaggtggctgcagctctgtgggaccaggctttgtacaagctttacagtgcagcagcaacccacagacatctgtatcatcaagctttccctacaccaaagtcggaatctactgaacaatcagaaaagaaaaaaatgtaatgcaaattttaagaaatacaggtgcatgaccaattgtcaattaaatattcagttttatgtctccatgcaaacattcaaagtgcttccatcagaacggagtaaaatactaaacacctctgaagactgcaaactggattagttcttttacttcagtgtttaataagcagatgtatgtatgcatggttatactattttgttaacatgtacaatttcctgatttttcttcaaaaatgctgttataaagtatttgtctatttatgataacagtacacgtgttctgcttgaatttactaaattctactactgggttataattaaatcatgtgatattccacgtttggatatgctcatttaatttctacagaaaaaattttaaattatttcacattagccatttgttaaaacacagcatcataactcagcaggctggatttaatctgtatcatcttatatatatcacaatcttatttttaagcacattttagagttccttagttgctttatcaaaaaccagatattgcttttacatggtttaatagaatataaacctcttgataaaaaatgcacaaaaaatcactttgtatatgtgagtttcactgcattgtatattttttcatttggtacacaaagaatgtattcttcataggtttattcttttaatatgtgaactattattaaagtttactctggttcctaagattaaaaacaaatgcttactgaatttgaaaaaaaaa

SEQ ID NO: 2 - DNA, 1688 bp, Homo sapiens
  source 1..1688: mol_type = other DNA
acttgacagcccgctgaggacgcagcgtcagctgacctggggagtcgcgattcgtgccggccggtcctggttctccggtcccgccgctcccgcagcagccatgtcgttcttcccggagctttactttaacgtggacaatggctacttggagggactggtgcgcggcctgaaggccggggtgctcagccaggccgactacctcaacctggtgcagtgcgagacgctagaggacttgaaactgcatctgcagagcactgattatggtaacttcctggccaacgaggcatcacctctgacggtgtcagtcatcgatgaccggctcaaggagaagatggtggtggagttccgccacatgaggaaccatgcctatgagccactcgccagcttcctagacttcattacttacagttacatgatcgacaacgtgatcctgctcatcacaggcacgctgcaccagcgctccatcgctgagctcgtgcccaagtgccacccactaggcagcttcgagcagatggaggccgtgaacattgctcagacacctgctgagctctacaatgccattctggtggacacgcctcttgcggcttttttccaggactgcatttcagagcaggaccttgacgagatgaacatcgagatcatccgcaacaccctctacaaggcctacctggagtccttctacaagttctgcaccctactgggcgggactacggctgatgccatgtgccccatcctggagtttgaagcagaccgccgcgccttcatcatcaccatcaattctttcggcacagagctgtccaaagaggaccgtgccaagctctttccacactgtgggcggctctaccctgagggcctggcgcagctggctcgggctgacgactatgaacaggtcaagaacgtggccgattactacccggagtacaagctgctcttcgagggtgcaggtagcaaccctggagacaagacgctggaggaccgattctttgagcacgaggtaaagctgaacaagttggccttcctgaaccagttccactttggtgtcttctatgccttcgtgaagctcaaggagcaggagtgtcgcaacatcgtgtggatcgctgaatgtatcgcccagcgccaccgcgccaaaatcgacaactacatccctatcttctagcgtcctggcccaaggctctcaattgcactctttgtgtgtgtgtgtgtgtgtgtgcgcgtgtgtgtgcgtgtgtgtgtatgtggtctgtgacaagcctgtggctcacctgcctgtccggggtgtagtacgctgtcctagcggctgcccagttctcctgaccctcttagagactgttcttaggcctgaaaaggggctgggcacccccccccaccaaggatggacgaagaccccctccagagcaaggaggccccctcagccctgtggttacagccgctgatgtatctaagaagcatgtcactttcatgttcctccctaactccctgacctgagaaccctggggcctgggggcagtttgagcctcctctcccttctgtgggtcgctcccagagccatggcccatgggaaggacagagtgtgtgtgtccttggggcctggggggatgttgctcctcagctccctccctcagccctgcccctctgagacaataaaactgccctctctaaggccaactgtcaaaaaaaaaaaaaaaaaa

SEQ ID NO: 3 - DNA, 10308 bp, Homo sapiens
  source 1..10308: mol_type = other DNA
ccctccctcgctcgctcctcgcaagctcccgctcgctccctgcccactcccggggggacgttccgtgccgcggccgccgcggccgctgcttctttcacactttagttgggagctgcgcgccgcgctcagttactggagagctggccgcgcgccgccgcctcccgcacgcttgcacgcgggcccggcttcggggttttgggttcttactccaagcggcggggaggagggggagccccggacacactgtggggaggaggaggaagaagaggaggagggaggaagaaaaaagacgaggaggacaggggcggggggcgggaggcttgccaccttcagcccccccgcgaacgcccaaggtgcacacatcttgaccaactcagcagcaaggtggattttctttgtgtttaaagaaaaaaaatgtccctgtgtctgtagagatgatttgcagttcagcccggctgaagctgaccgaatgagactatgggctgtgcctccgccaagcatgttgccactgttcaaaatgaagaggaagcccagaaagggaaaaactaccagaacggagatgtgtttggcgatgagtataggatcaaaccagtggaagaggtcaaatacatgaaaaatggggcagaagaagagcagaaaatagcagccaggaaccaagaaaacttggaaaaaagtgccagctcaaatgtaagacttaaaactaataaagaggttccgggattagttcatcaacccagagcaaacatgcacatctctgaaagccaacaagaattcttcagaatgctggatgaaaaaattgaaaagggtcgggattactgttcggaagaagaggatatcacatagcaccaattttaccactcaaaccaggagctactactgtgtaaataggttacaccccagttgaaatctttgcaaaggtcggttctattcagcgaacagcactatagcaaaagaagatcgttccatattgtacgccccattaaattacagtgtttcttaatgaacttgcaaaggaatattgctaaaaacaaacaaaaaaaactgttatcgaactttctttgttgctgctagttaaaacttgttgcaacttttcacttctcttgtgtccaggtatgcagcaaaattctgcaatttcaccttaaagatactgttggttttacagatgctctccaacctattttctataagatgaggtagtggtgaactcagataacaaacttctcttctaaactggttctgcttctaagacaagcatctcctgccctctctccttcctccccatctctcgcacgcagtctagagatggactgagccttgcttctcactggcagtgttgagctttggagatgggatggttgctatgccaagccttgtttctctgctcagaagaagtagagaagctattatcaattaaaagcatgctgtgatgtgactcctggaagtgacgtaggagtgagtggcaggttgtttgatttaataggtatcttaatcaagaattaagcttgcaacattggctttgctcagatgcagatggaagtgtgatcacaatcattttgaatccctcttcctcacttttttttctaagaaaataaacattttactgtttttatggatccttgtcttctcccattcatccagctcagtgttttaagatgatcctgggtgcagaagttgagccctcctttgcattgacactgataattagcctatagggctccctacccttccattaagaatctaccaagcattagcaaggctgaaagtggtctaagaggtgaggggacatcctatgactttttaggaaggcctgaaaccaccttgttacctttcattttgttagcaaataaaccatcctattttgtaactctcccccttcaaaatgctacatgagccttgccacttcctttttctcttacttccagcacactagacatagcaaaagtgtttgcctactcaaaaacataatacttttatgctgatgatggtatttggagatgtgaaagccaaaagcccctggcagtggtggggaatgttgactgagtgttcagcagagtttatttttccatactatatgaagagaatgatctcttctcaaagacagaagtgatatttttaacaaatattgtcacaagtaaatagcaatcaaaaggagaaaataacttttgtatttttttaatgtgtttgatagctttgacgagggttctctttgttactttcaggggagggcatcctattaaatgccacgccagcagtccgggtctgggtttgtcccacaaaatcacaggagcactgtatgttcctctcttttggagttgtgactttgaagggcctcaatattagccacactgccgcctgcagaaggtggagagttaagatgttctatgtcaatttgctcttgccgaaaagatgagcctcgattttaaaatctatccacatccaactgatggcaccattgatgtgcaaataatgagattccctatctccttttagacctgggacggcaaaagggaagggaaggaaacttagcagagtgctattgactatagattcacatattagcaacaaaatcccgtaattcttttggccaacagcagctattttggggagcagctgtggctgttacataaatagagatgcagccaaaattttaggccttttatcctgcttctagcagaaaaatgcagggagagtcaagtagtctagggtttcaggttgcctcccctcatatggtttttggccaagtgactaaaacagttttccacaactgtaaacaaactgctaagccccacctcaaacttgttcactggggactttgcttaccgttctgtgggtgaccttttccgggatttcttgttcttatcaagcaagaattaagcacatgctaaacgtcttccatttgacttctctactcggtgtctcagacagtgtcttcccagaaaaccaccaccctctacccaaagatgaaacatgctcatgtcatttttctcatggtcacatttaacagttttgacatgttatacttgcgcatagatccaagcgtttcttgggaacctgacttttgagtgtttaataaagccggaagtggtgttgccctgaaccagcagattttcacctgggttctggctccggtgtttaacactggatacatctttgatgtgcgaaagtgagttcatcttcagacacatttggtacatccagaaatagatccaagaaatggggtggttgagtgggtccgcacgaaatgcttgattatgtcagcaacacccaacactgtctgttttccatttgttggttttaatcataaaattgtcaagtgattcgtgtttgtactttatttttttgtgccttctgaaaggatctaaaacaaaaatattttgcctttttttccccacgtgtatctgaacattaagcagattggctcagacacaatgaaaaggataatccaatgtacgtgctggtgcactctgctagttgttatctctgtagggctcaggaagctggaaggaggaagggagggtaagtggcctggtgagtggaggtagaaaaatgatgagaaatgaactgagagcattaagcagagagggttgataggctggccgtgtccggggtgaaattggaaatccagctgcctagtggccagtgggtggggcaagactgtcaacgagatttacagctggcttacacatgccttatgtcctctgagttgtagagttgtaaaagttcagcagtgtgtgcacagctttcttttggttggcagagattcaggatcatggagtactgctctcataattgaagacgtgtttgttattggcagagaaccttaaaaaaggcctttacctcagcgatgcttcctagccccaggcttgcagagaacacagagtggtgttgtggtctatttagggacaaagaaggtataaagtccagagatgagaaaactgggtcagccctcagaaactgcagcagccacgcacacagaagcctgctggaagacaggtctctctccgtccacagtgcccatcatctgagcctgggctgggatgactcaacttagcaaagacggacccaggaggagtgctggttcttcagtctttgtactggccccatcctctcctcactgtaatgtgaggaagcacctctgtgtcagggctcacctgggcatccaaagcggccacgcccacaatccgacagcccccaggagcaggtccagggatgatgcagcccccttcttggtcccatgtgatgtcatcctgctttgttatcttcttataactttatcctgctataactttatcctcttcccagcctcatccctgtttttctgttagggcaagactcttccataagcctgctaaaaaacagagatgatacctcttacaaactttacctcatagcctgtgaagcaggttggcatgtggattacaagtcctgctttgacactgggcaagaatttaagattgttctatctctactagtcatagaaaagaaacattgttaaacatgttgagttttaaaggaagaaatattttcaaattcttaatccaagaaaatactgagttggaatcttagacttcgggactctgacacgttctttatgaaaggcaaaataattggtatctaaagttctctccttcctgcctcccctaaagaaaaaggatattagattgcacactataattttacataagatctgccttccacacttccctgctggaaggcattctcagagctttatgtcttcgtacctctcagagattggactttttcttgtttaaaaccccaaccaaaaaaaataataaggcatgattggtggggagggaatgtgtatttaggggcataataaaaaggtggctcgaagcaggaactttggcctcatggtgtcatgggtggatgcctggactccagtgtgcctgtgaggggctgggttaggcagtcggctgtcacactcacatgtgcctgcaataaaccttttggaatttcatgaacgaggtctatgaattgccttttgccaatgaatggatgtatttttccaaggggggaatagtatccttgactttggcagtcacctttttgtatgtctctagaaaggggtcaaaaaactatggtaaagatgaggcttatgagtgaatacctctgggacaaaccttaggactcacaagctatgccatgtttttcaggagactcttgtaccttatctggaatctaatcttgggagaagaggaaaaaggagctaatatttgtcatttatactcactctgtgccagatactgtgctaggcattttataattgttttgtgtcatcctcatgataatcctgtgaagtagatctattacccccgtgttctaaataatagatcttaagtgtggaatgcatctatccaacataaatgcccatgttgaaagaaggaaagatgtcattcaagtatttttcaaattctttttattatgactatgcccttcgcaacactgtgaaaacaacccctgggggcatctgccttccagaatctctctctggcttctcaccagcttggtttcctcatggggagtgttttatttggcctcccctatctgagctgcacacacaccaggggaggccactggctaacagtaggacttcagtgccctgaggaaaaggctttgggaatttaggcacacgtttctctccttggaatccttctagcatctggaaaaggaacctggtatttctgcagttagataccagatgcaaccagaacaggctttcgagtgctgtgatttctttcctgggttcccaagtcttgttgtttcatcacaaactgtatctttttaaggttaaaagtcttgaccttcatgggggtctgggacaatccgatctccaagcatggaggaaaggcaatgcctggaccactgacttgcattgaaatcctttcttgtgggctagggtttgatgtctctttttcatctttggactggggatctgcatcttccaggtccatttaagcactgaaactagatgcaaatctctttcgagaccttacatgttttagatagtcatgtaatgacttggatagacatttaaataacttgttccaaggtcggaagagcccaagaactcctcagagttcctcttcttgcttcttgaatcacatcctctaaagatgactcatgtctccatagcaactgttaaaggtgctgcctagacgggacccgctcccacctttacttactttgctcgaaggaaccaaaacaatgtccttgtgtaaaggggctgtcatatccagttttcctttgaaatctggcccccaagatcctgcttctttctaacctggcagctgtcatgtcccttcaagatgatgtgggaaatggcccttacaacttgggaaccacagaaattgctgtatttcgggaagattcacctctaaactgaaggcttcattctgatagtgtctgccctctctaccctgatttcgcccttctttgcttccatttttagcccaaggctttgaatttgattgagtaagacttagaggcagtataaagaacaccataaacttaggcagaggtcccttagggtctctagagttgaaaataattctacagccttagggggacctcttggcattgactctaaagggagagaatagcccctgtgtcctggcatttcagtctagaccttcaaggactgttctctcttgacaggcaagcaagcaaagaaagttttgcaatagatttcaagccagtttttccattcaaaccaagatgcaaattcataaaattactcttttcctggaatagatccaggcagctgccttattagaactttagattcggatctattttcttaacacacatacacatacacgcgcatacatacatatacagagagatacgtggagaaaggaaatttactctatcattgcaatacttcaagaaagagctgtattttgcctttctgtaatctccaagatagtgtctaggaaagtaatagtataactatagggataccgaaacaggaaaaaccagccatcactcttgagaaagtttgagttcgactcacatgggagaatcgaggtctgctactcgtcttgctttgtgccccatctgtgcctggatgccctactacatctgcttgactcgtctgggctgctagccggggtgttgtggctgacatcctttcctggccttacacacataatagacacatccctaacggcgtgtgcctggtccagccacatacagccaccacatgtgtcacacactgtccccctcatccatgtggacttgactggcatttcagcagctccactgggatgctctaaccccagtgtgtggagttggggtcccttcatctaggttgacccaggtatagcatttttagcattgcctttccagtcttgatgattcattcattgaactcatttatttctggagcccctggtacactccaggcactgcgctaattgccagcaaagcacaactgaactaaatccaccttcaaggaacctagccataacgagggaggcagcatggaagtaccctacaggggaaagtcctgagtgctgtgggagcatctcaccgtggcagccagcccagttttggcaatcaggggcttcctgaaagaggtgacatcaaagcccggatgtgtcagaggactgagggagagtgttactaaaggactttcaggctgagaggatagcacaggactcagcccaaaggagggccagtgtggactgtccagggccagcctgcagtacagaggctggagcttggacttgtagagggagagagaagagcaagggacgtggacggggcagtgagccaggctagccacagagggttcccggggctttgctggggattcagggagcataaataagagctttaggtggtgctgtgtcctctgcagcccactgctgaggtcctccagacaggtaaggtgtggtcacaatcagggccggggtttccctgctcactgcggcagtgcaggggtgcttgctgagatgattcatcccagggtgtcctctgtcccttacccagccccaactcctcttcctctgccaaaagctatttgaattcaaggactttaacctgggccggatctggtttggagacaaaggggacagctctgggtcagcatgaccttctttagagccactaaggcgaaaaataccgtttgggaccaggctggcctagacccagggatgagaatgcaccctaaaataaatatacgggaagcagcagagggcttccctgtctagtgtgtgatcctaactaaaggcagctctcttggacagccttcccctggattaggtcacatacacctggtggccaagcctctgctgggtcccaaatacacacccgagtcctgccaaagaaaggagatttttaaaaagcacagacaaattgtatgcaagtggaaaatacccataggcctagacagctgtggagggaagacctcgtgggtacctggaggctgccagagctgggagctctgcaggtatgagtcagggaaggctcagagacaagcagaatctctctatggagacaacttgcagtgccttttaggttttccaaataacctcggagttcagagcattgggtttttttctcccctccccacccccagaaaaataattagaaaaatgtttaggagaaaggaaaagaattagatgcatcagaataccagctataagccaacactgtttccagaaactcaagaaaaagctcaaacagaagacagttcccctgagaggctggaggcgttggtgctgaaggcaattttcctagctaaggggcactgggccttgctgcaccttggggctgaccttttttgcaaaacacccacccctgccctcctggcatactcaacagcaacgccagctttctggacccttggaaagatgttagctcaaacacccactttttccagatcttcctcttgctcttcactgaggaatttgtaattctgaggctagcgatgcccactcggatattccgcaggcccaggtgtttagattagaatttgtccagcggtaatcctgatgctggaaaccaacaaacatttggcctcatattcacccatttaaaaactagagcccctggcaggtccccttagggccatgtgttcatggaatataagccaagtttgccttaggcttgttcatggaatataagccaagtttacctctccccattttctgccctggcccacttcccactcacctccacctcattgccaggaagggatcaaaatgcctccatgccagttgttaatggctacatatttgcccttcccaagggtatttgcattttatttaggaacatggccttatattcaaggaaaatctagcatcaagattacgaggcatcacctctcaatcaggtctgggaggtatcttggggcattgctcttctgaacacctgcagaggcttcctcaggtgagtgtgggagcccggaagggtggcctccctaaccactctgcctgcacatgaattctccaaagcagtgggcccccatctgtttcaattacacatgcctgtcagcaaaaacttcgtgagatgcactctctctgtgtgtttattaatttatttaaagcatatatccctttacttttgtactactatattaggcacattataaaaagtatacagcatagaaactttaaatgaataagacacaaaatattataaacagaggttctggcattttctctctgaactcctgagggggaccttgggcacctcctggtatgtgcacaccccactttgaacacccttgctttagtgcaaatcagcacgcctaaatagcatcccaaaccactgtgcgacatttggcctgcagaataagaaagtcctaaggcagaatccccaggaacttcaaatctgggagatgaggaaagaaaactcactcacaaacaacataaatgtaaataattcaaaaccctaaaggagagctgtccctagacagtagtggctacaggtgctaagcaaaagtcaggtacactggacagagccatgcgattgattgttcatcttccctctctgggtctccagaaaaggacactggcaatatccccagctctctcctgattggaattctttgaaccctcgaagtgctccagcagtactaccccccacgtagcagtgggcccatgtcgcttgaatattatttttgatttgaccaccaagatgtaatgatgattctattccattttgaaaagggtctgagaaagtgtacaggtctaattatatatacatatttatacatgtgtatttttgtttattgttacattttgacattggactttctattaaataatttttaagagttgg

SEQ ID NO: 4 - DNA, 10668 bp, Homo sapiens
  source 1..10668: mol_type = other DNA
gaccacgcatggagaattttaccacccagagacacgcgagtggccctgtgcaagtttcaactgcgcggggggcggggaattccgcagacaggattctagaatttatgtcttgtgtggtaacatttcagccggtgggtggcggggattaggcgtgaagcggttcagcaggcagaggttctcggacgccctccggcgaagccacctgttgatgcttttgactttctgtccttgttcctcgtcccatctggagcatttccaattctggttttgcggagcagcaggtctgagcttgtccggcgagggtgggagttggtcccggcggagatccagtgggaagagccggcggctgcccgggcaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 5 - DNA, 1041 bp, Homo sapiens
  source 1..1041: mol_type = other DNA
catgcatacagcccagtctaacgtacaggatcaaaaagagaccttatttcggctggcaattaccttacccgcttgttcattatctgactcctgcactcctcccaagtgttcaatgttaaccagatggcaaagttaaccagacagaggaaatccacataattactctagatgaagccccctaactgcggattagaaggaaaagagatgaagggtgctgcactaataataacaaccattttcaaccaaggcaggattctgggcctagaatcttcagtgacaaattaccaggagaggaggagggaaatgaagagctggaggcctagggagatggtgtgaggagaggagactgtgcctttccacttcttaataattaatgttctcctgctgctctgagtctgcatcaccagtgattcagtttgagtcagacatccaacaacaactgagcaaaagaatcatctcagatccaagcaatggtgacgcgcagatagaagagactgaggccaacgaccaaagcaaaggacagcagaactgactgacacagctcagaaaatatccaaagctctgtatgctctggaaaggaagacaagaaatgctaaagactctggatattgctctgtttctataaggtcccacccatataccatctcttcttttggatttcctttggcagggttcccttgtatggaagttctcaggagaacaacactctcagagtcccactgtctcaaagaattcttaatggacagctatacttggccccaaggtttcaacctcacactgaagaaagtgacataagaaaacacaagtcccagtgctatcaggggctatctggaaaagctgcttaaacagcaagtgaaagaaggctctcccctaccccgttctagactcaccctgaagttatttgaaggatccttgagactcttttggattgctgcttggagaacattcctgtaacttgtccctgctccttgaactataaaagcagaagggaggtactgagttaaaacttacaaattattatcaaataaatcttcaatagtg

SEQ ID NO: 6 - DNA, 3230 bp, Homo sapiens
  source 1..3230: mol_type = other DNA
accacccctgggttccctcccgggtccgcagtggaaacactgccctctcccttcttgacccctagcccttccttccctccctccttccctcctgtcgccgtctcttctggcgccgctgctcccggaggagctcccggcacggcgatgggttctcgggcctccacgttactgcgggacgaagagctcgaggagatcaagaaggagaccggcttttcccacagtcaaatcactcgcctctacagccggttcaccagcctggacaaaggagagaatgggactctcagccgggaagatttccagaggattccagaacttgccatcaacccactgggggaccggatcatcaatgccttctttccagagggagaggaccaggtaaacttccgtggattcatgcgaactttggctcatttccgccccattgaggataatgaaaagagcaaagatgtgaatggacccgaaccactcaacagccgaagcaacaaactgcactttgcttttcgactatatgatttggataaagatgaaaagatctcccgtgatgagctgttacaggtgctacgcatgatggtcggagtaaatatctcagatgagcagctgggcagcatcgcagacaggaccattcaggaggctgatcaggatggggacagtgccatatctttcacagaatttgttaaggttttggagaaggtggatgtagaacagaaaatgagcatccgatttcttcactaaaggagaccaaactgttccttgcggtctagtatttaagaactggaacttgaaagtcctccttctaccaactccacctccaccccctcattccccttctcccaaagtactactgctgttgcatgacaaccccaaatatgttctgtcaacacaaacctgcctttggtgtataaacagggcattacagaatggtacaccctatatatttctgttcagtatccattcactagttcttcatttataaatatcatcttccccattctgctgctgaatgccacacatccatccagtctgagaaagtgagagaggcaatcatgccaagaacaagccagcaaagctctttcaccagatgtagactgtagccctgctgccttccctccagcgagtctgccagcatgcttcttcatcctttttatatgttctttgcttcctacttccctgtcttccaacatactgttcacttactctggcagtctttctgcttttcattaagcctcaaaatctcctctgttctacttggcaccacaagctatgtcctatatatgtatttctgacttggcaggatagttcaggggtctggcagtttttatttaccttcattattaaatgggcctctgggatgttgcctcttcaggagctttttggtaatcaatacttctctcagaagtatgagaccatcctctgcactctgctctgtcatcaaaggctgctgggtggagataccctttttgaaaggtggccttggtgagaggtatggagccaagtcttctaggttgcttgcccacatcactctatctctggcctctgattctcaactttgtacctgtgtggctcctcttgttagtgcaatgttgactgttgaaaaagcagcagtatgcttacaggtttgcttagtttggggacaccgttaccaccagaatggctgctctgacaatatgcctagggactttctcatggcttttatttaataaggaggctgggcaccctataaagcctcatgcattcacacctttgcagcatggtttatgcctcagtgttatgtgcactggaatgttttccacttcacatttccaagtagaaatattagtgttacggaagtgcctaatatcccagtccaaatttttttttttttttttttttttttttgagacagagtcttgctctgtcacccaggctggagtgcagtggtgcgatcgctcactgcaacctcagcctcctggatttaagtgattctcctgcctcagcctcccaagtagctgggattacaggtgtgcaccaccatgcccggctaattttttgtatttttagtggagacagggtttcaccatgttggccaggctggtctcgaactcctgacctcgtgatccgcctgcctcagcctcccaaagtgctgggattacaggtgtgagccaccacgcctggccccagtccaaaatatttaaagattgtttccttagtgtcttgaagttttgcacaaaattcttttttttgagatggagtctcactctgtcacccaggctggagtgcagtggcgtgatcttggctcactgcaacctctgcctcctgggttcaagcaattctcccacctcagcctcccaagtagctgggattacagacgtgtgccaccatacctgggtaatttttgcatttttagtggagagggagtttcaccatgttggccaggttggtcttgaactcctgacctcaggtgatcctcctgcctcggcctcccaaagtgctgggattacaggcatgagccaccgtgctcagccgcaaaattctttatgaattttacacttggcaaatgttaatgacggaagccatagtctgctcctaatacatgtccaaagcattgactgttgtgtcattagctgcctggttacattagctccctggcttcttgtttagaccactgctaatcccttaaaaacaagaggtctggcactagtagcacaacctaaggtggcattacagatctttgagcgagccacagcaacttttctgccaagtcagcttagtttagacttcagtgaatcaggctattgctatcctaatgtatgtctctatgagtgtatttagccacacatctgcccttggttgactttctgactcattgcttgcttgcttgtttccttgctttggaaaactattgaagattgctaaaaaataccactgcaaagtgatggaaaagggtggagaacaggggagtagccaggctggatggctcaaatataaatgaatgaggaattctttatgaagtatcagtcagattttatgattaagtgatgtaatataggaattatgtaaaagggaagaatgtctgatactgatctattagagaggtactttagaggcttcttgattggcataaagttcctaaggttatagattttccccccttttggctgtatagcaaagtgttttaatccacggttgtgccttattgttccattaaaattgtatcttcgatccatcaataaatacttgtggttgaaacaaaa

SEQ ID NO: 7 - DNA, 2678 bp, Homo sapiens
  source 1..2678: mol_type = other DNA
agcggaagtgctcgttgggggtgcacaaggcgcgttcgagcagcggcgaccgacgcggcgaaggagcgcgccatggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacatgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaaggctctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagaggaaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatgactgctgattccagtgccacagatgaacccacaaggacatagctgttccctaacttggtggatggctccagtttgcttttaacgaaaatcacaacttcaggagacatctgaaaagaatgatgtctctgaaagctgtcctttcagatgagggagaaatgaaggatttcacacttcagaatattttactaaaaacattccagtcttggccgggtgcggtggctcctgcctataatcccagcactttgggaggctgaggcaggaggatcacttgagcccaggagttcaagaccagcctgggaacacagcgagaccctctcattaaaaacaacaaaacaaaacaattccagtcttggagtagtctaacagaagaaaatgtaaaattatttgagtgtaaataatagatgtcagtatttatcatgatgggtcacatatagacatatgtacatattatatatatatatatatatatatatatatatatatatatatatatataagctcttttttctgaggctattttatagttatttttaaacataaagatacagaagtcttcttgacttctgattttcaaaaccattcctcagtatcttcaggcatttgacctcctgaatgtgcttggccctgggcttcagttatcctttgatgtcctgcaggggtggctaatgtgctggggtttttctgtgttaatagtcacagtattgttttattggtgaatagctgaaaaacagagggattaagtcatattccgggaaagagaattatagtttttatgcctcctgttgaataaatggtgtcctgattgcctggg

SEQ ID NO: 8 - DNA, 14069 bp, Homo sapiens
  source 1..14069: mol_type = other DNA
cgttaaatgcaaacgctgctctggctcatgtgtttgctccgaggtataggttttgttcgactgacgtatcagatagtcagagtggttaccacaccgacgttgtagcagctgcataataaatgactgaaagaatcatgttaggcatgcccacctaacctaacttgaatcatgcgaaaggggagctgttggaattcaaatagactttctggttcccagcagtcggcagtaatagaatgctttcaggaagatgacagaatcaggagaaagatgctgttttgcactatcttgatttgttacagcagccaacttattggcatgatggagtgacaggaaaaacagctggcatggaagatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 9 - DNA, 2385 bp, Homo sapiens
  source 1..2385: mol_type = other DNA
ggatgcgcggcgtggccacgccccttcagtgcttgtgacgcaggcgccctgggctttttgggcgcgaaaaagaagcagtcctgggttgtacccggcgcagctgggagcggctgcttcctccggggtcgtatctccgcccggcatggggctgctggacctttgcgaggaagtgttcggcaccgccgacctttaccgggtgctgggcgtgcgacgcgaggcctccgacggcgaggtccgacgaggctaccacaaggtgtccctgcaggtacacccggaccgggtgggtgagggcgacaaggaggacgccacccgccgcttccagatcctgggaaaagtctattccgttctcagtgacagagaacagagagcagtgtacgatgagcagggaacagtggacgaggactctcctgtgctcacccaagaccgagactgggaggcgtattggcggctactctttaaaaagatatctttagaggacattcaagcttttgaaaagacatacaaaggttcggaagaagagctggctgatattaagcaggcctatctggacttcaagggtgacatggatcagatcatggagtctgtgctttgcgtgcagtacacagaggaacccaggataaggaatatcattcagcaagctattgacgccggagaggtcccatcctataatgcctttgtcaaagaatcgaaacaaaagatgaatgcaaggaaaaggagggctcaggaagaggccaaagaagcagaaatgagcagaaaggagttggggcttgatgaaggcgtggatagcctgaaggcagccattcagagcagacaaaaggatcggcaaaaggaaatggacaattttctggctcagatggaagcaaagtactgcaaatcttccaaaggaggagggaaaaaatctgctctcaagaaagaaaagaaataatggaatttttctcttcaaaggtccttaggtgtaaattgatgccatcgtaggcaaggtgcaggcaggatttgaaggcaaaagtcaattcagctcttgagaaaaggtgtctttccagcctgaatttttcagattgactagaccaagcagaatctctcaacctgatcttagtatttcctagaaagcacttgacattgtgtgaggtctcacctgaaggaacttggtggtgacatttgggagggtggagggaggcagtgtccttcctgacagcacttgcctccatggatcttctgtacacagaactcttatctaggatgtggttctgttcatgctgctttctgcgatgtgcgtgtctgttagaataggctctctacccagctagaacaccttccagacacttgctggacagctatcttccacatacttcccagtttacatttggtcttaatgatcttgaatagatcctctcttcattttactcagccaggttttgtactgatgtacaggtgttaaattacttcaagcatttttgtaagaggtgtatataattcaataaaaaaggtaaaacatgatgattaagttctgggggctttgtaaatgatcccactaaaatgtgacctaggaaaaatatgaatggtgtttaggatgagagaaaaggggaaaacaatagccctggtcagctttataatagagagcctggtttccctagcatgaagagatgtatgttgtagtcctgccactgattactgaagtcctgccattaattgctgaatgtcttcagttgggccactgagcttgtctgaatctgttcccttttataaaagagttactacatcaaaacaaagagtgaaatccaaatttgtcaaactgtatgtattaaacatgtccagttttttggttaaaaaaaaattgcactggctttgagggaaaacacacagggtgagggaattgggctaaatgacttcttacaggcccctttctgattctttaactttgaaaggcaagccatattgatccagttgttatagtgaactcatggtaatggtttgtgagaacaatagagattttcatttctatgtagatgagttggtatgagaatatatggaatttttaagggactgtttaaatctttgatttgtagactattaaatataccgtatgcataaagtaagcctttagctctaaggtaaagacgacacgttttcggtttgtgactacaaataggttaaaaatagattttaattttattaaaaatataatttaatgcaggttgtttgaagcatctgtcttcatatgatggcattagaacaccttggtataataaaaagttaccgtaatttatgattatttgaatttatccattctgaaaattaataagatctaaaactggcatgacaatcaagatttgtatttagtgaaatttaaaataaatgtaagccatagttaaaaaaaaaaaaaaaaaa

SEQ ID NO: 10 - DNA, 4206 bp, Homo sapiens
  source 1..4206: mol_type = other DNA
cgccctcccgccgcgcgctcgggatcccgaccagtcctgaccgcacgggggccgcggccacggggcgcaggggccatggtgcgcggcaggatctcccggctctcggtccgggacgtgcgcttccccacgtcgcttgggggccacggcgcggacgccatgcacacggaccctgactactcggctgcctatgtcgtcatagaaactgatgcagaagatggaatcaaggggtgtggaattaccttcactctgggaaaaggcactgaagttgattggtccagaaaagggcgtggtgcacctggcgacagcggccgtcctaaacgcggtgtgggacttgtgggccaagcaggagggaaagcctgtctggaagttacttgtggacatggatcccaggatgctggtatcctgcatagatttcaggtacatcactgatgtcctgactgaggaggatgccctagaaatactgcagaaaggtcaaattggtaaaaaagaaagagagaagcaaatgctggcacaaggataccctgcttacacgacatcgtgcgcctggctggggtactcagatgacacgttgaagcagctctgtgcccaggcgctgaaggatggctggaccaggtttaaagtaaaggtgggtgctgatctccaggatgacatgcgaagatgccaaatcatccgagacatgattggaccggaaaagactttgatgatggatgccaaccagcgctgggatgtgcctgaggcggtggagtggatgtccaagctggccaagttcaagccattgtggattgaggagccaacctcccctgatgacattctggggcacgccaccatttccaaggcactggtcccattaggaattggcattgccacaggagaacagtgccacaatagagtgatatttaagcaactcctacaggcgaaggccctgcagttcctccagattgacagttgcagactgggcagtgtcaatgagaacctctcagtattgctgatggccaaaaagtttgaaattcctgtttgcccccatgctggtggagttggcctctgtgaactggtgcagcacctgattatatttgactacatatcagtttctgcaagccttgaaaatagggtgtgtgagtatgttgaccacctgcatgagcatttcaagtatcccgtgatgatccagcgggcttcctacatgcctcccaaggatcccggctactcaacagaaatgaaggaggaatctgtaaagaaacaccagtatccagatggtgaagtttggaagaaactccttcctgctcaagaaaattaagtgctcagccccaacaacttttttctttctgaagtgaaagggcttaaaatttcttggaaatagttttacaaaaatggatttaaaaaatcctaccgatcaagatgagttcagctagaagtcataccaccctcaggaatcagctaaagcaaaaagaacttttacctcggcatccagcccaacccctaaagactgacaatatccttcgagctcctttgaaagcaccctaaacagccatttccattttaatagttggatgcggattgtacccttcaatctgaaagtcttcagctttgaagtcatcaattttctcaacttttcgaagaatcctgagctttgggaaaggtctgggttctcgctgaagctaaaaacaaaataaggccattattttgccataattgtacgacctgttgtaattgctcctcatgtccgtgaaacaagtacacaggatgtgatcaacaaagttctattttacaggagtatgatcctgtcgataccttgccgtaggttatgtaacatgattggagcgcaaccagctgttctcttgcacagatcgagagtgaggggtattttgtgacattacacagcatcaggagcctggtgcctcatcaggtgtaagttcttataaccactcttggcaaatttattaaagacaggaacacagtcaatctgtaactcatagtagctctacgtttacttgaattccacaatccctaacccatctgtccctggcagaaagaaggaaagatgacatgcatggacagtgaacagaaagggatgaaagccaggattcctgggatgaacagacagtggcaattaggatgtgaagacaggtcacaacctattactatgtctaaaaacgaccagagcagagagccagagagaataagcctgaagtcacctccactcaaaagcagccaaactccctcaaaggagtaacttttaaaacctggatctaacctggaaggggctaaaaagtgtctggttctgagtttttttccttaaggctcatgaagcagatgaacttacatttttattgccatttcatatcaattgttggctgctataacttagggatttcaacagacttttgaagtttggacctaaatattgtacttaatgtaaaattaacaaaaaatatttatggccagggtggtggcttatgcctgtaattccagaattttcggaggctgaggcaggtggatcacttgaagtcaggagtttgagactagcctggccaacatgatgaaaccccatctctactaataatacaaaaattagctgggtgtggtggcatgtgcctgtaatcccagctacctgggaggctgaggcagaagaattgcttgaacccgggaggtggaggttgcagtgagctgagatcgcaccacggcacactccagcctggccgacagagaaagactccatctcaaaaaaaaaagaaaaggaaaaacatttgcacttcaattctccttcaagttaaaatgagttaaaatgcccccttttggacaatcccctggcttgaatgtggctcttccctctctggtactggtgcttagtacctcacagcacctgacatgttaagtgcccatggttgctgaggcagatgcctgccttgtcctgcccacctgcccaccacttctccctaaactgaagccccacatttggagcagtcatctttatcttggacacagcattgagcagatgcctgttccacagtcaaccttttatcaagagaaggtaccaaacccaaaagtataacatctaattcttacctgaattttcagtggctcgatgtgattcaggtaaatatgtgcatctcccaaagtgtgtataaagtcacctggctataaacccgggggagaaagcagaacagtatgttagtttcaattctttaaaacatcatttaaaaacattagaatatgcagacaccgcaaggctttttttaaaaaaataatttagtgtagcttttccatttttttgtagcaacagcatcttgttatgttgcccaggctggtattgaactccagacctcaagcaattgctcctgtctcagtctcccaaagtgctgggattacaggcatgagccaccatacccaacctcagcatagcttttgagaaaatccatagaagctgtatcacaaacaacctgtatagatctgttagtgcgtataccacagggccagaaaaccttccagaagaggaaggtttcaaagtaaaagctggttcatttcttacttacacatatcaaatttaaaagctaatcagagactaaactctgcaatttgttttcccatattaaagaactgaagagctcagtgtggtaggctggcaagtcacccttcccgagacagcccaccttcaggcccgtgatgtgcgcaatcatgtacgtgagcagggcgtagctggcgatgttgaaaggcacaccgaggcccatgtctcccgatctctggtacagctggcaggacagctcactgttcaccacatagaactggcagagggcatggcatggaggcagcgccatcagaggaagatctgaggaaccagcagaggaagataaggagggatggtggtttgaaagaccacagctaaaggcaaagtaaaacaggagagaaacagaagccaactcatatggtggagaccaggagagagagccactgggctgcagtgatgtccataacagcctctgcagcgatggcacggagctgagggagactatccatcggtgcaaggtttctgcaggtgtccatttacggctgaagcaatgctcttccatcagagctgaagggatctgggctacctcgtggcaccagattacaaatacagcaggaataattctgtttgccacaggaaactggtgcttctggtacaccctcctatattaaaagtctctattacatggcaaaaaaaaaaaaaaaaaa

SEQ ID NO: 11 - DNA, 1753 bp, Homo sapiens
  source 1..1753: mol_type = other DNA
agcggactgcgcatgtgcaggacccagcaggtctagagcttttctgtgtttctccggacttcgagccatggcggtgacggaagcgagcctgttgcgccagtgccccctgcttctgccccagaaccggtcgaaaaccgtgtatgagggattcatctcggctcagggaagagacttccaccttaggatagtgttgcctgaagatttacaactgaagaatgcaagattattatgtagttggcagctgagaacaatacttagtggataccatcgaatagtacaacagagaatgcagcactctcctgatctaatgagctttatgatggagttgaagatgcttttggaagttgccttaaagaatagacaagagctgtatgcactacctcctcctccccagttctactcaagccttattgaagagataggaactcttggttgggataaacttgtgtatgcggatacctgcttcagtaccatcaagttaaaagcagaagatgcttctggtagagagcatttaatcactctcaagttgaaggcaaagtatcctgcagaatcaccagattattttgtggattttcctgttccattttgtgcctcctggacacctcaggtaaattctcctcagagctccttaataagcatttatagtcagtttttggcagcaatagaatcactaaaggcattctgggatgttatggatgaaatcgatgagaagacctgggtacttgagccagaaaaacctccacggagtgcaacagcacgcagaattgcattaggtaataatgtttccataaatatagaggtagaccccaggcatcctactatgcttcctgagtgcttctttcttggagctgaccatgtggtaaaacccctgggaattaagctgagcaggaacatacatttgtgggatccagaaaatagtgtgttacaaaatttgaaagatgttttagaaattgattttccagctcgtgctatcctggaaaaatctgattttactatggattgtggaatttgttatgcttatcaacttgacggtaccattcctgatcaagtgtgtgataattctcagtgtggacaacctttccatcaaatatgcttatatgagtggctgagaggactactaactagtagacagagttttaacatcatatttggtgaatgtccatattgtagtaagccaattaccttaaaaatgtctggaaggaaacactgaaataagaatacaacatttcggtgaagagctggaaacttaaaaaattatcaaaaggaattttggtatcatcttcagagaaaaaataaagcaagaaatactaacatcaaaaggacaggtatgatgatgcgataataataaacatctgcgtttgtctcttcactaagagtaaactgggaaattgtaggccaaagtccagttgaactttctaagtctgtgatccccgtgctgactgtggaagtgtatttataccaagatggagatcttgacttcttgaatatatctggactggtaaaatcttgatgaggctcataaaatgagtttgggaattgtgtatagctgattttttgtgggaaactgtttacttcattcaaaggttcttgagactcttgatatttctgtcttctccttgtgctttcctatggaaaaaatacatatatagtttagtttgttagacgtgagttatccaagtatttattttgtgtagtgtgtaagaatgctaaataaaatgttatacaagatcaaaaaaaaaaaaaaaaaaaaaaa

SEQ ID NO: 12 - DNA, 3189 bp, Homo sapiens
  source 1..3189: mol_type = other DNA
ctatttgagtttgtggcgcgcgaggccctgcagtccgggttggcgcttgggtactggctgggtccgatgctgggtacgctgcgcgccatggagggcgaggacgtggaagacgaccagctgctgcagaagctcagggccagtcgccgccgcttccagaggcgcatgcagcggctgatagagaagtacaaccagcccttcgaggacaccccggtggtgcaaatggccacgctgacctacgagacgccacagggattgagaatttggggtggaagactaataaaggaaagaaacgaaggagagatccaggactcctccatgaagcccgcggacaggacagatggctccgtgcaagctgcagcctggggtcctgagcttccctcgcaccgcacagtcctgggagccgattcaaaaagcggtgaggtcgatgccacgtcagaccaggaagagtcagttgcttgggccttagcacctgcagtgcctcaaagccctttgaaaaatgaattaagaaggaaatacttgacccaagtggatatactgctacaaggtgcagagtattttgagtgtgcaggtaacagagctggaagggatgtacgtgtgactccgctgccttcactggcctcacctgccgtgcctgcccccggatactgcagtcgtatctccagaaagagtcctggtgacccagcgaaaccagcttcatctcccagagaatgggatcctttgcatccttcctccacagacatggccttagtacctagaaatgacagcctctccctacaagagaccagtagcagcagcttcttaagcagccagccctttgaagatgatgacatttgcaatgtgaccatcagtgacctgtacgcagggatgctgcactccatgagccggctgttgagcacaaagccatcaagcatcatctccaccaaaacgttcatcatgcaaaactggaactccaggaggaggcacagatataagagcaggatgaacaaaacatattgcaaaggagccagacgttctcagaggagctccaaggagaacttcataccctgctctgagcctgtgaaagggacaggggcattaagagattgcaagaacgtattagatgtttcttgccgtaagacaggtttaaaattggaaaaagcttttcttgaagtcaacagaccccaaatccataagttagatccaagttggaaggagcgcaaagtgacaccctcgaagtattcttccttgatttacttcgactccagtgcaacatataatcttgatgaggaaaatagatttaggacattaaaatggttaatttctcctgtaaaaatagtttccagaccaacaatacgacagggccatggagagaaccgtcagagggagattgaaatccgatttgatcagcttcatcgggaatattgcctgagtcccaggaaccagcctcgccggatgtgcctcccggactcctgggccatgaacatgtacagagggggtcctgcgagtcctggtggccttcagggcttagaaacccgcaggctgagtttaccttccagcaaagcaaaagcaaaaagtttaagtgaggcttttgaaaacctaggcaaaagatctctggaagcaggtaggtgcctgcccaagagcgattcatcttcatcacttccaaagaccaaccccacacacagcgcaactcgcccgcagcagacatctgaccttcacgttcagggaaatagttctggaatatttagaaagtcagtgtcacccagcaaaactctttcagtcccagataaagaagtgccaggccacggaaggaatcgttacgatgaaattaaagaagaatttgacaagcttcatcaaaagtattgcctcaaatctcctgggcagatgacagtgcctttatgtattggagtgtctacagataaagcaagtatggaagttcgatatcaaacagaaggcttcttaggaaaattaaatccagaccctcacttccagggtttccagaagttgccatcatcacccctggggtgcagaaaaagtctactgggctcaactgcaattgaggctccttcatctacatgtgttgctcgtgccatcacgagggatggcacgagggaccatcagttccctgcaaaaagacccaggctatcagaaccccagggctccggacgccagggcaattccctgggtgcctcagatggggtggacaacaccgtcagaccgggagaccagggcagctcttcacagcccaactcagaagagagaggagagaacacgtcttacaggatggaagagaaaagtgatttcatgctagaaaaattggaaactaaaagtgtgtagctaggttatttcggagtgttatttatcttcccacttgctctctgtttgtatttttgttttgtttttgattcttgagactgtgaggacttggttgacttctctgcccttaaagtaaatattagtgaaattggttccatcagagataacctcgagttcttggtgtagaaattatgtgaataaagttgctcaattagaatttttagggttctctttgataggcctgtttttctgatgtgtgtgttttttttgggggggggttatttgtttgtttgtttgtttgtttgtttgtttttgagacagtctctctctatcgcccaggctggagtgcggtggcacaatcttggctcactgcaacttccgcctcccgggttcaagcgattcttctgcctcagcctcccgagtagctgggattacaggcgcgcgccaccacgcctggctaatttttgtagttttagtagagacggggtttcaccatattgaccaggctggtctcgagctcctggcctcgtgatccatctgcctcggcctcccaaagtgctgggattataggcgtgagccactgctcccagccgtgtgttcttttttaaatttagatatgtccagagaatcctctctcctgtttcccatttcattcgagaatattgtttgcttgtgagacgtaagttcgagccctgcatgcaatgacccttgaaggaaaataaacagtcctggtggtcccagacgctcctgcagccacagcgcctgtgactcctcatgattcttactgaagctgttgatgacaggatatcatggtgacgtttttgtaatgaaatatttcacatattcagaatacattggtgaaactcatgctggagtaaatagttaatatatggccat

SEQ ID NO: 13 - DNA, 3485 bp, Homo sapiens
  source 1..3485: mol_type = other DNA
cttcttctttacctccgccttgttcctgtcctcaccacacggactgagactgatttgattaaagcaccagagtgtaatggccctcagagcagggctggtcctggggttccacaccctgatgaccctcctgagcccgcaggaggcaggggccaccaaggctgaccacatgggctcctacggacccgccttctaccagtcttacggcgcctcgggccagttcacccatgaatttgatgaggaacagctgttctctgtggacctgaagaaaagcgaggccgtgtggcgtctgcctgagtttggtgactttgcccgctttgacccgcagggcgggctggccggcatcgccgcaatcaaagcccatctggacatcctggtggagcgctccaaccgcagcagagccatcaacgtgcctccacgggtgaccgtgctccccaagtctcgggtggagctgggccagcccaacatcctcatctgcatcgtggacaacatcttcccccctgtgatcaatatcacctggctgcgcaacggccaaactgtcactgagggagtggcccagaccagcttctattcccagcctgaccatttgttccgcaagttccactacctgcccttcgtgccctcagccgaggacgtctatgactgccaggtggagcactggggcctggatgcgccactcctcaggcattgggagctccaggtgcctattccaccaccagatgccatggagaccctggtctgtgccctgggcctggccatcggcctggtgggcttcctcgtgggcaccgtcctcatcatcatgggcacatatgtgtccagtgtccccaggtaatgatccttctgagagaaatgacttgtgggagacaccctgcagatcctcatgggtttgtgacagcccctgcgtgctcagtgccctttaagtgcatcccgctgtgctgactttgagtgggatcaacatctgtcctacgggtcccctcttttttggccccagtattcatggcagggtttgttggacacctactagcttcccttcccattcaacacacacacacattcttgctctacccaaagctctggctggcagcactaaatgctttggtggtgtttgcactgtgtcctttccaggccttggccagttcttccaggggtgaggcatgtggtgctggggattggcagccatcctggggcccacacaggtgtgtcttgctccatttggcccattgtgtgttactttgtgaatgagccatttcacatggacttcatgaaatttgcctcctgagttcaggtttaccctgaaagggatgcagattatcctgttcctcacgaccccctcagctaacaacagttctgaagggtgctgggacaggacaggctcatggggactccactcctgcctgggtttactctgtatgaagaggccactggtatcctgccatgatgttatctcctttttctacttttccctagagtcccatgcatgataaagagaggcccaaggcttggataaggtggccacttccctcagtggagtcagtcatgttaggtaggaggtggtagagtcggtctgcgaggtatctcgtaagaggggaggtccacctagacacactctaaatatgtggcctagaagattttggtctacttttctgtgaacagaatttaaaacatacaaagagataaatcaccataccacatagtttatgtcaggaccaaaatgagcaatacagattacggttttcaaaccagaatgcacataagaactgcttgggatccttttaaaagtacaggcattggcctggtgcagtggctcattcctgtaatcccagcactttgggaggccaaggggacaggactgcttgaggccaagaggtggaaaccatcttgggctacatagagagaccccatctctacaaagaaagatttaaaaattaaccaggcatggtggctcgcacctgtattcccagccactggggaggctgaggccggaggagtgcttgagcccaggagttcaaggctgcagtgagccaagattgcgccactgcactccagcctaggtgacagagtgagaccctgtctctaaataaataaataaataaaatataaaaataacagtcatcacccagacctactgaattagaatctcgggagtgcagggggcagcaacagggaggctgtcttttctgagatggggtctcactctgtcaccaggctggagtgccatggcatgatctcagctcactgcaacctccacctcctgagttcaagccattctcctgcctcagcctcctgagtagctgggactacaggtgtgcgccactacactcagctaatttttgtattttaagtagagacggggtttcatcatgttggccaggatggcctccatctcttgacctcgtgatccacccaccttccctcccaaagtactggaattacaggcattagccactgtgcccagccgaggctgtcatttttaaccggctctggatgactctgatgcagccatcctggaccttggctgtggtctggtaactggaacccagtgacgtaatcaggtgccatcgggggtcatgggaaagggggatccccaaggtctgaggtggactaggaaggctttctgaagaacctgggtctgttagggcatcagccaatcaaggtacaagtaaatagaggcaaaatgagggtttgaactgtgagcagttggtcctggaaaagaaagaaaccaagagattatggggactcaatgggcttcttaagagagaataagttgaaatcaatgaccagaagaccctgatggaagtggaggagaatcatctcaggcaaactttttgtgtgccagtaacagaaaccctctttgtgtgatcacatgcaaagtataggatatttgcaatatagccatggggaggagtgcagggcccaagggtagattttagccaggcctcccaggaacagaactcggatccgaaaagcccagagaagctagagctgcccctccaacactctcggatccacatggtctgtgttctctagacccccctgcatgttagcggtgttctctctctgtggactgactgtccttctcagtgaacatgtccacccgacagctcctgagtttatatcatctcaaccctcacaacccacagaggctgtgtctcctagtcacagctttaaattactggaaaaataaatgactggccaaacttggagcaggtgtccatcccagccctgtgtagttagagcaggaatcaagatctcaacacaaatgtggctgccaagcactcagccccggggcgaggggtcaagttcttctcagagaaagaggaataagttggttctcagaagacatcacaagatacgtgtgtacccaacaatctctgatctctgctgatcttttgcttagacgttaacttgatgcatcattggaaaggtgtttctctcatctctgtcctaaggcttgataaagtcattaaaattgtgttcttttgactaaa

SEQ ID NO: 14 - DNA, 1312 bp, Homo sapiens
  source 1..1312: mol_type = other DNA
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 15 - DNA, 3006 bp, Homo sapiens
  source 1..3006: mol_type = other DNA
cgagttggaagaggtgagtcctgtctcaaaatggaggtaaaaccgccgcctggttgcccccagcccgactccggcagtcgccgtcgccactggggggaggagggccatgatccaaaggaaccagagcagctgagaaaactgtttattggtggtctgagctttgaaactacagatgatagtttaagagaacattttgagaaatggggcacactcacagattgtctggtaatgagagacccccaaacaaaacgttccaggggctttggttttgtgacttattcttgtgttacagaggtggatgcagcaatgcgtgctcgaccattcaaggttgatgggcgtgtagtggaaccaaagagagctgtttctagagaggattctgtgaagcctggtgcccatctaacagtgaagaaaatttttgttggcagtattaaagaagatacagaagaatataatttgagagactactttgaaaagtacggcaagattgaaaccatagaagttatggaagacaggcagagtggaaaaaagagaggatttgcttctgtaacttttgatgatcatgatacagttgataaaattgttgttcagaaataccacactattaatgggcataactgtgaagtgaaaaaggcccttgctaaacaagtgatgcagccggctggatcacagaggggtcgtggaggtggatctggcaattgtatgggtcacagaggaaactttggaggtggtggaggtaattttggccgtgatggaaactttggtggaagaggaggctatggtggtggaggtggtggcagcagaggtagttatggaggaggtgatgtggatataatggattaggaggtgatggtggcaactatggcagtggtcctggttatagtagtagaggcgggtatggtggtggtggaccaggatatggaaaccaaggtggtggatatggtggcggtgttggaggatatgatggttacaatgaaggaggaaattttgacggtagtaactatggtggtggtgggaactataatgattttggaaattacagtggacaacagcaatcaaattatggacacatgaaagggggcagttttggtggaagaagctcgggcagtccctatggtggtggttatggatctggtggtggaagtggtggatatggtagcagaaggttctaaaaacagcaggaaagggctacagttcttagcaggagagagagtgaggagttgtcaggaaagctacaggttactttgagacagtcgtcccaagtgcattagaggaactgtaaaaatctgtcacagaaggaacgatgatccataatcagaaaagttactgcagcttaaacaggaaacccttcttgttcaggactgtcatagccacagtttgcaggaagtgcagctatcgattaatgcaatgtagcgtcaattagatgtacattcctcaggtcttttatctgttgtagctttgtcttattctttttcttttcattacatcaggtatattgccctgtaaattgtggtagtggtaccaggaataaaaaattaaggaatttttaacttttcaatatctgtgtagttcagtttttctacattttagtacagaaactttaacaaaatgcagttttgaaggtgtttccttgtgagttaacaagtaaagaagatcaattgttaattactattttgtataaattttgctaaagttaactgtaaagaaacacctgctgacttgcagtttaaggggaatctattctccccatttccaaaccatgatatgaatggacgccgacatgtggagagaacagataatttgtgtgtttgcaatgtgtgttttaggtaaataggattgggtatttaaattagcatttgtgaatttaatagcattaagattaccttcaaataaaaaagtctcaaaatttctttttggtttttgtgcactttcttttaaaatgtaatcacatgattttagtgtgttagacttgctgagtcctagctgtgtttagaacatctccattctacatttaccttggtcaaatttgaactgctgccataggttttgggtgtaaagaatgtttactgccctccatttaaattctgaaaaggtatagtggatgttttccctctcctacattagaaaccattcttaaaaacttttcaaaatatagaaccattaagcctgctatatctgagcaaattagtgggtaccttttttccttttttaaagcacaagaggcccataaatcttgagttatttgcattagtttacattttttgatacaacttttcagaccaagagaataaaaatcatgcgttattaaacccctagctggctggcatgctttcctgtttgtactgtatacattttgctggatgaaaccaaggatagtttaggtataattgtccaaaataacctaactgcagcagaaatgtaggacagttgcttagtacaggcttctcacttcctacagacctgaattcaaatttggatagtctgagttattaaattcccaaagacaaagaacacactcttatttcttgtgtatatttcaacataaatcatgttgttaccaatttgttgggaaggccctggttgagaagagttttagataataaggctgtatatatatagatatatatagatatataccaatgtctatatatagagatattttatatatatatatacaggtatatatatgtgtgtgtatatatataggtatatacatatatacatatatatatatatatatatggatatatacccatgtctactgttttgcttcagctagtgcttacaatttcattcaagtcctgagtatgtgtcctgctgttactccttctttggtagttgaacgttgaattcaagtctttccttctgttttaagaagtactaagcaaacaagcaataaaaaggggaatggcgcatgctagtgtttgaatatgctctcttgttgctctaattctgtgcctccgtgcattaatatttggatgcatgcaatgccagcatggaaattggcct

SEQ ID NO: 16 - DNA, 1049 bp, Homo sapiens
  source 1..1049: mol_type = other DNA
aaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 17 - DNA, 7219 bp, Homo sapiens
  source 1..7219: mol_type = other DNA
ggaccccggcaagcccgcgcacttggcaggagctgtagctaccgccgtccgcgcctccaaggtttcacggcttcctcagcagagactcgggctcgtccgccatgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtggatggaagcatgcctaggggaagatctgcctcccaccacagaactggaggaggggcttaggaatggggtctaccttgccaaactggggaacttcttctctcccaaagtagtgtccctgaaaaaaatctatgatcgagaacagaccagatacaaggcgactggcctccactttagacacactgataatgtgattcagtggttgaatgccatggatgagattggattgcctaagattttttacccagaaactacagatatctatgatcgaaagaacatgccaagatgtatctactgtatccatgcactcagtttgtacctgttcaagctaggcctggcccctcagattcaagacctatatggaaaggttgacttcacagaagaagaaatcaacaacatgaagactgagttggagaagtatggcatccagatgcctgcctttagcaagattgggggcatcttggctaatgaactgtcagtggatgaagccgcattacatgctgctgttattgctattaatgaagctattgaccgtagaattccagccgacacatttgcagctttgaaaaatccgaatgccatgcttgtaaatcttgaagagcccttggcatccacttaccaggatatactttaccaggctaagcaggacaaaatgacaaatgctaaaaacaggacagaaaactcagagagagaaagagatgtttatgaggagctgctcacgcaagctgaaattcaaggcaatataaacaaagtcaatacattttctgcattagcaaatatcgacctggctttagaacaaggagatgcactggccttgttcagggctctgcagtcaccagccctggggcttcgaggactgcagcaacagaatagcgactggtacttgaagcagctcctgagtgataaacagcagaagagacagagtggtcagactgaccccctgcagaaggaggagctgcagtctggagtggatgctgcaaacagtgctgcccagcaatatcagagaagattggcagcagtagcactgattaatgctgcaatccagaagggtgttgctgagaagactgttttggaactgatgaatcccgaagcccagctgccccaggtgtatccatttgccgccgatctctatcagaaggagctggctaccctgcagcgacaaagtcctgaacataatctcacccacccagagctctctgtcgcagtggagatgttgtcatcggtggccctgatcaacagggcattggaatcaggagatgtgaatacagtgtggaagcaattgagcagttcagttactggtcttaccaatattgaggaagaaaactgtcagaggtatctcgatgagttgatgaaactgaaggctcaggcacatgcagagaataatgaattcattacatggaatgatatccaagcttgcgtggaccatgtgaacctggtggtgcaagaggaacatgagaggattttagccattggtttaattaatgaagccctggatgaaggtgatgcccaaaagactctgcaggccctacagattcctgcagctaaacttgagggagtccttgcagaagtggcccagcattaccaagacacgctgattagagcgaagagagagaaagcccaggaaatccaggatgagtcagctgtgttatggttggatgaaattcaaggtggaatctggcagtccaacaaagacacccaagaagcacagaagtttgccttaggaatctttgccattaatgaggcagtagaaagtggtgatgttggcaaaacactgagtgcccttcgctcccctgatgttggcttgtatggagtcatccctgagtgtggtgaaacttaccacagtgatcttgctgaagccaagaagaaaaaactggcagtaggagataataacagcaagtgggtgaagcactgggtaaaaggtggatattattattaccacaatctggagacccaggaaggaggatgggatgaacctccaaattttgtgcaaaattctatgcagctttctcgggaggagatccagagttctatctctggggtgactgccgcatataaccgagaacagctgtggctggccaatgaaggcctgatcaccaggctgcaggctcgctgccgtggatacttagttcgacaggaattccgatccaggatgaatttcctgaagaaacaaatccctgccatcacctgcattcagtcacagtggagaggatacaagcagaagaaggcatatcaagatcggttagcttacctgcgctcccacaaagatgaagttgtaaagattcagtccctggcaaggatgcaccaagctcgaaagcgctatcgagatcgcctgcagtacttccgggaccatataaatgacattatcaaaatccaggcttttattcgggcaaacaaagctcgggatgactacaagactctcatcaatgctgaggatcctcctatggttgtggtccgaaaatttgtccacctgctggaccaaagtgaccaggattttcaggaggagcttgaccttatgaagatgcgggaagaggttatcaccctcattcgttctaaccagcagctggagaatgacctcaatctcatggatatcaaaattggactgctagtgaaaaataagattacgttgcaggatgtggtttcccacagtaaaaaacttaccaaaaaaaataaggaacagttgtctgatatgatgatgataaataaacagaagggaggtctcaaggctttgagcaaggagaagagagagaagttggaagcttaccagcacctgttttatttattgcaaaccaatcccacctatctggccaagctcatttttcagatgccccagaacaagtccaccaagttcatggactctgtaatcttcacactctacaactacgcgtccaaccagcgagaggagtacctgctcctgcggctctttaagacagcactccaagaggaaatcaagtcgaaggtagatcagattcaagagattgtgacaggaaatcctacggttattaaaatggttgtaagtttcaaccgtggtgcccgtggccagaatgccctgagacagatcttggccccagtcgtgaaggaaattatggatgacaaatctctcaacatcaaaactgaccctgtggatatttacaaatcttgggttaatcagatggagtctcagacaggagaggcaagcaaactgccctatgatgtgacccctgagcaggcgctagctcatgaagaagtgaagacacggctagacagctccatcaggaacatgcgggctgtgacagacaagtttctctcagccattgtcagctctgtggacaaaatcccttatgggatgcgcttcattgccaaagtgctgaaggactcgttgcatgagaagttccctgatgctggtgaggatgagctgctgaagattattggtaacttgctttattatcgatacatgaatccagccattgttgctcctgatgcctttgacatcattgacctgtcagcaggaggccagcttaccacagaccaacgccgaaatctgggctccattgcaaaaatgcttcagcatgctgcttccaataagatgtttctgggagataatgcccacttaagcatcattaatgaatatctttcccagtcctaccagaaattcagacggtttttccaaactgcttgtgatgtcccagagcttcaggataaatttaatgtggatgagtactctgatttagtaaccctcaccaaaccagtaatctacatttccattggtgaaatcatcaacacccacactctcctgttggatcaccaggatgccattgctccggagcacaatgatccaatccacgaactgctggacgacctcggcgaggtgcccaccatcgagtccctgataggggaaagctctggcaatttaaatgacccaaataaggaggcactggctaagacggaagtgtctctcaccctgaccaacaagttcgacgtgcctggagatgagaatgcagaaatggatgctcgaaccatcttactgaatacaaaacgtttaattgtggatgtcatccggttccagccaggagagaccttgactgaaatcctagaaacaccagccaccagtgaacaggaagcagaacatcagagagccatgcagagacgtgctatccgtgatgccaaaacacctgacaagatgaaaaagtcaaaatctgtaaaggaagacagcaacctcactcttcaagagaagaaagagaagatccagacaggtttaaagaagctaacagagcttggaaccgtggacccaaagaacaaataccaggaactgatcaacgacattgccagggatattcggaatcagcggaggtaccgacagaggagaaaggccgaactagtgaaactgcaacagacatacgctgctctgaactctaaggccaccttttatggggagcaggtggattactataaaagctatatcaaaacctgcttggataacttagccagcaagggcaaagtctccaaaaagcctagggaaatgaaaggaaagaaaagcaaaaagatttctctgaaatatacagcagcaagactacatgaaaaaggagttcttctggaaattgaggacctgcaagtgaatcagtttaaaaatgttatatttgaaatcagtccaacagaagaagttggagacttcgaagtgaaagccaaattcatgggagttcaaatggagacttttatgttacattatcaggacctgctgcagctacagtatgaaggagttgcagtcatgaaattatttgatagagctaaagtaaatgtcaacctcctgatcttccttctcaacaaaaagttctacgggaagtaattgatcgtttgctgccagcccagaaggatgaaggaaagaagcacctcacagctcctttctaggtccttctttcctcattggaagcaaagacctagccaacaacagcacctcaatctgatacactcccgatgccacatttttaactcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcatttaaaaataaagttcctttatatttctgtcccatcaggaaaactgaaggatatggggaatcattggttatcttccattgtgtttttctttatggacaggagctaatggaagtgacagtcatgttcaaaggaagcatttctagaaaaaaggagataatgtttttaaatttcattatcaaacttgggcaattctgtttgtgtaactccccgactagtggatgggagagtcccattgctaaaattcagctactcagataaattcagaatgggtcaaggcacctgcctgtttttgttggtgcacagagattgacttgattcagagagacaattcactccatccctatggcagaggaatgggttagccctaatgtagaatgtcattgtttttaaaactgttttatatcttaagagtgccttattaaagtatagatgtatgtcttaaaatgtgggtgataggaattttaaagatttatataatgcatcaaaagccttagaataagaaaagctttttttaaattgctttatctgtatatctgaactcttgaaacttatagctaaaacactaggatttatctgcagtgttcagggagataattctgcctttaattgtctaaaacaaaaacaaaaccagccaacctatgttacacgtgagattaaaaccaattttttccccattttttctccttttttctcttgctgcccacattgtgcctttattttatgagccccagttttctgggcttagtttaaaaaaaaaatcaagtctaaacattgcatttagaaagcttttgttcttggataaaaagtcatacactttaaaaaaaaaaaaaactttttccaggaaaatatattgaaatcatgctgctgagcctctattttctttctttgatgttttgattcagtattcttttatcataaatttttagcatttaaaaattcactgatgtacattaagccaataaactgctttaatgaataacaaactatgtagtgtgtccctattataaatgcattggagaagtatttttatgagactctttactcaggtgcatggttacagcccacagggaggcatggagtgccatggaaggattcgccactacccagaccttgttttttgttgtattttggaagacaggttttttaaagaaacattttcctcagattaaaagatgatgctattacaactagcattgcctcaaaaactgggaccaaccaaagtgtgtcaaccctgtttccttaaaagaggctatgaatcccaaaggccacatccaagacaggcaataatgagcagagtttacagctcctttaataaaatgtgtcagtaattttaaggtttatagttccctcaacacaattgctaatgcagaatagtgtaaaatgcgcttcaagaatgttgatgatgatgatatagaattgtggctttagtagcacagaggatgccccaacaaactcatggcgttgaaaccacacagttctcattactgttatttattagctgtagcattctctgtctcctctctctcctcctttgaccttctcctcgaccagccatcatgacatttaccatgaatttacttcctcccaagagtttggactgcccgtcagattgttgctgcacatagttgcctttgtatctctgtatgaaataaaaggtcatttgttcatgttaaaaaaaaa

SEQ ID NO: 18 - DNA, 1354 bp, Homo sapiens
  source 1..1354: mol_type = other DNA
gcagtgcccgactccgcaggagcgccagggcggctcctgctcttcctggactccctgaagaggcgtttgtcgaaatgtccacagaaggaggatttggtggtactagccgcagtgatgcccagcaaagcctaaagtccttctggcttcgggtcatggaagaaatctggaatttagcagtgaaagatttctgaatgcaggaactcccactggctcgtattaagaagattatgaaactggatgaagatgtgaagatgatcagtgcagaggcccctgtgctctttgccagggcagcccagattttttatcacagagttgactcttcgagcctggattcacacagaggataacaactgccggacttatgtcgccatggcaattacgaaatttgatcaattggattttctcatcgatattgttctaagagatgaactgaaacctccaaagtgtcaggaggaggtgctgcagtctgtaactcctgctgagccagtccaatactatttcacgctggctcagcagcccaccgcccgtccaagtccagggacagcagcaaggccagaccaccgccagctccatgaccaccatgcagcctgggcagatcatcatcgcacagcttcagcagggccagaccacgcccgtgacgacgcaggttggagaaggtcagcaggtgcagattgtccaggcccagccacagggtcaagcccagtaggcccagagtggtactggatggaccgtgcaggtgatgtagcagatcctcactaacacaggagagatccagcagatcccggtgcagctgaaggctggccagctgcagtgtatccgcttagcccagtctgtatcaggcacccacgttctgcagggacagatccagacacttgccaccagcgctcaaccgattacacagacagaggtccagcaaagacagcagtagttcagccagttcacagatggacagcagctctaccagatccagcaagtatccatacctgcgggccaggacctgcccagcccatgtttttccagtcagtcaaccagccctctgatgggcaggccccccgggtgactggcggctgagggccggagctggcaaggccgaggacactcaacacaatttttgccgtacagccccaggtcatgaacacagccttcttccccagaggacccggccgacctcagctcctcctgcaggctaggacaatggcgcactaggcctcatgcctgggggccgagattctccaacagaaagatgcaatattttttgtttccttttttctccaaggaatcaatatttcaatatgttgagctgtgtgtccaatgctatgaaattaaaatattaaatcacaaaaaaaaaaaaaaa

SEQ ID NO: 19 - DNA, 3653 bp, Homo sapiens
  source 1..3653: mol_type = other DNA
aatgaatccaactactgtccttgtcctctcctctctaggtttgtggagactgaattccacaaacctagggacagggcactctctaggggcaagtcaactctcaattatagtgagggcaggttccccagttgccagcctacaccctggccagccacccaagggaatacctgctgctgctaaggcagtcaatgttgggagggtcagggaaggggagaggaagtagctgagtgtagagattatccaggctttccttcccgtcctctgtacagaaaggcagacatacactgactcctgaagtgccccaggatcatagttggttcttcttagggggagggagtgaaacggtggccttgctccaattccaggcctctggagaaaggagtgctcaactggagagtctcagaaccttttacagtacttcagcaggcccagcacccaaccttccccaccatacccctgccctgggaatgccccttaccacagtggcttccattcctgacatttgaaccagctcctgcacttgaaggacaaggcagccactgggcagctccgggaggcgggaggcgtgactccttcatatcaggccatctggaggagcaccagatttccctcttgtgaaacaaccccccacagtggggatttaatggtcaccacacaagccccaaaaaaccaggccctgcagagccatgacttcacaggggagaaaaccagcccaagaggctgccctcacccaacacttagctctaagtccagactcttaaaggttctgttaaagatctgcggccaggcgcagtggctcacatctgtaatcccagcactttgggaggccgaggtgggcggatcacgaggtcaggagagcgagaccatcctggctaacacagtgaagccctgtctctactaaaaatacaaaaaattagccaggtgtggtggcgggcacctgtagtaccagctacttgggaggctgaggcaggagaatggcgtgaacccaggaggcagagcttgcagtgagccgagatcacgccactgcactccaacctgggtgacagagcgagactccgtctcaaaaaaaaaaaaaaaaaaaaaagatttgcctagtgagcttggccaacccaagagccttcctgggctctcaggtttcttgaggcaaaacttctaaagatgcaaatagaatccttggtagctacaaagctcatctagccaaattataagtactatccaagcccaggattcattcttgaagatgagatgtaaaagcactgctgggcccttgcaaagggagagatcagagaaaataggagaacgaacaccagctcatgccaatctcagtctcatgagctacagagaaaaagagccaaaaaagtttatcctggctcagccaccttctggctttgtagctacaggcaagttatttaacttttctgaatgttggtttttccatctggaaaaaggagacaggaaaagctgccactggaggctaccttaagggtcaatgagtgaaatggggttgagtgcttaatgaactgttaaaacatgacacagatgggtgaggtgacatctgactgctcttcacagcagtccttgcatggtgagcagatgcaatcagtgtgctcgttcattcacttgttcatcaaacaccaacagtacccacttgtgccaggccttgtgctgggcaccgtaggggctatggaaggagtaagacacagtcatgctctcaaggaactcgctgtccagcagagtgataggacatgaatacatacttctagcaaaggctgagtagcattaggcaggtggccagatgaggtcagagctctggccctagtgccctgtagtaagttgcgagggagtaggaaatgaagacaaaacagcaagagcacaggtccagcacaagaaggtgaaggacatggaaagaggagcagagcatgataaagctacaaaagtagggggcaagtcatgaggtccgggtcactggggagccctggacaggctctgaatgggagatagactagactgacaaagcagctctttctggagacctcctgtggagggtcccctcttctacccttgggagacgaggtgtcattcacttcacgatacacatggggaaaggaagttggacaggggaaggcacttgtctaaggtcactcagtggtcatgccagtgcaaagcacccaaagcctggtccaggtcctgccacctcctgcgtgggctccatcttctcggctcagttcaagcctttattatcagagtccctctgtgtgccaggctctgtgctgaatgtccgtgtcggccacaaattgcactaacggtgcttactgccacttcctctccgaccttacctccaaagacccagccgggcctaaccctcacacacagccagaggaatccttcccaaacacagttaatcacatcgctcatccgtttaaaactggcttactagagccaacacaggataaaatccacaatcagtcccaaacccatctttctctgccgtctctagaccacattcactgagcaccaaggcctgcacgcttcgcagccagccctcctgctctcccccttgccctccagccacactggccctcaacccccaacactgggcccacctctgagccctgcatcaaacaccagcacccccttcgccgagctcattcttcatcattcctcagagctcagcttaagcatcaccttccttccctgcgacagggtcaggtcctttcctgtgcgctccgcagcaccctgcctgtccctcggagtcctcgcgagaggaactagataactgtgatccccgcgcacagcacggtgtctgcctaggacacagtgagcgcttcaacgcgctgaactaaacgaatgaaggggaacaccaactcagctgtcacccccgcccccaagtgtcactggctagaagatcttcccccggttcgccggcagcaccggtgggtcttgtcacctctgcgccccgcagccctccggaggctgcagttgctgtctgctgacaggcgtcccctcaggagaacgggagctcctccagcgcagggccctgctcagcacccctggtcccgggggcccccacggaggccgccgcgctcagtgaaactttgccgcgcgcagcagtggccggagacccgcgcggaccctctccccgcggggacccgccagggcgagtcgcccctcccccgcggcagacgacccgacctgggccaaggtcggccaagtgctcgccgcctgcccagcggcccttggacccacaggacccggacccgagtgcgcggaagcctccgagccgcggccaggtctcagaatgcgcggcggggcagcccggcccgtcaagcagcgaagccgaaactagaagcctgaggtctggcttggtcactgtttgctggggaactccgacgagcaactacccttcggggactcagtttcccccgggccaagtgagacggacagctgcggctcccgcgagaactcgcgcgggaacaggagctgcgggctccgcacagcgcccggcacgtagacccagtctactaacgggctggaaggtcgcgctcccgcctccgcgcctccctagaacctttctcacctcaagctcgggctcccggactctctcacctcacggcccctccttccaccttgggccgcccagcctcaaccgccct

SEQ ID NO: 20 - DNA, 1538 bp, Homo sapiens
  source 1..1538: mol_type = other DNA
ggctacgcaagaccttcagttccggattaggaggccccgccccccggcccgagggaggggcggagagacccgctcctgcgacttagggcgatgccaccttaaagggcttgacctcctcgaagccagaactgcggaagaggatggagaaagaaacgctagatagacccaggattcgaacccacaacagcttggatgcaaagctcattttgaattctgaagagctgggagtttagcagtggacgaccaaacaaaaaaataccagaagacggtgaaagcagcagctggaaatgagagaaaaattaagaaatagaaaatgctgtttcatgtattagggagtgcacatctttaaagagaagtgaagaacctttttggctaggcgctttctggggaccttggcagttatcaacaagtcactctgaaactaacagaaggtgaggagggagtaatccagaaaggaaactgccatttttgctagggcaagaaagtagacctagcaatgcaatgccatcaaggagttagctggctctagcgttctccgaactttgcaactcattttatattacattgtctgcgtcaagaaatttcaagtaaatgccttgagattttatatgtataaaatgtagtctctggccaggcggggtggctcacgcctgtaatcccagcactttgggaggctgaggcgagtagatcacgatgtcaggagatcaagaccatcctggctaacatggtgaaaccccgtctctactaaaaaatacaaaaaattagccgggcatgatggtgggcgcctgtagtcccagctactcgggaggctgagacaggagaatggtgtgaacccgggaggcagagcttgcagtgagccgagatcgcgccactgcactccagcctgggcgacagagcgagactccgtctcaaaaaaaaaaaaaaaaaaaaaagtctcagaagaagtgtcaagttgactaaatgatttttaagtcccttccactctactatatcacaataatttcccctattttaccttatttaattttcacaacaaccctgcagcctgtaagtaggtgactcctccatggtcagcaggtggtcagtggtcgaactatttaggactatctgatgctctttcctctgctcaccattgcaatacaatgagaaaaatgcaaatgaatctaagtacagtgttctctgggaatatgaagatagaactaaaaactgcctgagagatcagggcaggcttctagaaagttgtgtctaactagtcttgaggtttacataaaccacaccaggctgatgggactgggaagggcccactaagcagtgagaccattcccttttggagagtccttgcatcccctcccagatttcctctttgaggggaaggtgagagaggaggtaaaaggggtgaggaatggagaataactcattctggttcttgtttccccttttccacataaaagtatatttgtcttgtgttccatacaccagtccatactgatgtgatggtgttttttatgcttccttttgaataaacatttcattcttaa

SEQ ID NO: 21 - DNA, 3285 bp, Homo sapiens
  source 1..3285: mol_type = other DNA
ggtcagtttctggtcacatgattttcttctcgggctgcaaacaaagggaagcctgcaacaagttaagctgaagaccgaagcaagagctggttcaggtggcagccacagcagcctcagggacctcagcaactatggcctcctgcccagactctgataatagctgggtgcttgctggctccgagagcctgccagtggagacactgggcccggcatccaggatggacccagaatctgagagagccctgcaggcccctcacagcccctccaagacagatgggaaagaattagctgggaccatggatggagaagggacgctcttccagactgaaagccctcagtctggcagcattctaacagaggagactgaggtcaagggcaccctggaaggtgatgtttgtggtgtggagcctcctggcccaggagacacagtagtccagggagacctgcaggagaccaccgtggtgacaggcctgggaccagacacacaggacctggaaggccagagccctccacagagcctgccttcaacccccaaagcagcttggatcagggaggagggccgctgctccagcagtgacgatgacaccgacgtggacatggagggtctgcggagacggcggggccgggaggccggcccacctcagcccatggtgcccctggctgtggagaaccaggctgggggtgagggtgcaggcggggagctgggcatctccctcaacatgtgcctccttggggccctggttctgcttggcctgggggtcctcctcttctcaggtggcctctcagagtctgagactgggcccatggaggaagtggagcggcaggtcctcccagaccccgaggtgctggaagctgtgggggacaggcaggatgggctaagggaacagctgcaggccccagtgcctcctgacagtgtccccagcctgcaaaacatgggtcttctgctggacaagctggccaaggagaaccaggacatccggctgctgcaggcccagctgcaggcccaaaaggaagagcttcagagcctgatgcaccagcccaaagggctagaggaggagaatgcccagctccggggggctctgcagcagggcgaagccttccagcgggctctggagtcagagctgcagcagctgcgggcccggctccaggggctggaggccgactgtgtccggggcccagatggggtgtgcctcagtgggggtagaggcccacagggtgacaaggccatcagggagcaaggccccagggagcaggagccagaactcagcttcctgaagcagaaggaacagctggaggctgaggcacaggcattaaggcaagagttagagaggcagcgacggctgctggggtctgtacagcaggatctggagaggagcttgcaggatgccagccgcggggacccagctcatgctggcttggctgagctgggccacagattggcccagaaactgcagggcctggagaactggggccaggaccctggggtctctgccaatgcctcaaaggcctggcaccagaagtcccacttccagaattctagggagtggagtggaaaggaaaagtggtgggatgggcagagagaccggaaggctgagcactggaaacataagaaggaagaatctggccgggaaaggaagaagaactggggaggtcaggaggacagggagccagcaggaaggtggaaggagggcaggccaagggtggaggagtcggggagcaagaaggagggcaagcgacagggcccgaaggaacccccaaggaaaagtggtagcttccactcctctggagaaaagcagaagcaacctcggtggagggaagggactaaggacagccatgaccccctgccatcctgggcagagctgttgaggcccaagtaccgggcaccccagggctgctcaggtgtggacgagtgtgcccggcaggagggcctgactttctttggcacagagctagccccagtgcggcaacaggagctggcctctctgctaagaacatacttggcacggctgccctgggctgggcagctgaccaaggagctacccctctcacctgctttctttggtgaggatggcatcttccgtcatgaccgcctccgcttccgggattttgtggatgccctggaggacagcttggaggaggtggctgtgcaacagacaggtgatgatgatgaagtagatgactttgaggacttcatcttcagccacttctttggagacaaagcactgaagaagaggtcagggaagaaggacaagcactcacagagcccaagagctgcggggcccagggaggggcacagccatagccaccaccaccaccaccggggctgacaccctgccccacagggaatggccttggcctggcccagcccaagatcccagcgttatctaactcctggagggtggactctgtcctggcttgtttggtgtcctcagatatctttcacacagtagagcaaaatcaccagccctgcactgatgtcactttatgtagaaaaaggccttagctggacctgcgttgccgtctatgcaaatgcatgcaaatactccaggccctgggatgtgggcttgtgttttgtcactgtgaagggggagatgggagaggagcctgttttggggtggggtctggggaaggcaatctgattctgaagctaaagagctttcatcctcttgagtgtatgtccccatagtgggccccttgacccacatgctgaccggtgccttgggatttgactagagttgctggctcgaggcccagcacgaggacttaccctggggttttgttaggtttggaagcagctgtccctagggggtgaagtcccccccctttttttttttacccctgcttctcccacggcttcacctccctatgtgaactgtagactcagatcccaataaagtgctgttgcagctatgatgctaggtggtttctaagcacaggggacaccccacaccccctgcctgaatggatgggtccatcccaggcactggtacttgcccccttgttctgtatccccctttgcccttgccttgcccttccaacaaaccctaggcccttgagaagctgatacttctccttttgctcacagctgccttggccccacccctgggagatgtagcaaattgagtgtgggttttggagtctgagcctcaggctcaaatccaggccaagtgatcttgggcaagttaatctctgggaactttgggtttcttatcctcaaaaaaggcgatggaagggctggggaagtgattaaataaaagcaacgcaagaaaaaaaaaaaaaaaaaaa

SEQ ID NO: 22 - DNA, 1176 bp, Homo sapiens
  source 1..1176: mol_type = other DNA
aatagtgattaggaaaccttgaagcctgcccaacgatcgtgggcaggaggtggtttctggtttgttggggcgtgtgtatgtgtatttggggggactgaagggtacgtggggcgaaacaaaaccggccatggcagcagcggaggaggaggacgggggccccgaagggccaaatcgcgagcggggcggggcgggcgcgaccttcgaatgtaatatatgtttggagactgctcgggaagctgtggtcagtgtgtgtggccacctgtactgttggccatgtcttcatcagtggctggagacacggccagaacggcaagagtgtccagtatgtaaagctgggatcagcagagagaaggttgtcccgctttatgggcgagggagccagaagccccaggatcccagattaaaaactccaccccgcccccagggccagagaccagctccggagagcagagggggattccagccatttggtgataccgggggcttccacttctcatttggtgttggtgcttttccctttggctttttcaccaccgtcttcaatgcccatgagcctttccgccggggtacaggtgtggatctgggacagggtcacccagcctccagctggcaggattccctcttcctgtttctcgccatcttcttctttttttggctgctcagtatttgagctatgtctgcttcctgcccacctccagccagagaagaatcagtattgagggtccctgctgacccttccgtactcctggacccccttgacccctctatttctgttggctaaggccagccctggacattgtccaggaaggcctggggaggaggagtgaagtctgtgcatagatgggagagccttctgctcagaggctcactcagtaacgttgtttaattctctgccctggggaaggaggatggattgagagaatgtctttctcctctcctaagtctttgctttccctgatttcttgatttgatcttcaaaggtgggcaaagttccctctgactcttcccccactccccatcttactgatttaatttaatttttcactccccagagtctaatatggattctgactcttaagtgcttccgccccctcactacctcctttaatacaaattcaataaaaaaggtgaaatataaaaaaaaaaaaaaaaaaaaaaaaaaaaaaaaaaaaaaaa

SEQ ID NO: 23 - DNA, 5565 bp, Homo sapiens
  source 1..5565: mol_type = other DNA
ctcctgcacggcgagtgctggagcacgacgtaccgctcgctcggtcagggcgccccctccgcccgcctcctgcttcctcctccgctgcctgccgccgccgcctccaccattgtataatgctcggggcgcgcaggcagagaacggcggagtcttagcttcagcctcgcctgctgcccgctccccggcgccaccctcgggcccctggagcggggcactccgcatggagcgggagtagctgaggagtgggcggaaacccctcctgatgcgttagttcccaggtggagctgcatgtgatatatgttgggtaaaggaggaaaacggaagtttgatgagcatgaagatgggctggaaggcaaaatcgtgtctccctgtgacggtccatccaaggtgtcttacaccttacagcgccagactatcttcaacatttcccttatgaaactctataaccacaggcccctgacagagcccagcttgcaaaagaccgttttaattaacaacatgttgaggcggatccaggaggaactcaaacaggaaggcagcctgaggcccatgttcaccccctcctcccagcccaccaccgagcccagcgacagctaccgagaggccccgccggccttcagccacctggcgtccccgtcctcccacccctgcgacctcggaagcactacgcccctggaggcctgcctcaccccggcctcactgctcgaggacgacgatgacacgttttgcacctcccaggccatgcagcccacggctcccaccaaactgtcacctccagccctcttgccagaaaaggacagtttctcctctgccttggacgagatcgaggagctctgtcccacatctacctccacagaggcggccacggctgcgactgacagtgtgaaagggacctccagcgaggctggcacccagaaactcgacggtcctcaagagagccgcgcagatgactcaaaactgatggactctctgcctgggaattttgaaataacgacgtccacgggtttcctgacagacttgaccctggatgacatcctgtttgctgacattgatacgtccatgtatgattttgacccctgcacttcctcatcagggacagcctcaaaaatggcccctgtgtctgccgacgacctcctcaaaactctggctccttacagcagtcagcctgtcaccccaagtcagcctttcaaaatggacctcacagagctggaccacatcatggaggtgcttgttgggtcctaagacccagggacccagcgactatgcccacccagaccccagagcgttcccataaccctgacagttctccacactgtgcatgcacccttgcttgcctttttcagagaaaaagaaaattttacaacaggatcacactagtttttgctttgagcagagttggagtgccttcatccaagtatgaccacttttaatacacttttttgagtggttcctcagagacctactaccctggtataggaaagaatccatttgaagacaatgttgcaatgttgaatgacaaaaataaacagttcaagtgaagcacaaggattaagttggaaaagctgtaaattgcatgtgcatatttgtctattttttctataagttttattgcaagaggtaaagaagaaaactatatatatatatcttatttagataatctcagtaccttttctggcatttttgccctgtataggttgacttggcaattcggcctttttagaggcattaactactcctcgtaagtgttgcatttacatggctgtttagaaaactgctgcccaaatttattttatatttttgtacagattctgcagtttatgatattgtttttctaaaaacaaatgctgtttatacatatgagatagctattttgataggatttgctcacatagttcctgcaaacttcagatgtacaagttgcacttgtacttttatagagttgtaatgttttatatgtgtatggtgcaagagaaaattggatcaaatcaatctgcagttgatgtccccaaatgcaaacacaggcacacacatgcacacacccataaacacacacacagtgctttaagaaagggccaggtgatatcacacccaaatttcacaagcactgaccccctggcaccaacacccgccagtactgtgacttccaaagccagagccacatgtgctcatcaaacttgcattaagcagttggcgggagatggctgtggagctgggggtttaagtgatggttctcttttgctccctcttttgagggtaaagctactgtctttcttaagagtgtatttatgccaagtttgcgcttttaattgtttttattttgttttttaatgaaaacccagatctttcctttttggcataatttttatgatgacctgaaattttacatccgaacaaaattttacatccgaaaagcaaccaacttcttcatggaactcagccctgttgcaatgcttagggcccttaaagaagaaaatctccccagaaggcatccatcatgttgcttaattgtcttctgcagcttcctttccctagagctttccctgtgttgctaagagctgaaaatggcatcttcgtgatcaccacagtgagcttggctcgcctcggccggcccgggatgcactcttacaacatgtgtgactcttgaacctggagttcatcacattacgtcacagcttcccatctggttgctttcctgagtcagctacttcacacttgtcaaggctgttttaccccaaaactcagacaggactttctatgcatgttttccctcctccccccaattcccccccccatcaccttatctcccaggacacacttgagaagtagctttttattcctagtggtgtacatttaattttaaaaaggttgcaatgtatcatgcttgttgccgaaactgtttatggccttcttgtttcagttttttcttttcttccaatggtactttagctgttgagtgcaggttacaacctatattgttatgcagatggcttctttaggaataacttttatatttatttaaaaatttttaaattatgggatgttttgttgttgttgttgtctttgttgttggtcatttgtcaatattcagtcaccaattctgctcacttcttgccatggataaaattgggtctttctggctaattaaaaaagacaactttataaaatggcactttaagcaagccatagttagttttatttttgtaatgcacatggcaaagcaaagacgtttgtgatgaaggaactgctcatctaagcaaaagatttgagtatgatatgataaaggctttctacattctaatttactttttccccccacttgaatgtgttttaaaggctaattatcagctcagtagagcagtgagaaactgatcaaattgcacttgttctcctacaagcaacctccacgcagacacctcgtactgctacaggtgtgtcatttcctttaataggaccagggaccatgtaactgaggtgagggttgtagtagatgcttccagtgtcagtatgcctgttaattttaagagcttccctttcttgcagagaacaagtctgcccagattccatgctttctataactggaggacctggcaaacctgccgcatgctgcacacatctacctacgtacacatatacaatagtattgatgattctgaacaataacagggtaaaacagttggtttgccattgttaaaaactgatttacagtaacttacaacaactgtacttttgttggattagcaaatcatgtgtttaaacaaatcccatatgttgggcaacagttcaaataagcacggagaagtgttgcccaaacttggttctctgactcttatgtatttgtaaggctgggcttcaaaatcaaaacaaaaaccccaaaaacagcaggcaaatgctttttaactctgacaccgttgccataaatccctgatactcaaagtctaacaagaaagacatggaaaattagcagcccattttcagaaagatcaaaatgatctagggttctaattgcttttgcatcctattcttacaaagtgatgtcccaacagggaacagtaggagctggagtgggatctccaagtcccagtttgagtgtgggatgtgcttccagcagtgccttccctttatgaaagacatcacatggcatccagggccaggcaggcagcttgaggtgcctttacgagaaaaccgagctggggctgggagaggacagttattgacactgatgtgcaatgaagtgacaagatgagagcagaatcgtaagagctttgaatttgaagtgagtttttttccccccataagttatttattccttttttctgtgtaaatatatttattttactgtggagcgctaacatctggatcgtaacatgtgcagaatgtatggtaggaatgtattctcttgtaggaatgtaaatctgtattaaaagggggtccaagccaggcccccaggtcttctcattgtatgcacagtccgcattcatttttactcttctctaatatgggtctatttgaaatatgcaaaaggtatgaggaatgttttaatacctccaaatttttaagaaaagcatcaaagggttgatattttttaaagtttttttagtagcactttctctggatgacagaaggagcaaccacatgggcacccttgttcataccaaagggtgagcagtggccagagcctcctctgcacctctcgagtgtctttaccaattgagctttttatcgccatagccccttggagtgccccagctgccctgaggtcaatcaaggaaaatttcttaatgaaataagctccaaagagccaaagtatcaacttacagatcgtttttaaagcttaaatttatgaaccacctttgtggtaaacaatgaattatgaataccgcagggcagccttcttaaatgacaaatgtaaaaaaaaaaaaaaaaaagactctacttcgtgcagcaattgctactctatacgaattgtcttaatttgaaaaccttgctgttacaaattggacctttatacattttctgaaaacaatgaaaagagtatatttaaccttttctggctgtaaatggttaccttcctgtaactgccccgcacctggaggcatggagttgtgtgcatcctgcttatgtacaattgttttcagtgtttctaagaatgagtctgaatggttcttgaaaattagccaggatcaaatgctattgcagacaaagccaataaaaagttggacttcttttggggataacaagttttggaagagaaatgcaggccatatgtgcgcatgaccgagattttgaaaaaagatgtacatagtgacatgtttggtgcatggtttttgaggagggcttttgtcaaaaaggaggtataacctttcccccacagacctgagagctgtgccttttctatgcaatattacagacgttacatcggaacccagatggctgtattcacatgtaggtttgggctgtaatctaaacaattggacagattaaatgtacatggaaatgagcagtcttacttttgtagttttatattatacaataaacagttaaaagatgaaaaaaaaaaaaaaaaaa

SEQ ID NO: 24 - DNA, 1201 bp, Homo sapiens
  source 1..1201: mol_type = other DNA
taccacctcaggagagttccagggaagaaccccacccgcactccaatgaggtcacaatggctggagctctgaggggcccaggctccctgagccaggaggagaggagaaagtccaaggaaagatggctggcagtcacccctacttcaacctgcctgactccacacacccatcgccgccctccgctccacccagcctccgctggcaccagcgctgccagccctctggtgccaccaatggcctgctggtggccctgctgggtgggggcctgcctgctggcttcgtgggccccctttctcgtatggcttaccagggttccaacctgccctcgctggagctgctcatctgtcgatgcctcttccacctccctattgccctgctacttaaactgcgtggcgacccccttctgggacctcctgacatccgaggctgggcctgcttctgtgccctgctcaacgtcctcagcattggatgtgcctacagtgcagttcaggtggtgcccgctggcaacgctgccactgttcgcaaaggttcttccaccgtatgctccgctgtcctcaccctctgccttgagagccagggtctcggtggctacgagtggtgtggactgttgggcagcatcctaggactaatcatcattctgggacctggactctggacactacaggaggggaccacaggtgtctacaccaccctgggctatgtgcaggctttcctgggaggcctggcgctgtccctggggcttctggtctatcgttctctgcactttccctcctgcctcccaacagtggccttcctatctggcttggtggggctgctgggctgtgtgccaggcctctttgtgctgcagacccccgtgttgcccagtgacctcctgagttggagttgtgtgggggcagaggggatcctcgccttggtctccttcacatgtgtgggctatgcggtcaccaaggcccaccctgccctggtgtgcgctgtcctgcattccgaggtggttgtggcccttatactgcagtattatatgctccatgagactgtggcactttctgacatcatgggggcaggggttgtgctgggcagcattgccatcattacagcccggaacctcagctgtgagaggacagggaaggtggaggagtgagatagaacttgggagcccgggggttgggagggacagggataaataaagacaaagactgaagac

SEQ ID NO: 25 - DNA, 6335 bp, Homo sapiens
  source 1..6335: mol_type = other DNA
agtgctgagggagcaaagttcattttctcgggtaggagaagatgattctcttgcaacacgtgcggattgtgacaaaatctttcattaacaaggggagtttcggtgaagtggaggtttggggaaaggcgaggaagtcggtctggagcaagcaagcaaagtgcggaagctgtactgggattcttctagaaagtggggtgggaaaggagctagggagggcgtgtggagggacgagatctgtgtcagaacgtgcgtgtgagcggatacaaaacccgagagaggcgtgagcagcgctgtgtttgcgagcgggagcgaggggcgccggctggggtgtgtgctcctgagctcttcagaaaccaggctgctttcaggaacattgctgtggattcccagggcctattccactagaagcaagatggctgaactcaatactcatgtgaatgtcaaggaaaagatctatgcagttagatcagttgttcccaacaaaagcaataatgaaatagtcctggtgctccaacagtttgattttaatgtggataaagccgtgcaagcctttgtggatggcagtgcaattcaagttctaaaagaatggaatatgacaggaaaaaagaagaacaataaaagaaaaagaagcaagtccaagcagcatcaaggcaacaaagatgctaaagacaaggtggagaggcctgaggcagggcccctgcagccgcagccaccacagattcaaaacggccccatgaatggctgcgagaaggacagctcgtccacagattctgctaacgaaaaaccagcccttatccctcgtgagaaaaagatctcgatacttgaggaaccttcaaaggcacttcgtggggtcacagaaggcaacagactactgcaacagaaactatccttagatgggaaccccaaacctatacatggaacaacagagaggtcagatggcctacagtggtcagctgagcagccttgtaacccaagcaagcctaaggcaaaaacatctcctgttaagtccaatacccctgcagctcatcttgaaataaagccagatgagttggcaaagaaaagaggcccaaatattgagaaatcagtgaaggatttgcaacgctgcaccgtttctctaactagatatcgcgtcatgattaaggaagaagtggatagttccgtgaagaagatcaaagctgcctttgctgaattacacaactgcatcattgacaaagaagtttcattaatggcagaaatggataaagttaaagaagaagccatggaaatcctgactgctcgtcagaagaaagcagaagaactaaagagactcactgaccttgccagtcagatggcagagatgcagctggccgaactcagggcagaaattaagcactttgtcagcgagcgtaaatatgacgaggagctcgggaaagctgcccggttttcctgtgacatcgaacagctgaaggcccaaatcatgctctgcggagaaattacacatccaaagaacaactattcctcaagaactccctgcagctccctgctgcctctgctgaatgcgcacgcagcaacctctgggaaacagagtaacttttcccgaaaatcatccactcacaataagccctctgaaggcaaagcggcaaaccccaaaatggtgagcagtctccccagcaccgccgacccctctcaccagaccatgccggccaacaagcagaatggatcttctaaccaaagacggagatttaatccacagtatcataacaacaggctaaatgggcctgccaagtcgcagggcagtgggaatgaagccgagccactgggaaagggcaacagccgccacgaacacagaagacagccgcacaacggcttccggcccaaaaacaaaggcggtgccaaaaatcaagaggcttccttggggatgaagacccccgaggccccggcccattctgaaaagccccggcgaaggcagcacgctgcagacacctcggaggccaggcccttccggggtagtgtcggtagggtttcacagtgcaatctctgccccacgagaatagaagtttccacagatgcagcagttctctcagtcccggctgtgacgttggtggcctgagctaggaggaaaaagagcagttttcactcagttttggttccctgcccgaggtgctgacccaattcgctgccaaaagagtgtcaatcagaatatacaaatcccgtatggttgtgtcatcctctcttaatcatttttactaattctaataatcagctctagcttgcttcataattttcatggctttgcttgatctgttgatgctttctctcatcaagactttgcagcattttagccaggcagtatttactcattattaggaaaatcaagatgtggctgaagatcagaggctcagttagcaacctgtgttgtagcagtgatgtcagtccattgattgtctttagagagttaatgttacaaaaaagaattcttaataatcagacaaacatgatctgctgaggacacatgcgcttttgtagaatttaacatctggtgtttttctgaaaaaatatatatacatatattgctttatttgaaacaaattaaaatatgctgcatttgacacctggctagtttcttttattgatacccacctagttattgaatgtactgtttagtgctttcaaaaaaaactttagagactagaggttgtggtgcaaagctgtgtacaataaatactgtttctgttgagacaagtactctttcaggaaaatatatatatgccctttcaattagattacacaaatagatggatatgcaccctgatcatcttagacacaccatgtggcagttgggcagttggaaacattggttgcaagtactacaaacctcagctgagcctagcttcaacaataagaatttattggttcagtgagtgaaaagtccagttggtccaatttgatcaggatttcagctcccatctcttttttttttttcttttttctttttctttttttgagacagtcttgctttgtcacccaggtggagtgcaatggcatgatctcggctcactgcaacctccgcctcctgggttcaagccattctcctgcccggccttccgagtagctgggattacaggcatgtgccaccacgccctgctaatttttatattattagtacagacaggatttcaccatgttgtccaggctggtctcaaacccctgacctcaggtgatccacctgctttggcctccgaaactgccggaattacaggcatgagccaccgtacccggccagctcccatctcttaattatcttagctctcctttcctccttgggttgacattgcattcagaattgtggcaaagggccaccatgctcttaagactcaagtctattttccacactgtccagaggagagaagttatcctagtagcttctacacagagcaagtgtctttctcagaaatcccagcaaaggtcttgcataccattgctggtaggcctgtctcttaaaccaatcatgaaaggggggagagaagtgaatgggatgaactaatttgcatagactaattagagcccacccctggagcctgggtgtggccatcttcccagagttcctgagctagatggagaaggggtacttctcagaaagggaaaatggatacccaatggcccaaacccaaaatagccccagttcccctaactttgactacagggcagtccagtttgggtgccgcttccgttgcactcacatgtcctacatatctgttgactactcacaagtgcaaatgcttattctcaactcaacattaacattttttctggcaacccaggttcactggttctcttccacagagcggccctgagcagctgagcctgcaagccacgcaagcatctgtttcttcttttgccaagtacaggaggatgtttgctctctctgtagagagctttctgaggtctctgggtgtacccagagatttaatagaaattcttaacgttaagtcacattccaggaaggaaggaagagttgttcgttcaaataagaaagataaatgttcggcactgtaggccctgtttaccccatctgaggccctgaattcatatattacaagacggaaggattttgcacagttttttatgtagcaagattttgctcaccactgaaaaatgtcagtgtaaatgtgaccgctttaaagatgagtcaagtaattcttggaacagggaaaaaaatgaatttgccaggtcaggagttcacctgcctttgtcagagttgaacccaaccactcttgacctcgactcactcccttagggttaagaaagcccaaacacattcctgagcacagagcaaacactcccatgtcactgaaaagaaacaaaagaatgctaaaaagtgctcagacctgatcacattttttccaatattttttcctttttttttttttttttgagacagggtcttgctctatcacctaggctggagtgcatgtttttgagacagggtcttgctctatcacctaggctggagtgcatgatcatggctcactatagccttgaactcctgggctcaagccatcctcagcctcccaagttgcgaggactacaggtgtgcaccacaacgtcccagctactttttaaatttttagtagaaacgaggtctcactatggtgcccagactggtctcgaactccggagctcaagtgatcttcctgctttggcctcccaaagtgttagaattacaggcatgagccacctcgcctggctggtttttgcctttcttatagaccctgggcatgtaagcatttattagtttgcatttttgaaacagtaatttcaatattttagtgccaatgtcaggccgcttaaacactgtattacatatcttcatctgtctggtggaactattggtgtgatcctagagaactgagtcctattctgccattcatttaaagtgttttaaactctaatctctctacttaatgcacagtagtcagattattctcttaaacatttgcctagtagaggttaaaatagtttaatccttatgaagatggaataacttcaaactcacattgtggcacttagatcttccaccaagacttcatccgtgaaatccacacctccctgttgggttcccaattacattccaaatttacatttcttttgagaatctctgcatactccagctctgtcctgttgatcctattctagaagtgcttaatgcagcaagacacagaaagttaaacgcaaattgctgcaaaattcaccctcagtggaggactagaaacacaacatgtccaatttaaagctcagttcacaagcagttcaattctgctggcatcagaaaaggagattctaattaaacattcttagggaaggacatcaaatgaggttaatgggaaacgttaccagattaaaagcagttttttgacaaagtaacagatttggaaattctgactctctgaaagccttgatttgaacctcaaacttgatttcaccatgagaagtggggatcaagggcctgcgcagttcttttcctaaatcgattcggtgctccccaccccgacgcaggcacaggtccgcaaccagatagggagatgcctgaatttcaggctacctttgacaaagctttcttcctccctccctccctttgcacgctgcatcccacgctgcctgcttaaagcgccctcatgtgtctacagatggtaaaacgtttatttctcaacagacattccagtgatagcatccaatgacctatgtaacggcacccttttttgctgtacgcgttttttgagatggagtctcgctctgtcgcccaggctggagtgcagtggcgcggtctcggctcactgcaagctccgcctcccgggttcacgccattctcctgcctcagcctcccgagtagctgggactacaggcacccgccaccacacctggctaattttttgtatttttagtagatacagggtttcaccgtgttagccaggatggttttgatctcctgacctcgtgatccacccccctcggcctcccaaagtgctgggattacaggggtgagccactgcgcccggccccagtcacttgttcttaagtttcttaagcaaactataaaatagcaaaagaccaaaaaaaaaggaaaaaaagcagttcgcctaatacattgttccagcatttccttgaaagtactgagccatctcaattgctctgattttgtgagaaaattatgaagagttgcaaagtcccagtgattctcttgttacttagctaagaatttgaaatgtaatttaaatactattctttacaatccattataaggattttaaaatctttttgcttctttaataaattctaacaaagaaaaaaaaaaaaaa

SEQ ID NO: 26 - DNA, 3572 bp, Homo sapiens
  source 1..3572: mol_type = other DNA
agagccgaggccaggctgccctcgaagcggggcggggcgaagcggggcggggccgagcagggcggggcgggggcttgaggtgattcccaagccgcggggcggctccggtggtgcggggaaaccgaaagtgggcggcggccgcggcggggcccctggcggagacggcggcaggagctgggcccagagacgcggggacgggccgtgggcccccggaacgagattacctgctatgccatggcctgcacagaaactgcaagaattgctcagcttgtggctcgtcaaaggagttctaaaaggaaaaccgggcgtcaagttaattgtcagatgagagtgaagaaaaccatgccattttttctagaaggaaaaccaaaagcaaccctcagacaaccaggatttgcttcaaatttttctgttggcaaaaaacctaatccagcaccgttaagagacaaaggaaactctgttggagttaagcctgatgctgaaatgtctccttatatgctacacacaactcttggaaatgaagcattcaaagacattccagtgcaaaggcatgtgaccatgtccaccaacaacaggtttagcccaaaggcgtcccttcaaccacctttgcagatgcatctctcaagaacctctactaaggaaatgagtgataatttaaatcagactgttgaaaaacccaatgtcaagcctcctgcctcttacacttataaaatggatgaggttcaaaatcgcataaaggaaatactaaacaagcataacaatggcatttggatatctaagcttccacatttttacaaagagttatataaagaagaccttaatcaaggaattttacaacagtttgaacactggcctcatatttgcacggtggagaaaccttgcagtggtggccaagatttacttctttatccagctaagagaaagcagcttttgagaagtgaactggatactgagaaagtacctctatccccactacctggtcccaaacaaacaccaccgttgaaagggtgtccaacagttatggcaggagactttaaagaaaaagtggcagacctgctggtgaaatacacaagtggcctttgggccagtgcacttccgaaagcatttgaggaaatgtacaaagtgaaattccctgaggatgccttaaaaaatcttgcctcactttctgatgtatgcagcatagactacatttctggaaatccccagaaggccattctctatgctaaacttccattgcccactgacaaaatccaaaaggatgcagggcaagcacatggtgataatgatatcaaggctatggttgaacaagagtatttgcaggtagaagaaagcattgctgaaagtgctaatacctttatggaggacataacagttcctcctttaatgattccaactgaagcatcaccatctgtattggtggttgaactgagcaacacaaatgaagtggttatcaggtatgtgggcaaagactattctgctgctcaggaattaatggaagatgagatgaaggaatattacagtaagaatcctaagatcacaccagtccaggctgtgaatgttgggcagttgctggccgtaaatgccgaggaggacgcctggttacgggcacaggtcatctcaacagaagagaacaaaataaaggtatgctatgttgactatggttttagtgaaaatgttgaaaaaagcaaagcatacaaattaaacccgaagttttgttcactctcatttcaagctacaaaatgtaagcttgcaggcttggaagtcctaagcgatgaccctgatctagtgaaggtggttgaatctttaacttgtggaaagatctttgcagtggaaatacttgacaaagctgacattccacttgttgttctgtacgatacctcaggagaagatgatatcaatatcaatgccacctgcttgaaggctatatgtgacaagtcactagaggttcacctgcaggttgacgccatgtacacaaatgtcaaagtaactaatatttgctctgatgggacactctactgccaggtgccttgtaagggtctgaacaagctcagtgaccttctacgtaagatagaggactacttccattgcaagcacatgacctctgagtgctttgtttcattacccttctgtgggaaaatctgcctcttccattgcaaaggaaaatggttacgagtagagatcacaaatgttcacagcagccgggctcttgatgttcagttcctggactctggcactgtgacatctgtaaaagtgtcagagctcagggaaattccacctcggtttctacaagaaatgattgcaataccacctcaggccattaagtgctgtttagcagatcttccacaatctattggcatgtggacaccagatgcagtgctgtggttaagagattctgttttgaattgctcggactgtagcattaaggttacaaaagtggatgaaaccagagggatcgcacatgtttatttatttacccctaagaacttccctgaccctcatcgcagtattaatcgccagattacaaatgcagacttgtggaagcatcagaaggatgtgtttttgagtgccatatccagtggagctgactctcccaacagcaaaaatggcaacatgcccatgtcgggcaacactggagagaatttcagaaagaacctcacagatgtcatcaaaaagtccatggtggaccatacgagcgctttctccacagaggaactgccacctcctgtccacttatcaaagccaggggaacacatggatgtgtatgtgcctgtggcctgtcacccaggctacttcgtcatccagccttggcaggagatacataagttggaagttctgatggaagagatgattctatattacagcgtgtctgaagagcgccacatagcagtggagaaagaccaagtgtatgctgcaaaagtggaaaataagtggcacagggtgcttttaaaaggaatcctgaccaatggactggtatctgtgtatgagctggattatggcaaacacgaattagtcaacataagaaaagtacagcccctagtggacatgttccgaaagctgcccttccaagcagtcacagctcaacttgcaggagtgaagtgcaaccagtggtctgaggaggcttctatggtgtttcgaaatcatgtggagaagaaacctctggtggcactggtgcagacagtcattgaaaatgctaacccttgggaccggaaagtagtggtctacttagtggacacatcgttgccagacaccgatacctggattcatgattttatgtcagagtatctgatagagctttcaaaagttaattaatgactgcctctgaaaccttgacaactaattcagattttttagcaataacaaaatgtagtaggcttaaaaaaaatcttaactctgctacatggctctgactgctgtgggggattgaaaagaatatgcttatgtttgatgaaagatatttaacaagttttgttttaacagagttgacttttcaaagaaaattgtacttgaattattactataatattagaataaaaatgtttatcaatataaaaaaaaaaaaaaaaaaaaaaaaa

SEQ ID NO: 27 - DNA, 4476 bp, Homo sapiens
  source 1..4476: mol_type = other DNA
agcagagagaacacacgtccttgcggaagtgacggcagttccgagtccagtgggggcggtgggagcgatgagggtctgagacggtgggagcggttgtgtgaagatggagacattccatacaccaagcttgggtgatgaggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttcccagccagaagacagtcgtggtggaagcagggaaaaagcagaaggccccaaagaagagaaaaaagaaagatcctaatgaacctcagaaaccagtttcagcatatgctttattctttcgtgatacacaggctgccatcaagggacagaatcctaatgccacttttggtgaggtttcaaaaattgtggcctccatgtgggatagtcttggagaggagcaaaaacaggtatataagaggaaaactgaggctgccaagaaagagtatctgaaggcactggctgcttacaaagacaaccaggagtgtcaggccactgtggaaacagtggaattggatccagcaccaccatcacaaactccttctccacctcctatggctactgttgacccagcatctccagcaccagcttcaatagagccccctgccctgtccccatccattgttgttaactccaccctttcatcctatgtggcaaaccaggcatcttctggagctgggggtcagcccaatatcaccaagttgattattaccaaacaaatgttgccctcttctattactatgtctcaaggagggatggttactgttatcccagccacagtggtgacctcccgggggctccaactaggccaaaccagtacagctactatccagcccagtcaacaagcccagattgtcactcggtcagtgttgcaggcagcagcagctgctgctgctgctgcttctatgcaactgcctccaccccgactacagccccctccattacaacagatgccacagcccccgactcagcagcaagttaccattctgcagcagcctcctccactccaggccatgcaacagcctccacctcagaaagttcgaatcaatttacagcaacagcctcctcctctgcagatcaagagtgtgcctctacccactttgaaaatgcagactaccttagtcccaccaactgtggaaagtagtcctgagcggcctatgaacaacagccctgaggcccatacagtggaggcaccttctcctgagactatctgtgagatgatcacagatgtagttcctgaggttgagtctccttctcagatggatgttgaattggtgagtgggtctcctgtggcactctcaccccagcctcgatgtgtgaggtctggttgtgagaaccctcccattgtgagtaaggactgggacaatgaatactgcagcaatgagtgtgtggtgaagcactgcagggatgtattcttggcctgggtagcctctagaaattcaaacacagtggtgtttgtgaaatagtccttcctgttctccaagccagtgaagagttatctgctgggaaagtgtccaagagcctgtttttgaaacacaagctgggcttctggtagtgcctcatcacaacccatgatggctgttcatgtttcaccccttttcttccttcagcagaggccaggctatggagcagggccactgaatttgctgtaatctggagatgctttttactttcaaccataagcggtaatagcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgtgttttttgttttttttttttttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagttacacaagaaaaaa

SEQ ID NO: 28 - DNA, 4814 bp, Homo sapiens
  source 1..4814: mol_type = other DNA
cttcgtgccacgtcaccgcctgcgtcgcttccggaggcgcagcgggcgatgacgtagagggacgtgccctctatatgaggttggggagcggctgagtcggccttttccgcccgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgatgcaaaagaaagaaatccctgcgctttctgtctgtctttgtggcggcccagattgaattggggaatacatctttagcctggaaatgtaggctgcatgttaatggtaatgtaacttttgcagtgtaatgtttgaaaaatattaatgtagtttttgcttttacagtaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagagaatgccttttagtttaatgcatatttaaactaaattgatcctgtagtgttcctggagaagctagagcctgattgtaggctactactcatcaattaacttctacagtggagactacttctgggactggaatataaaaaagaatcaaaggttctgattttgagttgcaataaagggaaagaccatgctcatagcagtgccaacatctgaagtgtggagccttacccatttcatcacctacaacggaagtagttaactggaagagattaccaagagaataaaaagagactcattcagtggaagcaactttgtctcagcttatttcacataaagagagcgaagtcttttgggatgaatgttaattaaactccctggtaactagaacagggactggcaaactagcctatctgaccacctgttttgtacactttaaggtggttggttgcctttttaaatggttgaggggaaaagaataccttgtgggatatggaatttaagttcgagtccagttttattggaacgtggctatgcttattcatttatggattgactgtggctgttgtcagtgcatgagcagagttgtgtctaacagactagagcctgcaagtttgccagcccctgatttaaaagatgaaggtacacagaatgtgggctggctggtgggcaaaggggtaaaaatgttctctatattgtatctgaaaagatggggtgtctgaataagaaaatgcatctatttgacagacctggagcagttgctatctgctgctatggtttccaccacagatgcaagaagaacatgtccttgcgctttccgtctgtctaattgtggcagctgagattgaatagaggaatacaggaggaaaaaaagcgggaagagtttttgaggcaggtcggtcacccaggcttgtagtgcagtggcacaagcaactcactgcattctctgcatcctgtgctcaagccattttcccacctcagtctcactagttgctgggactgcaggcatgcacccctatgcccagctaatttttgtagagaccgagtatcgcttagttgcccagggtggtctcaactcctgggctcaaggagatctgcccacctcagcctcccaaagtgcaggcctagcctgggaggggaattttcaaaacgtgagttttgggaaatagtctatcagccttacctggttgattacacttgtaaaagaaagattaaaagcaggccagtgactctggtctgcttgaacatgtgaatgtagtggtttgagcaatctggagtttgccctagtgtcaaattccagactgtccatagtgtccaaaacctgaggcagatactaatgttaacccccagcaccccgtgattggaaacaaacctaaatacgtattgggaacttaatagcaattttaagcattctgatagattttttgtagggatggggtcatgccatgtggcccaggctggtctgaaaactctggcctcaagtgatctcaagctttggccttctaaagtgttgggattacaggtgtgaggcattgcacctggcttagcgttctgatttgacattgtaatgaaaagtgtgagtctcatctacagggccttttgtcctctgaaatgatagcaggaagggaattttcaggcagtggtcaaagctggggaaaccaggatagtgaagaaggccttgaggtgagagatggaagctaattggtgaactagccttggaagcctgaaacagacaagtagcaattcagagactttgtgggctccactgctccaacttgttttgaagattttcagttctgcagaagaggtatttccccagttgtcctttcagtgctcttagctgttttcccaacatccagatccaatcaaggctgggacatagcattttatcatgtctatttaagtcagaagtgatgaaccccagctgtttacctcatggtaaacctttgaagattccaggtagaatcttctcagactttgaagactgtctcattttatatctttttctcgttattcctagggtcaagacgttttgggcaagaataaggatgtgaacatcagaaagctcataacattttgtttttgatgctaagtttaacaaaggcatgctttagtagcctgtgggccctagggtttgttaaagtgtggagaacaactgagtggagcaagaggacttttctaggaaggtccttgtaatgtgacatttgaaaacaaatgaaggtgtggaagtaggccatgtggatatcaggacaaaccattccaggccaagacaacagcagttagtctggagtgtgatgtgttctgggaaaaaagtggccactttgctaacccaagaagacaggaagggttgtaaagcagtgggagtgtgcaaggaaggaagaccagacctcaaggaaaccacaggcgctctgagcagaagagttacatgatatgactcaaatttttaaaggatcactttggctgccaggtggcagggtaaaagcatagaataattgtgtataatgtgtttttaaggcaaagatagtggcttagtctagggtagtagactgaggtggtaggaaatgaagatagagacaacaggatatgctggtgggtgaggatggatttaatgttgatacaagtattttggtctgagcgtttggaagaaagttggcactgaggtgggaagtcgagtttagttttgttagttttggatgtgttaagtttgagatgctgattcttcagagaagtctaagctggagaactatatagagagtggaaagataacaatagacattgaaagccatgatacaggataaggtcatttggagagaggatagactgcattccaacatgagattggttgacaaagagaaaccaacaaaggtaattaagaggtgctcccactgcacttgtactcagaaggctgaggtaggattgttagaggccagcctgggcaccacagggagaccccatctctaaaatttagccaggaaccatggctcatgcctgtagccccaggaatttgggaggctgagtggggaggatcgcttgaggtcaggagtttgagaccagcctgggcaacatagggagacctaaaaaaattaattgggcatctgtagtcccagctactcaggcggctgagctgagaggatggcttgagtccgagagattgagggtgcagtgagctgtgatcataccactgcactccagcctgggcggcagtgagacactatctgaaaaaagtttaaaaattttaaaaaagaaggaactgcccctgaggtaagaaccaagggagggcctcccagaggtcaggtggaaaaagttttaggaaggaggaagtagtcaacagggttacctgttgcaaagtacttaagtaatatgaggcctgatagtggtaaacttgactaccgttggatttcactagtgggaaaggaagtctaattaaaatgcactcaagagactaacagtcgcaggcatgaaatacaatacaggtacatggttttttattatgtgtgcatctgcttcagtaataggtgtgaattactcatttggatcattaggagtttcaaaatctagttaaatgactagatttttgttgatgtaaattctgtcattctgaactgcagggattgtcagtaacttaactgcaaactaaactggtgataattatggtaaaattgcaagacgagcaataaatctcaaccaacttgagagaacactgataa

SEQ ID NO: 29 - DNA, 2914 bp, Homo sapiens
  source 1..2914: mol_type = other DNA
gatttcagttgaaagatgtgtttttgtgagtagagcaccgcagaagaactgaagactgttgtgtgctccccgcagaaggggctaccatgatcctttcctcctataacaccatccagtcggttttctgttgctgctgttgctgttcagtgcagaagcgacaaatgagaacacagataagcctgagcacagatgaagagcttccagaaaaatacacccagcgtcgcaggccgtggctcagccaattgtcaaataagaagcaatccaacacgggccgtgtgcagccgtcaaaacgaaagccactgcctcccctcccaccctctgaggttgctgaagagaagatccaagtcaaggcactttatgattttctgcccagagaaccctgtaatttagccttaaggagagcagaagaatacctgatactggagaaatacaatcctcactggtggaaggcaagagaccgtttggggaatgaaggcttaatcccaagcaactatgtgactgaaaacaaaataactaatttagaaatatatgagtggtaccatagaaacattaccagaaatcaggcagaacatctattgagacaagagtctaaagaaggtgcatttattgtcagagattcaagacatttaggatcctacacaatttccgtatttatgggagctagaagaagtacggaggctgccataaaacattatcagataaaaaagaatgactcaggacagtggtatgtggctgaaagacacgcctttcaatcaatccctgagttaatctggtatcaccagcacaatgcagccggtctcatgactcgtctccgatatccagttgggctgatgggcagttgtttaccagccacagctgggtttagctacgaaaagtgggagatagatccatctgagttggcttttataaaggagattggaagcggtcagtttggagtggtccatttaggtgaatggcggtcacatatccaggtagctatcaaggccatcaatgaaggctccatgtctgaagaggatttcattgaagaggccaaagtgatgatgaaattatctcattcaaagctagtgcaactttatggagtctgtatacagcggaagcccctttacattgtgacagagttcatggaaaatggctgcctgcttaactatctcagggagaataaaggaaagcttaggaaggaaatgctactgagtgtatgccaggatatatgtgaaggaatggaatatctggagaggaatggctatattcatagggatttggcggcaaggaattgtttggtcagttcaacatgcatagtaaaaatttcagactttggaatgacaaggtacgttttggatgatgagtatgtcagttcttttggagccaagttcccaatcaagtggtcccctcctgaagtttttcttttcaataagtacagcagtaaatctgatgtctggtcatttggagttttaatgtgggaagtttttacagaaggaaaaatgccttttgaaaataagtcaaatttgcaagtcgtggaagctatttctgaaggcttcaggctatatcgccctcacctggcaccaatgtccatatatgaagtcatgtacagctgctggcatgagaaacctgaaggccgccctacatttgccgagctgctgcgggctgtcacagagattgcggaaacctggtgaccggaaacagaatgccaacccaaagagtcatcttgcaaaactgtcatttattgtgaatatcttcaccatatggggtcacttatggtgaatatctttcttcagagttgctgactcttgaaaacagtgcaaagatcacagtttttaaaagttttaaaaatttaagaatattcacacaatcgtttttctatgtgtgagagggatttgcacactcttatttttctgtaaaatatttcacatcccaaatgtgaagaagtgaaaaagacttcgcagcagtcttcattgtggtgctcttcatgatcatagccccaggaacccttgaggttcttcttcacaaggctgagagtgcttccttcttgaagacgagtgacattcatcacttcagtgatccatgcatagaatatgaaaataaattcttccaactcatgggataaaggggactcccttgaagaatttcatgtttttgggctgtatagctctttacagaaaatgcacctttataaatcacatgaatgttagtattctggaaatgtcttttgttaatataatcttcccatgttatttaacaaattgtttttgcacatatctgattatattgaaagcagttttttgcattcgagttttaaacactgttataaaatgtagccaaagctcacctttgaacagatcccggtgacattctatttccaggaaaatccggaacctgattttagttctgtgattttacactttttacatgtgagattggacagtttcagaggccttattttgtcatactaagtgtctcctgtaattttcaggaagatgatttgttctttccagaagaggagacaaaagcaagatagccaaatgtgacatcaagctccattgtttcggaaatccaggattttgaattcgagatgaaacaaccagcaatcacagttaaatcttaactttgcctgcactctttgtaggaatgatcagaaatttatctttatcattctgagtgcttcaggagtacaataggaagaaagatactggagaaagcactaatgtaatcaccatgaagtctgacaacaggagcccattatttgcgtactgtcccaccctgtatcatggttctctgggaacaagctttatgattctcattagagtttatttgttgattgtcagtagttgcgacttttaaattatatttcccccactcaaagaatggtatctttatatatcaatgacattcaataaatgtgtattatttctaatgagaa

SEQ ID NO: 30 - DNA, 2741 bp, Homo sapiens
  source 1..2741: mol_type = other DNA
attaaataagagaaaaaagcataaaaggagtaggcttttttggcagatactgttatatgggatggacaggaggggaagacatgaaaatcaactaaaatgaaatacataaaaaatcagtaaagagcctgcttacgaaagtggaaaaacaacagctgggcggggtcgcagggtggcaaacgtacgcgggcacgtgcacgtgcttttggggccgacagacgcgccagttgcctaggcgcatgcgtctggctatcccagaagcacctgcgccttccggctcgtgctttcctcagtctcgcgcctttctctgcagctcgcgcctttctctgcagctcgcgcctttctctgcagctcgcgcctttctctgcagctcgcgcctttctctgcagctcgccccttcctctgcagctcgccccttcctctgcagctcccacctcactcccctcagcgttctttttcccacggtcttcccgttgccgctaacctaactaactctcagccatggcctccaacgaagatttctccatcacacaagacctggagatcccggcagatattgtggagctccacgacatcaatgtggagccccttcctatggaggacattccgacggaaagcgtccagtacgaggatgtggatggcaattggatctacggtggccacaaccatccgccattgatggtgttgcagccgctcttcacgaacacgggctatggcgaccacgaccaggaaatgcttatgttgcagacacaagaggaagtggtgggctattgcgactcagacaaccagctaggcaacgacttggaggaccagttggccctcccggatagcattgaagacgagcacttccagatgaccctggcctctctgtcggcctcggcggcatcaacatcaacatcaacccagagccgcagcaaaaagcccagcaaaaagcccagcggcaagagtgccaccagcactgaggccaacccggcaggcagcagctccagcctgggcacgaggaagtgggagcagaagcaaatgcaggtcaaaacgctggagggtgagttttccgtgactatgtggtcccctaacgataacaatgaccaaggggcagtgggtgaaggccaggctgaaaacccacctgattattccgagtacttgaaagggaagaaacttcctcctggggggttaccaggcattgatctctcagatcctaaacagctggcagaatttactaaagtgaagcccaaaaggtccaaaggagaacctcccaaaacagtcccttgctcttatagcggctgcgaaaagatgttccgggattacgccgccatgagaaaacatctccacatccacgggcccagagtccacgtatgtgcagaatgtggcaaagcttttcttgagagctcaaagctgagacgacaccagctggtccacaccggcgagaagccctttcagtgcacattcgaaggctgcgggaaacgcttttcccttgatttcaatttgcgcacacacttgcgcatccacaccggcgataagcccttcgtgtgccccttcgatgtttgcaacaggaagttcgctcagtcaaccaacctgaaaacccacatattaacgcatgtgaagaccaaaaacaacccgtgaaaaggagaagacccctctcagacttgggaattatcttccaggactgcggtagggaataaatatgcctctcaaagctttgtatgttgtttctaagagttttaaaaaaaaatgaatcctgcacatttaaggttcgtgttttgttagagtagtaaaaatagaatttaaacgtttttaaaaaggtaaaccttgacataagataatagtgctaagatgccatagcttgttctgtaactatttttgtaaagtttggtcccaacaggagaaaaattcgtagacttcacatcaagagacggttcttacaaactgtttaaaatgggacttttcacattcttagaaataggaagttcatttattgtttacaatgtttttaaaaaacttgttaaaaaattcaaagtgttcatgtttatacttttaggaatatgcttaataagtctatgtatggtttttctggaggttgataactttgggaaagatttactttaaaagagtgaacaattatatgcatacgtgaagtattttcctgcttaaaaaagttatataggtgttatttgttttaatcttggttgtagtcttggatgttaacacatcttgcattttagctgtattaggtcatgtagtattgatattaggtgatttaatagtactagtttaaacctattttagtcattttattttccccaaaatactaccagatgctgttgtttagtgtaatttctttgcctgttcagttaaagtagtgcttgcttgtagaatatattgtgtatatgttgactttaacacttaagaagtacatcctgtgtaatagaaaaagcaaaataaaacacctcttctaaagaaggaaaaaagtagtttgcctatatcagtacagaagttagaactaaagaaaagggggagggtgctactggcatctggtgagtggagggagatcaggaatgccactaaacatcctacaatgcacagacagccccacgaaacacataattatttggctaaaatgccaatagtgtcaggtgcaggggctcaggcctgtaatgtcaacactttgggaggccgaggtgggtggatcgtttgagctcaggaattgaacatcagcctgggcaacatggcataactcggtctctacc

SEQ ID NO: 31 - DNA, 6137 bp, Homo sapiens
  source 1..6137: mol_type = other DNA
gtcttttgtccctcggcggacaccgtttgccagccaaagctatgtctgcgcgctcaccgacttcatagggtgccgaattcttttttccccaggcttgccatggctagtcgaggggctcggcagcgcctgaagggcagcggggccagcagtggggatacggccccggctgcggacaagctgcgggagctgctgggcagccgagaggcgggcggcgcggagcaccggaccgagttatctgggaacaaagcaggacaagtctgggcacctgaaggatctactgctttcaagtgtctgctttcagcaaggttatgtgctgctctcctgagcaacatctctgactgtgatgaaacattcaactactgggagccaacacactacctcatctatggggaagggtttcagacttgggaatattccccagcatatgccattcgctcctatgcttacctgttgcttcatgcctggccagctgcatttcatgcaagaattctacaaactaataagattcttgtgttttactttttgcgatgtcttctggcttttgtgagctgtatttgtgaactttacttttacaaggctgtgtgcaagaagtttgggttgcacgtgagtcgaatgatgctagccttcttggttctcagcactggcatgttttgctcatcatcagcattccttcctagtagcttctgtatgtacactacgttgatagccatgactggatggtatatggacaagacttccattgctgtgctgggagtagcagctggggctatcttaggctggccattcagtgcagctcttggtttacccattgcctttgatttgctggtcatgaaacacaggtggaagagtttctttcattggtcgctgatggccctcatactatttctggtgcctgtggtggtcattgacagctactattatgggaagttggtgattgcaccactcaacattgttttgtataatgtctttactcctcatggacctgatctttatggtacagaaccctggtatttctatttaattaatggatttctgaatttcaatgtagcctttgctttggctctcctagtcctaccactgacttctcttatggaatacctgctgcagagatttcatgttcagaatttaggccacccgtattggcttaccttggctccaatgtatatttggtttataattttcttcatccagcctcacaaagaggagagatttcttttccctgtgtatccacttatatgtctctgtggcgctgtggctctctctgcacttcagaaatgttaccactttgtgtttcaacgatatcgcctggagcactatactgtgacatcgaattggctggcattaggaactgtcttcctgtttgggctcttgtcattttctcgctctgtggcactgttcagaggatatcacgggccccttgatttgtatccagaattttaccgaattgctacagacccaaccatccacactgtcccagaaggcagacctgtgaatgtctgtgtgggaaaagagtggtatcgatttcccagcagcttccttcttcctgacaattggcagcttcagttcattccatcagagttcagaggtcagttaccaaaaccttttgcagaaggacctctggccacccggattgttcctactgacatgaatgaccagaatctagaagagccatccagatatattgatatcagtaaatgccattatttagtggatttggacaccatgagagaaacaccccgggagccaaaatattcatccaataaagaagaatggatcagcttggcctatagaccattccttgatgcttctagatcttcaaagctgctgcgggcattctatgtccccttcctgtcagatcagtatacagtgtacgtaaactacaccatcctcaaaccccggaaagcaaagcaaatcaggaagaaaagtggaggttagcaacacacctgtggccccaaaggacaaccatcttgttaactattgattccagtgacctgactccctgcaagtcatcgcctgtaacatttgtaataaaggtcttctgacatgaatactggaatctgggtgctctgggctagtcaaagtctatttcaaagtctaatcaaagtcacatttgctccctgtgtgtgtctctgttctgcatgtaaactttttgcagctaggcagagaaaggccctaaagcacagatagatatattgctccacatctcattgtttttcctctgttcaattatttactagaccggagaagagcagaaccaacttacaggaagaattgaaaatcctggtactggatggctgtgataagctgttctccacactctggcctggcatctgagaactagcaagcctctcttaggccatatgggcttctccaccaaagctgtttggcagctcctagcagaccttcttattgaaatcctcatgctgaaaatgaacacagcctagttgccaacccacatgtccttttcacctccagcaagactaagcttctttaaagcacttcacaggactaggaccctgtcctggagctatctcaggaaaaaggtgaccatttgaggaactgtgacctaattttattataatgatgcctctaattttcatttcctttacaaccaactgtaactataaggttgtattgcttttttgttcagttttagcatgctattttttgaattctagactcctccatgtgaagatatcaacagacaaaactacaactgtataggacatatttggagaaaattctatcaattgatacatttggatgacatcacatttttaagtaatgtaatctgaggccattgctgaggaaattaagaattttcctttttttttaaccacccccagtgaaaaggatcagtgtatatttatagcacctattttttagttctgtctgttgtgaggcacatcctgcatggggcacttctagtcaaataggcaatgataaggacctaattaaaatgtgataagtgtatactattactttaaaagcctttacagtcagtacttcagtttacaaggcactttcacagcatctcgtttgatcctcacagtcacaacatgtggtagacaaggcaggtgatttttatccccattttacagataaggaaacaggctgcgggtggggagtgaggggaggtaaagatagttagttgcctaaggtcacacagccagtaagtaatagagctgggactggaacccaggtttccttactctcatctattgctcctccatattcctcactcaaccatgaaaacattacttgaaaggactgatgaggttaaccagagacctaactgatattgtaactttctattttaaggaagaattgtgtctgtatttgagttctttggagcctccagtctgcctgtgtgttagaccagcacagcagtgctgtgtgatgcagcctgacctgtggcaggaaagtagtgcttctgtttggaagtcatgttcttttgcagccacacaggatccaaatatcagtactattcctgtagtcaatctggggtcacattataggtgccttatttccctaagggtaactgatctgaatatctgcaaataggatgaatctatttttcagaagttccatctttcatttttctttttttttttgagacagagtctcattctgtcgcccatgctggagtgcagtggcgcgatctcggctcgctgcaacctctgcctcccaggttgaagcaattctcatgcctcagccacccgagtagctgggattacaggcatgcgccatcatgcccagctaatttatgtatttttagtagagttggagtttcaccatgttggccaggctggtcttggactcctgacctcaggtcatccacccgcctcagcctcccaaagtgctggtattacaggcgtgagccaccgcacccagccccatctttcattttcaaagagaagggcattctaataggaactggtgccaagagagaagaaaagaagtgataacagaagaaatggctagttacaatattaaaaagctcctctttgagatctcctctgcaggaatatcagagacggagttgaagcgctggagaggtaataggtctagacagtacagaacaataactggggagtgtgtgaggatagactgggctcccccttgcttgaaagatctctggcatttaattctcaattcttgattactattttccagtgtaaaactagcacatatgatctgactacaggacagagaattttaagtgaaacatttgccttacttgcagtaataatgtgctgttcttcacagtagctaaggccctctatgtttcccagaggtaaataagaatccaggaatggaggtccatctgtgatgaatggcttttttctaatcaaagtagtataatgctgttttatctgttttgtcatcttgtttttttttttttttaaaaaaacaaaaccttaattataatatagcgcaaagaaaggccaggactgatgcagggattccttggaaatatcagttcctatcacttttaaaacctgattttggatctctctgttctatgtatgtctttagtgagagcacaatacatggcagaacgctgtgccaaatgttataggtaaggaatatagaaatgaatgttttttgttgtgaaggtgttttcatgtgatattttataaacacattttaaaaaatctccatcactttttagtataggaaggatagctttgcctgggaaaaacagtttcaacacacctgctcagagtagcagttctccctcaaaaaagcagtgttcagcctgcactgactgttctgcttgccaaaaggaggaagcatgcaagatacttatttctccatagattgtggagtatagagggatgtgggactacagattattattttttttccccgagacagagtcttgctctgtcgcccaggttggaacacaatggcacgacctcagctcactgcaacctctgtctcccgggttcaagcaattctcctgcttcagcctcctgagtagctgggattacaggcacacaccaccaccgcactcagctaatttttgtatttttagtagaggtggggttttaccatgttggccaggctggtcttaaactcctgaccttgtaatcatcccgcctcggcctcctaaagtgctaggattacaggcatgagccaccgcacccggcccagataatttttaatagcctttgatcatggggtgagtgagggagtaggtatacttggcaaatgcatggttctctgatttctagctctaaagcagccttatctgaatccccaaatcttgtgatgctgagtaccattactgaaccagtctgcacggtaggcatctgctaccaaaatttacctcctacctggtaggtgtcatctgataagaaagaagacaggttattttaattttttgagataatcacagaaaattgcagcccatactctttattaccgaattcaagtttggaaatagaccctttgttttaaatcatgatgggtctttatcccaatcatttatctgggtcatttttccaactttggagttctaggaaagaaccttgaaaacctgatatgattctgcagcatgaggtctacggtgaccatttgggcaaagctccagtggcaatcatttattgtgttttgcatttcctgggatttattgaaataagaattcactgtgattatgtagtcttctggctagtatcaggcagctctgcttttaatttggttaattttattttctctgaagagggagaagaggtacaatttaatcttggcctccacaagcatattaaagctcacgtgttaatcagtgcattcttatgctcctacattaaatgccttgggtaaatggataaatggacatgtgcccagctttaattttttttgcaacagaaagatcagacttccgtatggcatcgttggatttcagaggctttctggtgtatctgtaaatctgaatgttgccttctgccagtctgtataaccaggtgattcatgctgcaaatgaaatcaggaagcagtaaagtgttaaagcaagagtattgtccaattcacttgtcttcctgatccttgtactttatttcacgtgtcggtgtttacattacatacttatatttcctgtgaaagaaagagttaaataaattgtagcagtttga

SEQ ID NO: 32 - DNA, 3531 bp, Homo sapiens
  source 1..3531: mol_type = other DNA
agcggagcgccaggcagcgcggagcggaggccaggcccacagccgctccgcctcccggcccgcagatccccgacggccgcaccgcgggctcctctggcccgcaagaacacgtgcatggcgtcctggggaaggcgctgagtgcggagtcgcggcgccgcacgcggcaccatggccctggagcaggcgctgcaggcggcgcggcagggcgagctggacgtgctgaggtcgctgcacgccgcaggcctcctggggccctcgctgcgcgacccgctggacgcgctgcccgtgcaccacgcggcccgcgctgggaagctgcactgtctgcgcttcctggtggaggaagccgccctccccgccgcggcccgcgcccgcaacggcgccacaccggcccacgacgcctccgccaccggccacctcgcctgcctgcagtggctgctgtcgcagggcggctgcagagtgcaggacaaagacaattctggtgccacagtcttgcatctggctgcccgcttcggccaccccgaggtggtgaactggctcttgcatcatggcggtggggaccccaccgcggccacagacatgggcgccctgcctatccactacgctgccgccaaaggagacttcccctccctgaggcttctcgtcgagcactaccctgagggagtgaatgcccaaaccaagaacggtgccacgcccctgtacctggcgtgccaggagggccacctggaggtgacccagtacctggtgcaggaatgcggcgcagacccgcacgcgcgcgcccacgacggcatgaccccgctgcacgccgcggcgcagatgggccacagcccagtcatcgtgtggttggtgagctgcaccgacgtgagcctgtccgagcaggacaaagacggcgccaccgccatgcacttcgcggcgagccgcggccacaccaaggtgctcagctggctgctgctgcacggcggggagatctcggctgacctgtggggcgggaccccgctgcacgacgccgccgagaacggggagctagagtgctgccagatcctggtagtgaacggcgcggagctggacgtccgcgaccgcgacgggtacacggccgccgacctgtcggacttcaacggccacagccactgcacccgctacctgcgcacggtggagaacctgagcgtggagcaccgcgtgctttcccgggatccatccgcagagctggaggctaagcagccggattcaggcatgtcctcacccaataccacggtgtcggtccagccgctgaactttgacctcagctcgcctaccagcaccctctccaactacgactcctgctcctccagccactccagcatcaagggccagcaccctccatgtgggctttccagcgctagagctgcagacatacagagctacatggacatgctgaacccggagctgggcctgcctcggggcacgattgggaagcccacacccccaccacccccacccagcttccccccgccacccccgcccccaggcacccaactgcccccacccccacctggctacccagctcccaagcctcctgtaggaccacaggcagctgacatctacatgcagaccaagaacaaactccgccacgtggagacagaggccctcaagaaggagctgagctcctgtgacggccacgacgggctgcggaggcaggactccagccgcaagccccgcgccttcagcaagcagcccagcacgggggactactaccggcagctgggccgctgccccggcgagacgctggccgcacgcccgggcatggcgcacagcgaggaggtgcgtgcccgccagcccgcgcgcgccggctgcccgcgcctcggccctgccgcccgcggctcactcgaaggcccctccgctcccccgcaggcggcgctgcttcctgggaaccatgttcctaacggctgcgccgcggaccccaaggcgtccagggagctgccaccgccgcccccaccgccgccgccgcccctgccggaggccgcgagttcgccaccgccggccccgcctctgcccctcgagagcgctggccctggctgcgggcagcgccgctcctcctcgtccaccggcagcaccaagtctttcaacatgatgtccccgacgggcgacaactcggagctactggctgagattaaggcaggcaagagcctgaagccgacgccccagagcaaggggctgaccacagtgttctcaggcatcgggcagccggccttccagcccgattcgccgctgccttctgtgtcacctgcactgtcaccagtccggagccccacaccgccagctgcggggtttcagccgctgctcaatggaagcttggttcccgtgccgcccactactcctgcgccgggagtgcagctggacgtggaggctctcatccccacgcacgatgagcagggccggcccatccccgagtggaagcgccaggtgatggtgcgcaagatgcagctgaagatgcaggaggaggaggagcagaggcggaaggaggaggaggaggaggcccggctggccagcatgcccgcctggaggcgggacctcctgcggaagaagctggaagaagagagggagcagaagcggaaagaggaggagcgacagaagcaggaggagctgcggcgggagaaggaacagtcagagaagctgcggacgctgggctacgatgagagcaagctggcgccctggcagcgacaggtcatcctgaagaagggggacatcgctaagtactagaggccgcagactcctgtccgcagcctcgcagctccgtggggccctccgccccagccccagccagccaggccctggtggaaaggctgggagccgcacagccctcccctcctgcgctggaaaccctccctgacccccaccctggccccccgtatccccagcccttggcaacactggagtgcacacgccgccacggttgcccagaaaaagtgcccaagctgctgacgcaaacaacaacaaatgctgcttatttgcatgccgacttacatatatttgcatgttcgttgactatcaaagagtgcagagctctccccagccccgtgggtggtgactttgttttcctgcggggctcagccccctccaggatgcagccccctcccccgcaccccggaaccggcgtcgctggcgcatcctgggtggaggcaggccccgagctcggggaaggggttttcccttcctctctgacccagatctgcgcgcggcctagcccgggcctcatttcttatccccgccaagggtttcctctcagtcatttgtttaccagaaacatgaaaactgcctgtctggccgggccgcacttgtggcccccgggaccccacctctggccccacctccctcaagtctgcgccccgtccccagccagacccactcgctgccgggaccctttcactgccccggtggagtgaatagaggatgaggggccctgaccctgtgtctccaactgctgcaccccatcccgaccctgtctccgccacctcgcagccccattaaagcgctctcatctgggctccggttcactca

SEQ ID NO: 33 - DNA, 2272 bp, Homo sapiens
  source 1..2272: mol_type = other DNA
ttgggcggtggaccgcccctcggccccggggtaggctgacacgggagggtcctcagctaaagccaaaagcagatcaaagtggtgggactcgcgtcgcggccgcggagacgtgaagctctcgaggctcctcccgctgcgggtcggcgctcgccctcgctctcctcgccctccgccccggccccggccccgcgcccgccatggagaagactgagctgatccagaaggccaagctggccgagcaggccgagcgctacgacgacatggccacctgcatgaaggcagtgaccgagcagggcgccgagctgtccaacgaggagcgcaacctgctctccgtggcctacaagaacgtggtcgggggccgcaggtccgcctggagggtcatctctagcatcgagcagaagaccgacacctccgacaagaagttgcagctgattaaggactatcgggagaaagtggagtccgagctgagatccatctgcaccacggtgctggaattgttggataaatatttaatagccaatgcaactaatccagagagtaaggtcttctatctgaaaatgaagggtgattacttccggtaccttgctgaagttgcgtgtggtgatgatcgaaaacaaacgatagataattcccaaggagcttaccaagaggcatttgatataagcaagaaagagatgcaacccacacacccaatccgcctggggcttgctcttaacttttctgtattttactatgagattcttaataacccagagcttgcctgcacgctggctaaaacggcttttgatgaggccattgctgaacttgatacactgaatgaagactcatacaaagacagcaccctcatcatgcagttgcttagagacaacctaacactttggacatcagacagtgcaggagaagaatgtgatgcggcagaaggggctgaaaactaaatccatacagggtgtcatccttctttccttcaagaaacctttttacacatctccattccttattccacttggatttcctatagcaaagaaacccattcatgtgtatggaatcaactgtttatagtcttttcacactgcagctttgggaaaacttcattccttgatttgtgtttgtcttggccttcctggtgtgcagtactgctgtagaaaagtattaatagcttcatttcatataaacataagtaactcccaaacacttatgtagaggactaaaaatgtatctggtatttaagtaatctgaaccagttctgcaagtgactgtgttttgtattactgtgaaaataagaaaatgtagttaattacaatttaaagagtattccacataacttcttaatttctacattccctcccttactcttcgggggtttcctttcagtaagcaacttttccatgctcttaatgtattcctttttagtaggaatccggaagtattagattgaatggaaaagcacttgccatctctgtctaggggtcacaaattgaaatggctcctgtatcacatacggaggtcttgtgtatctgtggcaacagggagtttccttattcactctttatttgctgctgtttaagttgccaacctcccctcccaataaaaattcacttacacctcctgcctttgtagttctggtattcactttactatgtgatagaagtagcatgttgctgccagaatacaagcattgcttttggcaaattaaagtgcatgtcatttcttaatacactagaaaggggaaataaattaaagtacacaagtccaagtctaaaactttagtacttttccatgcagatttgtgcacatgtgagagggtgtccagtttgtctagtgattgttatttagagagttggaccactattgtgtgttgctaatcattgactgtagtcccaaaaaagccttgtgaaaatgttatgccctatgtaacagcagagtaacataaaataaaagtacattttataaaccatttactatggctttgtaacaattgcatacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgatatctggttaagtgttatatggaataaagtggacattttaaaactagcaaagttaaaaaaaaaaaaaaaaaaaa

SEQ ID NO: 34 - DNA, 4510 bp, Homo sapiens
  source 1..4510: mol_type = other DNA
gcagagggggcggagagcgcccccgggggcggggcacgcaagtgacggcggcgcgggtggtggagcgctgggcggccaggctccctggctggccggtttgggcgtctgggccgtgaaggtgggacctcctgttccgggccgcaagtttccctctccagccgcccgccgttcgtagcatgtcccccagaactcggggagcgcaggcaggacaggcttagagaagacgcggtccccagcgcttgggccacggacgtcccaccccgctcctctgtcgctggagaaccgccgggccgagccactgggagaagcaggccagagccttccagggcctccggcccgtggacccgaggaggatgagctggctttttcccctgaccaagagcgcctcctcctccgcggctgggtcccccggtggcctcaccagcctccagcagcagaagcagcgcctgatcgagtccctccggaactcacactccagattgcttcctccacagtttcctcaggaaaaaccagtgatcagtgtttatccaccaatacgacatcacttaatggataaacaaggagtgtatgttacctctccattagtaaacaattttacaatgcactcagatcttggaaaaattattcagagtctgttggatgagttttggaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactatagattttcctggaaacatgaactgccaagagaggaatgggacacaaaaccaaacactgttttatatttatggtttgcaaactggcatttcatcagtggctaaattcacagatatcctatatagattgtatacagaactgagactgattttgtaccgattagaatgattgctatgatctttgagaaatttttctgcactatttgcactgaaatgtttatttattgttgataaattgtatcatatttaagttccactgctgttcctcttaccttgattaaatgcctatgcatgtacttttagctagtttttaatattttataaaacttcatttaaatttgtatttttaacttgaagttccatttctttatcaaggatggtatttagatttttttcctcttaaccttttttcaaaaactattttcaactgtgaggaaacccttatttttctttctttgtggataaaactttcaaaagcaatttaagatattcatagtgttaggaaacaccaaacctgcctatgtgccatctcacaaaagaaacttttaatacctacaataaatcaaaagaataaaccagctgttcttatatattgtttcatttttaaaactaaagatgcatttaagaagcaatacaagtaaatattttacctaataggaaaaaaaaaagttgcctttcatttaaaccattccaacagaaattcttatgctaatttaaaacatatatatatctggtaggtttgtggttggataggttttctaaattcctaatgttaaaaacaatctttatgttaatatacactaaatctatacacaaaaaaagtcagtgaacttttctgacctttactgtgagttaccttttcctaagaggaaagctatagtaataagtaaaatttaatttttaggcaatcctgatttttaatgaatttaattgagtgttcttgtatactacattgagcagtttgcttctataccgtgtcacaaaattcatgtatttcttgagaagccctaaaagctcataaaggaaaatgccgtgaactatgtagctcaggcttggtaaggtgccatctaaattacaaaacaaactaatgcataattttgcttaaatttcatcccagtatgattgtcttcccaacaccagcatatagtatagattgtctgtcttttttatattttttagttcttcctgtacatgtttttggcaataaagttataggaagaacaaaattattttgttagaattaaaacatgcttaatatttagtctgtttgtggagggcaggtattcacgtggactgagatacaatgttggatacagaaaataactttcattgtcttcctgacactgtgctaaggacatgctgttaaagcttcaaagtgaccagatgaggaaggaataattaattattactcctgatttgtagataactgaggtaagagtgtttcaaatttatgatagtcttttgggtattcagaaacctttccttatactgcactggccaccagagcttaattttcccagcagttacagcaatgggagatagaacagtctcaatcttttgccaaccatcaggttcctagaaaccaggtaggtgtatcccataacaagggaggagcataccacagcccctcatttgattaattcatttgatctatctatgttattaagtacctactaggaataaggcattgtggaaatactatacaaagataaacattgtttagatgcttatctactttccttttcaccagaaaaacagaaaaaaaagaaacattttcttacagagtaaaaatgttctacataatcacatgagtagttcatctcagtgttttttattctttaaagttgaactatcccagtttcattctataccattcattggataaccttgttacaacccagtcatgaaacagagcagtgtgatcagttatctgcatttaacaaatagacaaatcagtttacataaaggttatgtatgtcacccacgatgaaaagaatctgcatttgaatatgcccgtatgaatgtgggttctgtttttgcaacagagattaagtgaccattttttctaattttatggctatatattttcttcataaaaattggtcacatcggagaagcagtgccacaggaaaaatgaaatgcatgtgaaagtttgtattctgattttacaagatgagatagaaatcagaattaaagaggaatacttaggagttactaggctaatcagtgtacgaatttgtcataggtagagatttaaaggttaatatcttaaaatagaagaaaattctaaatcaatcaatcagtgagatataaactaaacagacccacttcaaagttgaaagaaatttctaggcataaattgagactaggaaatttatatcagaatagagggtgcttgacacatatatatgcttaaattgaaggacagctcagattcatttttaggagaagaaagtaaactaatgtgctcttaaagaataaaaatttattctatggtttctgtctctgatcatcaccttccattctataaaaagctcagttactgatttgctgggtcatggtcaaaattcttacctatttatttcatatcaactttaaaaaataaattacttgcattctatatattactaattgggaagtaatatgcctcaaatcagttttatactggattattccctatgctttaaaccactgctctcaataaaacacttcctgattaatgtttgattattagatattttagtcttgttggggatattttagtcttgttgggttagccatgctctgaagaatctgtgaaagtacagtaaagttttaataagcaataaatgtaaccttttatataaatctcagtgctaggttaacttctaataagcagacgaacatgttacataaattataatgtctgtcttgtaaaaaagttgaggggactaaaagtttatgactctgatatggaagttgtcatattaaaaaactacattttaaaacatcaaatatttatactatttgcttttcaaataaaagcatagtgctgtttggcata

SEQ ID NO: 35 - DNA, 11062 bp, Homo sapiens
  source 1..11062: mol_type = other DNA
gcagatcgggagcggtgccgagaaaaatttccttactagatgacatttcatcgcaatgtccgatcgtttggggcaaattaccaagggcaaggatgggaaaagcaagtactcgactctcagcctgtttgataagtataaaggaaaatcagtagacgcgattagatcctcagttattcctagacatggcttacagagtcttgggaaagttgctgcagcccggcgcatgccaccgcctgcaaacctgccaagcttgaagtctgaaaacaaaggaaacgaccccaacatcgtgatagtacccaaggacgggacgggatgggcaaacaagcaggatcagcaagacccaaagagttccagtgcgacggcctctcagccgccggagtcgctgccgcagccgggtttgcagaaatctgtctccaatttgcagaaaccgacacagtcaatcagtcaggagaatacaaattcagtgccaggtggaccaaagtcatgggcacagctgaatggaaagccagtaggacacgaaggtggtttaaggggctcaagccgactgttatccttctctcccgaggaatttccgacgctgaaagcagctggagggcaggacaaggctggcaaagaaaagggcgtcttagatctgtcgtatgggccaggaccaagcctccgccctcagaatgtgacaagctggagggagggcggtgggcgacacataatttctgccacgtctctgagcacctccccaactgagctgggcagcaggaactcgagtacgggagatggagccccctcctcggcatgtaccagcgattctaaggacccctctctccgcccggctcagcctgtccgaaaaggggcttcacagttcatgggaaatgtataccacccacctacataccatgacatgcttcctgcttttatgtgttcgccgaagtcatcagaaaaccagggtacagtggaacgaggctcttttccccttcctcagctccgccttgaacctcgagttccttttagacagttccagatgaatgaccaagacggaaaagaaaacaggctgggattgtctcgcccactccgcccactaaggcagctggtggagcgggcaccacggcccaccattatcaatgcggaaaacctgaagggccttgacgatctggacgccgatgccgatgatggctgggcaggcctccatgaagaagtggactattctgagaaactgaagttcagtgatgatgaagaggaggaagaagttgtgaaggacggcaggccaaagtggaacagttgggaccctaggaggcagcggcagttgtcaatgagctctgcagacagtgcggacgctaagcggactcgagaggaagggaaggactgggctgaagcagtgggtgcgtcccgtgtggtccgaaaggcgccagaccctcagccaccgcccaggaagcttcatggctgggcaccaggccctgactaccagaagtcatcaatgggcagcatgttccggcaacagtccatcgaggacaaggaggacaagcccccaccaaggcagaagttcattcagtcagagatgtccgaggcggtggagcgagcccgaaagcgccgggaagaagaggagcgccgagcccgggaggagaggctggccgcctgtgctgccaaactcaagcagctggaccagaagtgtaagcaggcacgaaaggcaggtgaggcccggaagcaggcagagaaggaagtgccctggtctccaagtgctgagaaggcatctccccaggaaaacggccctgctgtccacaaaggctccccagaattccctgcccaagagacccccaccacattcccagaagaggcacccacagtgtccccagcagtggcacagagcaacagcagtgaggaagaggccagagaggctgggtcccctgcacaggagttcaagtatcagaagtcccttcctccccgattccagcgccagcagcagcaacaacagcaggagcagctgtacaagatgcagcactggcagccggtgtaccccccgccgtcccacccccagcgcaccttttacccacaccacccccagatgttgggcttcgatcccaggtggatgatgatgccttcctacatggacccacgtatcacgcccactcggaccccggtggacttctacccctccgccctgcatccctcaggactgatgaagcccatgatgccccaggagtccctcaatgggacaggctgtcgctctgaggatcagaactgtgtgcccccactccaagaaagaaaagtgacccccatcgactcaccccctgtgtggagcccagagggctacatggcactgcagagcaagggctacccgctcccgcacccgaagtcgagtgacaccttggctatggacatgcgtgtcaggaatgaaagctctttctctgcctcactcggaagggcagggggcgtaagtgctcagcgcgatctctttgaggagagaggggaggagtacttgagtgcttttgacaagaaggcccaagcagactttgacagctgtatctcttctcaaagaataggccaggagcttttgtttccaccccaagaaaatgttcaggatgcaggtgctcctgggggtcacacccaaaacctcaggtgttccccattggagcctgactttgtcccagatgagaaaaagccagagtgtggcagttgggatgttagccaccagccagagaccgctgacacagcccatggtgttgagcgggagacaccccgggaggggacggcctttaacatctcctcctgggacaagaacgggagccccaacaaacagccatcctcggagcctgaatggactcccgagccccggagctccagcagccagcacccggagcagacgggcaggacccggaggtcgggacccatcaagaaaccagtcctgaaagccctcaaggtggaagacaaggagaaggagcttgagaagattaagcaggagctaggggaggagagtacccggctggccaaggagaaggagcagagccccacggcagaaaaggatgaggacgaagagaacgatgcctctctggccaactcctccaccaccactttggaggacaaaggccctggccatgccacttttggccgcgaggccaccaaatttgaagaggaggagaaacctgacaaggcctgggaagccagacccccacgagagtccagcgatgttccccccatgaagagaaataactggatctttattgatgaggagcaagcctttggggtcagaggacaggcccggggccggggccgtggtttcagagagttcacttttcgtggtcggcctgctggcggaaatgggagcggcctctgtggtgggggggtcctgggggcccgcagcatctactgcagcagtcagcgcagcggccgtggccggggcctgcgagagtttgcgcggccagaggactgccccagagccaagccccgacggagagttgccagtgagacccatagcgagggctcagagtatgaagaacttcccaagcgccgccggcagaggggctccgagaacgggaatgaaggctcgctcctggagagggaggagagcaccttgaagaagggcgactgcagagattcttggcggtccaacaaggggtgctctgaggaccacagcggtctagatgccaagagccgaggccctcgggcctttgggcgagccctccctccccggctgagcaattgcgggtatggacggagaaccttcgtctccaaagagtcaccccactggcagagcaaaagtccaggcagctcttggcaggaatatggcccttccgacacatgcggatcccggcgacctacagacagagactatgtcccagattcctacagacaccctgacgcatttggtggccggggctttgaggacagccgcgcggaggacaagagatccttcttccaagatgaacacgtggcagattctgaaaatgcagagaaccggcccttcaggagaaggcgccccccacgccaagataagccccctcgattccggcgcctccggcaagagcgggagtccctgggcctgtggggacccgaggaggagccccacctgctggcaggtcagtggccaggcaggcccaaactgtgttctggggacaagagtggcactgtgggccgcaggtcccctgagctctcctaccagaactcctccgatcacgccaatgaggagtgggagacggcctccgaaagcagcgacttcagcgagcggcgggagcggcgggaaggccctgggtccgagcccgactcccaggtggatggtggcctgtcgggggctagtttgggtgagaagaaggagctggccaagaggagcttctccagtcagagacccgtggttgacagacagagccgaaagctggagccgggagggtttggggagaagcccgttaggccaggtggtggtgacacctcccctcgctatgagagccaacagaatgggacgcctttgaaagtgaaaagatccccagacgaggccttgcctggaggtcttagtggctgcagcagtgggagtggccactccccctatgccctggagcgggcagcccatgccagtgctgaccttcccgaagcctccagtaaaaaggcagagaaggaggccaagttggctgctccgagggcaggtgaacagggagaggccatgaaacagtttgacctgaactatggaagtgccatcattgaaaattgcgggtccagccccggggaggagagtgaggtgggttctatggtgggcgaaggcttcatcgaagtcctgaccaagaagcagcgccgcctgctggaggaagagagaagaaagaaggagcaggccgtgcaggtgcctgtcaaaggtcgaggcctttcctcccgtattcctcctcgatttgcaaaaaagcagaacaacttatgtctggagcaaggtgacgtgaccgtgcctggcagcagcctgggcactgagatctgggagagcagcagccaggctctccctgtgcaggccccagccaacgactcctggaggaaagctgtcactgccttcagcagcaccgagactggctctgcggagcagggttttaagagcagccagggagatagtggcgttgacttgagtgccgagtctcgggagtcgtctgcgacctcctcgcagcgcagctccccatatgggactctgaagccagaggagatgagcgggcccggcctggcggaacccaaggccgacagccacaaggagcaggctccaaagccatctgagcagaaggattcagaacaaggctctggacagagcaaggagcacagaccaggacccatcggcaacgagcgttctctgaaaaacagaaagggctcggagggggccgagcggctgcaaggggctgtcgtcccgcctgttaacggggtggagattcacgtggactccgtgctgcctgtgccacccattgaatttggagtcagtccaaaagactccgatttcagcttgccacctggttctgcctctggtcctactgggagtccagttgttaaacttcaggatgccttggccagtaatgcagggttaacacagagtatccccatcctgcggcgggaccatcacatccagagggccatcggtctctccccaatgtccttccccaccgccgaccttactctgaagatggagtctgcgcgcaaggcttgggaaaactcccccagtttgccggagcagagctctccaggcggcgctggctcaggcatccagcctccatcctctgtgggtgcctccagcggggtcaactacagctccttcggtggagtgtccatgccacccatgcctgtggcctctgtagcaccttctgcttctatgccaggcagccacctcccgcccctgtacctggatggccatgtgtttgcaagtcagccccggctggttcctcaaacgatacctcagcagcagagttaccaacaggccgccgctgcccagcagatcccgatctcccttcacacatctctgcaggcacaagctcagcttggactgaggggtgggcttcctgtgtcccagtcccaggagatcttcagctccttgcagcccttcagatctcaggtgtacatgcaccccagcctgtcaccgcccagcaccatgatcctctctgggggcacagccttgaagcctccatactcggcgttcccaggcatgcagcccttggagatggtgaagccgcagtctggctcaccctaccagcccatgagcgggaaccaagccctggtctacgagggccagctcagccaggctgctggcctgggtgcctcccagatgttggactcccagctcccacagctgaccatgccactgcctcggtacggctccgggcagcagccactgatcctgccccagtctattcagctgccacctgggcagagcctctccgttggggccccccgaaggattcctccgcccgggtcccagccgccagtcctgaacaccagcagagagccctctcagatggagatgaaaggcttccactttgccgacagtaaacagaatgtcccttcaggaggccccgtgccatcgccacagacctacaggcctagctctgctagccccagtgggaagccctctggatcagcagttaacatgggctctgtgcagggacactacgtgcaacaggcaaaacaacgagtggatgagaaacccagcctgggagccgtgaagctgcaggaggccccctcggctgcctcccagatgaagcgaaccggagcgatcaagcctcgggctgtcaaagtggaggagagtaaggcctgacagtgcctggctgccacctcgcctctccctactgaggacggtgccgccatgcggcctcgacacagccgacactcgggagcctcaccagatccaccgtccaaatgcgtggcccagactgagagacctccctcctctccactcccgaaagctccgttgtcaaccagcttgcacccgtggatatatggcattgacccgcttgctttgatacgaaacaaaaaagcagacgactccttcatcccatctgctcctaccgtgactgtggagtgacgcctcctgtgcagtgcagatttgccctccctgcctcctccctgtcctgccgcgcagccagggcgccttctcagcagtgcttccggcccagccgcccatccctaggcacagtgatttggcagcagggtcattttactttgaggctttttgttttaaaatgtagccaaggtttttacaaaggggaaaggaaaagaaaacaaaaacgcaagctccatgtgtatagctgaacttttatatgtttcttgccagcccctccgctcccttccatctctagcctctgtcctgtttagtttgatacgtcactgcagtaccttaagaggtgactcttaagaatgcatcccctcctgattcctcagctggttcacccttgaggttatttgcaaaaagaaaaggaggttcttgagggcaccgattgcgagcattctggtgcctggctccccgcctgggaagcgatggggtgctcagagcagcaggcaggttgggggagggggggggtcatagttgggttccagctcctggcttgatgagcccagggcgcttacaggcagcccatgaagttgatgacagttttagcatgagaatcacacagggtccctgtcctgggctcctctaaagccagtggatgtgctgggcaccagagacaaatcatggagatggctgctggtggctcccaggttggcccagatggggtgagctgacataccacaggcccatcccaggccccgtgggctctgcttctggggctccataccctgccctgcaggggtgctgtgtttttcacacatttctttccctgaagccttctgtaacctgtcattttccttccttcctcttccggagcctgctgctttctctggacctgtctccacctcccacacagctcatcgtgaacaccacttggtgatggagggagtggacccgtgtgtggtccccaagtgaggccactgggagtttgtccttttcctcctttgcttcactcccagcagcagacccaggttgtcaggacaggagggcctgagctaagcagtaggcatcagtctcgtttgtcttcagacggcgggggcaggtccagggtgaggctgggtggagggctgaccaaggtccaaagggcctgcgcagcctccgggagggcagcttctccagccagaggcttgtgtgagccatcgtgtgctgggcttgtttttaagtaagaaacaaggaaatcactccagattctgtcattccaaggaaagggaaggggacagttcaggtttctcagctgttcttaggggtcactgagcgtctacctcctcctccagaggaggctggctcagaacacctagaggagggggccggggatgcaccccccaccagaggctgccttcagcgtctcacgggtgcaggacagcgctcaggcttgggctctaagctctgtgtctagtgtagaacatggggaaggagcatcttaggaactgctgaagtaacttcttactgctctcacaattctaaggaagcgggagaacggcctcctaccaacagcgcccaccccagagctgcctgggaaagggcagttttactgaaaggtgctttactgttcacctgcatctttcagcagctcccctcctgccctcacctggtcttttccctctttatcccaagcctttatgcttgagtcccttccccaggggctgcccacccgacagttccaggcattccctacctgagcttcttgtctgcttttccttctcccactgcaagcggctgcttgtggggcctgggatgagccctctctgtccccaccggccctccttgccaagccattcctgggtgagttcaggcctgcgggagccacacattcatctccacctggacacttgagccgcatggccagacccctcccacctgatgcggtggtgcgtgtgatttgtcaaaagaaagccttctggatgctgttaagatgtacccttcaggtgaacctggtatcagacccacagtacttgctgtttgagaaaaaataaaaacaaaaaggtcacctgttctccagcccttttctcttacctggtatttccttcctttctcctcccccaccccaaataaaaaaacaaaaaacactagaatttatttatatgtattgatgttgtaggtctaggtgaaaaaaaaagaagtaaatgtttcactgctctatttatatataatgtctgaattaattctgtgcaggaaaggccaggaaattgcatgtgaagttcggtgcagtcaccacctgtgtgtgacctgagctgcagtctcttcgctgagatgcaggttttaaatgagacttggggggctgagggcaggcctcaggcctcccagcgccccaacccctccttggtctaatgaaatgcagttcttagtgcagagatgttttaaggtgcaatatatctcttcctttcccgtggttttagagccaagctcaaggtagtaggacgtagggtcttattttgttttcaaacccccatcctcagagcgcagatacatgcagaggcttctgccaggataccacggggccttagtgggaacaggtggagaccagcacttccctttcctgctgctgaggtagggattggggggtcagaacccactcacttttgcctgttaaagttgccctcctgacgctggcagctctgccttggtcactggggatgcggctcgttgctcagccaccagtggccttgcggtattgtccaccatccactagagtgggatgaagtccagagtgtgggtatacatctcagatgcccatctacccactggggacttcaatgccagctgcatttggtttggttttcttaactgttggcttctccccacagcgttttttgtttttttttaaacattcatattgttttcaaacttggaattcatagacactctggctctaggttccttaagggggaaaacaaaagatgactttatttcacattcaagaaaatcagttcagttccaaagctgtggtccttccagccacttctagggacactggggaaccttgttaaacgttgacatcagtgctctccagccgtgctgtcaccctcctatcttctggatctgccttcgcgatggtcagtgacagcttctggaagctgagcacacacaggtgcacagccatgctgtggtctggcctgctacggcagcatggcagctctggtggagccttctcccttgccatttggttcccctgtgccaagtagctgcaggctgcccctcaaatcttcatttgtcccttttcacttcctgcagaacaagcctgggttagagggtctgctggaaatggcctttgaagaccaaggataccaggatgtgtgcactctgtcgtgttctgtgatgaatgggaaacgtaggcttccagaaagccagctctcttctgaaatgtgacggacctaagcaggaagtcatccaggacaggagtggctcagtgttggggatggacgctgtcgcccagccatgctccaccagggccaccaatgtgtagttggctggtggtcttcgggcatgtgagacctgctcttcactgtttccaccccacttggtggcctccaggatggtagtggcaccctcagagccccatcttcagcatgttctgaagcctcagagtggaaattcctgctaaggctctgtgtggacgcctttctcccgtgatctaaaggggacactgtactcaagcttttgacctcatgccttgtgtagtaaaaaaggatttgggggttttgtttggttcctgagagggttgtgttttgtttttgtttccttttgtttatgttttggcctttcctctttgtctttccatgtagaccagatatttgaaagggcagacgatggctagaggtgtaatgtgcagcttgtttatacggtattttgggaaacttaccttggatgggaaatcgaatcgtggattcaccaggccggtgctggcacactcaccctcgccctttccctccggttcagtacctattgtttctcctttcaaatatgtgattgtactagctctttccatatgaaagaattctccttatttaaataaaaaaagtttaaaaa

SEQ ID NO: 36 - DNA, 7689 bp, Homo sapiens
  source 1..7689: mol_type = other DNA
tcccacagtgcctggcccagaagccttgctaaatatttgaacaggattgcccaatacttttctgctgtgagaatgtaagatggatccagaagagcaggagctcttaaatgattacagatacagaagctactcttcagtgattgaaaaggctttgagaaattttgagtcctcgagtgaatgggcggatctcatatcttcacttggcaaactcaacaaggctcttcagagtaacctgaggtactccttgttgccaagacggctcctcatcagcaaaagattagctcagtgtttgcaccctgccctgcccagtggtgtccacttaaaagctctggaaacctacgagattatctttaaaatcgtggggaccaaatggctggccaaggacttgtttctgtacagctgcgggttatttcctctcctggcacacgcggcggtgtcggtgaggccggtgctgctcaccctgtacgagaagtacttcctcccactgcagaagctgctcctgcccagtctgcaggccttcatcgtgggcctgctgcccggccttgaagagggctccgagatctccgacagaacggatgctctgctcctgagactgtcgctggtggttggcaaagaggtgttttacaccgccctctgggggagcgtcctggccagcccgtccatccgcctccctgcctcagtcttcgtggtgggccacatcaacagggatgcccccggccgggagcagaagtacatgctggggaccaatcaccaactcacggtgaagtctttgcgtgcctccctgttggactcaaatgttcttgtgcaaagaaataatctggaaatcgttctgtttttcttcccattttatacctgtctggattccaatgagagagccatccccctcctcagatctgacatcgtgcgcattctctcagccgccacccagaccctactgagaagggacatgtccctgaacagaagactgtatgcatggttactaggctcagacataaaaggaaataccgttgtgccagaatctgaaatctcaaattcttatgaagaccagtcgtcttatttttttgaaaaatactccaaggatcttttagttgagggtttggctgagatattgcatcagaagttcatagatgctgacgtggaggaacgccatcatgcatacctgaagccttttcgcgtcctcatcagtctgcttgacaagccagaaatagggcctcaagtggttgggaatttgtttctcgaagtcatcagggccttttattcttactgcagagatgcccttggctctgatcttaaacttagctacacccagagtggaaattcgctgataagtgcaatcaaggaaaacagaaatgcctctgagattgtcaaaacggtaaatttgctgataacttctctaagcacagactttctctgggattatatgacaaggtgttttgaggaatgctttagaccagtgaagcagcgttacagcgtgaggaacagcgtcagccctccccccacggtctcggagctctgcgccctcctggtcttcctgctggatgtcattcctttggaactttactctgaggtgcaaacccagtatctccctcaggtgctcggctgcctggtgcagcctcttgctgaggacatggaggccttaagtttacctgaactcacgcatgccttgaagacgtgtttcaaggtgctcagcaaagtccagatgcctccttcctacctcgacacggagtccaccagcggaacctcgagtccagtaaaaggtgaaaacggcaaaataattttggaaacaaaggcagtgattcccggtgacgaagatgcttcgtttccccctctgaagtctgaggacagtgggatcgggctcagtgcctcgtcaccggagctctctgagcacttgagggttcctcgagtttctctggaaagggacgacgtttggaagaagggcgggagcatgcagaggacgtttctttgcatccaagagctaatcgccaactttgccagcaagaacatttttggagtacagctgacagcgtcaggagaagaaagcaagtccgaggagcctgcagggaagagggacagggatgggacgcagagcctggcagccaatgattccagcaggaagaactcttgggagcccaagcccatcactgtgcctcagttcaagcagatgctgtcagacttgttcacagcacgagggtctccattcaagacaaaaagttcagagtcaccatcgtcttcgcccagcagccctgccaggaaaaacgggggagaatgggatgttgagaaggtggtcattgacctggggggttccagggaggaacgcagggaggcctttgccgccgcctgccacctgctgctggattgtgccactttccctgtctacctgtccgaggaagagaccgagcagctctgtgcaacgctcttccagctgccaggagccggtgattccagttttccatcttggctgaagtccctcatgactatttgctgctgtgtgactgactgctacctccagaacgtggccatttccactctgctggaagtgataaaccattcccagtccctggcgcttgtcattgaagacaagatgaaacgctataagagctctggacacaaccctttttttggcaagctgcagatggtgacggttcctcccattgctccagggatattgaaagtcattgcagagaaaacagatttctatcagagggtggctcgtgtgctttggaatcagctgaacaaagagacccgggagcatcacgtcacctgcgtagaattgttctaccggctgcactgcctggcccctacggccaacatctgcgaggacatcatctgccatgccctcctggaccctgacaagggaacaaggctggaagctctgtttagattttccgtgatctggcatctgacaagagagatccaaggcagtcgagtaacatctcacaatcgctcctttgataggtccttgtttgtcgtgctggacagcctggcctgcacggatggtgccatcggtgcggcagcccagggctggctggtgcgtgcgctctccctcggggacgtggctcgcatcctcgaacccgtgctcctgctgctgctgcagccaaaaacccagagaacctccatccactgcctcaagcaggagaactcggccgatgacttgcaccgttggtttaacaggaagaaaacctctttcagagaggcatgcgcagtgcccgagcctcaggagagcggctctgaagagcacctgcctctgagccagttcaccacagtggaccgtgaagccatttgggccgaagtggagaaggagcccgagaagtacccgctgcgaggcgagctgagcgaggaagagctgccctactacgtggagcttccagacaggacggcccacggcgccccggacagcagcgagcacaccgagtctgcagatacaagctcctgccacacggacagcgagaacacgtcctccttctcctccccttcccacgacctgcaggagctgagcaacgaagagaactgctgtgcacccatccccatggggggcagggcgtaccccaagcgctcggccctgctggcggccttccagtcagaaagcttcaaggctggggccaagttaagcctggtgcgggtggactcggacaagacgcaggcttctgagtcgttctccagcgacgaggaggcggacttggagctccaggccctcaccacatccaggctgctaaagcagcagcgggaaaggcaggaggccgtcgaggccttgttcaagcacatcctgctctacctgcagccctacgactctcggcgggtcctctatgccttctcggtgctggaggctgtgctcaaaaccaaccctaaggaattcatcgaggctgtgtccaggactagcatggataccagctccaccgcgcacctcaacctcatctccaacctcctcgctcgccaccaggaggccctcattggccagagtttctacggaaagctccagacccaggtccccaacgtgtgcccccactctctgctcctggagctgctcacctacctctgcctgagcttcctgcgctcctactacccttgctatttgaaggtctcgcaccgagacattctcggcaaccgggacgtgcaggtcaaaagtgtcgaggttttgatcaggataatgatgcagctggtctcagtggccaagtcttcggaagggaagaacgtggagttcatccacagcttgctgcagaggtgcaaagttcaggagtttgtcctgctctccctgtcggcgtccatgtacacgagccagaagcgctacgggctggccaccgcccaccacggcagggccctgccagaggacagcctctttgaggagagtctcattaacttgggtcaggaccagatctggagtgagcacccgctgcagattgagctgctgaagctgctgcaggtgctgattgtcttggaacaccacctgggtcgggcccatgaggaggcggaaaaccagcccgacctgtcccgggagtggcagagagccctgaacttccagcaggccatcagcgccctgcagtacgtgcagccccaccccctcacctcccagggtcttctggtctctgcggtggtgaggggtctgcagcccgcctacggttacggcatgcatccggcctgggtgagcttggtcacgcattccttgccctacttcggaaagtccctgggctggacggtgacaccctttgttgtccagatttgcaaaaacttggatgacttggtcaagcagtatgaaagcgaatctgtgaagctctctgtcagcacaacctccaagagggaaaacatttctccagattatccactcacccttctagaaggtctaacgaccattagtcatttttgtcttttggaacaagccaaccaaaacaaaaagaccatggctgcaggtgatcctgccaacttgaggaatgccagaaatgccattttggaagagctgcctcgaactgttaacaccatggcccttctctggaatgttctcagaaaggaggagactcaaaagagacctgtcgatctcctaggggccacgaagggatcctcttccgtttactttaaaaccaccaaaaccataagacaaaaaattttagacttcttaaaccccttgacggcccatcttggggttcagttgacagcggctgttgcggcagtgtggagcagaaagaaagcccagcgtcacagtaagatgaagattatcccaacggcaagtgcatcccagctaacccttgtcgacttggtgtgtgcactcagcaccctgcagactgacacgctgctgcacctggtgaaggaggtggtgaagaggccaccccaagtcaaagggggtgatgagaaatcgcccctagtggacattcctgtgttgcagttttgctatgcttttctccaaaggctcccagtaccagccttgcaagagaacttttcttcactgttgggagtattgaaagagtctgtacagttgaatctagccccacctgggtattttctgcttctcagcatgctgaatgactttgtaacaagaactcccaacctggaaaacaagaaggaccaaaaagacctgcaggaaatcactcagaaaatcctagaagctgtggggaacattgccggctcttccttggagcaaaccagctggctaagcagaaacctggaagtgaaggcccaacctcaggcctctctagaagaatctgatgctgaggaggacctgtatgatgctgctgcagcttcagcaatggtgtcttcatccgccccgtcggtgtacagcgtgcaagccctctctctcctggcagaggtactggcttccctcctggacatggtttatcgaagtgatgagaaggagaaagctgtgccgttaatctcccgtctgctttactatgtttttccatacttacgcaaccacagtgcctacaatgctcccagcttccgggctggcgctcagctgctgagctccctgagtggctatgcctacacaaagcgagcctggaggaaggaggtcctggagctgtttctcgaccccgctttctttcagatggatacttcctgtgttcattggaagtccattattgaccatcttttgactcatgagaaaacaatgtttaaggatttaatgaacatgcagagcagttctttgaaactattctcaagttttgaacagaaagccatgctgttaaagcgccaggcttttgctgtcttcagtggagaacttgatcaataccacctttaccttccactgatacaagaacgcctgacagacaatctcagagttggacagacatccatagttgctgctcagatgtttctttttttcagagttttgctgctaagaatatctcctcaacatttgacttcattgtggccaataatggtctctgaattgattcagacattcacacagcttgaagaagatctaaaagatgaagatgagtcattgagaagcaccaacaaagtaaacagaacgaaagtttcagtcccggatgcaaatggaccctcagtgggggagataccccagagtgaactcatcttgtatttatcagcttgcaaattcttggacacagcgctttcttttccacctgacaagatgccattatttcaaatttataggtgggcatttattccagaagtggacacagagggccctgccttcctgtcggatgtagaggagaatcaccaagaatgcaaaccccacactgtcaggattctagaacttctaaaattaaagtttggggaaatcagtagctctgatgagatcaccatgaagagtgaattcccgcttctgcgccaacattctgtttccagcatcaggcagttgatgccattcttcatgactctaaatggtgcatttaagacccagagacagctgcctgctgatagcccaggaactccattcttggactttcctgtcacagatagcccaaggatcttaaaacaactggaagaatgcatcgaatatgattttctggaacatccagaatgttaaccatgtgagagagaatatgtttaatccatgtattggtactttactgaaaaccaggttatattctaaagaagaaagaaggcaggatagtgcttttgaacaagcctatttccattttgaaagtagatttcaggctaggtgcggtggctcacacctgtaatctcagcactttgggaggccaaggcaggcagatcacttgaggtcaggagttcgagaccagcctgaccaacatggtgagaccctgtctctactaaaaatacaaaaattagctgggtgtggtggcggcgcctgtaatcccagctacttgggaggctaaggcatgagaattgcttgaacccaggaggtggaggctgcagtgagccgagatcacgacactgcactccagctgtgtgacagaatgagaccatctccaaaaaaaaaaaaaagtagatttcagataatttactgttcagcaacaggacacacctccctaaatgccttgtaatatatttgaatctgattctgcatttcttcctcaatttatgtaatgaaaataaaattaatatatcatctaacagtagcacaaaatttgtaatatgaagtaaagtatgaagataatgaagaagttgttttctttgttgaagcagttatatgggtctttctcagtatatttctcttttctctaaaagtttaaacttattaaaagaatgttatttttaacctttcaaaaaaaaaaa

SEQ ID NO: 37 - DNA, 1138 bp, Homo sapiens
  source 1..1138: mol_type = other DNA
gctctggccggccccggcgattggtcaccgcccgctaggggacagccctggcctcctctgattggcaagcgctggccacctccccacaccccttgcgaacgctcccctagtggagaaaaggagtagctattagccaattcggcagggcccgctttttagaagcttgatttcctttgaagatgaaagactagcggaagctctgcctctttccccagtgggcgagggaactcggggcgattggctgggaactgtatccacccaaatgtcaccgatttcttcctatgcaggaaatgagcagacccatcaataagaaatttctcagcctggccgaaaatggttggccccacgaagccacgacaactggaggcaaagagggttgctcaacgccccgcctcattggaaaaccaaatcagatctgggacctatatagcgtggcggaggcggggcgatgattgtcgcgctcgcacccactgcagctgcgcacagtcgcatttctttccccgcccctgagaccctgcagcaccatctgtcatggcggctgggctgtttggtttgagcgctcgccgtcttttggcggcagcggcgacgcgagggctcccggccgcccgcgtccgctgggaatctagcttctccaggactgtggtcgccccgtccgctgtggcgggaaagcggcccccagaaccgaccacaccgtggcaagaggacccagaacccgaggacgaaaacttgtatgagaagaacccagactcccatggttatgacaaggaccccgttttggacgtctggaacatgcgacttgtcttcttctttggcgtctccatcatcctggtccttggcagcacctttgtggcctatctgcctgactacaggtgcacagggtgtccaagagcgtgggatgggatgaaagagtggtcccgccgcgaagctgagaggcttgtgaaataccgagaggccaatggccttcccatcatggaatccaactgcttcgaccccagcaagatccagctgccagaggatgagtgaccagttgctaagtggggctcaagaagcaccgccttccccaccccctgcctgccattctgacctcttctcagagcacctaattaaaggggctgaaagtctgaaaaaaaaaaaaaaa

SEQ ID NO: 38 - DNA, 1377 bp, Homo sapiens
  source 1..1377: mol_type = other DNA
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaacttactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 39 - DNA, 1779 bp, Homo sapiens
  source 1..1779: mol_type = other DNA
cggcgtgccctggggcggcgcgggcgcaggggcgcgtgcgcggcgggctgtcgttggctggagcagcggctgcgcgggtcgcggtgctgtgaggtctgcgggcgctggcaaatccggcccaggatgtagagctggcagtgcctgacggcgcgtctgacgcggagttgggtggggtagagagtagggggcggtagtcgggggtggtgggagaaggaggaggcggcgaatcacttataaatggcgccgaagcaggacccgaagcctaaattccaggaggttgggatgaatgggttccggagagcagagtactcaaatacgtggacaccaatttgcagaaacagcgagaacttcaaaaagccaatcaggagcagtatgcagaggggaagatgagaggggctgccccaggaaagaagacatctggtctgcaacagaaaaatgttgaagtgaaaacgaaaaagaacaaacagaaaacacctggaaatggagatggtggcagtaccagtgagacccctcagcctcctcggaagaaaagggcccgggtagatcctactgttgaaaatgaggaaacattcatgaacagagttgaagttaaagtaaagattcctgaagagctaaaaccgtggcttgttgatgactgggacttaattaccaggcaaaaacagctcttttatcttcctgccaagaagaatgtggattccattcttgaggattatgcaaattacaagaaatctcgtggaaacacagataataaggagtatgcggttaatgaagttgtggcagggataaaagaatacttcaacgtaatgttgggtacccagctactctataaatttgagagaccacagtatgctgaaattcttgcagatcatcccgatgcacccatgtcccaggtgtatggagcgccacatctcctgagattatttgtacgaattggagcaatgttggcttatacacctctggatgagaagagccttgctttattactcaattatcttcacgatttcctaaagtacctggcaaagaattctgcaactttgttcagtgccagcgattatgaagtggctcctcctgagtaccatcggaaagctgtgtgagaggcactctcactcacttatgtttggatctccgtaaacacatttttgttcttagtctatctcttgtacaaacgatgtgctttgaagatgttagtgtataacaattgatgtttgttttctgtttgattttaaacagagaaaaaataaaagggggtaatagctccttttttcttctttctttttttttttcatttcaaaattgctgccagtgttttcaatgatggacaacagagggatatgctgtagagtgttttattgcctagttgacaaagctgcttttgaatgctggtggttctattcctttgacactacgcacttttataatacatgttaatgctatatgacaaaatgctctgattcctagtgccaaaggttcaattcagtgtatataactgaacacactcatccatttgtgcttttgtttttttttatggtgcttaaagtaaagagcccatcctttgcaagtcatccatgttgttacttaggcattttatcttggctcaaattgttgaagaatggtggcttgtttcatggtttttgtatttgtgtctaatgcacgttttaacatgatagacgcaatgcattgtgtagctagttttctggaaaagtcaatcttttaggaattgtttttcagatcttcaataaattttttctttaaatttcaaagaacaaaaaaaaaaaaaaa

SEQ ID NO: 40 - DNA, 7827 bp, Homo sapiens
  source 1..7827: mol_type = other DNA
gtagtcttgacgtgagctagctggcatggcggcctgcattgcagcggggcactgggctgcaatgggcctaggccggagtttccaagccgccaggactctgctccccccgccggcctctatcgcctgcagggtccacgcggggcctgtccggcagcagagcactgggccttccgagcccggtgcgttccaaccgccgccgaaaccggtcatcgtggacaagcaccgccccgtggaaccggaacgcaggttcttgagtcctgaattcattcctcgaaggggaagaacagatcctctgaaatttcaaatagaaagaaaagatatgttagaaaggagaaaagtactccacattccagagttctatgttggaagtattcttcgtgttactacagctgacccatatgccagtggaaaaatcagccagtttctggggatttgcattcagagatcaggaagaggacttggagctactttcatccttaggaatgttatcgaaggacaaggtgtcgagatttgctttgaactttataatcctcgggtccaggagattcaggtggtcaaattagagaaacggctggatgatagcttgctatacttacgagatgcccttcctgaatatagcacttttgatgtgaatatgaagccagtagtacaagagcctaaccaaaaagttcctgttaatgagctgaaagtaaaaatgaagcctaagccctggtctaaacgctgggaacgtccaaattttaatattaaaggaatcagatttgatctttgtttaactgaacagcaaatgaaagaagctcagaagtggaatcagccatggcttgaatttgatatgatgagggaatatgatacttcaaaaattgaagctgcaatatggaaggaaattgaagcgtcgaaaaggtcttgattctgagaatgaatttggttagttgcagaagatacattggctctaagaggatatattttgagaccaatttaatttcatttataagaacatagtaattaagtgaactaagcattcattgttttattaatactttttttctaaaataaaacttgtacaccagtttattactctaaaaagagaattacacatgccaaatggaccaatgtccatttgcttattggaggcaaagctacaatagaagtcagagcatcaccagaatggtctttaatgagcatggaacctgagcaaagggaataggtgggatgaattttttttttaattgtgaaacaattcataagcacaatatgatttacagaataataaacattcatgtacccactatcaggttaagaaatagaacatttattaatatgtaggaatgttaagaaataaaacatttaataagatctcagaagactccagtaaatctgcaattgtatctctctcctttttaaatgtaaatatcatcttgacttgttaattattcccttgcatttcttttagtttactgccaacacatatattcttcaacaatatatttaattttgaaaaacctgaaaaaaaaaacctgttagcaagtataaaggggcagtattactattattgcatgaaggcttcaagggaaacgttacagtctttgggtcatagtctggcttcagcttcctctgagagtttacagaggccaattttgagcaaattcatggctaaggttatgagtgagttctgctaaacagaaggctcaccacaaggtatctggcaggattatactgggtagctggatgttgcagaaatgtggttagaggaagtaaactgttttttgatgctcacagcatgatgaatcaaactctgtatcttaggattaggttaaaacaatacctttggtatgatatgagtgttgttgctgatccatgcagcatggattggaaagctggggtataagcacacatgctaaagaaaaacatgtaatttggtccatactcacctggatatactgttcctcaggttaaaaaatacagtactatcctaaatcttgaaggcaactctcagcctatccattgagttaccttcagatctgccctctggttcctagctgtcttgggactaacttctttcctgcgctcagctgttttctggattccatgttttccattttattgagtactaacttgttttgctgcagcacatcctttggtagcttctagaggaagtttgtgtggaggtaaaatttttgagaccttgcatgtctcatgtttgattgatactttatacgtttaggtaggaggtaattttccttcaggactttaaaaatattgttgctccattttctttgtttctattgttgtattgagaaatccaatgccattttgatttccccatcataaatttcatgatgatgtgtcttggtgtgggtctatatttatccattgtattgggttttaggtgaacccttccagatagtaactcatttctgtcagttctgggaaacacttagcattggttgatgatttattctctgctgctttgttctcccaactattatttggatgttggatatccagcactgggtatctattttcttacctccctcccttgaccccagtctctgttttttagctctttagctcaatcttccaactctttgctattgtattttaaaatcttaagaccccttcttgatttgtagaagttccttttcttacaaccaaaaagcctttatctatggatttgttcacagataaggggtattcaatatagtgtatttttttttcatttaaaattgtttgcgcatctatttcctccaaatttctttctgtatttattttttgttgtctatatttcagacttttccaggatatctgataatctttggctgtcttcttatggttgaaagagggactaaaaagcttggaaagcctttgggttgtgggaaggggctgtctttaggattatctgaatgggcttttttgggagtcccctcctccacatgaatattttggttttgtcagattccctagaatagaggcttccaatctccttcctggaggggtctgtccaggaaggagattgtctaggggtctgtcagacagcagctttcagctacttccttgatctttttcactaatgattatatagtcatctaactactgtcaacaagtaatagatatcctatccttcacttgtttagattatttgctgagataacctctcaaaagaacctctcaaaataaaaggttaacaagagcctatatcttatatttttcttctctttatcttgttagaagatagctattaaaacctgttctttttctgtcttgataaacacacttcaatcttggtagaatggtagatgggacagtatattttaggacctaaagctctgcaaatgtatgatcagcttgtaagtacaggtgctcaaaaacatgtaaacaatcatgctttttactctgtaggaatatctttaaaattcttgtgaatttttccccagaagtaaagcaaatcttcccccagaaataaaattaaatgtgcataatctaaagctttttttttttattgtggtaggatatatatataaaacataatttgccattgtaaacattttaaatttacaagtcagaggcattaattacatcacaatgttgtgaaattattactactatttccaaaattttctcatcaccccaaactgaaactctgtaactgttgagcaataacctcattcctgtatctctcccaaccccaggtaacctcaaatctttctttttatctttgagacaaggtctcattctatcactcaggtaggagtgcagtggtgtgatcatagctcattgcagcctcaaaatcctgggctcaagcaatcctccttgagtagctaagactataggcacacattaactgcgcctggctgattttgttttttgtagagatgtggtcttgctatgtttcccatgctggtcttgagttcctggcctcaagcagtccttaagattcatccatgttgtggcatgtgtcagaatttcatttgtttttatgactaaataatattccattgtatgtatatacattttgttcatccatcttctgatgaacactgggatatgtctaccttttggctattgtgaataatgctgcagtaaacattgacataacaagtatgtatttgattgcctgtttctaagttcttttgggtatacatcttgagtagaattgctagataatgtcatgttttatttctcttgtgatttcttcttcgatcccctggttgagtgtgttaatttctacatgtttatgaatttcccactgtttttttgttattgatttccaagttcattccattgtgattagagaagatacttagtatgattttaatgtttttgagaattggtgtgtggcctgatagatggtctgtcctggagaatgttcctcatacacttgagcaaaatatttatcatgctattgttgactgtagttttctatatgtctcttaggtcaaggtggtttacaatgtgttaaggttctctttttttaaaaaaatttttgcacagagtatctttttctatgtgttccatgtatttgtgtctttggagctatagtctcttgtagacagcatatcactatcttgttttgttttgttttttctgtccattctgccaatttctgccttttgattggaaaatttaatccatttgcatttaaagtaattaaggaaggactttcttctaccatttaacacttcttctatatgtcatatacttttttggcccctcatttcctctttatggccttcttttctgtttttttgtagtgaactagtctgattctctttccactcccctttgtgtatatttgttagatgttttatttgtggttgctatggggattatagttaacatcctacacttaaaacaatctaatttaaactgataccaatttaccttcaatagcatacaaaatctctactcctgtaaagctctgcccctgccccccttatgttattgatggcacaaattgcctaataaataatttatagttatttgtatgagtttgtcttttaaatcatttaggaaataaaaagtggagttagaaaacagtatgatagtaatactgacttttatatttgtcaatatatttatcttattttggatccttatttcattatatagatttgagttactgtctagtgcccttccatttcggcccaaaggattcccttatgcatttcttgcagggcaagtctaattgtaataaactccctcagcttttgttttatctgagaatgtcttgatttctcccttatttttgatggataattttgccagatacatgaatttttggtaacagtatttttctttcagcactttaaatatgtcatcccactaccttctgacttcatggtttctcatgagatattagatgttataaaatttgaggattcctcattcttgatgagtcagttctgtcttattgcttttcggatttgctcagcttttgtcttttgacagtttgattataacgcggctcagtgtgggtctctgagtttatcccacttagagtttgttgagtttcttggagtcatagatttatgtcttttatcaaattttggacatatttggctattatttcttcaatttttttcactgcttctttcttttccttctgaaatattcttaatgtatatgttggtctgtttgatgctgtctcaccagtttcttaggctgtgttctcttttgttcctcagacttgattattgcagttgcccttctttttatttttttcaagtttgttgattcttctccctgttcagatcaactgttgaactcctctagtgaatttatttcagttactgtacttttcagctccaagatttatctttggttcctttttataacgtctgtgtctttattgatattctcattttgttcatatgtctctttcttcctttagttctttgtccatgttttcctttagctctttgggcttatttaagacaattgtttaaagtctttgcatagtaagtccaatgtctgtgtttcttcagggatggttttcattattttgttttcaatgagccatactttcctgtgtctttgtatgctgtctttttgttgttgaaaactgtatgtttgaacatcataacgtggtggccctgaaaatcagatattccccccttcctgagagttagttttatttttattattgaagattgtagcagtctattgctacatgtgcagtcatttccaaactatttttgcaaagactgtattccttctgtgtgtcatcactgaagtctctgttccttagtttgtgtttaatagtttgacatagatttccttgaaaggagttaaaactagcagaaaaatctctctcccagtctttccagtctttgtagattggttctgtgctgggcttttccattaatacttagccaggcttgtactgagcctaacaatcaggcccaaaagcgtagggtctttgcagatcttgtctgagcatgcttcttgctgtgtatgcacgtagttttctaaatctccctgtatgtgctgttgaatattctaatttcccaaagaaactcctttgcagctttttctcacagaacatagatggttttttggatatcttgaccatagtctttcgacccaggtgtttgcggttgttagttcaccttacacttttttcaagcattgcctactgcttacgatgagtgctctgtcaatcctttaagtagccccagacaggctaccagagacttaaacaagaatttgtaagttctgctcagcttcctctagaaatggggatcagggtccaagacagaatgcagttgctgatttcaagactgctgcaacaccagggagcttgtgggggaagggcaagcagaaatgtcacaaagctttcttgccattttaaagttgcctgttcttgactcagcatttgcttcattgctataaactttttactgtttttcagagttctgataaaattggctatgcctgttcctgctttaaaaaatatatatatattttttagggattggggtctcactatactgaccaggctggtcttgaacttctggcctcaagccatcctctcatttcagcttcccaaagtgctgcaattacacgcgtgaaccaccacacccagcccctgcttgtttttcaatgtgcctactccaccatgttgctcaagtatgtatattttctaaactaccttgtagtgttgtgatgggaaataaatccctgagccttttgaataactcagagagatcaaaaacttagtttatcctattcgaaggattagaaaaatgatatatctttcactttttcagggataggctcctcattagaaggctcctatgtgccgatgctgtacaagacatttcatttctcttaatgtttacaacaagcttgttgccaaggctgatcttgaactcctggcctcaaacgatcctcccagctcagtctcacaaagtgttgggatgtctggccaactaatgactatcttaactcttgtgtttcaatgtttatgccttcttttatcttgactgattgtatgactatgtcttctagaacaatgttgaacagaaatggtgagagcagacatccttgctttaatatttcaccattatatatgatgttaggtatagatttttctcacagatgccttttatcagattgaggaatttatattcctactttgccgaaaggtttttgtagtatgagggggtgctgaattttgtcaaacactttttcggtaataattgagatgattggttctgcagtcatcgagatgtggattttctcctttattctgttcgtgagtgattacactggttgactaatgttaaaacaaccttactttccaggaataaaccctattatcttttttataca

SEQ ID NO: 41 - DNA, 1821 bp, Homo sapiens
  source 1..1821: mol_type = other DNA
tgcgggtacggacagcgcatgagcttatgttgagggcggagcccagaccagcccttcgtcctatcctgcccttccagcacctctcagccgtaacttaaactacacttcccagaagcctcctcagccagggacttccgttgtcgtcagcggaagcggtgacagatcatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccatttaatttcttcttagaagtttaactcctttggagaatgtgggccttgaataggatcctctgggtccctcttaatctgacagatgagcagacgaggtgcatggcctgggttgcagcttgagagaaccaaaatattcaaaccagatgacttccaaaatgtggggaaagggatggaaaatgaacctgagatggagtccttaatcacgggataaagccctgtgcatctccctcatttcctacaggtaaaagacagtaaagaaattcaggtcacaggccttgggagttcataggaaggagatgtccagtgctgtccagtagaacttt

SEQ ID NO: 42 - DNA, 5159 bp, Homo sapiens
  source 1..5159: mol_type = other DNA
ggtcccggaagtgcgccagtcgtaccttcgcggccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaatcagttaaataagacttgtctcggtcacctgtgccctgtccagactcgaggcagtggtaacactgcacagtgctatgtggcttctctttgaggatttttgggttttgtaactaaattcttgctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatccattttatccattttaactcatgtaatcctcatgtgagtattcctgtttaacacttgagtaaactgaggcacagagaacataagttgcatgccatagtcacacactgtgaaagtgaaaagagaatgtgtgcaaaacacgtcacagtcctggtttctgagtaaaggcaggctgttatctttagaatcaagctatcacagggagataggcaatgctgtgggtgttggaggaaggtgagagcctgttgctaacaatttcctggttttaaagctaaggctgattttattgggaagatctcacatgtgtgtggcccctgagagttcccagtgccttttatttgcagtccttccatttggacctcctagctgccccatcaggtcatctccagggctcagaggggtgagaccatttcccaaggtcacagaaccagctctctagtcaccaccctgcctctccctctcacccagagtcagtaccagttttatggctttattacaaactgctgggtccctcccattttcaacttgattgatgggatgtcatcccttatcctgtctgacatttgcctctggcctggttgctagaagtttgccccaggggcaagagttgaaatttggcttcctgaggtgggctttgtggtttgcgtccctaaagtgagcccactactggttgcttgtccatggccaacaccagaaatcccctgagcactacctgggtctcattccaagaaggaagagggtcaggagacctggggagtctcatattccaagttcttctttctttctgggagcagtgggcagttcatggtgttagggcactcacccccacagactggcaaaccctgcaggacttccgtggctgaggctgtgaccggaggccaggaatgccgttgggtggattgtgagtgaatgggccctttgagctgccctctagagagcaaatccagtttcctggagctcctgaatgaatatctgtactggctcgctcagatgcagaagctccattgaccatgaggccttgtgaacatcagtggccacaggcccagtgtgctgcttggcactgcactagtttaggacctgcagcatgtaggtagcgtcctagtgtttataatacaaagctgctctgcacagcttttctgattcttcttgcaatctcctgaggattatctgccccatttttaaaacgaggtggaatacccaaggtcatgtagccagtgagtgctctggaaagccaaagcagctcatcccttcctggggaccacactgctctgctccaccagaccacactatgaaataggaataagtgctcctgttgcaggactgctgggaaaacaggtggtgtgggacttaagtcaccataattttgaagacttgcatgcagagggctccaggaattgtagacattaaggaatttcactttcagttctacccactacttaagtacttgtcatgtactcttagaggaggccagtaatgatcagaaccattttactttaaaattaataatattgtattagagaatatattaaatggttatattgggttatgttaggatatatacttgaatggaaatacatgtactattagcaatcatatttcatttatccctgtaattagacaagaaagcataatatagctctactcatgggtacacataccagtgtataagatttttagaagtttactttttaaaaataaaagcaaaatgtaagatcttaaaaaaaaaaaaaaaaaa

SEQ ID NO: 43 - DNA, 5416 bp, Homo sapiens
  source 1..5416: mol_type = other DNA
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcaggtaattaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 44 - DNA, 1940 bp, Homo sapiens
  source 1..1940: mol_type = other DNA
gagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggaaaaaaaaaaaaaaaaaa

SEQ ID NO: 45 - DNA, 1309 bp, Homo sapiens
  source 1..1309: mol_type = other DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 46 - DNA, 2321 bp, Homo sapiens
  source 1..2321: mol_type = other DNA
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 47 - DNA, 1229 bp, Homo sapiens
  source 1..1229: mol_type = other DNA
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 48 - DNA, 5234 bp, Homo sapiens
  source 1..5234: mol_type = other DNA
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 49 - DNA, 1869 bp, Homo sapiens
  source 1..1869: mol_type = other DNA
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 50 - DNA, 2288 bp, Homo sapiens
  source 1..2288: mol_type = other DNA
ggggccgaacgtggtataaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataatgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcatattgggctttaggctgtaggtcaagtttatacatcttaattatggtggaattcctatgtagagtctaaaaagccaggtacttggtgctacagtcagtctccctgcagagggttaaggcgcagactacctgcagtgaggaggtactgcttgtagcatatagagcctctccctagctttggttatggaggctttgaggttttgcaaacctgaccaatttaagccataagatctggtcaaagggatacccttcccactaaggacttggtttctcaggaaattatatgtacagtgcttgctggcagttagatgtcaggacaatctaagctgagaaaaccccttctctgcccaccttaacagacctctagggttcttaacccagcaatcaagtttgcctatcctagaggtggcggatttgatcatttggtgtgttgggcaatttttgttttactgtctggttccttctgcgtgaattaccaccaccaccacttgtgcatctcagtcttgtgtgttgtctggttacgtattccctgggtgataccattcaatgtcttaatgtacttgtggctcagacctgagtgcaaggtggaaataaacatcaaacatcttttcattatcccta

SEQ ID NO: 51 - DNA, 2439 bp, Homo sapiens
  source 1..2439: mol_type = other DNA
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 52 - DNA, 1196 bp, Homo sapiens
  source 1..1196: mol_type = other DNA
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 53 - DNA, 987 bp, Homo sapiens
  source 1..987: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 54 - DNA, 3003 bp, Homo sapiens
  source 1..3003: mol_type = other DNA
ctttctccttccccttcttccgggctcccgtcccggctcatcacccggcctgtggcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 55 - DNA, 2803 bp, Homo sapiens
  source 1..2803: mol_type = other DNA
actgcagccccgctcgactccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaatccatttgaaatattttactgttgtgactttagtcatatttgttgacctaaaaatcaaatgtaatctttgtattgtgttacatcaaaatccagatattttgtatagtttcttttttctttttcttttcttttttttttttgagacaggatcggtgcagtagtacaatcacagctcactgcagcctcaaactcctgggcagctcaggtgatcttcctgactcagccttctgagtagttggggctacaggtgtgcaccaccatgcccagctcatttattttgtaattgtagggacagggtctcactgtgttgcctaggctggtctcaagtgatcctccctccttggcctcccaaggtgctggaattataggtgtgaacaaaccaaaaaaaaaaaaaa

SEQ ID NO: 56 - DNA, 1435 bp, Homo sapiens
  source 1..1435: mol_type = other DNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

(19) United States
(12) Patent Application Publication
(10) Pub. No.: US 2026/0202409 A1
(43) Pub. Date: Jul. 16, 2026

(54) CHEMICAL COMPOSITIONS AND METHODS OF USE

(71) Applicant: Liquid Biopsy Research LLC, Charlestown (KN)

(72) Inventors: Irvin Mark Modlin, Woodbridge, CT (US); Mark Kidd, New Haven, CT (US); Ignat Drozdov, Stratford Upon Avon (GB)

(73) Assignee: Liquid Biopsy Research LLC, Charlestown (KN)

(21) Appl. No.: 19/290,017

(22) Filed: Aug. 4, 2025

Related U.S. Application Data
(63) Continuation of application No. 17/490,433, filed on Sep. 30, 2021, now Pat. No. 12,399,179.
Application No. 17/490,433 is a continuation of application No. 15/984,935, filed on May 21, 2018, now abandoned.
(60) Provisional application No. 62/511,058, filed on May 25, 2017.

(51) Int. Cl. G01N 33/5751; A61K 39/395; A61P 17/00; A61P 35/00; C12Q 1/6886; G01N 33/53; G01N 33/58

(52) U.S. Cl. CPC G01N 33/5751; C12Q 1/6886; G01N 33/5308; G01N 33/582; A61K 39/3955; A61P 17/00; A61P 35/00; C12Q 2600/106; C12Q 2600/118; C12Q 2600/158; G01N 2800/52; G01N 2800/56; G01N 2800/60; G01N 2800/7028

(57) ABSTRACT

The present invention is directed to methods for detecting a melanoma, methods for determining whether a melanoma is stable or progressive, methods for evaluating the extent of surgery resection in a subject having a melanoma, and methods for determining a response by a subject having a melanoma to a therapy.

### CROSS-REFERENCE TO RELATED APPLICATIONS

This application is a Continuation of U.S. patent application Ser. No. 17/490,433, filed on Sep. 30, 2021, which is a continuation of Ser. No. 15/984,935, filed on May 21, 2018, which claims the benefit of and priority to U.S. Provisional Application No. 62/511,058, filed on May 25, 2017. The contents of each of the aforementioned patent application are hereby incorporated by reference in their entireties for all purposes.

### INCORPORATION BY REFERENCE OF SEQUENCE LISTING

The contents of the electronic sequence listing (LBIO-001_C01US_SeqList.xml; Size: 253,314 bytes; and Date of Creation: Aug. 4, 2025) are herein incorporated by reference in its entirety.

### FIELD OF THE INVENTION

The present invention relates to melanoma detection.

### BACKGROUND OF THE INVENTION

Melanoma is a common (~24-35/100,000 incidence—US), highly aggressive, skin cancer with an incidence that continues to rise. The most common, cutaneous melanomas, are associated with UV exposure and immune dysregulation. As a group, melanoma is known to carry the highest mutational burden (>10 mutations/Mb). Major mutations include BRAF (~50%), N-Ras (~20%) and NF-1 (~5%), which together, comprise 75% of all mutations. Melanomas are addicted to MAPK pathway activation, regardless of whether tumors exhibit mutations in genes coding for proteins in this pathway. This provides the rationale for targeted therapy e.g., BRAF v600E agents, in this tumor. Other gain-of-function and loss-of-function mutations e.g., in RASopathy genes and amplification of cyclin D1/cdk4 and/or mutation/loss of the tumor suppressor PTEN, also characterize the tumor. This makes melanoma one of the most aggressive and therapy-resistant cancers.

Five-year survival rates range from 95-100% for stage I, 65-93% for stage II, to 41-71% and 9-28% for stage III and IV, respectively. Surgery, immunotherapy and targeted therapies provide the basis for management, with chemotherapy and radiation as adjuncts. Surgery, however, has a critical role in melanoma care (diagnosis, cure and palliation). Sentinel lymph node biopsy has become widespread, as it provides prognostic information. Melanoma, however, lacks a clinically useful non-invasive e.g., blood-based biomarker of disease activity to help guide patient management by providing predictive or prognostic information.

Blood-based factors include lactate dehydrogenase (LDH), detecting mutations in circulating tumor (ct) DNA, measurements of circulating tumor cells (CTCs) and circulating mRNA. LDH is typically used to identify aggressive tumor behavior and predict recurrence but its metrics are very low e.g., 30-50% accurate. It is also non-specific for melanoma. Mutations in target genes, like BRAF, can be detected in the blood in ctDNA but its utility as an indicator of therapeutic efficacy is limited e.g., 45-70% accurate. CTCs do not appear to be an accurate marker in melanomas and there is no consensus as to their clinical utility. Circulating microNAs (miRNA) have been detected but there is no evidence yet for clinical usefulness.

For melanomas, there are no multi-mRNA circulating biomarkers that function as a diagnostic or as a prognostic for disease recurrence. A biomarker that can be used to monitor the efficacy of surgery or drug therapy in melanomas is currently lacking.

### SUMMARY OF THE INVENTION

Among other things, disclosed herein is a 28-gene expression tool for melanoma. It has high sensitivity and specificity (>95%) for the detection of melanoma and can differentiate aggressive untreated disease from stable, treated disease.

One aspect of the present disclosure relates to a method for detecting a melanoma in a subject in need thereof, comprising: (1) determining the expression level of at least 29 biomarkers from a test sample from the subject by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a first predetermined cutoff value; and (5) producing a report, wherein the report identifies the presence of a melanoma in the subject when the score is equal to or greater than the first predetermined cutoff value or identifies the absence of a melanoma in the subject when the score is below the first predetermined cutoff value, wherein the first predetermined cutoff value is 20 on a scale of 0 to 100.

In some embodiments, the method further comprises treating the subject identified as having a melanoma with surgery or drug therapy.

Another aspect of the present disclosure relates to a method for determining whether a melanoma in a subject is stable or progressive, comprising: (1) determining the expression level of at least 29 biomarkers from a test sample from the subject by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a second predetermined cutoff value; and (5) producing a report, wherein the report identifies that the melanoma is progressive when the normalized expression level is equal to or greater than the second predetermined cutoff value or identifies that the melanoma is stable when the normalized expression level is below the second predetermined cutoff value, wherein the second predetermined cutoff value is 50 on a scale of 0 to 100.

Another aspect of the present disclosure relates to a method for evaluating the extent of surgical resection in a subject having a melanoma, comprising: (1) determining the expression level of at least 29 biomarkers from a test sample from the subject after the surgical resection by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a third predetermined cutoff value; and (5) producing a report, wherein the report identifies that the surgical resection does not remove the entire melanoma when the normalized expression level is equal to or greater than the third predetermined cutoff value or identifies that the surgical resection removes the entire melanoma when the normalized expression level is below the third predetermined cutoff value, wherein the third predetermined cutoff value is 20 on a scale of 0 to 100.

In some embodiments, the report further identifies that the risk of melanoma recurrence is high when the normalized expression level is equal to or greater than the third predetermined cutoff value or identifies that the risk of melanoma recurrence is low when the normalized expression level is below the third predetermined cutoff value.

Yet another aspect of the present disclosure relates to a method for determining a response by a subject having a melanoma to a therapy, comprising: (1) determining a first expression level of at least 28 biomarkers from a first test sample from the subject at a first time point by contacting the first test sample with a plurality of agents specific to detect the expression of the at least 28 biomarkers, wherein the 28 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (2) determining a second expression level of the at least 28 biomarkers from a second test sample from the subject at a second time point by contacting the second test sample with a plurality of agents specific to detect the expression of the at least 28 biomarkers, wherein the second time point is after the first time point and after the administration of the therapy to the subject; (3) comparing the first expression level with the second expression level; and (4) producing a report, wherein the report identifies that the subject is responsive to the therapy when the second expression level is significantly decreased as compared to the first expression level.

In some embodiments, the first time point is prior to the administration of the therapy to the subject. In some embodiments, the first time point is after the administration of the therapy to the subject. In some embodiments, the therapy comprises an immunotherapy or a targeted therapy (e.g., a BRAF inhibitor).

In some embodiments, the at least one housekeeping gene is selected from the group consisting of ALG9, SEPN, YWHAQ, VPS37A, PRRC2B, DOPEY2, NDUFB11, ND4, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, HPRT1, TOX4, and TPT1.

In some embodiments, the at least one housekeeping gene comprises TOX4 and TPT1.

In some embodiments, the normalized expression level is obtained by: (1) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TOX4, thereby obtaining a first normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TPT1, thereby obtaining a second normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; and (3) averaging the first normalized expression level and the second normalized expression level to obtain the normalized expression level.

In some embodiments, the method can have a specificity, sensitivity, and/or accuracy of at least 75%, 80%, 85%, 90%, 91%, 92%, 93%, 94%, 95%, 96%, 97%, 98%, or 99%. In some embodiments, the method has a sensitivity of greater than 90%. In some embodiments, the method has a specificity of greater than 90%.

In some embodiments, the biomarker is RNA, cDNA, or protein. When the biomarker is RNA, the RNA can be reverse transcribed to produce cDNA, and the produced cDNA expression level is detected. In some embodiments, the expression level of the biomarker is detected by forming a complex between the biomarker and a labeled probe or primer. When the biomarker is RNA or cDNA, the RNA or cDNA can be detected by forming a complex between the RNA or cDNA and a labeled nucleic acid probe or primer. When the biomarker is protein, the protein can be detected by forming a complex between the protein and a labeled antibody. In some embodiments, the label is a fluorescent label.

In some embodiments, the test sample is blood, serum, plasma, or neoplastic tissue.

In some embodiments, the first predetermined cutoff value can be derived from a plurality of reference samples obtained from subjects free of a neoplastic disease. The second predetermined cutoff value can be derived from a plurality of reference samples obtained from subjects whose melanomas are being adequately controlled by therapies like immune therapy. The third predetermined cutoff value can be derived from a plurality of reference samples obtained from subjects whose melanomas have been completely removed by surgery and they are considered “disease free.” In some embodiments, each reference sample can be blood, serum, plasma, or non-neoplastic tissue.

In some embodiments, the subject in need thereof is a subject diagnosed with a melanoma, a subject having at least one melanoma symptom, or a subject having a predisposition or familial history for developing a melanoma. In some embodiments, the subject is a human.

In some embodiments, the algorithm is XGB, RF, glmnet, cforest, CART, treebag, knn, nnet, SVM-radial, SVM-linear, NB, NNET, or mlp.

### BRIEF DESCRIPTION OF THE DRAWINGS

FIG. 1 is a graph showing visualization of the melanoma score as a system of three contributors to the clinical picture—Control, Response, and Progression. Samples towards each of the corner represent pure representations of each clinical group. Samples in the middle are in the area of both algorithmic and clinical uncertainty.

FIG. 2 is a graph showing the metrics for the test in the test set ranged from 87-100%.

FIGS. 3A-3C are a set of graphs showing the evaluation of the circulating melanoma gene test (Melanomx) in test set 2. Values were significantly higher in melanoma samples than in controls (FIG. 3A). Patients who were responding to therapy had values similar to controls. Receiver operator curve analysis of test set 2 identifying the AUC for differentiating melanoma from controls was >0.95 (FIG. 3B). The metrics for the test ranged from 78-92% (FIG. 3C).

FIGS. 4A-4B are a set of graphs showing the effect of surgery on the Melanomx. Levels were significantly decreased by surgery (p<0.0001) (FIG. 4A). Values in the NED (no evidence of disease after surgery) group were significantly lower than in those with residual disease after surgery (p=0.0007) (FIG. 4B).

FIG. 5 is a graph showing the effect of therapy on the Melanomx score. Levels were significantly decreased by immunotherapy (ipilimumab) or a BRAF inhibitor (Vemurafenib).

FIGS. 6A-6B are a set of graphs showing Melanomx score in 3 different melanoma cell lines. FIG. 6A identifies the cell lines demonstrate elevated expression—Melanomx score ranging from 40 (A375) to 95 (Hs294). FIG. 6B identifies that spiking these cells into blood from a subject that does not have a melanoma, resulted in detectable gene expression and scores. A minimum of 1 cell/ml of blood could be consistently identified.

FIGS. 7A-7B are a set of graphs showing expression in tumor tissue and its correlation with blood samples collected at the same time. In FIG. 7A, the Melanomx score ranged 40-97 in melanoma tumor tissue. In contrast, normal epithelium exhibited values<20. In FIG. 7B, gene expression in tumor tissue is compared to matched blood samples. This is highly concordant (correlation ~0.80).

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

Early signs of melanoma include changes to the shape or color of existing moles or, in the case of nodular melanoma, the appearance of a new lump anywhere on the skin. At later stages, the mole may itch, ulcerate or bleed. Visual inspection is the most common diagnostic technique. Melanoma can be divided into the following types: lentigo maligna, lentigo maligna melanoma, superficial spreading melanoma, acral lentiginous melanoma, mucosal melanoma, nodular melanoma, polypoid melanoma, and desmoplastic melanoma.

Measurements of circulating melanoma transcripts—the Melanomx—identify melanomas and decreases in the Melanomx score correlate with the efficacy of therapeutic interventions such as immunotherapy and targeted therapy. Targeted gene expression profile of RNA can be isolated from the peripheral blood of patients with melanoma. This expression profile is evaluated in an algorithm and converted to an output (prediction). It can identify active disease, provide an assessment of treatment responses, or predict risk of relapse, in conjunction with standard clinical assessment and imaging.

In one aspect, the present disclosure provides a method for detecting a melanoma in a subject in need thereof, including: (1) determining the expression level of at least 29 biomarkers from a test sample from the subject by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a first predetermined cutoff value; and (5) producing a report, wherein the report identifies the presence of a melanoma in the subject when the score is equal to or greater than the first predetermined cutoff value or identifies the absence of a melanoma in the subject when the score is below the first predetermined cutoff value, wherein the first predetermined cutoff value is 20 on a scale of 0 to 100.

In some embodiments, the at least one housekeeping gene is selected from the group consisting of ALG9, SEPN, YWHAQ, VPS37A, PRRC2B, DOPEY2, NDUFB11, ND4, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, HPRT1, TOX4, and TPT1.

In some embodiments, the at least one housekeeping gene comprises TOX4 and TPT1. In some embodiments, the normalized expression level is obtained by: (1) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TOX4, thereby obtaining a first normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TPT1, thereby obtaining a second normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; and (3) averaging the first normalized expression level and the second normalized expression level to obtain the normalized expression level.

Among the provided methods are those that are able to classify or detect a melanoma. In some embodiments, the provided methods can identify or classify a melanoma in a human blood sample. In some examples, the methods can provide such information with a specificity, sensitivity, and/or accuracy of at least 75%, 80%, 85%, 90%, 91%, 92%, 93%, 94%, 95%, 96%, 97%, 98%, or 99%.

The agents can be any agents for detection of the biomarkers, and typically are isolated polynucleotides or isolated polypeptides or proteins, such as antibodies, for example, those that specifically hybridize to or bind to the at least 29 biomarkers.

The biomarker can be RNA, cDNA, or protein. When the biomarker is RNA, the RNA can be reverse transcribed to produce cDNA (such as by RT-PCR), and the produced cDNA expression level is detected. The expression level of the biomarker can be detected by forming a complex between the biomarker and a labeled probe or primer. When the biomarker is RNA or cDNA, the RNA or cDNA detected by forming a complex between the RNA or cDNA and a labeled nucleic acid probe or primer. The complex between the RNA or cDNA and the labeled nucleic acid probe or primer can be a hybridization complex.

When the biomarker is protein, the protein can be detected by forming a complex between the protein and a labeled antibody. The label can be any label, for example a fluorescent label, chemiluminescence label, radioactive label, etc. The protein level can be measured by methods including, but not limited to, immunoprecipitation, ELISA, Western blot analysis, or immunohistochemistry using an agent, e.g., an antibody, that specifically detects the protein encoded by the gene.

In some embodiments, the methods are performed by contacting the test sample with one of the provided agents, more typically with a plurality of the provided agents, for example, a set of polynucleotides that specifically bind to the at least 29 biomarkers. In some embodiments, the set of polynucleotides includes DNA, RNA, cDNA, PNA, genomic DNA, or synthetic oligonucleotides. In some embodiments, the methods include the step of isolating RNA from the test sample prior to detection, such as by RT-PCR, e.g., QPCR. Thus, in some embodiments, detection of the melanoma biomarkers, such as expression levels thereof, includes detecting the presence, absence, or amount of RNA. In one example, the RNA is detected by PCR or by hybridization.

In some embodiments, the polynucleotides include sense and antisense primers, such as a pair of primers that is specific to each of the at least 29 biomarkers. In one aspect of this embodiment, the detection of the at least 29 biomarkers is carried out by PCR, typically quantitative or real-time PCR. For example, in one aspect, detection is carried out by producing cDNA from the test sample by reverse transcription; then amplifying the cDNA using the pairs of sense and antisense primers that specifically hybridize to the panel of at least 28 biomarkers, and detecting products of the amplification.

The test sample can be any biological fluid obtained from the subject. Preferably, the test sample is blood, serum, plasma or neoplastic tissue.

The first predetermined cutoff value can be derived from a plurality of reference samples obtained from subjects free of a neoplastic disease. Each reference sample can be any biological fluid obtained from a subject not having, showing symptoms of or diagnosed with a neoplastic disease. In some embodiments, the reference sample is blood, serum, plasma, or non-neoplastic tissue.

The subject in need thereof can be a subject diagnosed with a melanoma, a subject having at least one melanoma symptom or a subject having a predisposition or familial history for developing a melanoma. The subject can be any mammal. Preferably, the subject is human. The terms “subject” and “patient” are used interchangeably herein.

The score is the Melanomx score, which has a scale of 0 to 100. The Melanomx score is the product of a classifier built from predictive classification algorithms, e.g., XGB, RF, glmnet, cforest, CART, treebag, knn, nnet, SVM-radial, SVM-linear, NB, NNET, or mlp. The algorithm analyzes the data (i.e., expression levels) and then assigns a score.

The method can further include treating the subject identified as having a melanoma with surgery, drug therapy, radiation therapy, or a combination thereof. The drug therapy can be an immunotherapy, a targeted therapy, a chemotherapy, or a combination thereof. In some embodiments, the drug therapy includes an immunotherapy. Examples of immunotherapies for treating a melanoma include, but are not limited to, Imlygic (T-VEC), Yervoy in combination with Opdivo, Opdivo (nivolumab), Keytruda (pembrolizumab), Yervoy (ipilimumab), Interleukin-2 (IL-2), and Interferon alpha 2-b. In some embodiments, the drug therapy includes a targeted therapy such as a BRAF inhibitor. Examples of targeted therapies for treating a melanoma include, but are not limited to, Zelboraf in combination with Cotellic (cobimetinib), Tafinlar in combination with Mekinist, Tafinlar (dabrafenib), Mekinist (trametinib), and Zelboraf (vemurafenib). In some embodiments, the drug therapy includes a chemotherapy. In some embodiments, the chemotherapy includes dacarbazine.

The present disclosure also provides a method for determining whether a melanoma in a subject is stable or progressive, including: (1) determining the expression level of at least 29 biomarkers from a test sample from the subject by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a second predetermined cutoff value; and (5) producing a report, wherein the report identifies that the melanoma is progressive when the normalized expression level is equal to or greater than the second predetermined cutoff value or identifies that the melanoma is stable when the normalized expression level is below the second predetermined cutoff value, wherein the second predetermined cutoff value is 50 on a scale of 0 to 100.

The second predetermined cutoff value can be derived from a plurality of reference samples obtained from subjects whose melanomas are being adequately controlled by therapies like immune therapy.

Surgical resection is a procedure that removes melanoma tissues from the subject in need thereof. The present disclosure also provides a method for evaluating the extent of surgical resection in a subject having a melanoma, including: (1) determining the expression level of at least 29 biomarkers from a test sample from the subject after the surgical resection by contacting the test sample with a plurality of agents specific to detect the expression of the at least 29 biomarkers, wherein the at least 29 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, and at least one housekeeping gene; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of the at least one housekeeping gene, thereby obtaining a normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) inputting each normalized expression level into an algorithm to generate a score; (4) comparing the score with a third predetermined cutoff value; and (5) producing a report, wherein the report identifies that the surgical resection does not remove the entire melanoma when the normalized expression level is equal to or greater than the third predetermined cutoff value or identifies that the surgical resection removes the entire melanoma when the normalized expression level is below the third predetermined cutoff value, wherein the third predetermined cutoff value is 20 on a scale of 0 to 100.

The third predetermined cutoff value can be derived from a plurality of reference samples obtained from subjects whose melanoma disease has been completely removed by surgery and they are considered “disease free.”

When it is determined that the surgical resection does not remove the entire melanoma, the subject is at risk of melanoma recurrence. Accordingly, in some embodiments, the report further identifies that the risk of melanoma recurrence is high when the normalized expression level is equal to or greater than the third predetermined cutoff value or identifies that the risk of melanoma recurrence is low when the normalized expression level is below the third predetermined cutoff value.

The present disclosure also provides a method for determining a response by a subject having a melanoma to a therapy, comprising: (1) determining a first expression level of at least 28 biomarkers from a first test sample from the subject at a first time point by contacting the first test sample with a plurality of agents specific to detect the expression of the at least 28 biomarkers, wherein the 28 biomarkers comprise ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (2) determining a second expression level of the at least 28 biomarkers from a second test sample from the subject at a second time point by contacting the second test sample with a plurality of agents specific to detect the expression of the at least 28 biomarkers, wherein the second time point is after the first time point and after the administration of the therapy to the subject; (3) comparing the first expression level with the second expression level; and (4) determining that the subject is responsive to the therapy when the second expression level is significantly decreased as compared to the first expression level.

In some embodiments, the methods can predict treatment responsiveness to, or determine whether a patient has become clinically stable following, or is responsive or non-responsive to, a melanoma treatment, such as a surgical intervention or drug therapy (for example, an immunotherapy or targeted therapy). In some cases, the methods can do so with a specificity, sensitivity, and/or accuracy of at least 75%, 80%, 85%, 90%, 91%, 92%, 93%, 94%, 95%, 96%, 97%, 98%, or 99%. In some cases, it can differentiate between treated and untreated melanoma with a specificity, sensitivity, and/or accuracy of at least 75%, 80%, 85%, 90%, 91%, 92%, 93%, 94%, 95%, 96%, 97%, 98%, or 99%.

In some embodiments, the first and second test samples can be of the same type. In some embodiments, the first and second test samples can be of different types.

In some embodiments, the therapy can be a drug therapy. The drug therapy can be an immunotherapy, a targeted therapy, a chemotherapy, or a combination thereof. In some embodiments, the therapy can be a radiation therapy.

In some embodiments, the first time point is prior to the administration of the therapy to the subject. In some embodiments, the first time point is after the administration of the therapy to the subject. The second time point can be a few days, a few weeks, or a few months after the first time point. For example, the second time point can be at least 1 day, at least 7 days, at least 14 days, at least 30 days, at least 60 days, or at least 90 days after the first time point.

In some embodiments, the second expression level is significantly decreased as compared to the first expression level when the second expression level is at least 10% less than the first expression level. In some embodiments, the second expression level is significantly decreased as compared to the first expression level when the second expression level is at least 20% less than the first expression level. In some embodiments, the second expression level is significantly decreased as compared to the first expression level when the second expression level is at least 30% less than the first expression level. In some embodiments, the second expression level is significantly decreased as compared to the first expression level when the second expression level is at least 40% less than the first expression level. In some embodiments, the second expression level is significantly decreased as compared to the first expression level when the second expression level is at least 50% less than the first expression level. In some embodiments, the second expression level is significantly decreased as compared to the first expression level when the second expression level is at least 60% less than the first expression level. In some embodiments, the second expression level is significantly decreased as compared to the first expression level when the second expression level is at least 70% less than the first expression level. In some embodiments, the second expression level is significantly decreased as compared to the first expression level when the second expression level is at least 80% less than the first expression level. In some embodiments, the second expression level is significantly decreased as compared to the first expression level when the second expression level is at least 90% less than the first expression level.

In some embodiments, the method further comprises determining a third expression level of the at least 28 biomarkers from a third test sample from the subject at a third time point by contacting the third test sample with a plurality of agents specific to detect the expression of the at least 28 biomarkers, wherein the third time point is after the second time point. The method can further comprise creating a plot showing the trend of the expression level change.

The present disclosure also provides an assay comprising: (1) determining the expression level of biomarkers consisting essentially of the following 30 biomarkers from a test sample from a patient diagnosed of a melanoma or a subject suspected of having a melanoma: ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, TOX4, and TPT1, wherein the expression level is measured by contacting the test sample with a plurality of agents specific to detect the expression of the 30 biomarkers; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TOX4, thereby obtaining a first normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TPT1, thereby obtaining a second normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (4) averaging the first normalized expression level and the second normalized expression level to obtain a normalized expression level for each biomarker; (5) inputting each normalized expression level into an algorithm to generate a score; and (6) comparing the score with a first predetermined cutoff value.

The present disclosure also provides an assay comprising: (1) determining the expression level of biomarkers consisting of the following 30 biomarkers from a test sample from a patient diagnosed of a melanoma or a subject suspected of having a melanoma: ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2, TOX4, and TPT1, wherein the expression level is measured by contacting the test sample with a plurality of agents specific to detect the expression of the 30 biomarkers; (2) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TOX4, thereby obtaining a first normalized expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (3) normalizing the expression level of each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2 to the expression level of TPT1, thereby obtaining a second normalized expression level of each of ATL1, ATP6V0D, C1ORF21, CFLAR, CFLAR-AS1, CHP1, DDX55, DMD, DNAJC9, ENOSF1, FANCL, HJUJRP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, and YY2; (4) averaging the first normalized expression level and the second normalized expression level to obtain a normalized expression level for each biomarker; (5) inputting each normalized expression level into an algorithm to generate a score; and (6) comparing the score with a first predetermined cutoff value.

The sequence information of the melanoma biomarkers and housekeepers is shown in Table 1.

TABLE 1
Melanoma Biomarker/Housekeeper Sequence Information

| | | | SEQ |
|---|---|---|---|
| Gene | RefSeq | | ID |
| Name | Accession | Sequence | NO: |
| ATL1 | NM_001127713.1 | AGTTTGAGGC TGCACTGCAC ACCTGCACAA CCTGCCTTCT | 1 |
| | | ACTTAGTTCT TCTGAGACAT TTCTGAAAGT CTGAATTCCT | |
| | | AGGACTGCTC AATGACCTTT GTCCCTGTTG GGCACACGCA | |
| | | GTGTCTCATC GCTGGTATTG CACCTTTAAT GAGACCAGGA | |
| | | GTTCCGCAAA AGTAAAACAA AGGGGTCACA GACTGGTTCT | |
| | | GGTTCTACCA CTTCCTCAGT ATGTGCTCTG GGACAAAACA | |
| | | GCATATTTGG CAACATCTCG GTGTCCTTAT CTGCAGCTTG | |
| | | AAGAGGCGCC ACAGCAACAT CCTCAGAGTC TGAGCGAACT | |
| | | GCGCCCAGCG CGGGCACGGA GCCTCCCACC GCCAGCAACC | |
| | | TGCGGCCCCG GAGAAGGCAG CGAGCGCAGT GACAGCGCCT | |
| | | CACCGCCACC AGCTCCTGGA CCACCATGGC CAAGAACCGC | |
| | | AGGGACAGAA ACAGTTGGGG TGGATTTTCG GAAAAGACAT | |
| | | ATGAATGGAG CTCAGAAGAG GAGGAGCCAG TGAAAAAGGC | |
| | | AGGACCAGTC CAAGTCCTCA TTGTCAAAGA TGACCATTCC | |
| | | TTTGAGTTAG ATGAAACTGC ATTAAATCGG ATCCTTCTCT | |
| | | CGGAGGCTGT CAGAGACAAG GAGGTTGTTG CTGTATCTGT | |
| | | TGCTGGAGCA TTTAGAAAAG GAAAATCATT CCTGATGGAC | |
| | | TTCATGTTGA GATACATGTA CAACCAGGAA TCAGTTGATT | |
| | | GGGTTGGAGA CTACAATGAA CCATTGACTG GTTTTTCATG | |
| | | GAGAGGTGGA TCTGAGCGAG AGACCACAGG AATTCAGATA | |
| | | TGGAGTGAAA TCTTCCTTAT CAATAAACCT GATGGTAAAA | |
| | | AGGTTGCAGT GTTATTGATG GATACTCAGG GAACCTTTGA | |
| | | TAGTCAGTCA ACTTTGAGAG ATTCAGCCAC AGTATTTGCC | |
| | | CTTAGCACAA TGATCAGCTC AATACAGGTA TATAACTTAT | |
| | | CCCAAAATGT CCAGGAGGAT GATCTTCAGC ACCTCCAGCT | |
| | | TTTCACTGAG TATGGCAGAC TGGCAATGGA GGAAACATTC | |
| | | CTGAAGCCAT TTCAGAGTCT GATATTTCTT GTTCGAGACT | |
| | | GGAGTTTCCC ATACGAATTT TCATATGGAG CCGATGGTGG | |
| | | TGCCAAATTC TTGGAAAAAC GCCTCAAGGT CTCAGGGAAC | |
| | | CAGCATGAAG AACTACAGAA CGTCAGAAAA CACATCCATT | |
| | | CCTGTTTCAC CAACATTTCC TGTTTTCTGC TACCTCATCC | |
| | | TGGCTTAAAA GTAGCTACCA ATCCAAACTT TGATGGAAAA | |
| | | TTGAAAGAAA TAGATGATGA ATTCATCAAA AACTTGAAAA | |
| | | TACTGATTCC TTGGCTACTT AGTCCCGAGA GCCTAGATAT | |
| | | TAAAGAGATC AATGGGAATA AAATCACCTG CCGGGGTCTG | |
| | | GTGGAGTACT TCAAGGCTTA TATAAAGATC TATCAAGGTG | |
| | | AAGAATTACC ACATCCCAAA TCCATGTTAC AGGCCACAGC | |
| | | AGAAGCTAAC AATTTAGCAG CCGTGGCAAC TGCCAAGGAC | |
| | | ACATACAACA AAAAAATGGA AGAGATTTGT GGTGGTGACA | |
| | | AACCATTTCT GGCCCCAAAT GACTTGCAGA CCAAACACCT | |
| | | GCAACTTAAG GAAGAATCTG TGAAGCTATT CCGAGGGGTG | |
| | | AAGAAGATGG GTGGGGAAGA ATTTAGCCGG CGTTACCTGC | |
| | | AGCAGTTGGA GAGTGAAATA GATGAACTTT ACATCCAATA | |
| | | TATCAAGCAC AATGATAGCA AAAATATCTT CCATGCAGCT | |
| | | CGTACCCCAG CCACACTGTT TGTAGTCATC TTTATCACAT | |
| | | ATGTGATTGC TGGTGTGACT GGATTCATTG GTTTGGACAT | |
| | | CATAGCTAGC CTATGCAATA TGATAATGGG ACTGACCCTT | |
| | | ATCACCCTGT GCACTTGGGC ATATATCCGG TACTCTGGAG | |
| | | AATACCGAGA GCTGGGAGCT GTAATAGACC AGGTGGCTGC | |
| | | AGCTCTGTGG GACCAGGCTT TGTACAAGCT TTACAGTGCA | |
| | | GCAGCAACCC ACAGACATCT GTATCATCAA GCTTTCCCTA | |
| | | CACCAAAGTC GGAATCTACT GAACAATCAG AAAAGAAAAA | |
| | | AATGTAATGC AAATTTTAAG AAATACAGGT GCATGACCAA | |
| | | TTGTCAATTA AATATTCAGT TTTATGTCTC CATGCAAACA | |
| | | TTCAAAGTGC TTCCATCAGA ACGGAGTAAA ATACTAAACA | |
| | | CCTCTGAAGA CTGCAAACTG GATTAGTTCT TTTACTTCAG | |
| | | TGTTTAATAA GCAGATGTAT GTATGCATGG TTATACTATT | |
| | | TTGTTAACAT GTACAATTTC CTGATTTTTC TTCAAAAATG | |
| | | CTGTTATAAA GTATTTGTCT ATTTATGATA ACAGTACACG | |
| | | TGTTCTGCTT GAATTTACTA AATTCTACTA CTGGGTTATA | |
| | | ATTAAATCAT GTGATATTCC ACGTTTGGAT ATGCTCATTT | |
| | | AATTTCTACA GAAAAAATTT TAAATTATTT CACATTAGCC | |
| | | ATTTGTTAAA ACACAGCATC ATAACTCAGC AGGCTGGATT | |
| | | TAATCTGTAT CATCTTATAT ATATCACAAT CTTATTTTTA | |
| | | AGCACATTTT AGAGTTCCTT AGTTGCTTTA TCAAAAACCA | |
| | | GATATTGCTT TTACATGGTT TAATAGAATA TAAACCTCTT | |
| | | GATAAAAAAT GCACAAAAAA TCACTTTGTA TATGTGAGTT | |
| | | TCACTGCATT GTATATTTTT TCATTTGGTA CACAAAGAAT | |
| | | GTATTCTTCA TAGGTTTATT CTTTTAATAT GTGAACTATT | |
| | | ATTAAAGTTT ACTCTGGTTC CTAAGATTAA AAACAAATGC | |
| | | TTACTGAATT TGAAAAAAAA A | |
| ATP6V0D | NM_004691.4 | ACTTGACAGC CCGCTGAGGA CGCAGCGTCA GCTGACCTGG | 2 |
| | | GGAGTCGCGA TTCGTGCCGG CCGGTCCTGG TTCTCCGGTC | |
| | | CCGCCGCTCC CGCAGCAGCC ATGTCGTTCT TCCCGGAGCT | |
| | | TTACTTTAAC GTGGACAATG GCTACTTGGA GGGACTGGTG | |
| | | CGCGGCCTGA AGGCCGGGGT GCTCAGCCAG GCCGACTACC | |
| | | TCAACCTGGT GCAGTGCGAG ACGCTAGAGG ACTTGAAACT | |
| | | GCATCTGCAG AGCACTGATT ATGGTAACTT CCTGGCCAAC | |
| | | GAGGCATCAC CTCTGACGGT GTCAGTCATC GATGACCGGC | |
| | | TCAAGGAGAA GATGGTGGTG GAGTTCCGCC ACATGAGGAA | |
| | | CCATGCCTAT GAGCCACTCG CCAGCTTCCT AGACTTCATT | |
| | | ACTTACAGTT ACATGATCGA CAACGTGATC CTGCTCATCA | |
| | | CAGGCACGCT GCACCAGCGC TCCATCGCTG AGCTCGTGCC | |
| | | CAAGTGCCAC CCACTAGGCA GCTTCGAGCA GATGGAGGCC | |
| | | GTGAACATTG CTCAGACACC TGCTGAGCTC TACAATGCCA | |
| | | TTCTGGTGGA CACGCCTCTT GCGGCTTTTT TCCAGGACTG | |
| | | CATTTCAGAG CAGGACCTTG ACGAGATGAA CATCGAGATC | |
| | | ATCCGCAACA CCCTCTACAA GGCCTACCTG GAGTCCTTCT | |
| | | ACAAGTTCTG CACCCTACTG GGCGGGACTA CGGCTGATGC | |
| | | CATGTGCCCC ATCCTGGAGT TTGAAGCAGA CCGCCGCGCC | |
| | | TTCATCATCA CCATCAATTC TTTCGGCACA GAGCTGTCCA | |
| | | AAGAGGACCG TGCCAAGCTC TTTCCACACT GTGGGCGGCT | |
| | | CTACCCTGAG GGCCTGGCGC AGCTGGCTCG GGCTGACGAC | |
| | | TATGAACAGG TCAAGAACGT GGCCGATTAC TACCCGGAGT | |
| | | ACAAGCTGCT CTTCGAGGGT GCAGGTAGCA ACCCTGGAGA | |
| | | CAAGACGCTG GAGGACCGAT TCTTTGAGCA CGAGGTAAAG | |
| | | CTGAACAAGT TGGCCTTCCT GAACCAGTTC CACTTTGGTG | |
| | | TCTTCTATGC CTTCGTGAAG CTCAAGGAGC AGGAGTGTCG | |
| | | CAACATCGTG TGGATCGCTG AATGTATCGC CCAGCGCCAC | |
| | | CGCGCCAAAA TCGACAACTA CATCCCTATC TTCTAGCGTC | |
| | | CTGGCCCAAG GCTCTCAATT GCACTCTTTG TGTGTGTGTG | |
| | | TGTGTGTGTG CGCGTGTGTG TGCGTGTGTG TGTATGTGGT | |
| | | CTGTGACAAG CCTGTGGCTC ACCTGCCTGT CCGGGGTGTA | |
| | | GTACGCTGTC CTAGCGGCTG CCCAGTTCTC CTGACCCTCT | |
| | | TAGAGACTGT TCTTAGGCCT GAAAAGGGGC TGGGCACCCC | |
| | | CCCCCACCAA GGATGGACGA AGACCCCCTC CAGAGCAAGG | |
| | | AGGCCCCCTC AGCCCTGTGG TTACAGCCGC TGATGTATCT | |
| | | AAGAAGCATG TCACTTTCAT GTTCCTCCCT AACTCCCTGA | |
| | | CCTGAGAACC CTGGGGCCTG GGGGCAGTTT GAGCCTCCTC | |
| | | TCCCTTCTGT GGGTCGCTCC CAGAGCCATG GCCCATGGGA | |
| | | AGGACAGAGT GTGTGTGTCC TTGGGGCCTG GGGGGATGTT | |
| | | GCTCCTCAGC TCCCTCCCTC AGCCCTGCCC CTCTGAGACA | |
| | | ATAAAACTGC CCTCTCTAAG GCCAACTGTC AAAAAAAAAA | |
| | | AAAAAAAA | |
| C1ORF21 | NM_030806.3 | CCCTCCCTCG CTCGCTCCTC GCAAGCTCCC GCTCGCTCCC | 3 |
| | | TGCCCACTCC CGGGGGGACG TTCCGTGCCG CGGCCGCCGC | |
| | | GGCCGCTGCT TCTTTCACAC TTTAGTTGGG AGCTGCGCGC | |
| | | CGCGCTCAGT TACTGGAGAG CTGGCCGCGC GCCGCCGCCT | |
| | | CCCGCACGCT TGCACGCGGG CCCGGCTTCG GGGTTTTGGG | |
| | | TTCTTACTCC AAGCGGCGGG GAGGAGGGGG AGCCCCGGAC | |
| | | ACACTGTGGG GAGGAGGAGG AAGAAGAGGA GGAGGGAGGA | |
| | | AGAAAAAAGA CGAGGAGGAC AGGGGCGGGG GGCGGGAGGC | |
| | | TTGCCACCTT CAGCCCCCCC GCGAACGCCC AAGGTGCACA | |
| | | CATCTTGACC AACTCAGCAG CAAGGTGGAT TTTCTTTGTG | |
| | | TTTAAAGAAA AAAAATGTCC CTGTGTCTGT AGAGATGATT | |
| | | TGCAGTTCAG CCCGGCTGAA GCTGACCGAA TGAGACTATG | |
| | | GGCTGTGCCT CCGCCAAGCA TGTTGCCACT GTTCAAAATG | |
| | | AAGAGGAAGC CCAGAAAGGG AAAAACTACC AGAACGGAGA | |
| | | TGTGTTTGGC GATGAGTATA GGATCAAACC AGTGGAAGAG | |
| | | GTCAAATACA TGAAAAATGG GGCAGAAGAA GAGCAGAAAA | |
| | | TAGCAGCCAG GAACCAAGAA AACTTGGAAA AAAGTGCCAG | |
| | | CTCAAATGTA AGACTTAAAA CTAATAAAGA GGTTCCGGGA | |
| | | TTAGTTCATC AACCCAGAGC AAACATGCAC ATCTCTGAAA | |
| | | GCCAACAAGA ATTCTTCAGA ATGCTGGATG AAAAAATTGA | |
| | | AAAGGGTCGG GATTACTGTT CGGAAGAAGA GGATATCACA | |
| | | TAGCACCAAT TTTACCACTC AAACCAGGAG CTACTACTGT | |
| | | GTAAATAGGT TACACCCCAG TTGAAATCTT TGCAAAGGTC | |
| | | GGTTCTATTC AGCGAACAGC ACTATAGCAA AAGAAGATCG | |
| | | TTCCATATTG TACGCCCCAT TAAATTACAG TGTTTCTTAA | |
| | | TGAACTTGCA AAGGAATATT GCTAAAAACA AACAAAAAAA | |
| | | ACTGTTATCG AACTTTCTTT GTTGCTGCTA GTTAAAACTT | |
| | | GTTGCAACTT TTCACTTCTC TTGTGTCCAG GTATGCAGCA | |
| | | AAATTCTGCA ATTTCACCTT AAAGATACTG TTGGTTTTAC | |
| | | AGATGCTCTC CAACCTATTT TCTATAAGAT GAGGTAGTGG | |
| | | TGAACTCAGA TAACAAACTT CTCTTCTAAA CTGGTTCTGC | |
| | | TTCTAAGACA AGCATCTCCT GCCCTCTCTC CTTCCTCCCC | |
| | | ATCTCTCGCA CGCAGTCTAG AGATGGACTG AGCCTTGCTT | |
| | | CTCACTGGCA GTGTTGAGCT TTGGAGATGG GATGGTTGCT | |
| | | ATGCCAAGCC TTGTTTCTCT GCTCAGAAGA AGTAGAGAAG | |
| | | CTATTATCAA TTAAAAGCAT GCTGTGATGT GACTCCTGGA | |
| | | AGTGACGTAG GAGTGAGTGG CAGGTTGTTT GATTTAATAG | |
| | | GTATCTTAAT CAAGAATTAA GCTTGCAACA TTGGCTTTGC | |
| | | TCAGATGCAG ATGGAAGTGT GATCACAATC ATTTTGAATC | |
| | | CCTCTTCCTC ACTTTTTTTT CTAAGAAAAT AAACATTTTA | |
| | | CTGTTTTTAT GGATCCTTGT CTTCTCCCAT TCATCCAGCT | |
| | | CAGTGTTTTA AGATGATCCT GGGTGCAGAA GTTGAGCCCT | |
| | | CCTTTGCATT GACACTGATA ATTAGCCTAT AGGGCTCCCT | |
| | | ACCCTTCCAT TAAGAATCTA CCAAGCATTA GCAAGGCTGA | |
| | | AAGTGGTCTA AGAGGTGAGG GGACATCCTA TGACTTTTTA | |
| | | GGAAGGCCTG AAACCACCTT GTTACCTTTC ATTTTGTTAG | |
| | | CAAATAAACC ATCCTATTTT GTAACTCTCC CCCTTCAAAA | |
| | | TGCTACATGA GCCTTGCCAC TTCCTTTTTC TCTTACTTCC | |
| | | AGCACACTAG ACATAGCAAA AGTGTTTGCC TACTCAAAAA | |
| | | CATAATACTT TTATGCTGAT GATGGTATTT GGAGATGTGA | |
| | | AAGCCAAAAG CCCCTGGCAG TGGTGGGGAA TGTTGACTGA | |
| | | GTGTTCAGCA GAGTTTATTT TTCCATACTA TATGAAGAGA | |
| | | ATGATCTCTT CTCAAAGACA GAAGTGATAT TTTTAACAAA | |
| | | TATTGTCACA AGTAAATAGC AATCAAAAGG AGAAAATAAC | |
| | | TTTTGTATTT TTTTAATGTG TTTGATAGCT TTGACGAGGG | |
| | | TTCTCTTTGT TACTTTCAGG GGAGGGCATC CTATTAAATG | |
| | | CCACGCCAGC AGTCCGGGTC TGGGTTTGTC CCACAAAATC | |
| | | ACAGGAGCAC TGTATGTTCC TCTCTTTTGG AGTTGTGACT | |
| | | TTGAAGGGCC TCAATATTAG CCACACTGCC GCCTGCAGAA | |
| | | GGTGGAGAGT TAAGATGTTC TATGTCAATT TGCTCTTGCC | |
| | | GAAAAGATGA GCCTCGATTT TAAAATCTAT CCACATCCAA | |
| | | CTGATGGCAC CATTGATGTG CAAATAATGA GATTCCCTAT | |
| | | CTCCTTTTAG ACCTGGGACG GCAAAAGGGA AGGGAAGGAA | |
| | | ACTTAGCAGA GTGCTATTGA CTATAGATTC ACATATTAGC | |
| | | AACAAAATCC CGTAATTCTT TTGGCCAACA GCAGCTATTT | |
| | | TGGGGAGCAG CTGTGGCTGT TACATAAATA GAGATGCAGC | |
| | | CAAAATTTTA GGCCTTTTAT CCTGCTTCTA GCAGAAAAAT | |
| | | GCAGGGAGAG TCAAGTAGTC TAGGGTTTCA GGTTGCCTCC | |
| | | CCTCATATGG TTTTTGGCCA AGTGACTAAA ACAGTTTTCC | |
| | | ACAACTGTAA ACAAACTGCT AAGCCCCACC TCAAACTTGT | |
| | | TCACTGGGGA CTTTGCTTAC CGTTCTGTGG GTGACCTTTT | |
| | | CCGGGATTTC TTGTTCTTAT CAAGCAAGAA TTAAGCACAT | |
| | | GCTAAACGTC TTCCATTTGA CTTCTCTACT CGGTGTCTCA | |
| | | GACAGTGTCT TCCCAGAAAA CCACCACCCT CTACCCAAAG | |
| | | ATGAAACATG CTCATGTCAT TTTTCTCATG GTCACATTTA | |
| | | ACAGTTTTGA CATGTTATAC TTGCGCATAG ATCCAAGCGT | |
| | | TTCTTGGGAA CCTGACTTTT GAGTGTTTAA TAAAGCCGGA | |
| | | AGTGGTGTTG CCCTGAACCA GCAGATTTTC ACCTGGGTTC | |
| | | TGGCTCCGGT GTTTAACACT GGATACATCT TTGATGTGCG | |
| | | AAAGTGAGTT CATCTTCAGA CACATTTGGT ACATCCAGAA | |
| | | ATAGATCCAA GAAATGGGGT GGTTGAGTGG GTCCGCACGA | |
| | | AATGCTTGAT TATGTCAGCA ACACCCAACA CTGTCTGTTT | |
| | | TCCATTTGTT GGTTTTAATC ATAAAATTGT CAAGTGATTC | |
| | | GTGTTTGTAC TTTATTTTTT TGTGCCTTCT GAAAGGATCT | |
| | | AAAACAAAAA TATTTTGCCT TTTTTTCCCC ACGTGTATCT | |
| | | GAACATTAAG CAGATTGGCT CAGACACAAT GAAAAGGATA | |
| | | ATCCAATGTA CGTGCTGGTG CACTCTGCTA GTTGTTATCT | |
| | | CTGTAGGGCT CAGGAAGCTG GAAGGAGGAA GGGAGGGTAA | |
| | | GTGGCCTGGT GAGTGGAGGT AGAAAAATGA TGAGAAATGA | |
| | | ACTGAGAGCA TTAAGCAGAG AGGGTTGATA GGCTGGCCGT | |
| | | GTCCGGGGTG AAATTGGAAA TCCAGCTGCC TAGTGGCCAG | |
| | | TGGGTGGGGC AAGACTGTCA ACGAGATTTA CAGCTGGCTT | |
| | | ACACATGCCT TATGTCCTCT GAGTTGTAGA GTTGTAAAAG | |
| | | TTCAGCAGTG TGTGCACAGC TTTCTTTTGG TTGGCAGAGA | |
| | | TTCAGGATCA TGGAGTACTG CTCTCATAAT TGAAGACGTG | |
| | | TTTGTTATTG GCAGAGAACC TTAAAAAAGG CCTTTACCTC | |
| | | AGCGATGCTT CCTAGCCCCA GGCTTGCAGA GAACACAGAG | |
| | | TGGTGTTGTG GTCTATTTAG GGACAAAGAA GGTATAAAGT | |
| | | CCAGAGATGA GAAAACTGGG TCAGCCCTCA GAAACTGCAG | |
| | | CAGCCACGCA CACAGAAGCC TGCTGGAAGA CAGGTCTCTC | |
| | | TCCGTCCACA GTGCCCATCA TCTGAGCCTG GGCTGGGATG | |
| | | ACTCAACTTA GCAAAGACGG ACCCAGGAGG AGTGCTGGTT | |
| | | CTTCAGTCTT TGTACTGGCC CCATCCTCTC CTCACTGTAA | |
| | | TGTGAGGAAG CACCTCTGTG TCAGGGCTCA CCTGGGCATC | |
| | | CAAAGCGGCC ACGCCCACAA TCCGACAGCC CCCAGGAGCA | |
| | | GGTCCAGGGA TGATGCAGCC CCCTTCTTGG TCCCATGTGA | |
| | | TGTCATCCTG CTTTGTTATC TTCTTATAAC TTTATCCTGC | |
| | | TATAACTTTA TCCTCTTCCC AGCCTCATCC CTGTTTTTCT | |
| | | GTTAGGGCAA GACTCTTCCA TAAGCCTGCT AAAAAACAGA | |
| | | GATGATACCT CTTACAAACT TTACCTCATA GCCTGTGAAG | |
| | | CAGGTTGGCA TGTGGATTAC AAGTCCTGCT TTGACACTGG | |
| | | GCAAGAATTT AAGATTGTTC TATCTCTACT AGTCATAGAA | |
| | | AAGAAACATT GTTAAACATG TTGAGTTTTA AAGGAAGAAA | |
| | | TATTTTCAAA TTCTTAATCC AAGAAAATAC TGAGTTGGAA | |
| | | TCTTAGACTT CGGGACTCTG ACACGTTCTT TATGAAAGGC | |
| | | AAAATAATTG GTATCTAAAG TTCTCTCCTT CCTGCCTCCC | |
| | | CTAAAGAAAA AGGATATTAG ATTGCACACT ATAATTTTAC | |
| | | ATAAGATCTG CCTTCCACAC TTCCCTGCTG GAAGGCATTC | |
| | | TCAGAGCTTT ATGTCTTCGT ACCTCTCAGA GATTGGACTT | |
| | | TTTCTTGTTT AAAACCCCAA CCAAAAAAAA TAATAAGGCA | |
| | | TGATTGGTGG GGAGGGAATG TGTATTTAGG GGCATAATAA | |
| | | AAAGGTGGCT CGAAGCAGGA ACTTTGGCCT CATGGTGTCA | |
| | | TGGGTGGATG CCTGGACTCC AGTGTGCCTG TGAGGGGCTG | |
| | | GGTTAGGCAG TCGGCTGTCA CACTCACATG TGCCTGCAAT | |
| | | AAACCTTTTG GAATTTCATG AACGAGGTCT ATGAATTGCC | |
| | | TTTTGCCAAT GAATGGATGT ATTTTTCCAA GGGGGGAATA | |
| | | GTATCCTTGA CTTTGGCAGT CACCTTTTTG TATGTCTCTA | |
| | | GAAAGGGGTC AAAAAACTAT GGTAAAGATG AGGCTTATGA | |
| | | GTGAATACCT CTGGGACAAA CCTTAGGACT CACAAGCTAT | |
| | | GCCATGTTTT TCAGGAGACT CTTGTACCTT ATCTGGAATC | |
| | | TAATCTTGGG AGAAGAGGAA AAAGGAGCTA ATATTTGTCA | |
| | | TTTATACTCA CTCTGTGCCA GATACTGTGC TAGGCATTTT | |
| | | ATAATTGTTT TGTGTCATCC TCATGATAAT CCTGTGAAGT | |
| | | AGATCTATTA CCCCCGTGTT CTAAATAATA GATCTTAAGT | |
| | | GTGGAATGCA TCTATCCAAC ATAAATGCCC ATGTTGAAAG | |
| | | AAGGAAAGAT GTCATTCAAG TATTTTTCAA ATTCTTTTTA | |
| | | TTATGACTAT GCCCTTCGCA ACACTGTGAA AACAACCCCT | |
| | | GGGGGCATCT GCCTTCCAGA ATCTCTCTCT GGCTTCTCAC | |
| | | CAGCTTGGTT TCCTCATGGG GAGTGTTTTA TTTGGCCTCC | |
| | | CCTATCTGAG CTGCACACAC ACCAGGGGAG GCCACTGGCT | |
| | | AACAGTAGGA CTTCAGTGCC CTGAGGAAAA GGCTTTGGGA | |
| | | ATTTAGGCAC ACGTTTCTCT CCTTGGAATC CTTCTAGCAT | |
| | | CTGGAAAAGG AACCTGGTAT TTCTGCAGTT AGATACCAGA | |
| | | TGCAACCAGA ACAGGCTTTC GAGTGCTGTG ATTTCTTTCC | |
| | | TGGGTTCCCA AGTCTTGTTG TTTCATCACA AACTGTATCT | |
| | | TTTTAAGGTT AAAAGTCTTG ACCTTCATGG GGGTCTGGGA | |
| | | CAATCCGATC TCCAAGCATG GAGGAAAGGC AATGCCTGGA | |
| | | CCACTGACTT GCATTGAAAT CCTTTCTTGT GGGCTAGGGT | |
| | | TTGATGTCTC TTTTTCATCT TTGGACTGGG GATCTGCATC | |
| | | TTCCAGGTCC ATTTAAGCAC TGAAACTAGA TGCAAATCTC | |
| | | TTTCGAGACC TTACATGTTT TAGATAGTCA TGTAATGACT | |
| | | TGGATAGACA TTTAAATAAC TTGTTCCAAG GTCGGAAGAG | |
| | | CCCAAGAACT CCTCAGAGTT CCTCTTCTTG CTTCTTGAAT | |
| | | CACATCCTCT AAAGATGACT CATGTCTCCA TAGCAACTGT | |
| | | TAAAGGTGCT GCCTAGACGG GACCCGCTCC CACCTTTACT | |
| | | TACTTTGCTC GAAGGAACCA AAACAATGTC CTTGTGTAAA | |
| | | GGGGCTGTCA TATCCAGTTT TCCTTTGAAA TCTGGCCCCC | |
| | | AAGATCCTGC TTCTTTCTAA CCTGGCAGCT GTCATGTCCC | |
| | | TTCAAGATGA TGTGGGAAAT GGCCCTTACA ACTTGGGAAC | |
| | | CACAGAAATT GCTGTATTTC GGGAAGATTC ACCTCTAAAC | |
| | | TGAAGGCTTC ATTCTGATAG TGTCTGCCCT CTCTACCCTG | |
| | | ATTTCGCCCT TCTTTGCTTC CATTTTTAGC CCAAGGCTTT | |
| | | GAATTTGATT GAGTAAGACT TAGAGGCAGT ATAAAGAACA | |
| | | CCATAAACTT AGGCAGAGGT CCCTTAGGGT CTCTAGAGTT | |
| | | GAAAATAATT CTACAGCCTT AGGGGGACCT CTTGGCATTG | |
| | | ACTCTAAAGG GAGAGAATAG CCCCTGTGTC CTGGCATTTC | |
| | | AGTCTAGACC TTCAAGGACT GTTCTCTCTT GACAGGCAAG | |
| | | CAAGCAAAGA AAGTTTTGCA ATAGATTTCA AGCCAGTTTT | |
| | | TCCATTCAAA CCAAGATGCA AATTCATAAA ATTACTCTTT | |
| | | TCCTGGAATA GATCCAGGCA GCTGCCTTAT TAGAACTTTA | |
| | | GATTCGGATC TATTTTCTTA ACACACATAC ACATACACGC | |
| | | GCATACATAC ATATACAGAG AGATACGTGG AGAAAGGAAA | |
| | | TTTACTCTAT CATTGCAATA CTTCAAGAAA GAGCTGTATT | |
| | | TTGCCTTTCT GTAATCTCCA AGATAGTGTC TAGGAAAGTA | |
| | | ATAGTATAAC TATAGGGATA CCGAAACAGG AAAAACCAGC | |
| | | CATCACTCTT GAGAAAGTTT GAGTTCGACT CACATGGGAG | |
| | | AATCGAGGTC TGCTACTCGT CTTGCTTTGT GCCCCATCTG | |
| | | TGCCTGGATG CCCTACTACA TCTGCTTGAC TCGTCTGGGC | |
| | | TGCTAGCCGG GGTGTTGTGG CTGACATCCT TTCCTGGCCT | |
| | | TACACACATA ATAGACACAT CCCTAACGGC GTGTGCCTGG | |
| | | TCCAGCCACA TACAGCCACC ACATGTGTCA CACACTGTCC | |
| | | CCCTCATCCA TGTGGACTTG ACTGGCATTT CAGCAGCTCC | |
| | | ACTGGGATGC TCTAACCCCA GTGTGTGGAG TTGGGGTCCC | |
| | | TTCATCTAGG TTGACCCAGG TATAGCATTT TTAGCATTGC | |
| | | CTTTCCAGTC TTGATGATTC ATTCATTGAA CTCATTTATT | |
| | | TCTGGAGCCC CTGGTACACT CCAGGCACTG CGCTAATTGC | |
| | | CAGCAAAGCA CAACTGAACT AAATCCACCT TCAAGGAACC | |
| | | TAGCCATAAC GAGGGAGGCA GCATGGAAGT ACCCTACAGG | |
| | | GGAAAGTCCT GAGTGCTGTG GGAGCATCTC ACCGTGGCAG | |
| | | CCAGCCCAGT TTTGGCAATC AGGGGCTTCC TGAAAGAGGT | |
| | | GACATCAAAG CCCGGATGTG TCAGAGGACT GAGGGAGAGT | |
| | | GTTACTAAAG GACTTTCAGG CTGAGAGGAT AGCACAGGAC | |
| | | TCAGCCCAAA GGAGGGCCAG TGTGGACTGT CCAGGGCCAG | |
| | | CCTGCAGTAC AGAGGCTGGA GCTTGGACTT GTAGAGGGAG | |
| | | AGAGAAGAGC AAGGGACGTG GACGGGGCAG TGAGCCAGGC | |
| | | TAGCCACAGA GGGTTCCCGG GGCTTTGCTG GGGATTCAGG | |
| | | GAGCATAAAT AAGAGCTTTA GGTGGTGCTG TGTCCTCTGC | |
| | | AGCCCACTGC TGAGGTCCTC CAGACAGGTA AGGTGTGGTC | |
| | | ACAATCAGGG CCGGGGTTTC CCTGCTCACT GCGGCAGTGC | |
| | | AGGGGTGCTT GCTGAGATGA TTCATCCCAG GGTGTCCTCT | |
| | | GTCCCTTACC CAGCCCCAAC TCCTCTTCCT CTGCCAAAAG | |
| | | CTATTTGAAT TCAAGGACTT TAACCTGGGC CGGATCTGGT | |
| | | TTGGAGACAA AGGGGACAGC TCTGGGTCAG CATGACCTTC | |
| | | TTTAGAGCCA CTAAGGCGAA AAATACCGTT TGGGACCAGG | |
| | | CTGGCCTAGA CCCAGGGATG AGAATGCACC CTAAAATAAA | |
| | | TATACGGGAA GCAGCAGAGG GCTTCCCTGT CTAGTGTGTG | |
| | | ATCCTAACTA AAGGCAGCTC TCTTGGACAG CCTTCCCCTG | |
| | | GATTAGGTCA CATACACCTG GTGGCCAAGC CTCTGCTGGG | |
| | | TCCCAAATAC ACACCCGAGT CCTGCCAAAG AAAGGAGATT | |
| | | TTTAAAAAGC ACAGACAAAT TGTATGCAAG TGGAAAATAC | |
| | | CCATAGGCCT AGACAGCTGT GGAGGGAAGA CCTCGTGGGT | |
| | | ACCTGGAGGC TGCCAGAGCT GGGAGCTCTG CAGGTATGAG | |
| | | TCAGGGAAGG CTCAGAGACA AGCAGAATCT CTCTATGGAG | |
| | | ACAACTTGCA GTGCCTTTTA GGTTTTCCAA ATAACCTCGG | |
| | | AGTTCAGAGC ATTGGGTTTT TTTCTCCCCT CCCCACCCCC | |
| | | AGAAAAATAA TTAGAAAAAT GTTTAGGAGA AAGGAAAAGA | |
| | | ATTAGATGCA TCAGAATACC AGCTATAAGC CAACACTGTT | |
| | | TCCAGAAACT CAAGAAAAAG CTCAAACAGA AGACAGTTCC | |
| | | CCTGAGAGGC TGGAGGCGTT GGTGCTGAAG GCAATTTTCC | |
| | | TAGCTAAGGG GCACTGGGCC TTGCTGCACC TTGGGGCTGA | |
| | | CCTTTTTTGC AAAACACCCA CCCCTGCCCT CCTGGCATAC | |
| | | TCAACAGCAA CGCCAGCTTT CTGGACCCTT GGAAAGATGT | |
| | | TAGCTCAAAC ACCCACTTTT TCCAGATCTT CCTCTTGCTC | |
| | | TTCACTGAGG AATTTGTAAT TCTGAGGCTA GCGATGCCCA | |
| | | CTCGGATATT CCGCAGGCCC AGGTGTTTAG ATTAGAATTT | |
| | | GTCCAGCGGT AATCCTGATG CTGGAAACCA ACAAACATTT | |
| | | GGCCTCATAT TCACCCATTT AAAAACTAGA GCCCCTGGCA | |
| | | GGTCCCCTTA GGGCCATGTG TTCATGGAAT ATAAGCCAAG | |
| | | TTTGCCTTAG GCTTGTTCAT GGAATATAAG CCAAGTTTAC | |
| | | CTCTCCCCAT TTTCTGCCCT GGCCCACTTC CCACTCACCT | |
| | | CCACCTCATT GCCAGGAAGG GATCAAAATG CCTCCATGCC | |
| | | AGTTGTTAAT GGCTACATAT TTGCCCTTCC CAAGGGTATT | |
| | | TGCATTTTAT TTAGGAACAT GGCCTTATAT TCAAGGAAAA | |
| | | TCTAGCATCA AGATTACGAG GCATCACCTC TCAATCAGGT | |
| | | CTGGGAGGTA TCTTGGGGCA TTGCTCTTCT GAACACCTGC | |
| | | AGAGGCTTCC TCAGGTGAGT GTGGGAGCCC GGAAGGGTGG | |
| | | CCTCCCTAAC CACTCTGCCT GCACATGAAT TCTCCAAAGC | |
| | | AGTGGGCCCC CATCTGTTTC AATTACACAT GCCTGTCAGC | |
| | | AAAAACTTCG TGAGATGCAC TCTCTCTGTG TGTTTATTAA | |
| | | TTTATTTAAA GCATATATCC CTTTACTTTT GTACTACTAT | |
| | | ATTAGGCACA TTATAAAAAG TATACAGCAT AGAAACTTTA | |
| | | AATGAATAAG ACACAAAATA TTATAAACAG AGGTTCTGGC | |
| | | ATTTTCTCTC TGAACTCCTG AGGGGGACCT TGGGCACCTC | |
| | | CTGGTATGTG CACACCCCAC TTTGAACACC CTTGCTTTAG | |
| | | TGCAAATCAG CACGCCTAAA TAGCATCCCA AACCACTGTG | |
| | | CGACATTTGG CCTGCAGAAT AAGAAAGTCC TAAGGCAGAA | |
| | | TCCCCAGGAA CTTCAAATCT GGGAGATGAG GAAAGAAAAC | |
| | | TCACTCACAA ACAACATAAA TGTAAATAAT TCAAAACCCT | |
| | | AAAGGAGAGC TGTCCCTAGA CAGTAGTGGC TACAGGTGCT | |
| | | AAGCAAAAGT CAGGTACACT GGACAGAGCC ATGCGATTGA | |
| | | TTGTTCATCT TCCCTCTCTG GGTCTCCAGA AAAGGACACT | |
| | | GGCAATATCC CCAGCTCTCT CCTGATTGGA ATTCTTTGAA | |
| | | CCCTCGAAGT GCTCCAGCAG TACTACCCCC CACGTAGCAG | |
| | | TGGGCCCATG TCGCTTGAAT ATTATTTTTG ATTTGACCAC | |
| | | CAAGATGTAA TGATGATTCT ATTCCATTTT GAAAAGGGTC | |
| | | TGAGAAAGTG TACAGGTCTA ATTATATATA CATATTTATA | |
| | | CATGTGTATT TTTGTTTATT GTTACATTTT GACATTGGAC | |
| | | TTTCTATTAA ATAATTTTTA AGAGTTGG | |
| CFLAR | NM_001127183.2 | GACCACGCAT GGAGAATTTT ACCACCCAGA GACACGCGAG | 4 |
| | | TGGCCCTGTG CAAGTTTCAA CTGCGCGGGG GGCGGGGAAT | |
| | | TCCGCAGACA GGATTCTAGA ATTTATGTCT TGTGTGGTAA | |
| | | CATTTCAGCC GGTGGGTGGC GGGGATTAGG CGTGAAGCGG | |
| | | TTCAGCAGGC AGAGGTTCTC GGACGCCCTC CGGCGAAGCC | |
| | | ACCTGTTGAT GCTTTTGACT TTCTGTCCTT GTTCCTCGTC | |
| | | CCATCTGGAG CATTTCCAAT TCTGGTTTTG CGGAGCAGCA | |
| | | GGTCTGAGCT TGTCCGGCGA GGGTGGGAGT TGGTCCCGGC | |
| | | GGAGATCCAG TGGGAAGAGC CGGCGGCTGC CCGGGCAACT | |
| | | CCCCCACTGG AAAGGATTCT GAAAGAAATG AAGTCAGCCC | |
| | | TCAGAAATGA AGTTGACTGC CTGCTGGCTT TCTGTTGACT | |
| | | GGCCCGGAGC TGTACTGCAA GACCCTTGTG AGCTTCCCTA | |
| | | GTCTAAGAGT AGGATGTCTG CTGAAGTCAT CCATCAGGTT | |
| | | GAAGAAGCAC TTGATACAGA TGAGAAGGAG ATGCTGCTCT | |
| | | TTTTGTGCCG GGATGTTGCT ATAGATGTGG TTCCACCTAA | |
| | | TGTCAGGGAC CTTCTGGATA TTTTACGGGA AAGAGGTAAG | |
| | | CTGTCTGTCG GGGACTTGGC TGAACTGCTC TACAGAGTGA | |
| | | GGCGATTTGA CCTGCTCAAA CGTATCTTGA AGATGGACAG | |
| | | AAAAGCTGTG GAGACCCACC TGCTCAGGAA CCCTCACCTT | |
| | | GTTTCGGACT ATAGAGTGCT GATGGCAGAG ATTGGTGAGG | |
| | | ATTTGGATAA ATCTGATGTG TCCTCATTAA TTTTCCTCAT | |
| | | GAAGGATTAC ATGGGCCGAG GCAAGATAAG CAAGGAGAAG | |
| | | AGTTTCTTGG ACCTTGTGGT TGAGTTGGAG AAACTAAATC | |
| | | TGGTTGCCCC AGATCAACTG GATTTATTAG AAAAATGCCT | |
| | | AAAGAACATC CACAGAATAG ACCTGAAGAC AAAAATCCAG | |
| | | AAGTACAAGC AGTCTGTTCA AGGAGCAGGG ACAAGTTACA | |
| | | GGAATGTTCT CCAAGCAGCA ATCCAAAAGA GTCTCAAGGA | |
| | | TCCTTCAAAT AACTTCAGGC TCCATAATGG GAGAAGTAAA | |
| | | GAACAAAGAC TTAAGGAACA GCTTGGCGCT CAACAAGAAC | |
| | | CAGTGAAGAA ATCCATTCAG GAATCAGAAG CTTTTTTGCC | |
| | | TCAGAGCATA CCTGAAGAGA GATACAAGAT GAAGAGCAAG | |
| | | CCCCTAGGAA TCTGCCTGAT AATCGATTGC ATTGGCAATG | |
| | | AGACAGAGCT TCTTCGAGAC ACCTTCACTT CCCTGGGCTA | |
| | | TGAAGTCCAG AAATTCTTGC ATCTCAGTAT GCATGGTATA | |
| | | TCCCAGATTC TTGGCCAATT TGCCTGTATG CCCGAGCACC | |
| | | GAGACTACGA CAGCTTTGTG TGTGTCCTGG TGAGCCGAGG | |
| | | AGGCTCCCAG AGTGTGTATG GTGTGGATCA GACTCACTCA | |
| | | GGGCTCCCCC TGCATCACAT CAGGAGGATG TTCATGGGAG | |
| | | ATTCATGCCC TTATCTAGCA GGGAAGCCAA AGATGTTTTT | |
| | | TATTCAGAAC TATGTGGTGT CAGAGGGCCA GCTGGAGGAC | |
| | | AGCAGCCTCT TGGAGGTGGA TGGGCCAGCG ATGAAGAATG | |
| | | TGGAATTCAA GGCTCAGAAG CGAGGGCTGT GCACAGTTCA | |
| | | CCGAGAAGCT GACTTCTTCT GGAGCCTGTG TACTGCGGAC | |
| | | ATGTCCCTGC TGGAGCAGTC TCACAGCTCA CCATCCCTGT | |
| | | ACCTGCAGTG CCTCTCCCAG AAACTGAGAC AAGAAAGAAA | |
| | | ACGCCCACTC CTGGATCTTC ACATTGAACT CAATGGCTAC | |
| | | ATGTATGATT GGAACAGCAG AGTTTCTGCC AAGGAGAAAT | |
| | | ATTATGTCTG GCTGCAGCAC ACTCTGAGAA AGAAACTTAT | |
| | | CCTCTCCTAC ACATAAGAAA CCAAAAGGCT GGGCGTAGTG | |
| | | GCTCACACCT GTAATCCCAG CACTTTGGGA GGCCAAGGAG | |
| | | GGCAGATCAC TTCAGGTCAG GAGTTCGAGA CCAGCCTGGC | |
| | | CAACATGGTA AACGCTGTCC CTAGTAAAAA TACAAAAATT | |
| | | AGCTGGGTGT GGGTGTGGGT ACCTGTATTC CCAGTTACTT | |
| | | GGGAGGCTGA GGTGGGAGGA TCTTTTGAAC CCAGGAGTTC | |
| | | AGGGTCATAG CATGCTGTGA TTGTGCCTAC GAATAGCCAC | |
| | | TGCATACCAA CCTGGGCAAT ATAGCAAGAT CCCATCTCTT | |
| | | TAAAAAAAAA AAAAAAGGAC AGGAACTATC TTACTCAATG | |
| | | TATTAGTCAT GTTTCTCTAG AGGGACAGAA CTAATAGGAT | |
| | | ACATGTATAT AAAAAGGGGA GTTTATTAAG GAGTATTGAC | |
| | | TCACATGATC ACAGGGTTAG GTCCCACAAT AGGTCATCTG | |
| | | CAAGCAAGGA AGCCAATTCA AGTCCCAAAG CTGAAGAACT | |
| | | TGGAGTCCAA TGTTTGAGGG CAGGAAGCAT TCAGCATGAG | |
| | | AGAAAGATGG AGGCCAGAAG ACTACACCAG TCTAGTCTTT | |
| | | CCATGTTTTG CCTGCTTTTA TTCTGGCAGT GCTGGCAGCT | |
| | | GATTAGATGG TGCCCACCCA GATTGAGGAT GGTCTGCCTT | |
| | | TCCCAGTCCA CTGACTCAAA TGTTAAATCT CCTTTGGCAG | |
| | | CACCCTCACA GATGTACCCG GGAACACTTT GCATCCTTCT | |
| | | ATTCAATCAA GTTGATACTC AGTATTAACC ATCACAGTCC | |
| | | ATTTGGGCAA CTATACCAAA TTACCATAGA CCAGGTGACT | |
| | | TAAACAGCAG TTATTTCTCA CAGTTCCGGA GGCTGGGAAA | |
| | | TCCAACATCT AAGTGGTAGC ATATCTGGTG TCTGGTAAGG | |
| | | CATGCTTCCA GATCTTACCA GATGTCAGTC TTTTGATGTT | |
| | | CTCACATGGC AGAAAAAGAG GATGCAAACT CTCAAGTATA | |
| | | TCTTTAAGGG CACAAATTCC ATTCATGAGG GCTCTACCCT | |
| | | CATCACCTAA TTACCTCCCA AAGGCCCCAC CTTCTGATAC | |
| | | TGTCACTTTG GGGATACTGT CTCCCCTTTG AATTCTGGGG | |
| | | GGAATACAAA CATTCAGTTT GTAACAATAG CCTTATGATT | |
| | | TAGAGGTTAC TTGTTCATTC ACCTAGACCT CAAATTGCAT | |
| | | TTTACAGCTA GTCAAGTATA TCTTTCTCTG ATTTGATAGT | |
| | | GTGACCTAAA AGGGGACCAT TGTTTGAAAT ATCATTAGAG | |
| | | TTGCTTATTA TTATTATTAT TATTATTATT ATTATTATTA | |
| | | TTATTATTAT TGAGACAGAG TTTCATTCTG CTGCCCAGGC | |
| | | TGGAGTGCAG TGGCATCATC TTGGCTCATT GCAACCTCTG | |
| | | CCTTCTGGGT TCAAGCGATT CTCCTGCCTC AGCCTCCCGA | |
| | | GTAGCTGGGA TTACAGGCTC CTGCCACCAC ACCCGGCTAA | |
| | | TTTTTGTATT TTTAGTGGAG ACAGGGTTTC CACCATGTTG | |
| | | GCCAGCGTGG TCTTGAACTC CTGACCTCAG GTGATTCACC | |
| | | AGCCTCGGCC TCCCAAAGTG CTGGGATTAC AGGTGTGAGC | |
| | | CACTGCACCT GGCCTATTAT TATTTTTAAA TTTTTTTTTT | |
| | | TTAATTGATC ATTCTTGGGT GTTTCTCACA GAGGGTGATT | |
| | | TGGCAGGGTC ACAGGACAAT AGTGGAGGGA AGGTCAGCAG | |
| | | ATAAACAAGT GAACAAAGGT CTCTGGTTTT CCTAGGCAGA | |
| | | GGACCCTGCG GCCTTCCGCA GTGTTTGTGT CCCTGGGTAC | |
| | | TTGAGATTAG GGAGTGGTGA TGACTCTTAA GGAGCATGCT | |
| | | GCCTTCAAGC ATCTGTTTAA CAAAGCACAT CTTGCACTGC | |
| | | CCTTAATCCA TTTAACCCTG AGTGGACACA GCACATGTTT | |
| | | CAGAGAGCAC AGGGTTGGGG GTAAGGTCAT AGATCAACAG | |
| | | CATCCTAAGG CAGAAGAATT TTTCTTAGTA CAGAACAAAA | |
| | | TGAAGTCTCC CATGTCTACT TCTTTCTACA CAGACACAGC | |
| | | AACAATCTGA TTTCTCTATC TTTTCCCCAC CTTTCCCCCT | |
| | | TTTCTATTCC ACAAAACCGC CATCGTCATC ATGGCCTGTT | |
| | | CTCAATGAGC TGTTGGGTAC ACCTCCCAGA CGGGGTGGCG | |
| | | GCTGGGCAGA GGGGCTCCTC ACTTCCCAGA TGGGGCGGCC | |
| | | AGGCGGACGC GCCCCCCACC TCCCTCCCGG ACGGGATAGC | |
| | | TGGCCGGGCG GGGGCTGACC CCCCACCTCC CTCCCCGACG | |
| | | GGGCGGCTGG CCGGGCGGGG GCTGACCCCC ACGCCTCCCT | |
| | | CCCGGACGGG GCGGCTGCCA GGCGGAGGGG CTCCTCACTT | |
| | | CTCAGACGGG GTGGCTGCTG GGCGGAGACG CTCCTCACTT | |
| | | CCCAGACAGG GTGGCTGTCG GGCGGAGGGG CTCCTCACTT | |
| | | CTCAGACGGG GCAGCTGCGG GCGGAGGGGC TCCTCACTTC | |
| | | TCAGACGGGG TGGCCGGGCA GAGAAGCTCC TCACATCCCA | |
| | | GACGGGGGGG CGGGGCAGAG GCGCTCCCCA CATCTCAGAC | |
| | | GATGGGCGGC CGGGCAGAGA CGCTCCTCAC TTCATCCCAG | |
| | | ACGGGGTGGC GGCCGGGCAG AAGCTGTAAT CTCGGCACCC | |
| | | TGGGGGGCCA AGGCAGGCGG CTGGGAGGCG GAGGCCGTAG | |
| | | CCAGCTGAGA TCACACCACT GCACTCCAGC CTGGGCAACA | |
| | | TTGAGCACTG AGTGGACGAG ACTCTGCCCG CAATCCCGGC | |
| | | ACCTCGGGAG GCCGAGGCTG GCAGATCACT CGCAGTCAGG | |
| | | AGCTGGAGAC CAGCCCGGCC AACACAGTGA AACCCTGTCT | |
| | | CCACCAAAAA AATACGAAAA CCAGTCAGGC GTGGCGGCGC | |
| | | CCGCAATGGC AGGCACGCGG CAGGCCGAGG CGGGAGAATC | |
| | | AGGCAGGGAG GCTGCAGTGA GCCGAGATGG CAGCAGTACA | |
| | | GTCCAGCTTC GGCTCGGCAT CAGAGGGAGA CCGTGGGGAG | |
| | | AGGGAGAAGA GAGGGAGGGG GAGAGGGCTA TTTTTAAAAT | |
| | | TTTTTAAAAT TGCTGAACAG GGGTACCTCT GGGCAGTGTG | |
| | | TCAGAATACC ACTTTTTAAA TATTTTATGA TTTATTTATT | |
| | | TTTCTATTTC TTGAGGTTTT AACTGATGTG TATCTGTATG | |
| | | TCTATTTGTG TATATTTTGT CATGATCATG TAACAGAGTC | |
| | | TGAAAAGTGT CGAAGAGACA GTTTTCAGGA ACAACAAGCA | |
| | | ATTATTCCTA CTTTCCAAGT TATTTTGATG CCATGGTGGC | |
| | | TCATACCTAT AATCTGAGTA CTTTGGGAGG CTGAGGTGGA | |
| | | CTGATCACTT GAGCCCAGGA GTTTGAGACC AGCCTGGGCA | |
| | | ACATAGCAAG ACTCCATCTC TACAAAAAAA GACAAAATTT | |
| | | AGCTGAGCGT GGTGGCGTGT TCCTGTAGTC CCAGCTACTT | |
| | | GGGAGGCTGA AGTGAGTGGA TCCCCTGAGC CCAGAGAGGT | |
| | | CAAGGTTGTG ATGAGCTGTG ATCACACCAC TGCACTTCAG | |
| | | CATGGGAGAC AGAGTGAGAC CCTGTTTCAG AAAAAATAAA | |
| | | TAAATAAAAC CACCAGCACC ACAAACAACA ACAAAAAGTT | |
| | | ATTTTGTACT TGTTTTGAGC ACAGGACTCC TGAGGGTATC | |
| | | TTTGCATTTA ATATTACATA GGGGTGCCAG TGGGAAGTAA | |
| | | TGTGTATGCT TGGCCTCATG AGCTAAAACC CTGTGTTAAT | |
| | | TATGACAGAA GGAAAGTGTG TGAGAGAGAT CTTAACTACC | |
| | | TAGCAGCTCT AGCTGCCATC TTGAACCATG AAGATACGGG | |
| | | CCACACGTAG GGGTAGCTGG GTAGTGAGCA GCAAGAAGCC | |
| | | TTGTTGGATG AGGGCACGAA GGAGCAGAAT CACTGGAATC | |
| | | ACTGTGTCAG CCCTAATTAC CTACCTCTGG ACTTTTATGT | |
| | | GAGGGGAAAA AAAATTGACA GTTTATATTT ATCTCAACCT | |
| | | AGTTAACCCA AGTGATGCAT TGTTATGAGA TTAAAATGTT | |
| | | TGGAGGCCGG GTGCGGTGGC TCACGCCTAT AATCCCAGCC | |
| | | CTTTGGGAGG CCAAGGCGGG CGGATCACGA GGTCAGGAGA | |
| | | TCAAGACCAT CCTGGCTAAC ATGTAAAACC CCGTCTCTAC | |
| | | TAAAAATACA AAAAATTAGC CAGGCGTTGT GGCGGTCGCC | |
| | | TGTAGTCCCT GCTATTTGGG AGGCCGAGGC AAGAGAACGG | |
| | | CATGAACCTG GGAGGTGGAG CTTGCAGCGA GCTGAGATCT | |
| | | TGCCACTGCA CTCCAGCCTG GGCGACAGTG CGAGACTCTG | |
| | | TCTCAAAAAT AAATAAATAA ATAAATAATA AATAAAATGT | |
| | | TTGGAATGTT GGCTTCATCC CTGGGATGCA AGGCTGGTTC | |
| | | AACATACGCA AATCAAGAAA CATAATTCAT CACATAAACA | |
| | | GAACTAAAGA CAAAAACCAC ATGATTATCT CAATAGATAC | |
| | | AGAAAAGGCC TTCAATAAAA TTCAACGTTG CTTCATGTTA | |
| | | AAAACTCTCA ATAAACTAGG TATTGATGGA AAATATCTCA | |
| | | AAATAATAAC CATTTATGAC AAACCCACAG CCATTATCAT | |
| | | ACTGAATGGG CAAAAGCTGG AAGCATTCCC CTTGAAAACT | |
| | | GGCACAAGAC AGGGATGCCG TCTCACCACT CCTATTTAAC | |
| | | ATAGTATTGG AAGTTCTGGC CAAGAAAATC AGGCAAGAGA | |
| | | AACAAATAAG GGGTATTCAA ATAGGAAAAG AGGAAGTAAA | |
| | | ACTGTGTTTG CAGATGACAT GATACTATAT CTAGAAAACC | |
| | | CCATTATCTC CACCCAAAAG TTCCTTAAGC TGATAAGCAA | |
| | | CTTCAGCAAA GTCTCAGGAT ACAAAATCAA TGTGCAGAAA | |
| | | TCACAAGCAT TCTATACACC AACAATACAC AAGCAGAGAG | |
| | | CCAAATCATG AATGAACTCC CATTCACAGT TGCTAGAAAG | |
| | | AGAATAAAAT ACCTAGGAAT ACAGCTAATA AGATGTGAAG | |
| | | GATCTCTTCA AGGAGAACTA CAAACCACTG CTCAAGGAAA | |
| | | TAAGAGAGGA CACAAATGAA AAAACATTCC ATTCTCGTGG | |
| | | ATAGGAAGAA TCAATATCAT GAAAATGGCC ATACTACCCA | |
| | | AAGTAATTTA TAGGTTCATT GCTATTCCCA TTAAACTACT | |
| | | ATTGACATTC TTCACAGAAT TAGAAAAAAA CTACTTTAAA | |
| | | ATTCAAATGG AACCAAAAAA GAGCCCGTAT AACCAAGACA | |
| | | ACAATAAGCA AAAAGAACAA AGCTGGAAGC ATCACACTAC | |
| | | CCAACTTCAA AGTATACTGC AAGGCTACAG TAGCCAAAAT | |
| | | GGCATGGTAC TGGTACAAAA ACAGACACAT AGACCAATGG | |
| | | AACAGAATAG AGACCAGAGA AAGAAGACCA CACATCTACA | |
| | | GCCATCTGAT CATCGACAAA CCTGACAAAA ACAAGCAATG | |
| | | GGGAAAAGAT TCCCTATTTA ATAAATGGTG CTGGGAAAAC | |
| | | TGGCTAGCCA TATGCAGAAA ATTGAAACTG ACCCCTTCCT | |
| | | TACACCTTAT ACAAAAATTA ACTCAAGATT AAAGACTTAA | |
| | | TGTAAAACCT AAAACTATAA AAACCCTAGA AGAAAATCTA | |
| | | TTTAATACCA TTCAAGACAT AGGCACAAGC AAAGGTTTCA | |
| | | TGACAAAAAC ATCAAAAGCA ATTGCAACAA AAGCAAAAAT | |
| | | TACAAATGGG ATCTAATTAA ACTAAAGAGC TCCTGCACAG | |
| | | CAAAAGAAAC TATCATTAGA GTGAACAGGC AACCTACAGA | |
| | | ATGGGAGAAC ATTTTTGCAA TCTATCCATC TGACAAAGGT | |
| | | CTAATATCCA GAACCTACAA GGAACTTAAA ACAAATTTAC | |
| | | AAGGAAAAAA ACAACCCCAT CAAAAAGTGG ACAAAGGACA | |
| | | TGAACAGACA CTTCTCAAAA GAAGACATTT ATGTGGCCAA | |
| | | CAAACATATA AAAAAAAGCT CAACCTTACT GATCATTAGA | |
| | | GAAATGCAAA GGAGAACCAC AATGAGATAC CATCTCATGC | |
| | | CGGTCAGAAT GGTGATTATT AAAAAGTCAA AAAACAACAG | |
| | | ATGCTGGCGA GGCTGTGGAG AAGTAGGAAC ACTTTTACAT | |
| | | TGTTGGTGGG AATGTAAATT AGTTCAACCG TTGTGGAAGT | |
| | | GTGTGTGGCT ATTCCTCAAA GATCTAGAAC TAGAAATACT | |
| | | ATTTGTCCCA GCAATCCCAT TACTGGGTAT ATACCCAAAG | |
| | | GAATATAAAC CATTTTATTA TAAAGATACA TGCACATTTT | |
| | | TGTTCATTGC AGCACTCTTC ACAATAGCAA AGACACAATA | |
| | | GCAAATGCCC ATCAAAGATA GACTGGATAA AGAAAATGTG | |
| | | GTACATATAC ACCATGGAAT ACTGTGCAGT GCAGCCATTA | |
| | | CAGCTTTTGG TGATACAGTG AATCAGATTT TTCATTAATT | |
| | | CTTTTAATTG GTTATTACTG AACGTGAAAA AGTAATGTTT | |
| | | GTATTGAAAT CTTGAGTCTG GCCATGTTTC TATTTTAAAT | |
| | | TCATAAAGAA TTCTAACAAG AGGAATTCCA AGAATGTCAT | |
| | | AAATGGATGT TTCTCCATGG ATGAAGGAAC TGTTTTATTC | |
| | | ACTTGCTGAT AATTCAGCCT AATCCAGTTT GACATCATAT | |
| | | AGATAAGTAG TTGAATTATG GATTTAAAAT ACATATCATT | |
| | | TTCTAACTCC AAAGGTAATA CTTATTTAAA TGGTTTTGAA | |
| | | AATATAGAAA GGCACAATTT CTTTTTAAAT CTGTTATTCT | |
| | | CCACCACCAC TCAATCTGTC TATCATCTAT CTCTCCATTC | |
| | | ATTCTTCCAT TTGTTTATAT CTGTTAATCT TTGTATGTGT | |
| | | TCATGTATAG CTTTTACATG ATTGGAATCA TAATGCATAT | |
| | | TCCATTTTGA AGTCTGCTTT TTTTTACACA AAAATATGTT | |
| | | GTGAATATTT TCCTATATTA TGAAATATCA TTAGCTGAGC | |
| | | TTTTAGAATT GACTGCATGT TTTGGTACCA TTTAGATATA | |
| | | GTTTAAGATA CTTAGAAGTT ATGTGGCTTT GCCACTATGG | |
| | | ATGAATCTTA TTTACTCAAT ATTAATTACT TACAAATAAC | |
| | | CTCACCTAAA CACTACTCAG CCATAAAAAG GAATGAATTA | |
| | | ATGACATTCA CAGCAACCTG GAGACTATTA CTCTAAAGGA | |
| | | AGTAACTGAG GAATGGAAAA CCAAACATTG TATGTTCTCA | |
| | | CTCATAAGTG GGAGATAAGC TATGAGGATG CAAAGGCATA | |
| | | AGAAGGATAC AATGGACTTT GGGGACTTAG GGGAAAGGGT | |
| | | GGGAGGGGGG TGAAGGATAA AAGAATACAA ATTGGGTTCA | |
| | | GTGTATACTG CTCAGGTGAT GGGTGCACCA GAATCTCACA | |
| | | AGTAACCACT TAATTACTTA CGCATGTAAC CAGATACCAC | |
| | | CTGTTCCCCA AACACCTATG GAAATAATTT TGTTTTTTTT | |
| | | TTTAAAAAAG GAATGAGATC ATGTCCTTTG CAGGGACATG | |
| | | GATGAAGCTG GAAGCCATTA TCCTCAGCAA ACTAACAGAG | |
| | | GAGCAGGAAA CCAAACACCA CATGTTCTCA CTTGTAAGCG | |
| | | GAAGCTGAAC AATGAGAACA CACGGACACA GGGATGAGAT | |
| | | CAACACACAC TGGGGCCTGA TGCAGGGGCC GTAGCGGGGA | |
| | | GAGCATCAGG ATAACTAGCT AATGCATGTG GGGCTTAATA | |
| | | CCTAGGTGAT AGGTTGATAG GTGCAGCAAA CCACCATGGG | |
| | | ACACGTTTAC CTATGTAACA AACCCGCACA TCCTGCACTT | |
| | | GTATCCAGAA CTTAAAATAT TTTAAAAATC TTTAGAGAAT | |
| | | ACAAAAAAAA AAAAAAAGAT TCTTCAATGC ATACACAATA | |
| | | AAATTGCAGT TCAGTCAAAC ATTGGAAGTC TTTCTCTGAC | |
| | | TGTCTAGTTG GTATCTTCAT TTTCAGCTTC TTCAAGATCC | |
| | | CACTCCAAAC ACTGTTAGCT CAGCCAAATT GAACAGCTCA | |
| | | TATCTCCTAC CTCTGGATCT TTGGTTCTGG TGATTGTATA | |
| | | TTTCTGGACC ATCTGGAACC CCAGCATATC ACCCTACCCC | |
| | | ACATCTCCAC ATCCCCAAAA TATAACCATA CTTCAAGGGC | |
| | | AGTTCAAATA CCATCTCCTT CTATCCTCCA TGAAGTCAGT | |
| | | TATCTCTTCC ATTGGAATTA TCGCCCCCTC TCCTGAACAG | |
| | | TACTATTTCG TGTGAATCTC CTCCAAGCCT TCTTTTCATT | |
| | | TTATATCTCA TGCTGTAATT CTTGGAAAGT ATGCTGTAGC | |
| | | TCAAGTGCAG AATTCTCATC AGTTTTATCT TTATATCTCT | |
| | | CCTAAACACT TTACCTGATG AAGAGCCTGG CATACACATA | |
| | | AATATATATT GAATGAATCA GTGATGGATT GAAAAGAGAA | |
| | | ATGATGGATC TCCTAAATTT TAACTTTTAT AAAATATTTT | |
| | | GATACATTCA TGACCTTACT TTAGCAAGCA ATGAACGTGA | |
| | | TGTAAACTAT TGTTGATATA GTTTTTATAT TGGAAGTGTA | |
| | | AGTAGTTTGT GGCATGGGAT TGTGACATAT CCTAGGTTTC | |
| | | CTCATCTTCT TTTTATTGAA ATGTAATTCA CAAGCCATAA | |
| | | AATTTGCCCC TTTAAAGTAA ATGATGCAGT GGATTTTAGT | |
| | | ATATTTACAG AGTTGTGCAA TCATCACCAC TATCTAATTC | |
| | | CAGAACATTT CCATCTACCT AGAAACTCCA TACCAGTGAG | |
| | | CTGCCACTCT AATCCTCCTC TTCCCCCAGC CTCTAGAAAC | |
| | | AATAATCCAT TTTCTGTCTC TATGATTTGC CTGTTCTAGA | |
| | | TATTTTATAA AAATAAACAT GTGGCCTTTC GTGTCTGACT | |
| | | TCCTTCACTT AAAAAAAAAA AAAAAAAA | |
| CFLAR- | NR_040030.1 | CATGCATACA GCCCAGTCTA ACGTACAGGA TCAAAAAGAG | 5 |
| AS1 | | ACCTTATTTC GGCTGGCAAT TACCTTACCC GCTTGTTCAT | |
| | | TATCTGACTC CTGCACTCCT CCCAAGTGTT CAATGTTAAC | |
| | | CAGATGGCAA AGTTAACCAG ACAGAGGAAA TCCACATAAT | |
| | | TACTCTAGAT GAAGCCCCCT AACTGCGGAT TAGAAGGAAA | |
| | | AGAGATGAAG GGTGCTGCAC TAATAATAAC AACCATTTTC | |
| | | AACCAAGGCA GGATTCTGGG CCTAGAATCT TCAGTGACAA | |
| | | ATTACCAGGA GAGGAGGAGG GAAATGAAGA GCTGGAGGCC | |
| | | TAGGGAGATG GTGTGAGGAG AGGAGACTGT GCCTTTCCAC | |
| | | TTCTTAATAA TTAATGTTCT CCTGCTGCTC TGAGTCTGCA | |
| | | TCACCAGTGA TTCAGTTTGA GTCAGACATC CAACAACAAC | |
| | | TGAGCAAAAG AATCATCTCA GATCCAAGCA ATGGTGACGC | |
| | | GCAGATAGAA GAGACTGAGG CCAACGACCA AAGCAAAGGA | |
| | | CAGCAGAACT GACTGACACA GCTCAGAAAA TATCCAAAGC | |
| | | TCTGTATGCT CTGGAAAGGA AGACAAGAAA TGCTAAAGAC | |
| | | TCTGGATATT GCTCTGTTTC TATAAGGTCC CACCCATATA | |
| | | CCATCTCTTC TTTTGGATTT CCTTTGGCAG GGTTCCCTTG | |
| | | TATGGAAGTT CTCAGGAGAA CAACACTCTC AGAGTCCCAC | |
| | | TGTCTCAAAG AATTCTTAAT GGACAGCTAT ACTTGGCCCC | |
| | | AAGGTTTCAA CCTCACACTG AAGAAAGTGA CATAAGAAAA | |
| | | CACAAGTCCC AGTGCTATCA GGGGCTATCT GGAAAAGCTG | |
| | | CTTAAACAGC AAGTGAAAGA AGGCTCTCCC CTACCCCGTT | |
| | | CTAGACTCAC CCTGAAGTTA TTTGAAGGAT CCTTGAGACT | |
| | | CTTTTGGATT GCTGCTTGGA GAACATTCCT GTAACTTGTC | |
| | | CCTGCTCCTT GAACTATAAA AGCAGAAGGG AGGTACTGAG | |
| | | TTAAAACTTA CAAATTATTA TCAAATAAAT CTTCAATAGT G | |
| CHP1 | NM_007236.4 | ACCACCCCTG GGTTCCCTCC CGGGTCCGCA GTGGAAACAC | 6 |
| | | TGCCCTCTCC CTTCTTGACC CCTAGCCCTT CCTTCCCTCC | |
| | | CTCCTTCCCT CCTGTCGCCG TCTCTTCTGG CGCCGCTGCT | |
| | | CCCGGAGGAG CTCCCGGCAC GGCGATGGGT TCTCGGGCCT | |
| | | CCACGTTACT GCGGGACGAA GAGCTCGAGG AGATCAAGAA | |
| | | GGAGACCGGC TTTTCCCACA GTCAAATCAC TCGCCTCTAC | |
| | | AGCCGGTTCA CCAGCCTGGA CAAAGGAGAG AATGGGACTC | |
| | | TCAGCCGGGA AGATTTCCAG AGGATTCCAG AACTTGCCAT | |
| | | CAACCCACTG GGGGACCGGA TCATCAATGC CTTCTTTCCA | |
| | | GAGGGAGAGG ACCAGGTAAA CTTCCGTGGA TTCATGCGAA | |
| | | CTTTGGCTCA TTTCCGCCCC ATTGAGGATA ATGAAAAGAG | |
| | | CAAAGATGTG AATGGACCCG AACCACTCAA CAGCCGAAGC | |
| | | AACAAACTGC ACTTTGCTTT TCGACTATAT GATTTGGATA | |
| | | AAGATGAAAA GATCTCCCGT GATGAGCTGT TACAGGTGCT | |
| | | ACGCATGATG GTCGGAGTAA ATATCTCAGA TGAGCAGCTG | |
| | | GGCAGCATCG CAGACAGGAC CATTCAGGAG GCTGATCAGG | |
| | | ATGGGGACAG TGCCATATCT TTCACAGAAT TTGTTAAGGT | |
| | | TTTGGAGAAG GTGGATGTAG AACAGAAAAT GAGCATCCGA | |
| | | TTTCTTCACT AAAGGAGACC AAACTGTTCC TTGCGGTCTA | |
| | | GTATTTAAGA ACTGGAACTT GAAAGTCCTC CTTCTACCAA | |
| | | CTCCACCTCC ACCCCCTCAT TCCCCTTCTC CCAAAGTACT | |
| | | ACTGCTGTTG CATGACAACC CCAAATATGT TCTGTCAACA | |
| | | CAAACCTGCC TTTGGTGTAT AAACAGGGCA TTACAGAATG | |
| | | GTACACCCTA TATATTTCTG TTCAGTATCC ATTCACTAGT | |
| | | TCTTCATTTA TAAATATCAT CTTCCCCATT CTGCTGCTGA | |
| | | ATGCCACACA TCCATCCAGT CTGAGAAAGT GAGAGAGGCA | |
| | | ATCATGCCAA GAACAAGCCA GCAAAGCTCT TTCACCAGAT | |
| | | GTAGACTGTA GCCCTGCTGC CTTCCCTCCA GCGAGTCTGC | |
| | | CAGCATGCTT CTTCATCCTT TTTATATGTT CTTTGCTTCC | |
| | | TACTTCCCTG TCTTCCAACA TACTGTTCAC TTACTCTGGC | |
| | | AGTCTTTCTG CTTTTCATTA AGCCTCAAAA TCTCCTCTGT | |
| | | TCTACTTGGC ACCACAAGCT ATGTCCTATA TATGTATTTC | |
| | | TGACTTGGCA GGATAGTTCA GGGGTCTGGC AGTTTTTATT | |
| | | TACCTTCATT ATTAAATGGG CCTCTGGGAT GTTGCCTCTT | |
| | | CAGGAGCTTT TTGGTAATCA ATACTTCTCT CAGAAGTATG | |
| | | AGACCATCCT CTGCACTCTG CTCTGTCATC AAAGGCTGCT | |
| | | GGGTGGAGAT ACCCTTTTTG AAAGGTGGCC TTGGTGAGAG | |
| | | GTATGGAGCC AAGTCTTCTA GGTTGCTTGC CCACATCACT | |
| | | CTATCTCTGG CCTCTGATTC TCAACTTTGT ACCTGTGTGG | |
| | | CTCCTCTTGT TAGTGCAATG TTGACTGTTG AAAAAGCAGC | |
| | | AGTATGCTTA CAGGTTTGCT TAGTTTGGGG ACACCGTTAC | |
| | | CACCAGAATG GCTGCTCTGA CAATATGCCT AGGGACTTTC | |
| | | TCATGGCTTT TATTTAATAA GGAGGCTGGG CACCCTATAA | |
| | | AGCCTCATGC ATTCACACCT TTGCAGCATG GTTTATGCCT | |
| | | CAGTGTTATG TGCACTGGAA TGTTTTCCAC TTCACATTTC | |
| | | CAAGTAGAAA TATTAGTGTT ACGGAAGTGC CTAATATCCC | |
| | | AGTCCAAATT TTTTTTTTTT TTTTTTTTTT TTTTTGAGAC | |
| | | AGAGTCTTGC TCTGTCACCC AGGCTGGAGT GCAGTGGTGC | |
| | | GATCGCTCAC TGCAACCTCA GCCTCCTGGA TTTAAGTGAT | |
| | | TCTCCTGCCT CAGCCTCCCA AGTAGCTGGG ATTACAGGTG | |
| | | TGCACCACCA TGCCCGGCTA ATTTTTTGTA TTTTTAGTGG | |
| | | AGACAGGGTT TCACCATGTT GGCCAGGCTG GTCTCGAACT | |
| | | CCTGACCTCG TGATCCGCCT GCCTCAGCCT CCCAAAGTGC | |
| | | TGGGATTACA GGTGTGAGCC ACCACGCCTG GCCCCAGTCC | |
| | | AAAATATTTA AAGATTGTTT CCTTAGTGTC TTGAAGTTTT | |
| | | GCACAAAATT CTTTTTTTTG AGATGGAGTC TCACTCTGTC | |
| | | ACCCAGGCTG GAGTGCAGTG GCGTGATCTT GGCTCACTGC | |
| | | AACCTCTGCC TCCTGGGTTC AAGCAATTCT CCCACCTCAG | |
| | | CCTCCCAAGT AGCTGGGATT ACAGACGTGT GCCACCATAC | |
| | | CTGGGTAATT TTTGCATTTT TAGTGGAGAG GGAGTTTCAC | |
| | | CATGTTGGCC AGGTTGGTCT TGAACTCCTG ACCTCAGGTG | |
| | | ATCCTCCTGC CTCGGCCTCC CAAAGTGCTG GGATTACAGG | |
| | | CATGAGCCAC CGTGCTCAGC CGCAAAATTC TTTATGAATT | |
| | | TTACACTTGG CAAATGTTAA TGACGGAAGC CATAGTCTGC | |
| | | TCCTAATACA TGTCCAAAGC ATTGACTGTT GTGTCATTAG | |
| | | CTGCCTGGTT ACATTAGCTC CCTGGCTTCT TGTTTAGACC | |
| | | ACTGCTAATC CCTTAAAAAC AAGAGGTCTG GCACTAGTAG | |
| | | CACAACCTAA GGTGGCATTA CAGATCTTTG AGCGAGCCAC | |
| | | AGCAACTTTT CTGCCAAGTC AGCTTAGTTT AGACTTCAGT | |
| | | GAATCAGGCT ATTGCTATCC TAATGTATGT CTCTATGAGT | |
| | | GTATTTAGCC ACACATCTGC CCTTGGTTGA CTTTCTGACT | |
| | | CATTGCTTGC TTGCTTGTTT CCTTGCTTTG GAAAACTATT | |
| | | GAAGATTGCT AAAAAATACC ACTGCAAAGT GATGGAAAAG | |
| | | GGTGGAGAAC AGGGGAGTAG CCAGGCTGGA TGGCTCAAAT | |
| | | ATAAATGAAT GAGGAATTCT TTATGAAGTA TCAGTCAGAT | |
| | | TTTATGATTA AGTGATGTAA TATAGGAATT ATGTAAAAGG | |
| | | GAAGAATGTC TGATACTGAT CTATTAGAGA GGTACTTTAG | |
| | | AGGCTTCTTG ATTGGCATAA AGTTCCTAAG GTTATAGATT | |
| | | TTCCCCCCTT TTGGCTGTAT AGCAAAGTGT TTTAATCCAC | |
| | | GGTTGTGCCT TATTGTTCCA TTAAAATTGT ATCTTCGATC | |
| | | CATCAATAAA TACTTGTGGT TGAAACAAAA | |
| DDX55 | NM_020936.2 | AGCGGAAGTG CTCGTTGGGG GTGCACAAGG CGCGTTCGAG | 7 |
| | | CAGCGGCGAC CGACGCGGCG AAGGAGCGCG CCATGGAGCA | |
| | | TGTGACAGAG GGCTCCTGGG AGTCGCTGCC TGTGCCGCTG | |
| | | CACCCGCAGG TGCTGGGCGC GCTGCGGGAG CTGGGCTTCC | |
| | | CGTACATGAC GCCGGTGCAG TCCGCAACCA TCCCTCTGTT | |
| | | CATGCGAAAC AAAGATGTCG CTGCAGAAGC GGTCACAGGT | |
| | | AGTGGCAAAA CACTCGCTTT TGTCATCCCC ATCCTGGAAA | |
| | | TTCTTCTGAG AAGAGAAGAG AAGTTAAAAA AGAGTCAGGT | |
| | | TGGAGCCATA ATCATCACCC CCACTCGAGA GCTGGCCATT | |
| | | CAAATAGACG AGGTCCTGTC GCATTTCACG AAGCACTTCC | |
| | | CCGAGTTCAG CCAGATTCTT TGGATCGGAG GCAGGAATCC | |
| | | TGGAGAAGAT GTTGAGAGGT TTAAGCAACA AGGTGGGAAC | |
| | | ATCATTGTGG CCACTCCAGG CCGCTTGGAG GACATGTTCC | |
| | | GGAGGAAGGC CGAAGGCTTG GATCTGGCCA GCTGTGTGCG | |
| | | ATCCCTGGAT GTCCTGGTGT TGGATGAGGC AGACAGACTT | |
| | | CTGGACATGG GGTTTGAGGC AAGCATCAAC ACCATTCTGG | |
| | | AGTTTTTGCC AAAGCAGAGG AGAACAGGCC TTTTCTCTGC | |
| | | CACTCAGACG CAGGAAGTGG AGAACCTGGT GAGAGCGGGC | |
| | | CTCCGGAACC CTGTCCGGGT CTCAGTGAAG GAGAAGGGCG | |
| | | TGGCAGCCAG CAGTGCCCAG AAGACCCCCT CCCGCCTGGA | |
| | | AAACTACTAC ATGGTATGCA AGGCAGATGA GAAATTTAAT | |
| | | CAGCTGGTCC ATTTTCTTCG CAATCATAAG CAGGAGAAAC | |
| | | ACCTGGTCTT CTTCAGCACC TGTGCCTGTG TGGAATACTA | |
| | | TGGGAAGGCT CTGGAAGTGC TGGTGAAGGG CGTGAAGATT | |
| | | ATGTGCATTC ACGGAAAGAT GAAATATAAA CGCAATAAGA | |
| | | TCTTCATGGA GTTCCGCAAA TTGCAAAGTG GGATTTTAGT | |
| | | GTGCACTGAT GTGATGGCCC GGGGAATTGA TATTCCTGAA | |
| | | GTCAACTGGG TTTTGCAGTA TGACCCTCCC AGCAATGCAA | |
| | | GTGCCTTCGT GCATCGCTGC GGTCGCACAG CTCGCATTGG | |
| | | CCACGGGGGC AGCGCTCTGG TGTTCCTCCT GCCCATGGAA | |
| | | GAGTCATACA TCAATTTCCT TGCAATTAAC CAAAAATGCC | |
| | | CCCTGCAGGA GATGAAGCCC CAGAGAAACA CAGCGGACCT | |
| | | TCTGCCAAAA CTCAAGTCCA TGGCCCTGGC TGACAGAGCT | |
| | | GTGTTTGAAA AGGGCATGAA AGCTTTTGTG TCATATGTCC | |
| | | AAGCTTATGC AAAGCATGAA TGCAACCTGA TTTTCAGATT | |
| | | AAAGGATCTT GATTTTGCCA GCCTTGCTCG AGGTTTTGCC | |
| | | CTGCTGAGGA TGCCCAAGAT GCCAGAATTG AGAGGAAAGC | |
| | | AGTTTCCAGA TTTTGTGCCC GTGGACGTTA ATACCGACAC | |
| | | GATTCCATTT AAAGATAAAA TCAGAGAAAA GCAGAGGCAG | |
| | | AAACTCCTGG AGCAACAAAG AAGAGAGAAA ACAGAAAATG | |
| | | AAGGGAGAAG AAAATTCATA AAAAATAAAG CTTGGTCAAA | |
| | | GCAGAAGGCC AAAAAAGAAA AGAAGAAAAA AATGAATGAG | |
| | | AAAAGGAAAA GGGAAGAGGG TTCTGATATT GAAGATGAGG | |
| | | ACATGGAAGA ACTTCTTAAT GACACAAGAC TCTTGAAAAA | |
| | | ACTTAAGAAA GGCAAAATAA CTGAAGAAGA ATTTGAGAAG | |
| | | GGCTTGTTGA CAACTGGCAA AAGAACAATC AAGACAGTGG | |
| | | ATTTAGGGAT CTCAGATTTG GAAGATGACT GCTGATTCCA | |
| | | GTGCCACAGA TGAACCCACA AGGACATAGC TGTTCCCTAA | |
| | | CTTGGTGGAT GGCTCCAGTT TGCTTTTAAC GAAAATCACA | |
| | | ACTTCAGGAG ACATCTGAAA AGAATGATGT CTCTGAAAGC | |
| | | TGTCCTTTCA GATGAGGGAG AAATGAAGGA TTTCACACTT | |
| | | CAGAATATTT TACTAAAAAC ATTCCAGTCT TGGCCGGGTG | |
| | | CGGTGGCTCC TGCCTATAAT CCCAGCACTT TGGGAGGCTG | |
| | | AGGCAGGAGG ATCACTTGAG CCCAGGAGTT CAAGACCAGC | |
| | | CTGGGAACAC AGCGAGACCC TCTCATTAAA AACAACAAAA | |
| | | CAAAACAATT CCAGTCTTGG AGTAGTCTAA CAGAAGAAAA | |
| | | TGTAAAATTA TTTGAGTGTA AATAATAGAT GTCAGTATTT | |
| | | ATCATGATGG GTCACATATA GACATATGTA CATATTATAT | |
| | | ATATATATAT ATATATATAT ATATATATAT ATATATATAT | |
| | | ATAAGCTCTT TTTTCTGAGG CTATTTTATA GTTATTTTTA | |
| | | AACATAAAGA TACAGAAGTC TTCTTGACTT CTGATTTTCA | |
| | | AAACCATTCC TCAGTATCTT CAGGCATTTG ACCTCCTGAA | |
| | | TGTGCTTGGC CCTGGGCTTC AGTTATCCTT TGATGTCCTG | |
| | | CAGGGGTGGC TAATGTGCTG GGGTTTTTCT GTGTTAATAG | |
| | | TCACAGTATT GTTTTATTGG TGAATAGCTG AAAAACAGAG | |
| | | GGATTAAGTC ATATTCCGGG AAAGAGAATT ATAGTTTTTA | |
| | | TGCCTCCTGT TGAATAAATG GTGTCCTGAT TGCCTGGG | |
| DMD | NM_000109.3 | CGTTAAATGC AAACGCTGCT CTGGCTCATG TGTTTGCTCC | 8 |
| | | GAGGTATAGG TTTTGTTCGA CTGACGTATC AGATAGTCAG | |
| | | AGTGGTTACC ACACCGACGT TGTAGCAGCT GCATAATAAA | |
| | | TGACTGAAAG AATCATGTTA GGCATGCCCA CCTAACCTAA | |
| | | CTTGAATCAT GCGAAAGGGG AGCTGTTGGA ATTCAAATAG | |
| | | ACTTTCTGGT TCCCAGCAGT CGGCAGTAAT AGAATGCTTT | |
| | | CAGGAAGATG ACAGAATCAG GAGAAAGATG CTGTTTTGCA | |
| | | CTATCTTGAT TTGTTACAGC AGCCAACTTA TTGGCATGAT | |
| | | GGAGTGACAG GAAAAACAGC TGGCATGGAA GATGAAAGAG | |
| | | AAGATGTTCA AAAGAAAACA TTCACAAAAT GGGTAAATGC | |
| | | ACAATTTTCT AAGTTTGGGA AGCAGCATAT TGAGAACCTC | |
| | | TTCAGTGACC TACAGGATGG GAGGCGCCTC CTAGACCTCC | |
| | | TCGAAGGCCT GACAGGGCAA AAACTGCCAA AAGAAAAAGG | |
| | | ATCCACAAGA GTTCATGCCC TGAACAATGT CAACAAGGCA | |
| | | CTGCGGGTTT TGCAGAACAA TAATGTTGAT TTAGTGAATA | |
| | | TTGGAAGTAC TGACATCGTA GATGGAAATC ATAAACTGAC | |
| | | TCTTGGTTTG ATTTGGAATA TAATCCTCCA CTGGCAGGTC | |
| | | AAAAATGTAA TGAAAAATAT CATGGCTGGA TTGCAACAAA | |
| | | CCAACAGTGA AAAGATTCTC CTGAGCTGGG TCCGACAATC | |
| | | AACTCGTAAT TATCCACAGG TTAATGTAAT CAACTTCACC | |
| | | ACCAGCTGGT CTGATGGCCT GGCTTTGAAT GCTCTCATCC | |
| | | ATAGTCATAG GCCAGACCTA TTTGACTGGA ATAGTGTGGT | |
| | | TTGCCAGCAG TCAGCCACAC AACGACTGGA ACATGCATTC | |
| | | AACATCGCCA GATATCAATT AGGCATAGAG AAACTACTCG | |
| | | ATCCTGAAGA TGTTGATACC ACCTATCCAG ATAAGAAGTC | |
| | | CATCTTAATG TACATCACAT CACTCTTCCA AGTTTTGCCT | |
| | | CAACAAGTGA GCATTGAAGC CATCCAGGAA GTGGAAATGT | |
| | | TGCCAAGGCC ACCTAAAGTG ACTAAAGAAG AACATTTTCA | |
| | | GTTACATCAT CAAATGCACT ATTCTCAACA GATCACGGTC | |
| | | AGTCTAGCAC AGGGATATGA GAGAACTTCT TCCCCTAAGC | |
| | | CTCGATTCAA GAGCTATGCC TACACACAGG CTGCTTATGT | |
| | | CACCACCTCT GACCCTACAC GGAGCCCATT TCCTTCACAG | |
| | | CATTTGGAAG CTCCTGAAGA CAAGTCATTT GGCAGTTCAT | |
| | | TGATGGAGAG TGAAGTAAAC CTGGACCGTT ATCAAACAGC | |
| | | TTTAGAAGAA GTATTATCGT GGCTTCTTTC TGCTGAGGAC | |
| | | ACATTGCAAG CACAAGGAGA GATTTCTAAT GATGTGGAAG | |
| | | TGGTGAAAGA CCAGTTTCAT ACTCATGAGG GGTACATGAT | |
| | | GGATTTGACA GCCCATCAGG GCCGGGTTGG TAATATTCTA | |
| | | CAATTGGGAA GTAAGCTGAT TGGAACAGGA AAATTATCAG | |
| | | AAGATGAAGA AACTGAAGTA CAAGAGCAGA TGAATCTCCT | |
| | | AAATTCAAGA TGGGAATGCC TCAGGGTAGC TAGCATGGAA | |
| | | AAACAAAGCA ATTTACATAG AGTTTTAATG GATCTCCAGA | |
| | | ATCAGAAACT GAAAGAGTTG AATGACTGGC TAACAAAAAC | |
| | | AGAAGAAAGA ACAAGGAAAA TGGAGGAAGA GCCTCTTGGA | |
| | | CCTGATCTTG AAGACCTAAA ACGCCAAGTA CAACAACATA | |
| | | AGGTGCTTCA AGAAGATCTA GAACAAGAAC AAGTCAGGGT | |
| | | CAATTCTCTC ACTCACATGG TGGTGGTAGT TGATGAATCT | |
| | | AGTGGAGATC ACGCAACTGC TGCTTTGGAA GAACAACTTA | |
| | | AGGTATTGGG AGATCGATGG GCAAACATCT GTAGATGGAC | |
| | | AGAAGACCGC TGGGTTCTTT TACAAGACAT CCTTCTCAAA | |
| | | TGGCAACGTC TTACTGAAGA ACAGTGCCTT TTTAGTGCAT | |
| | | GGCTTTCAGA AAAAGAAGAT GCAGTGAACA AGATTCACAC | |
| | | AACTGGCTTT AAAGATCAAA ATGAAATGTT ATCAAGTCTT | |
| | | CAAAAACTGG CCGTTTTAAA AGCGGATCTA GAAAAGAAAA | |
| | | AGCAATCCAT GGGCAAACTG TATTCACTCA AACAAGATCT | |
| | | TCTTTCAACA CTGAAGAATA AGTCAGTGAC CCAGAAGACG | |
| | | GAAGCATGGC TGGATAACTT TGCCCGGTGT TGGGATAATT | |
| | | TAGTCCAAAA ACTTGAAAAG AGTACAGCAC AGATTTCACA | |
| | | GGCTGTCACC ACCACTCAGC CATCACTAAC ACAGACAACT | |
| | | GTAATGGAAA CAGTAACTAC GGTGACCACA AGGGAACAGA | |
| | | TCCTGGTAAA GCATGCTCAA GAGGAACTTC CACCACCACC | |
| | | TCCCCAAAAG AAGAGGCAGA TTACTGTGGA TTCTGAAATT | |
| | | AGGAAAAGGT TGGATGTTGA TATAACTGAA CTTCACAGCT | |
| | | GGATTACTCG CTCAGAAGCT GTGTTGCAGA GTCCTGAATT | |
| | | TGCAATCTTT CGGAAGGAAG GCAACTTCTC AGACTTAAAA | |
| | | GAAAAAGTCA ATGCCATAGA GCGAGAAAAA GCTGAGAAGT | |
| | | TCAGAAAACT GCAAGATGCC AGCAGATCAG CTCAGGCCCT | |
| | | GGTGGAACAG ATGGTGAATG AGGGTGTTAA TGCAGATAGC | |
| | | ATCAAACAAG CCTCAGAACA ACTGAACAGC CGGTGGATCG | |
| | | AATTCTGCCA GTTGCTAAGT GAGAGACTTA ACTGGCTGGA | |
| | | GTATCAGAAC AACATCATCG CTTTCTATAA TCAGCTACAA | |
| | | CAATTGGAGC AGATGACAAC TACTGCTGAA AACTGGTTGA | |
| | | AAATCCAACC CACCACCCCA TCAGAGCCAA CAGCAATTAA | |
| | | AAGTCAGTTA AAAATTTGTA AGGATGAAGT CAACCGGCTA | |
| | | TCAGGTCTTC AACCTCAAAT TGAACGATTA AAAATTCAAA | |
| | | GCATAGCCCT GAAAGAGAAA GGACAAGGAC CCATGTTCCT | |
| | | GGATGCAGAC TTTGTGGCCT TTACAAATCA TTTTAAGCAA | |
| | | GTCTTTTCTG ATGTGCAGGC CAGAGAGAAA GAGCTACAGA | |
| | | CAATTTTTGA CACTTTGCCA CCAATGCGCT ATCAGGAGAC | |
| | | CATGAGTGCC ATCAGGACAT GGGTCCAGCA GTCAGAAACC | |
| | | AAACTCTCCA TACCTCAACT TAGTGTCACC GACTATGAAA | |
| | | TCATGGAGCA GAGACTCGGG GAATTGCAGG CTTTACAAAG | |
| | | TTCTCTGCAA GAGCAACAAA GTGGCCTATA CTATCTCAGC | |
| | | ACCACTGTGA AAGAGATGTC GAAGAAAGCG CCCTCTGAAA | |
| | | TTAGCCGGAA ATATCAATCA GAATTTGAAG AAATTGAGGG | |
| | | ACGCTGGAAG AAGCTCTCCT CCCAGCTGGT TGAGCATTGT | |
| | | CAAAAGCTAG AGGAGCAAAT GAATAAACTC CGAAAAATTC | |
| | | AGAATCACAT ACAAACCCTG AAGAAATGGA TGGCTGAAGT | |
| | | TGATGTTTTT CTGAAGGAGG AATGGCCTGC CCTTGGGGAT | |
| | | TCAGAAATTC TAAAAAAGCA GCTGAAACAG TGCAGACTTT | |
| | | TAGTCAGTGA TATTCAGACA ATTCAGCCCA GTCTAAACAG | |
| | | TGTCAATGAA GGTGGGCAGA AGATAAAGAA TGAAGCAGAG | |
| | | CCAGAGTTTG CTTCGAGACT TGAGACAGAA CTCAAAGAAC | |
| | | TTAACACTCA GTGGGATCAC ATGTGCCAAC AGGTCTATGC | |
| | | CAGAAAGGAG GCCTTGAAGG GAGGTTTGGA GAAAACTGTA | |
| | | AGCCTCCAGA AAGATCTATC AGAGATGCAC GAATGGATGA | |
| | | CACAAGCTGA AGAAGAGTAT CTTGAGAGAG ATTTTGAATA | |
| | | TAAAACTCCA GATGAATTAC AGAAAGCAGT TGAAGAGATG | |
| | | AAGAGAGCTA AAGAAGAGGC CCAACAAAAA GAAGCGAAAG | |
| | | TGAAACTCCT TACTGAGTCT GTAAATAGTG TCATAGCTCA | |
| | | AGCTCCACCT GTAGCACAAG AGGCCTTAAA AAAGGAACTT | |
| | | GAAACTCTAA CCACCAACTA CCAGTGGCTC TGCACTAGGC | |
| | | TGAATGGGAA ATGCAAGACT TTGGAAGAAG TTTGGGCATG | |
| | | TTGGCATGAG TTATTGTCAT ACTTGGAGAA AGCAAACAAG | |
| | | TGGCTAAATG AAGTAGAATT TAAACTTAAA ACCACTGAAA | |
| | | ACATTCCTGG CGGAGCTGAG GAAATCTCTG AGGTGCTAGA | |
| | | TTCACTTGAA AATTTGATGC GACATTCAGA GGATAACCCA | |
| | | AATCAGATTC GCATATTGGC ACAGACCCTA ACAGATGGCG | |
| | | GAGTCATGGA TGAGCTAATC AATGAGGAAC TTGAGACATT | |
| | | TAATTCTCGT TGGAGGGAAC TACATGAAGA GGCTGTAAGG | |
| | | AGGCAAAAGT TGCTTGAACA GAGCATCCAG TCTGCCCAGG | |
| | | AGACTGAAAA ATCCTTACAC TTAATCCAGG AGTCCCTCAC | |
| | | ATTCATTGAC AAGCAGTTGG CAGCTTATAT TGCAGACAAG | |
| | | GTGGACGCAG CTCAAATGCC TCAGGAAGCC CAGAAAATCC | |
| | | AATCTGATTT GACAAGTCAT GAGATCAGTT TAGAAGAAAT | |
| | | GAAGAAACAT AATCAGGGGA AGGAGGCTGC CCAAAGAGTC | |
| | | CTGTCTCAGA TTGATGTTGC ACAGAAAAAA TTACAAGATG | |
| | | TCTCCATGAA GTTTCGATTA TTCCAGAAAC CAGCCAATTT | |
| | | TGAGCAGCGT CTACAAGAAA GTAAGATGAT TTTAGATGAA | |
| | | GTGAAGATGC ACTTGCCTGC ATTGGAAACA AAGAGTGTGG | |
| | | AACAGGAAGT AGTACAGTCA CAGCTAAATC ATTGTGTGAA | |
| | | CTTGTATAAA AGTCTGAGTG AAGTGAAGTC TGAAGTGGAA | |
| | | ATGGTGATAA AGACTGGACG TCAGATTGTA CAGAAAAAGC | |
| | | AGACGGAAAA TCCCAAAGAA CTTGATGAAA GAGTAACAGC | |
| | | TTTGAAATTG CATTATAATG AGCTGGGAGC AAAGGTAACA | |
| | | GAAAGAAAGC AACAGTTGGA GAAATGCTTG AAATTGTCCC | |
| | | GTAAGATGCG AAAGGAAATG AATGTCTTGA CAGAATGGCT | |
| | | GGCAGCTACA GATATGGAAT TGACAAAGAG ATCAGCAGTT | |
| | | GAAGGAATGC CTAGTAATTT GGATTCTGAA GTTGCCTGGG | |
| | | GAAAGGCTAC TCAAAAAGAG ATTGAGAAAC AGAAGGTGCA | |
| | | CCTGAAGAGT ATCACAGAGG TAGGAGAGGC CTTGAAAACA | |
| | | GTTTTGGGCA AGAAGGAGAC GTTGGTGGAA GATAAACTCA | |
| | | GTCTTCTGAA TAGTAACTGG ATAGCTGTCA CCTCCCGAGC | |
| | | AGAAGAGTGG TTAAATCTTT TGTTGGAATA CCAGAAACAC | |
| | | ATGGAAACTT TTGACCAGAA TGTGGACCAC ATCACAAAGT | |
| | | GGATCATTCA GGCTGACACA CTTTTGGATG AATCAGAGAA | |
| | | AAAGAAACCC CAGCAAAAAG AAGACGTGCT TAAGCGTTTA | |
| | | AAGGCAGAAC TGAATGACAT ACGCCCAAAG GTGGACTCTA | |
| | | CACGTGACCA AGCAGCAAAC TTGATGGCAA ACCGCGGTGA | |
| | | CCACTGCAGG AAATTAGTAG AGCCCCAAAT CTCAGAGCTC | |
| | | AACCATCGAT TTGCAGCCAT TTCACACAGA ATTAAGACTG | |
| | | GAAAGGCCTC CATTCCTTTG AAGGAATTGG AGCAGTTTAA | |
| | | CTCAGATATA CAAAAATTGC TTGAACCACT GGAGGCTGAA | |
| | | ATTCAGCAGG GGGTGAATCT GAAAGAGGAA GACTTCAATA | |
| | | AAGATATGAA TGAAGACAAT GAGGGTACTG TAAAAGAATT | |
| | | GTTGCAAAGA GGAGACAACT TACAACAAAG AATCACAGAT | |
| | | GAGAGAAAGC GAGAGGAAAT AAAGATAAAA CAGCAGCTGT | |
| | | TACAGACAAA ACATAATGCT CTCAAGGATT TGAGGTCTCA | |
| | | AAGAAGAAAA AAGGCTCTAG AAATTTCTCA TCAGTGGTAT | |
| | | CAGTACAAGA GGCAGGCTGA TGATCTCCTG AAATGCTTGG | |
| | | ATGACATTGA AAAAAAATTA GCCAGCCTAC CTGAGCCCAG | |
| | | AGATGAAAGG AAAATAAAGG AAATTGATCG GGAATTGCAG | |
| | | AAGAAGAAAG AGGAGCTGAA TGCAGTGCGT AGGCAAGCTG | |
| | | AGGGCTTGTC TGAGGATGGG GCCGCAATGG CAGTGGAGCC | |
| | | AACTCAGATC CAGCTCAGCA AGCGCTGGCG GGAAATTGAG | |
| | | AGCAAATTTG CTCAGTTTCG AAGACTCAAC TTTGCACAAA | |
| | | TTCACACTGT CCGTGAAGAA ACGATGATGG TGATGACTGA | |
| | | AGACATGCCT TTGGAAATTT CTTATGTGCC TTCTACTTAT | |
| | | TTGACTGAAA TCACTCATGT CTCACAAGCC CTATTAGAAG | |
| | | TGGAACAACT TCTCAATGCT CCTGACCTCT GTGCTAAGGA | |
| | | CTTTGAAGAT CTCTTTAAGC AAGAGGAGTC TCTGAAGAAT | |
| | | ATAAAAGATA GTCTACAACA AAGCTCAGGT CGGATTGACA | |
| | | TTATTCATAG CAAGAAGACA GCAGCATTGC AAAGTGCAAC | |
| | | GCCTGTGGAA AGGGTGAAGC TACAGGAAGC TCTCTCCCAG | |
| | | CTTGATTTCC AATGGGAAAA AGTTAACAAA ATGTACAAGG | |
| | | ACCGACAAGG GCGATTTGAC AGATCTGTTG AGAAATGGCG | |
| | | GCGTTTTCAT TATGATATAA AGATATTTAA TCAGTGGCTA | |
| | | ACAGAAGCTG AACAGTTTCT CAGAAAGACA CAAATTCCTG | |
| | | AGAATTGGGA ACATGCTAAA TACAAATGGT ATCTTAAGGA | |
| | | ACTCCAGGAT GGCATTGGGC AGCGGCAAAC TGTTGTCAGA | |
| | | ACATTGAATG CAACTGGGGA AGAAATAATT CAGCAATCCT | |
| | | CAAAAACAGA TGCCAGTATT CTACAGGAAA AATTGGGAAG | |
| | | CCTGAATCTG CGGTGGCAGG AGGTCTGCAA ACAGCTGTCA | |
| | | GACAGAAAAA AGAGGCTAGA AGAACAAAAG AATATCTTGT | |
| | | CAGAATTTCA AAGAGATTTA AATGAATTTG TTTTATGGTT | |
| | | GGAGGAAGCA GATAACATTG CTAGTATCCC ACTTGAACCT | |
| | | GGAAAAGAGC AGCAACTAAA AGAAAAGCTT GAGCAAGTCA | |
| | | AGTTACTGGT GGAAGAGTTG CCCCTGCGCC AGGGAATTCT | |
| | | CAAACAATTA AATGAAACTG GAGGACCCGT GCTTGTAAGT | |
| | | GCTCCCATAA GCCCAGAAGA GCAAGATAAA CTTGAAAATA | |
| | | AGCTCAAGCA GACAAATCTC CAGTGGATAA AGGTTTCCAG | |
| | | AGCTTTACCT GAGAAACAAG GAGAAATTGA AGCTCAAATA | |
| | | AAAGACCTTG GGCAGCTTGA AAAAAAGCTT GAAGACCTTG | |
| | | AAGAGCAGTT AAATCATCTG CTGCTGTGGT TATCTCCTAT | |
| | | TAGGAATCAG TTGGAAATTT ATAACCAACC AAACCAAGAA | |
| | | GGACCATTTG ACGTTCAGGA AACTGAAATA GCAGTTCAAG | |
| | | CTAAACAACC GGATGTGGAA GAGATTTTGT CTAAAGGGCA | |
| | | GCATTTGTAC AAGGAAAAAC CAGCCACTCA GCCAGTGAAG | |
| | | AGGAAGTTAG AAGATCTGAG CTCTGAGTGG AAGGCGGTAA | |
| | | ACCGTTTACT TCAAGAGCTG AGGGCAAAGC AGCCTGACCT | |
| | | AGCTCCTGGA CTGACCACTA TTGGAGCCTC TCCTACTCAG | |
| | | ACTGTTACTC TGGTGACACA ACCTGTGGTT ACTAAGGAAA | |
| | | CTGCCATCTC CAAACTAGAA ATGCCATCTT CCTTGATGTT | |
| | | GGAGGTACCT GCTCTGGCAG ATTTCAACCG GGCTTGGACA | |
| | | GAACTTACCG ACTGGCTTTC TCTGCTTGAT CAAGTTATAA | |
| | | AATCACAGAG GGTGATGGTG GGTGACCTTG AGGATATCAA | |
| | | CGAGATGATC ATCAAGCAGA AGGCAACAAT GCAGGATTTG | |
| | | GAACAGAGGC GTCCCCAGTT GGAAGAACTC ATTACCGCTG | |
| | | CCCAAAATTT GAAAAACAAG ACCAGCAATC AAGAGGCTAG | |
| | | AACAATCATT ACGGATCGAA TTGAAAGAAT TCAGAATCAG | |
| | | TGGGATGAAG TACAAGAACA CCTTCAGAAC CGGAGGCAAC | |
| | | AGTTGAATGA AATGTTAAAG GATTCAACAC AATGGCTGGA | |
| | | AGCTAAGGAA GAAGCTGAGC AGGTCTTAGG ACAGGCCAGA | |
| | | GCCAAGCTTG AGTCATGGAA GGAGGGTCCC TATACAGTAG | |
| | | ATGCAATCCA AAAGAAAATC ACAGAAACCA AGCAGTTGGC | |
| | | CAAAGACCTC CGCCAGTGGC AGACAAATGT AGATGTGGCA | |
| | | AATGACTTGG CCCTGAAACT TCTCCGGGAT TATTCTGCAG | |
| | | ATGATACCAG AAAAGTCCAC ATGATAACAG AGAATATCAA | |
| | | TGCCTCTTGG AGAAGCATTC ATAAAAGGGT GAGTGAGCGA | |
| | | GAGGCTGCTT TGGAAGAAAC TCATAGATTA CTGCAACAGT | |
| | | TCCCCCTGGA CCTGGAAAAG TTTCTTGCCT GGCTTACAGA | |
| | | AGCTGAAACA ACTGCCAATG TCCTACAGGA TGCTACCCGT | |
| | | AAGGAAAGGC TCCTAGAAGA CTCCAAGGGA GTAAAAGAGC | |
| | | TGATGAAACA ATGGCAAGAC CTCCAAGGTG AAATTGAAGC | |
| | | TCACACAGAT GTTTATCACA ACCTGGATGA AAACAGCCAA | |
| | | AAAATCCTGA GATCCCTGGA AGGTTCCGAT GATGCAGTCC | |
| | | TGTTACAAAG ACGTTTGGAT AACATGAACT TCAAGTGGAG | |
| | | TGAACTTCGG AAAAAGTCTC TCAACATTAG GTCCCATTTG | |
| | | GAAGCCAGTT CTGACCAGTG GAAGCGTCTG CACCTTTCTC | |
| | | TGCAGGAACT TCTGGTGTGG CTACAGCTGA AAGATGATGA | |
| | | ATTAAGCCGG CAGGCACCTA TTGGAGGCGA CTTTCCAGCA | |
| | | GTTCAGAAGC AGAACGATGT ACATAGGGCC TTCAAGAGGG | |
| | | AATTGAAAAC TAAAGAACCT GTAATCATGA GTACTCTTGA | |
| | | GACTGTACGA ATATTTCTGA CAGAGCAGCC TTTGGAAGGA | |
| | | CTAGAGAAAC TCTACCAGGA GCCCAGAGAG CTGCCTCCTG | |
| | | AGGAGAGAGC CCAGAATGTC ACTCGGCTTC TACGAAAGCA | |
| | | GGCTGAGGAG GTCAATACTG AGTGGGAAAA ATTGAACCTG | |
| | | CACTCCGCTG ACTGGCAGAG AAAAATAGAT GAGACCCTTG | |
| | | AAAGACTCCA GGAACTTCAA GAGGCCACGG ATGAGCTGGA | |
| | | CCTCAAGCTG CGCCAAGCTG AGGTGATCAA GGGATCCTGG | |
| | | CAGCCCGTGG GCGATCTCCT CATTGACTCT CTCCAAGATC | |
| | | ACCTCGAGAA AGTCAAGGCA CTTCGAGGAG AAATTGCGCC | |
| | | TCTGAAAGAG AACGTGAGCC ACGTCAATGA CCTTGCTCGC | |
| | | CAGCTTACCA CTTTGGGCAT TCAGCTCTCA CCGTATAACC | |
| | | TCAGCACTCT GGAAGACCTG AACACCAGAT GGAAGCTTCT | |
| | | GCAGGTGGCC GTCGAGGACC GAGTCAGGCA GCTGCATGAA | |
| | | GCCCACAGGG ACTTTGGTCC AGCATCTCAG CACTTTCTTT | |
| | | CCACGTCTGT CCAGGGTCCC TGGGAGAGAG CCATCTCGCC | |
| | | AAACAAAGTG CCCTACTATA TCAACCACGA GACTCAAACA | |
| | | ACTTGCTGGG ACCATCCCAA AATGACAGAG CTCTACCAGT | |
| | | CTTTAGCTGA CCTGAATAAT GTCAGATTCT CAGCTTATAG | |
| | | GACTGCCATG AAACTCCGAA GACTGCAGAA GGCCCTTTGC | |
| | | TTGGATCTCT TGAGCCTGTC AGCTGCATGT GATGCCTTGG | |
| | | ACCAGCACAA CCTCAAGCAA AATGACCAGC CCATGGATAT | |
| | | CCTGCAGATT ATTAATTGTT TGACCACTAT TTATGACCGC | |
| | | CTGGAGCAAG AGCACAACAA TTTGGTCAAC GTCCCTCTCT | |
| | | GCGTGGATAT GTGTCTGAAC TGGCTGCTGA ATGTTTATGA | |
| | | TACGGGACGA ACAGGGAGGA TCCGTGTCCT GTCTTTTAAA | |
| | | ACTGGCATCA TTTCCCTGTG TAAAGCACAT TTGGAAGACA | |
| | | AGTACAGATA CCTTTTCAAG CAAGTGGCAA GTTCAACAGG | |
| | | ATTTTGTGAC CAGCGCAGGC TGGGCCTCCT TCTGCATGAT | |
| | | TCTATCCAAA TTCCAAGACA GTTGGGTGAA GTTGCATCCT | |
| | | TTGGGGGCAG TAACATTGAG CCAAGTGTCC GGAGCTGCTT | |
| | | CCAATTTGCT AATAATAAGC CAGAGATCGA AGCGGCCCTC | |
| | | TTCCTAGACT GGATGAGACT GGAACCCCAG TCCATGGTGT | |
| | | GGCTGCCCGT CCTGCACAGA GTGGCTGCTG CAGAAACTGC | |
| | | CAAGCATCAG GCCAAATGTA ACATCTGCAA AGAGTGTCCA | |
| | | ATCATTGGAT TCAGGTACAG GAGTCTAAAG CACTTTAATT | |
| | | ATGACATCTG CCAAAGCTGC TTTTTTTCTG GTCGAGTTGC | |
| | | AAAAGGCCAT AAAATGCACT ATCCCATGGT GGAATATTGC | |
| | | ACTCCGACTA CATCAGGAGA AGATGTTCGA GACTTTGCCA | |
| | | AGGTACTAAA AAACAAATTT CGAACCAAAA GGTATTTTGC | |
| | | GAAGCATCCC CGAATGGGCT ACCTGCCAGT GCAGACTGTC | |
| | | TTAGAGGGGG ACAACATGGA AACTCCCGTT ACTCTGATCA | |
| | | ACTTCTGGCC AGTAGATTCT GCGCCTGCCT CGTCCCCTCA | |
| | | GCTTTCACAC GATGATACTC ATTCACGCAT TGAACATTAT | |
| | | GCTAGCAGGC TAGCAGAAAT GGAAAACAGC AATGGATCTT | |
| | | ATCTAAATGA TAGCATCTCT CCTAATGAGA GCATAGATGA | |
| | | TGAACATTTG TTAATCCAGC ATTACTGCCA AAGTTTGAAC | |
| | | CAGGACTCCC CCCTGAGCCA GCCTCGTAGT CCTGCCCAGA | |
| | | TCTTGATTTC CTTAGAGAGT GAGGAAAGAG GGGAGCTAGA | |
| | | GAGAATCCTA GCAGATCTTG AGGAAGAAAA CAGGAATCTG | |
| | | CAAGCAGAAT ATGACCGTCT AAAGCAGCAG CACGAACATA | |
| | | AAGGCCTGTC CCCACTGCCG TCCCCTCCTG AAATGATGCC | |
| | | CACCTCTCCC CAGAGTCCCC GGGATGCTGA GCTCATTGCT | |
| | | GAGGCCAAGC TACTGCGTCA ACACAAAGGC CGCCTGGAAG | |
| | | CCAGGATGCA AATCCTGGAA GACCACAATA AACAGCTGGA | |
| | | GTCACAGTTA CACAGGCTAA GGCAGCTGCT GGAGCAACCC | |
| | | CAGGCAGAGG CCAAAGTGAA TGGCACAACG GTGTCCTCTC | |
| | | CTTCTACCTC TCTACAGAGG TCCGACAGCA GTCAGCCTAT | |
| | | GCTGCTCCGA GTGGTTGGCA GTCAAACTTC GGACTCCATG | |
| | | GGTGAGGAAG ATCTTCTCAG TCCTCCCCAG GACACAAGCA | |
| | | CAGGGTTAGA GGAGGTGATG GAGCAACTCA ACAACTCCTT | |
| | | CCCTAGTTCA AGAGGAAGAA ATACCCCTGG AAAGCCAATG | |
| | | AGAGAGGACA CAATGTAGGA AGTCTTTTCC ACATGGCAGA | |
| | | TGATTTGGGC AGAGCGATGG AGTCCTTAGT ATCAGTCATG | |
| | | ACAGATGAAG AAGGAGCAGA ATAAATGTTT TACAACTCCT | |
| | | GATTCCCGCA TGGTTTTTAT AATATTCATA CAACAAAGAG | |
| | | GATTAGACAG TAAGAGTTTA CAAGAAATAA ATCTATATTT | |
| | | TTGTGAAGGG TAGTGGTATT ATACTGTAGA TTTCAGTAGT | |
| | | TTCTAAGTCT GTTATTGTTT TGTTAACAAT GGCAGGTTTT | |
| | | ACACGTCTAT GCAATTGTAC AAAAAAGTTA TAAGAAAACT | |
| | | ACATGTAAAA TCTTGATAGC TAAATAACTT GCCATTTCTT | |
| | | TATATGGAAC GCATTTTGGG TTGTTTAAAA ATTTATAACA | |
| | | GTTATAAAGA AAGATTGTAA ACTAAAGTGT GCTTTATAAA | |
| | | AAAAAGTTGT TTATAAAAAC CCCTAAAAAC AAAACAAACA | |
| | | CACACACACA CACATACACA CACACACACA AAACTTTGAG | |
| | | GCAGCGCATT GTTTTGCATC CTTTTGGCGT GATATCCATA | |
| | | TGAAATTCAT GGCTTTTTCT TTTTTTGCAT ATTAAAGATA | |
| | | AGACTTCCTC TACCACCACA CCAAATGACT ACTACACACT | |
| | | GCTCATTTGA GAACTGTCAG CTGAGTGGGG CAGGCTTGAG | |
| | | TTTTCATTTC ATATATCTAT ATGTCTATAA GTATATAAAT | |
| | | ACTATAGTTA TATAGATAAA GAGATACGAA TTTCTATAGA | |
| | | CTGACTTTTT CCATTTTTTA AATGTTCATG TCACATCCTA | |
| | | ATAGAAAGAA ATTACTTCTA GTCAGTCATC CAGGCTTACC | |
| | | TGCTTGGTCT AGAATGGATT TTTCCCGGAG CCGGAAGCCA | |
| | | GGAGGAAACT ACACCACACT AAAACATTGT CTACAGCTCC | |
| | | AGATGTTTCT CATTTTAAAC AACTTTCCAC TGACAACGAA | |
| | | AGTAAAGTAA AGTATTGGAT TTTTTTAAAG GGAACATGTG | |
| | | AATGAATACA CAGGACTTAT TATATCAGAG TGAGTAATCG | |
| | | GTTGGTTGGT TGATTGATTG ATTGATTGAT ACATTCAGCT | |
| | | TCCTGCTGCT AGCAATGCCA CGATTTAGAT TTAATGATGC | |
| | | TTCAGTGGAA ATCAATCAGA AGGTATTCTG ACCTTGTGAA | |
| | | CATCAGAAGG TATTTTTTAA CTCCCAAGCA GTAGCAGGAC | |
| | | GATGATAGGG CTGGAGGGCT ATGGATTCCC AGCCCATCCC | |
| | | TGTGAAGGAG TAGGCCACTC TTTAAGTGAA GGATTGGATG | |
| | | ATTGTTCATA ATACATAAAG TTCTCTGTAA TTACAACTAA | |
| | | ATTATTATGC CCTCTTCTCA CAGTCAAAAG GAACTGGGTG | |
| | | GTTTGGTTTT TGTTGCTTTT TTAGATTTAT TGTCCCATGT | |
| | | GGGATGAGTT TTTAAATGCC ACAAGACATA ATTTAAAATA | |
| | | AATAAACTTT GGGAAAAGGT GTAAAACAGT AGCCCCATCA | |
| | | CATTTGTGAT ACTGACAGGT ATCAACCCAG AAGCCCATGA | |
| | | ACTGTGTTTC CATCCTTTGC ATTTCTCTGC GAGTAGTTCC | |
| | | ACACAGGTTT GTAAGTAAGT AAGAAAGAAG GCAAATTGAT | |
| | | TCAAATGTTA CAAAAAAACC CTTCTTGGTG GATTAGACAG | |
| | | GTTAAATATA TAAACAAACA AACAAAAATT GCTCAAAAAA | |
| | | GAGGAGAAAA GCTCAAGAGG AAAAGCTAAG GACTGGTAGG | |
| | | AAAAAGCTTT ACTCTTTCAT GCCATTTTAT TTCTTTTTGA | |
| | | TTTTTAAATC ATTCATTCAA TAGATACCAC CGTGTGACCT | |
| | | ATAATTTTGC AAATCTGTTA CCTCTGACAT CAAGTGTAAT | |
| | | TAGCTTTTGG AGAGTGGGCT GACATCAAGT GTAATTAGCT | |
| | | TTTGGAGAGT GGGTTTTGTC CATTATTAAT AATTAATTAA | |
| | | TTAACATCAA ACACGGCTTC TCATGCTATT TCTACCTCAC | |
| | | TTTGGTTTTG GGGTGTTCCT GATAATTGTG CACACCTGAG | |
| | | TTCACAGCTT CACCACTTGT CCATTGCGTT ATTTTCTTTT | |
| | | TCCTTTATAA TTCTTTCTTT TTCCTTCATA ATTTTCAAAA | |
| | | GAAAACCCAA AGCTCTAAGG TAACAAATTA CCAAATTACA | |
| | | TGAAGATTTG GTTTTTGTCT TGCATTTTTT TCCTTTATGT | |
| | | GACGCTGGAC CTTTTCTTTA CCCAAGGATT TTTAAAACTC | |
| | | AGATTTAAAA CAAGGGGTTA CTTTACATCC TACTAAGAAG | |
| | | TTTAAGTAAG TAAGTTTCAT TCTAAAATCA GAGGTAAATA | |
| | | GAGTGCATAA ATAATTTTGT TTTAATCTTT TTGTTTTTCT | |
| | | TTTAGACACA TTAGCTCTGG AGTGAGTCTG TCATAATATT | |
| | | TGAACAAAAA TTGAGAGCTT TATTGCTGCA TTTTAAGCAT | |
| | | AATTAATTTG GACATTATTT CGTGTTGTGT TCTTTATAAC | |
| | | CACCAAGTAT TAAACTGTAA ATCATAATGT AACTGAAGCA | |
| | | TAAACATCAC ATGGCATGTT TTGTCATTGT TTTCAGGTAC | |
| | | TGAGTTCTTA CTTGAGTATC ATAATATATT GTGTTTTAAC | |
| | | ACCAACACTG TAACATTTAC GAATTATTTT TTTAAACTTC | |
| | | AGTTTTACTG CATTTTCACA ACATATCAGA CTTCACCAAA | |
| | | TATATGCCTT ACTATTGTAT TATAGTACTG CTTTACTGTG | |
| | | TATCTCAATA AAGCACGCAG TTATGTTAC | |
| DNAJC9 | NM_015190.4 | GGATGCGCGG CGTGGCCACG CCCCTTCAGT GCTTGTGACG | 9 |
| | | CAGGCGCCCT GGGCTTTTTG GGCGCGAAAA AGAAGCAGTC | |
| | | CTGGGTTGTA CCCGGCGCAG CTGGGAGCGG CTGCTTCCTC | |
| | | CGGGGTCGTA TCTCCGCCCG GCATGGGGCT GCTGGACCTT | |
| | | TGCGAGGAAG TGTTCGGCAC CGCCGACCTT TACCGGGTGC | |
| | | TGGGCGTGCG ACGCGAGGCC TCCGACGGCG AGGTCCGACG | |
| | | AGGCTACCAC AAGGTGTCCC TGCAGGTACA CCCGGACCGG | |
| | | GTGGGTGAGG GCGACAAGGA GGACGCCACC CGCCGCTTCC | |
| | | AGATCCTGGG AAAAGTCTAT TCCGTTCTCA GTGACAGAGA | |
| | | ACAGAGAGCA GTGTACGATG AGCAGGGAAC AGTGGACGAG | |
| | | GACTCTCCTG TGCTCACCCA AGACCGAGAC TGGGAGGCGT | |
| | | ATTGGCGGCT ACTCTTTAAA AAGATATCTT TAGAGGACAT | |
| | | TCAAGCTTTT GAAAAGACAT ACAAAGGTTC GGAAGAAGAG | |
| | | CTGGCTGATA TTAAGCAGGC CTATCTGGAC TTCAAGGGTG | |
| | | ACATGGATCA GATCATGGAG TCTGTGCTTT GCGTGCAGTA | |
| | | CACAGAGGAA CCCAGGATAA GGAATATCAT TCAGCAAGCT | |
| | | ATTGACGCCG GAGAGGTCCC ATCCTATAAT GCCTTTGTCA | |
| | | AAGAATCGAA ACAAAAGATG AATGCAAGGA AAAGGAGGGC | |
| | | TCAGGAAGAG GCCAAAGAAG CAGAAATGAG CAGAAAGGAG | |
| | | TTGGGGCTTG ATGAAGGCGT GGATAGCCTG AAGGCAGCCA | |
| | | TTCAGAGCAG ACAAAAGGAT CGGCAAAAGG AAATGGACAA | |
| | | TTTTCTGGCT CAGATGGAAG CAAAGTACTG CAAATCTTCC | |
| | | AAAGGAGGAG GGAAAAAATC TGCTCTCAAG AAAGAAAAGA | |
| | | AATAATGGAA TTTTTCTCTT CAAAGGTCCT TAGGTGTAAA | |
| | | TTGATGCCAT CGTAGGCAAG GTGCAGGCAG GATTTGAAGG | |
| | | CAAAAGTCAA TTCAGCTCTT GAGAAAAGGT GTCTTTCCAG | |
| | | CCTGAATTTT TCAGATTGAC TAGACCAAGC AGAATCTCTC | |
| | | AACCTGATCT TAGTATTTCC TAGAAAGCAC TTGACATTGT | |
| | | GTGAGGTCTC ACCTGAAGGA ACTTGGTGGT GACATTTGGG | |
| | | AGGGTGGAGG GAGGCAGTGT CCTTCCTGAC AGCACTTGCC | |
| | | TCCATGGATC TTCTGTACAC AGAACTCTTA TCTAGGATGT | |
| | | GGTTCTGTTC ATGCTGCTTT CTGCGATGTG CGTGTCTGTT | |
| | | AGAATAGGCT CTCTACCCAG CTAGAACACC TTCCAGACAC | |
| | | TTGCTGGACA GCTATCTTCC ACATACTTCC CAGTTTACAT | |
| | | TTGGTCTTAA TGATCTTGAA TAGATCCTCT CTTCATTTTA | |
| | | CTCAGCCAGG TTTTGTACTG ATGTACAGGT GTTAAATTAC | |
| | | TTCAAGCATT TTTGTAAGAG GTGTATATAA TTCAATAAAA | |
| | | AAGGTAAAAC ATGATGATTA AGTTCTGGGG GCTTTGTAAA | |
| | | TGATCCCACT AAAATGTGAC CTAGGAAAAA TATGAATGGT | |
| | | GTTTAGGATG AGAGAAAAGG GGAAAACAAT AGCCCTGGTC | |
| | | AGCTTTATAA TAGAGAGCCT GGTTTCCCTA GCATGAAGAG | |
| | | ATGTATGTTG TAGTCCTGCC ACTGATTACT GAAGTCCTGC | |
| | | CATTAATTGC TGAATGTCTT CAGTTGGGCC ACTGAGCTTG | |
| | | TCTGAATCTG TTCCCTTTTA TAAAAGAGTT ACTACATCAA | |
| | | AACAAAGAGT GAAATCCAAA TTTGTCAAAC TGTATGTATT | |
| | | AAACATGTCC AGTTTTTTGG TTAAAAAAAA ATTGCACTGG | |
| | | CTTTGAGGGA AAACACACAG GGTGAGGGAA TTGGGCTAAA | |
| | | TGACTTCTTA CAGGCCCCTT TCTGATTCTT TAACTTTGAA | |
| | | AGGCAAGCCA TATTGATCCA GTTGTTATAG TGAACTCATG | |
| | | GTAATGGTTT GTGAGAACAA TAGAGATTTT CATTTCTATG | |
| | | TAGATGAGTT GGTATGAGAA TATATGGAAT TTTTAAGGGA | |
| | | CTGTTTAAAT CTTTGATTTG TAGACTATTA AATATACCGT | |
| | | ATGCATAAAG TAAGCCTTTA GCTCTAAGGT AAAGACGACA | |
| | | CGTTTTCGGT TTGTGACTAC AAATAGGTTA AAAATAGATT | |
| | | TTAATTTTAT TAAAAATATA ATTTAATGCA GGTTGTTTGA | |
| | | AGCATCTGTC TTCATATGAT GGCATTAGAA CACCTTGGTA | |
| | | TAATAAAAAG TTACCGTAAT TTATGATTAT TTGAATTTAT | |
| | | CCATTCTGAA AATTAATAAG ATCTAAAACT GGCATGACAA | |
| | | TCAAGATTTG TATTTAGTGA AATTTAAAAT AAATGTAAGC | |
| | | CATAGTTAAA AAAAAAAAAA AAAAA | |
| ENOSF1 | NM_001126123.3 | CGCCCTCCCG CCGCGCGCTC GGGATCCCGA CCAGTCCTGA | 10 |
| | | CCGCACGGGG GCCGCGGCCA CGGGGCGCAG GGGCCATGGT | |
| | | GCGCGGCAGG ATCTCCCGGC TCTCGGTCCG GGACGTGCGC | |
| | | TTCCCCACGT CGCTTGGGGG CCACGGCGCG GACGCCATGC | |
| | | ACACGGACCC TGACTACTCG GCTGCCTATG TCGTCATAGA | |
| | | AACTGATGCA GAAGATGGAA TCAAGGGGTG TGGAATTACC | |
| | | TTCACTCTGG GAAAAGGCAC TGAAGTTGAT TGGTCCAGAA | |
| | | AAGGGCGTGG TGCACCTGGC GACAGCGGCC GTCCTAAACG | |
| | | CGGTGTGGGA CTTGTGGGCC AAGCAGGAGG GAAAGCCTGT | |
| | | CTGGAAGTTA CTTGTGGACA TGGATCCCAG GATGCTGGTA | |
| | | TCCTGCATAG ATTTCAGGTA CATCACTGAT GTCCTGACTG | |
| | | AGGAGGATGC CCTAGAAATA CTGCAGAAAG GTCAAATTGG | |
| | | TAAAAAAGAA AGAGAGAAGC AAATGCTGGC ACAAGGATAC | |
| | | CCTGCTTACA CGACATCGTG CGCCTGGCTG GGGTACTCAG | |
| | | ATGACACGTT GAAGCAGCTC TGTGCCCAGG CGCTGAAGGA | |
| | | TGGCTGGACC AGGTTTAAAG TAAAGGTGGG TGCTGATCTC | |
| | | CAGGATGACA TGCGAAGATG CCAAATCATC CGAGACATGA | |
| | | TTGGACCGGA AAAGACTTTG ATGATGGATG CCAACCAGCG | |
| | | CTGGGATGTG CCTGAGGCGG TGGAGTGGAT GTCCAAGCTG | |
| | | GCCAAGTTCA AGCCATTGTG GATTGAGGAG CCAACCTCCC | |
| | | CTGATGACAT TCTGGGGCAC GCCACCATTT CCAAGGCACT | |
| | | GGTCCCATTA GGAATTGGCA TTGCCACAGG AGAACAGTGC | |
| | | CACAATAGAG TGATATTTAA GCAACTCCTA CAGGCGAAGG | |
| | | CCCTGCAGTT CCTCCAGATT GACAGTTGCA GACTGGGCAG | |
| | | TGTCAATGAG AACCTCTCAG TATTGCTGAT GGCCAAAAAG | |
| | | TTTGAAATTC CTGTTTGCCC CCATGCTGGT GGAGTTGGCC | |
| | | TCTGTGAACT GGTGCAGCAC CTGATTATAT TTGACTACAT | |
| | | ATCAGTTTCT GCAAGCCTTG AAAATAGGGT GTGTGAGTAT | |
| | | GTTGACCACC TGCATGAGCA TTTCAAGTAT CCCGTGATGA | |
| | | TCCAGCGGGC TTCCTACATG CCTCCCAAGG ATCCCGGCTA | |
| | | CTCAACAGAA ATGAAGGAGG AATCTGTAAA GAAACACCAG | |
| | | TATCCAGATG GTGAAGTTTG GAAGAAACTC CTTCCTGCTC | |
| | | AAGAAAATTA AGTGCTCAGC CCCAACAACT TTTTTCTTTC | |
| | | TGAAGTGAAA GGGCTTAAAA TTTCTTGGAA ATAGTTTTAC | |
| | | AAAAATGGAT TTAAAAAATC CTACCGATCA AGATGAGTTC | |
| | | AGCTAGAAGT CATACCACCC TCAGGAATCA GCTAAAGCAA | |
| | | AAAGAACTTT TACCTCGGCA TCCAGCCCAA CCCCTAAAGA | |
| | | CTGACAATAT CCTTCGAGCT CCTTTGAAAG CACCCTAAAC | |
| | | AGCCATTTCC ATTTTAATAG TTGGATGCGG ATTGTACCCT | |
| | | TCAATCTGAA AGTCTTCAGC TTTGAAGTCA TCAATTTTCT | |
| | | CAACTTTTCG AAGAATCCTG AGCTTTGGGA AAGGTCTGGG | |
| | | TTCTCGCTGA AGCTAAAAAC AAAATAAGGC CATTATTTTG | |
| | | CCATAATTGT ACGACCTGTT GTAATTGCTC CTCATGTCCG | |
| | | TGAAACAAGT ACACAGGATG TGATCAACAA AGTTCTATTT | |
| | | TACAGGAGTA TGATCCTGTC GATACCTTGC CGTAGGTTAT | |
| | | GTAACATGAT TGGAGCGCAA CCAGCTGTTC TCTTGCACAG | |
| | | ATCGAGAGTG AGGGGTATTT TGTGACATTA CACAGCATCA | |
| | | GGAGCCTGGT GCCTCATCAG GTGTAAGTTC TTATAACCAC | |
| | | TCTTGGCAAA TTTATTAAAG ACAGGAACAC AGTCAATCTG | |
| | | TAACTCATAG TAGCTCTACG TTTACTTGAA TTCCACAATC | |
| | | CCTAACCCAT CTGTCCCTGG CAGAAAGAAG GAAAGATGAC | |
| | | ATGCATGGAC AGTGAACAGA AAGGGATGAA AGCCAGGATT | |
| | | CCTGGGATGA ACAGACAGTG GCAATTAGGA TGTGAAGACA | |
| | | GGTCACAACC TATTACTATG TCTAAAAACG ACCAGAGCAG | |
| | | AGAGCCAGAG AGAATAAGCC TGAAGTCACC TCCACTCAAA | |
| | | AGCAGCCAAA CTCCCTCAAA GGAGTAACTT TTAAAACCTG | |
| | | GATCTAACCT GGAAGGGGCT AAAAAGTGTC TGGTTCTGAG | |
| | | TTTTTTTCCT TAAGGCTCAT GAAGCAGATG AACTTACATT | |
| | | TTTATTGCCA TTTCATATCA ATTGTTGGCT GCTATAACTT | |
| | | AGGGATTTCA ACAGACTTTT GAAGTTTGGA CCTAAATATT | |
| | | GTACTTAATG TAAAATTAAC AAAAAATATT TATGGCCAGG | |
| | | GTGGTGGCTT ATGCCTGTAA TTCCAGAATT TTCGGAGGCT | |
| | | GAGGCAGGTG GATCACTTGA AGTCAGGAGT TTGAGACTAG | |
| | | CCTGGCCAAC ATGATGAAAC CCCATCTCTA CTAATAATAC | |
| | | AAAAATTAGC TGGGTGTGGT GGCATGTGCC TGTAATCCCA | |
| | | GCTACCTGGG AGGCTGAGGC AGAAGAATTG CTTGAACCCG | |
| | | GGAGGTGGAG GTTGCAGTGA GCTGAGATCG CACCACGGCA | |
| | | CACTCCAGCC TGGCCGACAG AGAAAGACTC CATCTCAAAA | |
| | | AAAAAAGAAA AGGAAAAACA TTTGCACTTC AATTCTCCTT | |
| | | CAAGTTAAAA TGAGTTAAAA TGCCCCCTTT TGGACAATCC | |
| | | CCTGGCTTGA ATGTGGCTCT TCCCTCTCTG GTACTGGTGC | |
| | | TTAGTACCTC ACAGCACCTG ACATGTTAAG TGCCCATGGT | |
| | | TGCTGAGGCA GATGCCTGCC TTGTCCTGCC CACCTGCCCA | |
| | | CCACTTCTCC CTAAACTGAA GCCCCACATT TGGAGCAGTC | |
| | | ATCTTTATCT TGGACACAGC ATTGAGCAGA TGCCTGTTCC | |
| | | ACAGTCAACC TTTTATCAAG AGAAGGTACC AAACCCAAAA | |
| | | GTATAACATC TAATTCTTAC CTGAATTTTC AGTGGCTCGA | |
| | | TGTGATTCAG GTAAATATGT GCATCTCCCA AAGTGTGTAT | |
| | | AAAGTCACCT GGCTATAAAC CCGGGGGAGA AAGCAGAACA | |
| | | GTATGTTAGT TTCAATTCTT TAAAACATCA TTTAAAAACA | |
| | | TTAGAATATG CAGACACCGC AAGGCTTTTT TTAAAAAAAT | |
| | | AATTTAGTGT AGCTTTTCCA TTTTTTTGTA GCAACAGCAT | |
| | | CTTGTTATGT TGCCCAGGCT GGTATTGAAC TCCAGACCTC | |
| | | AAGCAATTGC TCCTGTCTCA GTCTCCCAAA GTGCTGGGAT | |
| | | TACAGGCATG AGCCACCATA CCCAACCTCA GCATAGCTTT | |
| | | TGAGAAAATC CATAGAAGCT GTATCACAAA CAACCTGTAT | |
| | | AGATCTGTTA GTGCGTATAC CACAGGGCCA GAAAACCTTC | |
| | | CAGAAGAGGA AGGTTTCAAA GTAAAAGCTG GTTCATTTCT | |
| | | TACTTACACA TATCAAATTT AAAAGCTAAT CAGAGACTAA | |
| | | ACTCTGCAAT TTGTTTTCCC ATATTAAAGA ACTGAAGAGC | |
| | | TCAGTGTGGT AGGCTGGCAA GTCACCCTTC CCGAGACAGC | |
| | | CCACCTTCAG GCCCGTGATG TGCGCAATCA TGTACGTGAG | |
| | | CAGGGCGTAG CTGGCGATGT TGAAAGGCAC ACCGAGGCCC | |
| | | ATGTCTCCCG ATCTCTGGTA CAGCTGGCAG GACAGCTCAC | |
| | | TGTTCACCAC ATAGAACTGG CAGAGGGCAT GGCATGGAGG | |
| | | CAGCGCCATC AGAGGAAGAT CTGAGGAACC AGCAGAGGAA | |
| | | GATAAGGAGG GATGGTGGTT TGAAAGACCA CAGCTAAAGG | |
| | | CAAAGTAAAA CAGGAGAGAA ACAGAAGCCA ACTCATATGG | |
| | | TGGAGACCAG GAGAGAGAGC CACTGGGCTG CAGTGATGTC | |
| | | CATAACAGCC TCTGCAGCGA TGGCACGGAG CTGAGGGAGA | |
| | | CTATCCATCG GTGCAAGGTT TCTGCAGGTG TCCATTTACG | |
| | | GCTGAAGCAA TGCTCTTCCA TCAGAGCTGA AGGGATCTGG | |
| | | GCTACCTCGT GGCACCAGAT TACAAATACA GCAGGAATAA | |
| | | TTCTGTTTGC CACAGGAAAC TGGTGCTTCT GGTACACCCT | |
| | | CCTATATTAA AAGTCTCTAT TACATGGCAA AAAAAAAAAA | |
| | | AAAAAA | |
| FANCL | NM_001114636.1 | AGCGGACTGC GCATGTGCAG GACCCAGCAG GTCTAGAGCT | 11 |
| | | TTTCTGTGTT TCTCCGGACT TCGAGCCATG GCGGTGACGG | |
| | | AAGCGAGCCT GTTGCGCCAG TGCCCCCTGC TTCTGCCCCA | |
| | | GAACCGGTCG AAAACCGTGT ATGAGGGATT CATCTCGGCT | |
| | | CAGGGAAGAG ACTTCCACCT TAGGATAGTG TTGCCTGAAG | |
| | | ATTTACAACT GAAGAATGCA AGATTATTAT GTAGTTGGCA | |
| | | GCTGAGAACA ATACTTAGTG GATACCATCG AATAGTACAA | |
| | | CAGAGAATGC AGCACTCTCC TGATCTAATG AGCTTTATGA | |
| | | TGGAGTTGAA GATGCTTTTG GAAGTTGCCT TAAAGAATAG | |
| | | ACAAGAGCTG TATGCACTAC CTCCTCCTCC CCAGTTCTAC | |
| | | TCAAGCCTTA TTGAAGAGAT AGGAACTCTT GGTTGGGATA | |
| | | AACTTGTGTA TGCGGATACC TGCTTCAGTA CCATCAAGTT | |
| | | AAAAGCAGAA GATGCTTCTG GTAGAGAGCA TTTAATCACT | |
| | | CTCAAGTTGA AGGCAAAGTA TCCTGCAGAA TCACCAGATT | |
| | | ATTTTGTGGA TTTTCCTGTT CCATTTTGTG CCTCCTGGAC | |
| | | ACCTCAGGTA AATTCTCCTC AGAGCTCCTT AATAAGCATT | |
| | | TATAGTCAGT TTTTGGCAGC AATAGAATCA CTAAAGGCAT | |
| | | TCTGGGATGT TATGGATGAA ATCGATGAGA AGACCTGGGT | |
| | | ACTTGAGCCA GAAAAACCTC CACGGAGTGC AACAGCACGC | |
| | | AGAATTGCAT TAGGTAATAA TGTTTCCATA AATATAGAGG | |
| | | TAGACCCCAG GCATCCTACT ATGCTTCCTG AGTGCTTCTT | |
| | | TCTTGGAGCT GACCATGTGG TAAAACCCCT GGGAATTAAG | |
| | | CTGAGCAGGA ACATACATTT GTGGGATCCA GAAAATAGTG | |
| | | TGTTACAAAA TTTGAAAGAT GTTTTAGAAA TTGATTTTCC | |
| | | AGCTCGTGCT ATCCTGGAAA AATCTGATTT TACTATGGAT | |
| | | TGTGGAATTT GTTATGCTTA TCAACTTGAC GGTACCATTC | |
| | | CTGATCAAGT GTGTGATAAT TCTCAGTGTG GACAACCTTT | |
| | | CCATCAAATA TGCTTATATG AGTGGCTGAG AGGACTACTA | |
| | | ACTAGTAGAC AGAGTTTTAA CATCATATTT GGTGAATGTC | |
| | | CATATTGTAG TAAGCCAATT ACCTTAAAAA TGTCTGGAAG | |
| | | GAAACACTGA AATAAGAATA CAACATTTCG GTGAAGAGCT | |
| | | GGAAACTTAA AAAATTATCA AAAGGAATTT TGGTATCATC | |
| | | TTCAGAGAAA AAATAAAGCA AGAAATACTA ACATCAAAAG | |
| | | GACAGGTATG ATGATGCGAT AATAATAAAC ATCTGCGTTT | |
| | | GTCTCTTCAC TAAGAGTAAA CTGGGAAATT GTAGGCCAAA | |
| | | GTCCAGTTGA ACTTTCTAAG TCTGTGATCC CCGTGCTGAC | |
| | | TGTGGAAGTG TATTTATACC AAGATGGAGA TCTTGACTTC | |
| | | TTGAATATAT CTGGACTGGT AAAATCTTGA TGAGGCTCAT | |
| | | AAAATGAGTT TGGGAATTGT GTATAGCTGA TTTTTTGTGG | |
| | | GAAACTGTTT ACTTCATTCA AAGGTTCTTG AGACTCTTGA | |
| | | TATTTCTGTC TTCTCCTTGT GCTTTCCTAT GGAAAAAATA | |
| | | CATATATAGT TTAGTTTGTT AGACGTGAGT TATCCAAGTA | |
| | | TTTATTTTGT GTAGTGTGTA AGAATGCTAA ATAAAATGTT | |
| | | ATACAAGATC AAAAAAAAAA AAAAAAAAAA AAA | |
| HJURP | NM_018410.4 | CTATTTGAGT TTGTGGCGCG CGAGGCCCTG CAGTCCGGGT | 12 |
| | | TGGCGCTTGG GTACTGGCTG GGTCCGATGC TGGGTACGCT | |
| | | GCGCGCCATG GAGGGCGAGG ACGTGGAAGA CGACCAGCTG | |
| | | CTGCAGAAGC TCAGGGCCAG TCGCCGCCGC TTCCAGAGGC | |
| | | GCATGCAGCG GCTGATAGAG AAGTACAACC AGCCCTTCGA | |
| | | GGACACCCCG GTGGTGCAAA TGGCCACGCT GACCTACGAG | |
| | | ACGCCACAGG GATTGAGAAT TTGGGGTGGA AGACTAATAA | |
| | | AGGAAAGAAA CGAAGGAGAG ATCCAGGACT CCTCCATGAA | |
| | | GCCCGCGGAC AGGACAGATG GCTCCGTGCA AGCTGCAGCC | |
| | | TGGGGTCCTG AGCTTCCCTC GCACCGCACA GTCCTGGGAG | |
| | | CCGATTCAAA AAGCGGTGAG GTCGATGCCA CGTCAGACCA | |
| | | GGAAGAGTCA GTTGCTTGGG CCTTAGCACC TGCAGTGCCT | |
| | | CAAAGCCCTT TGAAAAATGA ATTAAGAAGG AAATACTTGA | |
| | | CCCAAGTGGA TATACTGCTA CAAGGTGCAG AGTATTTTGA | |
| | | GTGTGCAGGT AACAGAGCTG GAAGGGATGT ACGTGTGACT | |
| | | CCGCTGCCTT CACTGGCCTC ACCTGCCGTG CCTGCCCCCG | |
| | | GATACTGCAG TCGTATCTCC AGAAAGAGTC CTGGTGACCC | |
| | | AGCGAAACCA GCTTCATCTC CCAGAGAATG GGATCCTTTG | |
| | | CATCCTTCCT CCACAGACAT GGCCTTAGTA CCTAGAAATG | |
| | | ACAGCCTCTC CCTACAAGAG ACCAGTAGCA GCAGCTTCTT | |
| | | AAGCAGCCAG CCCTTTGAAG ATGATGACAT TTGCAATGTG | |
| | | ACCATCAGTG ACCTGTACGC AGGGATGCTG CACTCCATGA | |
| | | GCCGGCTGTT GAGCACAAAG CCATCAAGCA TCATCTCCAC | |
| | | CAAAACGTTC ATCATGCAAA ACTGGAACTC CAGGAGGAGG | |
| | | CACAGATATA AGAGCAGGAT GAACAAAACA TATTGCAAAG | |
| | | GAGCCAGACG TTCTCAGAGG AGCTCCAAGG AGAACTTCAT | |
| | | ACCCTGCTCT GAGCCTGTGA AAGGGACAGG GGCATTAAGA | |
| | | GATTGCAAGA ACGTATTAGA TGTTTCTTGC CGTAAGACAG | |
| | | GTTTAAAATT GGAAAAAGCT TTTCTTGAAG TCAACAGACC | |
| | | CCAAATCCAT AAGTTAGATC CAAGTTGGAA GGAGCGCAAA | |
| | | GTGACACCCT CGAAGTATTC TTCCTTGATT TACTTCGACT | |
| | | CCAGTGCAAC ATATAATCTT GATGAGGAAA ATAGATTTAG | |
| | | GACATTAAAA TGGTTAATTT CTCCTGTAAA AATAGTTTCC | |
| | | AGACCAACAA TACGACAGGG CCATGGAGAG AACCGTCAGA | |
| | | GGGAGATTGA AATCCGATTT GATCAGCTTC ATCGGGAATA | |
| | | TTGCCTGAGT CCCAGGAACC AGCCTCGCCG GATGTGCCTC | |
| | | CCGGACTCCT GGGCCATGAA CATGTACAGA GGGGGTCCTG | |
| | | CGAGTCCTGG TGGCCTTCAG GGCTTAGAAA CCCGCAGGCT | |
| | | GAGTTTACCT TCCAGCAAAG CAAAAGCAAA AAGTTTAAGT | |
| | | GAGGCTTTTG AAAACCTAGG CAAAAGATCT CTGGAAGCAG | |
| | | GTAGGTGCCT GCCCAAGAGC GATTCATCTT CATCACTTCC | |
| | | AAAGACCAAC CCCACACACA GCGCAACTCG CCCGCAGCAG | |
| | | ACATCTGACC TTCACGTTCA GGGAAATAGT TCTGGAATAT | |
| | | TTAGAAAGTC AGTGTCACCC AGCAAAACTC TTTCAGTCCC | |
| | | AGATAAAGAA GTGCCAGGCC ACGGAAGGAA TCGTTACGAT | |
| | | GAAATTAAAG AAGAATTTGA CAAGCTTCAT CAAAAGTATT | |
| | | GCCTCAAATC TCCTGGGCAG ATGACAGTGC CTTTATGTAT | |
| | | TGGAGTGTCT ACAGATAAAG CAAGTATGGA AGTTCGATAT | |
| | | CAAACAGAAG GCTTCTTAGG AAAATTAAAT CCAGACCCTC | |
| | | ACTTCCAGGG TTTCCAGAAG TTGCCATCAT CACCCCTGGG | |
| | | GTGCAGAAAA AGTCTACTGG GCTCAACTGC AATTGAGGCT | |
| | | CCTTCATCTA CATGTGTTGC TCGTGCCATC ACGAGGGATG | |
| | | GCACGAGGGA CCATCAGTTC CCTGCAAAAA GACCCAGGCT | |
| | | ATCAGAACCC CAGGGCTCCG GACGCCAGGG CAATTCCCTG | |
| | | GGTGCCTCAG ATGGGGTGGA CAACACCGTC AGACCGGGAG | |
| | | ACCAGGGCAG CTCTTCACAG CCCAACTCAG AAGAGAGAGG | |
| | | AGAGAACACG TCTTACAGGA TGGAAGAGAA AAGTGATTTC | |
| | | ATGCTAGAAA AATTGGAAAC TAAAAGTGTG TAGCTAGGTT | |
| | | ATTTCGGAGT GTTATTTATC TTCCCACTTG CTCTCTGTTT | |
| | | GTATTTTTGT TTTGTTTTTG ATTCTTGAGA CTGTGAGGAC | |
| | | TTGGTTGACT TCTCTGCCCT TAAAGTAAAT ATTAGTGAAA | |
| | | TTGGTTCCAT CAGAGATAAC CTCGAGTTCT TGGTGTAGAA | |
| | | ATTATGTGAA TAAAGTTGCT CAATTAGAAT TTTTAGGGTT | |
| | | CTCTTTGATA GGCCTGTTTT TCTGATGTGT GTGTTTTTTT | |
| | | TGGGGGGGGG TTATTTGTTT GTTTGTTTGT TTGTTTGTTT | |
| | | GTTTTTGAGA CAGTCTCTCT CTATCGCCCA GGCTGGAGTG | |
| | | CGGTGGCACA ATCTTGGCTC ACTGCAACTT CCGCCTCCCG | |
| | | GGTTCAAGCG ATTCTTCTGC CTCAGCCTCC CGAGTAGCTG | |
| | | GGATTACAGG CGCGCGCCAC CACGCCTGGC TAATTTTTGT | |
| | | AGTTTTAGTA GAGACGGGGT TTCACCATAT TGACCAGGCT | |
| | | GGTCTCGAGC TCCTGGCCTC GTGATCCATC TGCCTCGGCC | |
| | | TCCCAAAGTG CTGGGATTAT AGGCGTGAGC CACTGCTCCC | |
| | | AGCCGTGTGT TCTTTTTTAA ATTTAGATAT GTCCAGAGAA | |
| | | TCCTCTCTCC TGTTTCCCAT TTCATTCGAG AATATTGTTT | |
| | | GCTTGTGAGA CGTAAGTTCG AGCCCTGCAT GCAATGACCC | |
| | | TTGAAGGAAA ATAAACAGTC CTGGTGGTCC CAGACGCTCC | |
| | | TGCAGCCACA GCGCCTGTGA CTCCTCATGA TTCTTACTGA | |
| | | AGCTGTTGAT GACAGGATAT CATGGTGACG TTTTTGTAAT | |
| | | GAAATATTTC ACATATTCAG AATACATTGG TGAAACTCAT | |
| | | GCTGGAGTAA ATAGTTAATA TATGGCCAT | |
| HLA- | NM_002119.3 | CTTCTTCTTT ACCTCCGCCT TGTTCCTGTC CTCACCACAC | 13 |
| DOA | | GGACTGAGAC TGATTTGATT AAAGCACCAG AGTGTAATGG | |
| | | CCCTCAGAGC AGGGCTGGTC CTGGGGTTCC ACACCCTGAT | |
| | | GACCCTCCTG AGCCCGCAGG AGGCAGGGGC CACCAAGGCT | |
| | | GACCACATGG GCTCCTACGG ACCCGCCTTC TACCAGTCTT | |
| | | ACGGCGCCTC GGGCCAGTTC ACCCATGAAT TTGATGAGGA | |
| | | ACAGCTGTTC TCTGTGGACC TGAAGAAAAG CGAGGCCGTG | |
| | | TGGCGTCTGC CTGAGTTTGG TGACTTTGCC CGCTTTGACC | |
| | | CGCAGGGCGG GCTGGCCGGC ATCGCCGCAA TCAAAGCCCA | |
| | | TCTGGACATC CTGGTGGAGC GCTCCAACCG CAGCAGAGCC | |
| | | ATCAACGTGC CTCCACGGGT GACCGTGCTC CCCAAGTCTC | |
| | | GGGTGGAGCT GGGCCAGCCC AACATCCTCA TCTGCATCGT | |
| | | GGACAACATC TTCCCCCCTG TGATCAATAT CACCTGGCTG | |
| | | CGCAACGGCC AAACTGTCAC TGAGGGAGTG GCCCAGACCA | |
| | | GCTTCTATTC CCAGCCTGAC CATTTGTTCC GCAAGTTCCA | |
| | | CTACCTGCCC TTCGTGCCCT CAGCCGAGGA CGTCTATGAC | |
| | | TGCCAGGTGG AGCACTGGGG CCTGGATGCG CCACTCCTCA | |
| | | GGCATTGGGA GCTCCAGGTG CCTATTCCAC CACCAGATGC | |
| | | CATGGAGACC CTGGTCTGTG CCCTGGGCCT GGCCATCGGC | |
| | | CTGGTGGGCT TCCTCGTGGG CACCGTCCTC ATCATCATGG | |
| | | GCACATATGT GTCCAGTGTC CCCAGGTAAT GATCCTTCTG | |
| | | AGAGAAATGA CTTGTGGGAG ACACCCTGCA GATCCTCATG | |
| | | GGTTTGTGAC AGCCCCTGCG TGCTCAGTGC CCTTTAAGTG | |
| | | CATCCCGCTG TGCTGACTTT GAGTGGGATC AACATCTGTC | |
| | | CTACGGGTCC CCTCTTTTTT GGCCCCAGTA TTCATGGCAG | |
| | | GGTTTGTTGG ACACCTACTA GCTTCCCTTC CCATTCAACA | |
| | | CACACACACA TTCTTGCTCT ACCCAAAGCT CTGGCTGGCA | |
| | | GCACTAAATG CTTTGGTGGT GTTTGCACTG TGTCCTTTCC | |
| | | AGGCCTTGGC CAGTTCTTCC AGGGGTGAGG CATGTGGTGC | |
| | | TGGGGATTGG CAGCCATCCT GGGGCCCACA CAGGTGTGTC | |
| | | TTGCTCCATT TGGCCCATTG TGTGTTACTT TGTGAATGAG | |
| | | CCATTTCACA TGGACTTCAT GAAATTTGCC TCCTGAGTTC | |
| | | AGGTTTACCC TGAAAGGGAT GCAGATTATC CTGTTCCTCA | |
| | | CGACCCCCTC AGCTAACAAC AGTTCTGAAG GGTGCTGGGA | |
| | | CAGGACAGGC TCATGGGGAC TCCACTCCTG CCTGGGTTTA | |
| | | CTCTGTATGA AGAGGCCACT GGTATCCTGC CATGATGTTA | |
| | | TCTCCTTTTT CTACTTTTCC CTAGAGTCCC ATGCATGATA | |
| | | AAGAGAGGCC CAAGGCTTGG ATAAGGTGGC CACTTCCCTC | |
| | | AGTGGAGTCA GTCATGTTAG GTAGGAGGTG GTAGAGTCGG | |
| | | TCTGCGAGGT ATCTCGTAAG AGGGGAGGTC CACCTAGACA | |
| | | CACTCTAAAT ATGTGGCCTA GAAGATTTTG GTCTACTTTT | |
| | | CTGTGAACAG AATTTAAAAC ATACAAAGAG ATAAATCACC | |
| | | ATACCACATA GTTTATGTCA GGACCAAAAT GAGCAATACA | |
| | | GATTACGGTT TTCAAACCAG AATGCACATA AGAACTGCTT | |
| | | GGGATCCTTT TAAAAGTACA GGCATTGGCC TGGTGCAGTG | |
| | | GCTCATTCCT GTAATCCCAG CACTTTGGGA GGCCAAGGGG | |
| | | ACAGGACTGC TTGAGGCCAA GAGGTGGAAA CCATCTTGGG | |
| | | CTACATAGAG AGACCCCATC TCTACAAAGA AAGATTTAAA | |
| | | AATTAACCAG GCATGGTGGC TCGCACCTGT ATTCCCAGCC | |
| | | ACTGGGGAGG CTGAGGCCGG AGGAGTGCTT GAGCCCAGGA | |
| | | GTTCAAGGCT GCAGTGAGCC AAGATTGCGC CACTGCACTC | |
| | | CAGCCTAGGT GACAGAGTGA GACCCTGTCT CTAAATAAAT | |
| | | AAATAAATAA AATATAAAAA TAACAGTCAT CACCCAGACC | |
| | | TACTGAATTA GAATCTCGGG AGTGCAGGGG GCAGCAACAG | |
| | | GGAGGCTGTC TTTTCTGAGA TGGGGTCTCA CTCTGTCACC | |
| | | AGGCTGGAGT GCCATGGCAT GATCTCAGCT CACTGCAACC | |
| | | TCCACCTCCT GAGTTCAAGC CATTCTCCTG CCTCAGCCTC | |
| | | CTGAGTAGCT GGGACTACAG GTGTGCGCCA CTACACTCAG | |
| | | CTAATTTTTG TATTTTAAGT AGAGACGGGG TTTCATCATG | |
| | | TTGGCCAGGA TGGCCTCCAT CTCTTGACCT CGTGATCCAC | |
| | | CCACCTTCCC TCCCAAAGTA CTGGAATTAC AGGCATTAGC | |
| | | CACTGTGCCC AGCCGAGGCT GTCATTTTTA ACCGGCTCTG | |
| | | GATGACTCTG ATGCAGCCAT CCTGGACCTT GGCTGTGGTC | |
| | | TGGTAACTGG AACCCAGTGA CGTAATCAGG TGCCATCGGG | |
| | | GGTCATGGGA AAGGGGGATC CCCAAGGTCT GAGGTGGACT | |
| | | AGGAAGGCTT TCTGAAGAAC CTGGGTCTGT TAGGGCATCA | |
| | | GCCAATCAAG GTACAAGTAA ATAGAGGCAA AATGAGGGTT | |
| | | TGAACTGTGA GCAGTTGGTC CTGGAAAAGA AAGAAACCAA | |
| | | GAGATTATGG GGACTCAATG GGCTTCTTAA GAGAGAATAA | |
| | | GTTGAAATCA ATGACCAGAA GACCCTGATG GAAGTGGAGG | |
| | | AGAATCATCT CAGGCAAACT TTTTGTGTGC CAGTAACAGA | |
| | | AACCCTCTTT GTGTGATCAC ATGCAAAGTA TAGGATATTT | |
| | | GCAATATAGC CATGGGGAGG AGTGCAGGGC CCAAGGGTAG | |
| | | ATTTTAGCCA GGCCTCCCAG GAACAGAACT CGGATCCGAA | |
| | | AAGCCCAGAG AAGCTAGAGC TGCCCCTCCA ACACTCTCGG | |
| | | ATCCACATGG TCTGTGTTCT CTAGACCCCC CTGCATGTTA | |
| | | GCGGTGTTCT CTCTCTGTGG ACTGACTGTC CTTCTCAGTG | |
| | | AACATGTCCA CCCGACAGCT CCTGAGTTTA TATCATCTCA | |
| | | ACCCTCACAA CCCACAGAGG CTGTGTCTCC TAGTCACAGC | |
| | | TTTAAATTAC TGGAAAAATA AATGACTGGC CAAACTTGGA | |
| | | GCAGGTGTCC ATCCCAGCCC TGTGTAGTTA GAGCAGGAAT | |
| | | CAAGATCTCA ACACAAATGT GGCTGCCAAG CACTCAGCCC | |
| | | CGGGGCGAGG GGTCAAGTTC TTCTCAGAGA AAGAGGAATA | |
| | | AGTTGGTTCT CAGAAGACAT CACAAGATAC GTGTGTACCC | |
| | | AACAATCTCT GATCTCTGCT GATCTTTTGC TTAGACGTTA | |
| | | ACTTGATGCA TCATTGGAAA GGTGTTTCTC TCATCTCTGT | |
| | | CCTAAGGCTT GATAAAGTCA TTAAAATTGT GTTCTTTTGA | |
| | | CTAAA | |
| HLA- | NM_019111.4 | TTTTAATGGT CAGACTCTAT TACACCCCAC ATTCTCTTTT | 14 |
| DRA | | CTTTTATTCT TGTCTGTTCT GCCTCACTCC CGAGCTCTAC | |
| | | TGACTCCCAA CAGAGCGCCC AAGAAGAAAA TGGCCATAAG | |
| | | TGGAGTCCCT GTGCTAGGAT TTTTCATCAT AGCTGTGCTG | |
| | | ATGAGCGCTC AGGAATCATG GGCTATCAAA GAAGAACATG | |
| | | TGATCATCCA GGCCGAGTTC TATCTGAATC CTGACCAATC | |
| | | AGGCGAGTTT ATGTTTGACT TTGATGGTGA TGAGATTTTC | |
| | | CATGTGGATA TGGCAAAGAA GGAGACGGTC TGGCGGCTTG | |
| | | AAGAATTTGG ACGATTTGCC AGCTTTGAGG CTCAAGGTGC | |
| | | ATTGGCCAAC ATAGCTGTGG ACAAAGCCAA CCTGGAAATC | |
| | | ATGACAAAGC GCTCCAACTA TACTCCGATC ACCAATGTAC | |
| | | CTCCAGAGGT AACTGTGCTC ACAAACAGCC CTGTGGAACT | |
| | | GAGAGAGCCC AACGTCCTCA TCTGTTTCAT AGACAAGTTC | |
| | | ACCCCACCAG TGGTCAATGT CACGTGGCTT CGAAATGGAA | |
| | | AACCTGTCAC CACAGGAGTG TCAGAGACAG TCTTCCTGCC | |
| | | CAGGGAAGAC CACCTTTTCC GCAAGTTCCA CTATCTCCCC | |
| | | TTCCTGCCCT CAACTGAGGA CGTTTACGAC TGCAGGGTGG | |
| | | AGCACTGGGG CTTGGATGAG CCTCTTCTCA AGCACTGGGA | |
| | | GTTTGATGCT CCAAGCCCTC TCCCAGAGAC TACAGAGAAC | |
| | | GTGGTGTGTG CCCTGGGCCT GACTGTGGGT CTGGTGGGCA | |
| | | TCATTATTGG GACCATCTTC ATCATCAAGG GATTGCGCAA | |
| | | AAGCAATGCA GCAGAACGCA GGGGGCCTCT GTAAGGCACA | |
| | | TGGAGGTGAT GGTGTTTCTT AGAGAGAAGA TCACTGAAGA | |
| | | AACTTCTGCT TTAATGGCTT TACAAAGCTG GCAATATTAC | |
| | | AATCCTTGAC CTCAGTGAAA GCAGTCATCT TCAGCATTTT | |
| | | CCAGCCCTAT AGCCACCCCA AGTGTGGATA TGCCTCTTCG | |
| | | ATTGCTCCGT ACTCTAACAT CTAGCTGGCT TCCCTGTCTA | |
| | | TTGCCTTTTC CTGTATCTAT TTTCCTCTAT TTCCTATCAT | |
| | | TTTATTATCA CCATGCAATG CCTCTGGAAT AAAACATACA | |
| | | GGAGTCTGTC TCTGCTATGG AATGCCCCAT GGGGCATCTC | |
| | | TTGTGTACTT ATTGTTTAAG GTTTCCTCAA ACTGTGATTT | |
| | | TTCTGAACAC AATAAACTAT TTTGATGATC TTGGGTGGAA | |
| | | AAAAAAAAAA AAAAAAAAAA AAAAAAAAAA AA | |
| HNRNPA3P1 | NR_002726.2 | CGAGTTGGAA GAGGTGAGTC CTGTCTCAAA ATGGAGGTAA | 15 |
| | | AACCGCCGCC TGGTTGCCCC CAGCCCGACT CCGGCAGTCG | |
| | | CCGTCGCCAC TGGGGGGAGG AGGGCCATGA TCCAAAGGAA | |
| | | CCAGAGCAGC TGAGAAAACT GTTTATTGGT GGTCTGAGCT | |
| | | TTGAAACTAC AGATGATAGT TTAAGAGAAC ATTTTGAGAA | |
| | | ATGGGGCACA CTCACAGATT GTCTGGTAAT GAGAGACCCC | |
| | | CAAACAAAAC GTTCCAGGGG CTTTGGTTTT GTGACTTATT | |
| | | CTTGTGTTAC AGAGGTGGAT GCAGCAATGC GTGCTCGACC | |
| | | ATTCAAGGTT GATGGGCGTG TAGTGGAACC AAAGAGAGCT | |
| | | GTTTCTAGAG AGGATTCTGT GAAGCCTGGT GCCCATCTAA | |
| | | CAGTGAAGAA AATTTTTGTT GGCAGTATTA AAGAAGATAC | |
| | | AGAAGAATAT AATTTGAGAG ACTACTTTGA AAAGTACGGC | |
| | | AAGATTGAAA CCATAGAAGT TATGGAAGAC AGGCAGAGTG | |
| | | GAAAAAAGAG AGGATTTGCT TCTGTAACTT TTGATGATCA | |
| | | TGATACAGTT GATAAAATTG TTGTTCAGAA ATACCACACT | |
| | | ATTAATGGGC ATAACTGTGA AGTGAAAAAG GCCCTTGCTA | |
| | | AACAAGTGAT GCAGCCGGCT GGATCACAGA GGGGTCGTGG | |
| | | AGGTGGATCT GGCAATTGTA TGGGTCACAG AGGAAACTTT | |
| | | GGAGGTGGTG GAGGTAATTT TGGCCGTGAT GGAAACTTTG | |
| | | GTGGAAGAGG AGGCTATGGT GGTGGAGGTG GTGGCAGCAG | |
| | | AGGTAGTTAT GGAGGAGGTG ATGTGGATAT AATGGATTAG | |
| | | GAGGTGATGG TGGCAACTAT GGCAGTGGTC CTGGTTATAG | |
| | | TAGTAGAGGC GGGTATGGTG GTGGTGGACC AGGATATGGA | |
| | | AACCAAGGTG GTGGATATGG TGGCGGTGTT GGAGGATATG | |
| | | ATGGTTACAA TGAAGGAGGA AATTTTGACG GTAGTAACTA | |
| | | TGGTGGTGGT GGGAACTATA ATGATTTTGG AAATTACAGT | |
| | | GGACAACAGC AATCAAATTA TGGACACATG AAAGGGGGCA | |
| | | GTTTTGGTGG AAGAAGCTCG GGCAGTCCCT ATGGTGGTGG | |
| | | TTATGGATCT GGTGGTGGAA GTGGTGGATA TGGTAGCAGA | |
| | | AGGTTCTAAA AACAGCAGGA AAGGGCTACA GTTCTTAGCA | |
| | | GGAGAGAGAG TGAGGAGTTG TCAGGAAAGC TACAGGTTAC | |
| | | TTTGAGACAG TCGTCCCAAG TGCATTAGAG GAACTGTAAA | |
| | | AATCTGTCAC AGAAGGAACG ATGATCCATA ATCAGAAAAG | |
| | | TTACTGCAGC TTAAACAGGA AACCCTTCTT GTTCAGGACT | |
| | | GTCATAGCCA CAGTTTGCAG GAAGTGCAGC TATCGATTAA | |
| | | TGCAATGTAG CGTCAATTAG ATGTACATTC CTCAGGTCTT | |
| | | TTATCTGTTG TAGCTTTGTC TTATTCTTTT TCTTTTCATT | |
| | | ACATCAGGTA TATTGCCCTG TAAATTGTGG TAGTGGTACC | |
| | | AGGAATAAAA AATTAAGGAA TTTTTAACTT TTCAATATCT | |
| | | GTGTAGTTCA GTTTTTCTAC ATTTTAGTAC AGAAACTTTA | |
| | | ACAAAATGCA GTTTTGAAGG TGTTTCCTTG TGAGTTAACA | |
| | | AGTAAAGAAG ATCAATTGTT AATTACTATT TTGTATAAAT | |
| | | TTTGCTAAAG TTAACTGTAA AGAAACACCT GCTGACTTGC | |
| | | AGTTTAAGGG GAATCTATTC TCCCCATTTC CAAACCATGA | |
| | | TATGAATGGA CGCCGACATG TGGAGAGAAC AGATAATTTG | |
| | | TGTGTTTGCA ATGTGTGTTT TAGGTAAATA GGATTGGGTA | |
| | | TTTAAATTAG CATTTGTGAA TTTAATAGCA TTAAGATTAC | |
| | | CTTCAAATAA AAAAGTCTCA AAATTTCTTT TTGGTTTTTG | |
| | | TGCACTTTCT TTTAAAATGT AATCACATGA TTTTAGTGTG | |
| | | TTAGACTTGC TGAGTCCTAG CTGTGTTTAG AACATCTCCA | |
| | | TTCTACATTT ACCTTGGTCA AATTTGAACT GCTGCCATAG | |
| | | GTTTTGGGTG TAAAGAATGT TTACTGCCCT CCATTTAAAT | |
| | | TCTGAAAAGG TATAGTGGAT GTTTTCCCTC TCCTACATTA | |
| | | GAAACCATTC TTAAAAACTT TTCAAAATAT AGAACCATTA | |
| | | AGCCTGCTAT ATCTGAGCAA ATTAGTGGGT ACCTTTTTTC | |
| | | CTTTTTTAAA GCACAAGAGG CCCATAAATC TTGAGTTATT | |
| | | TGCATTAGTT TACATTTTTT GATACAACTT TTCAGACCAA | |
| | | GAGAATAAAA ATCATGCGTT ATTAAACCCC TAGCTGGCTG | |
| | | GCATGCTTTC CTGTTTGTAC TGTATACATT TTGCTGGATG | |
| | | AAACCAAGGA TAGTTTAGGT ATAATTGTCC AAAATAACCT | |
| | | AACTGCAGCA GAAATGTAGG ACAGTTGCTT AGTACAGGCT | |
| | | TCTCACTTCC TACAGACCTG AATTCAAATT TGGATAGTCT | |
| | | GAGTTATTAA ATTCCCAAAG ACAAAGAACA CACTCTTATT | |
| | | TCTTGTGTAT ATTTCAACAT AAATCATGTT GTTACCAATT | |
| | | TGTTGGGAAG GCCCTGGTTG AGAAGAGTTT TAGATAATAA | |
| | | GGCTGTATAT ATATAGATAT ATATAGATAT ATACCAATGT | |
| | | CTATATATAG AGATATTTTA TATATATATA TACAGGTATA | |
| | | TATATGTGTG TGTATATATA TAGGTATATA CATATATACA | |
| | | TATATATATA TATATATATG GATATATACC CATGTCTACT | |
| | | GTTTTGCTTC AGCTAGTGCT TACAATTTCA TTCAAGTCCT | |
| | | GAGTATGTGT CCTGCTGTTA CTCCTTCTTT GGTAGTTGAA | |
| | | CGTTGAATTC AAGTCTTTCC TTCTGTTTTA AGAAGTACTA | |
| | | AGCAAACAAG CAATAAAAAG GGGAATGGCG CATGCTAGTG | |
| | | TTTGAATATG CTCTCTTGTT GCTCTAATTC TGTGCCTCCG | |
| | | TGCATTAATA TTTGGATGCA TGCAATGCCA GCATGGAAAT | |
| | | TGGCCT | |
| IL23A | NM_016584.2 | AAAACAACAG GAAGCAGCTT ACAAACTCGG TGAACAACTG | 16 |
| | | AGGGAACCAA ACCAGAGACG CGCTGAACAG AGAGAATCAG | |
| | | GCTCAAAGCA AGTGGAAGTG GGCAGAGATT CCACCAGGAC | |
| | | TGGTGCAAGG CGCAGAGCCA GCCAGATTTG AGAAGAAGGC | |
| | | AAAAAGATGC TGGGGAGCAG AGCTGTAATG CTGCTGTTGC | |
| | | TGCTGCCCTG GACAGCTCAG GGCAGAGCTG TGCCTGGGGG | |
| | | CAGCAGCCCT GCCTGGACTC AGTGCCAGCA GCTTTCACAG | |
| | | AAGCTCTGCA CACTGGCCTG GAGTGCACAT CCACTAGTGG | |
| | | GACACATGGA TCTAAGAGAA GAGGGAGATG AAGAGACTAC | |
| | | AAATGATGTT CCCCATATCC AGTGTGGAGA TGGCTGTGAC | |
| | | CCCCAAGGAC TCAGGGACAA CAGTCAGTTC TGCTTGCAAA | |
| | | GGATCCACCA GGGTCTGATT TTTTATGAGA AGCTGCTAGG | |
| | | ATCGGATATT TTCACAGGGG AGCCTTCTCT GCTCCCTGAT | |
| | | AGCCCTGTGG GCCAGCTTCA TGCCTCCCTA CTGGGCCTCA | |
| | | GCCAACTCCT GCAGCCTGAG GGTCACCACT GGGAGACTCA | |
| | | GCAGATTCCA AGCCTCAGTC CCAGCCAGCC ATGGCAGCGT | |
| | | CTCCTTCTCC GCTTCAAAAT CCTTCGCAGC CTCCAGGCCT | |
| | | TTGTGGCTGT AGCCGCCCGG GTCTTTGCCC ATGGAGCAGC | |
| | | AACCCTGAGT CCCTAAAGGC AGCAGCTCAA GGATGGCACT | |
| | | CAGATCTCCA TGGCCCAGCA AGGCCAAGAT AAATCTACCA | |
| | | CCCCAGGCAC CTGTGAGCCA ACAGGTTAAT TAGTCCATTA | |
| | | ATTTTAGTGG GACCTGCATA TGTTGAAAAT TACCAATACT | |
| | | GACTGACATG TGATGCTGAC CTATGATAAG GTTGAGTATT | |
| | | TATTAGATGG GAAGGGAAAT TTGGGGATTA TTTATCCTCC | |
| | | TGGGGACAGT TTGGGGAGGA TTATTTATTG TATTTATATT | |
| | | GAATTATGTA CTTTTTTCAA TAAAGTCTTA TTTTTGTGGC | |
| | | TAAAAAAAA | |
| IQGAP1 | NM_003870.3 | GGACCCCGGC AAGCCCGCGC ACTTGGCAGG AGCTGTAGCT | 17 |
| | | ACCGCCGTCC GCGCCTCCAA GGTTTCACGG CTTCCTCAGC | |
| | | AGAGACTCGG GCTCGTCCGC CATGTCCGCC GCAGACGAGG | |
| | | TTGACGGGCT GGGCGTGGCC CGGCCGCACT ATGGCTCTGT | |
| | | CCTGGATAAT GAAAGACTTA CTGCAGAGGA GATGGATGAA | |
| | | AGGAGACGTC AGAACGTGGC TTATGAGTAC CTTTGTCATT | |
| | | TGGAAGAAGC GAAGAGGTGG ATGGAAGCAT GCCTAGGGGA | |
| | | AGATCTGCCT CCCACCACAG AACTGGAGGA GGGGCTTAGG | |
| | | AATGGGGTCT ACCTTGCCAA ACTGGGGAAC TTCTTCTCTC | |
| | | CCAAAGTAGT GTCCCTGAAA AAAATCTATG ATCGAGAACA | |
| | | GACCAGATAC AAGGCGACTG GCCTCCACTT TAGACACACT | |
| | | GATAATGTGA TTCAGTGGTT GAATGCCATG GATGAGATTG | |
| | | GATTGCCTAA GATTTTTTAC CCAGAAACTA CAGATATCTA | |
| | | TGATCGAAAG AACATGCCAA GATGTATCTA CTGTATCCAT | |
| | | GCACTCAGTT TGTACCTGTT CAAGCTAGGC CTGGCCCCTC | |
| | | AGATTCAAGA CCTATATGGA AAGGTTGACT TCACAGAAGA | |
| | | AGAAATCAAC AACATGAAGA CTGAGTTGGA GAAGTATGGC | |
| | | ATCCAGATGC CTGCCTTTAG CAAGATTGGG GGCATCTTGG | |
| | | CTAATGAACT GTCAGTGGAT GAAGCCGCAT TACATGCTGC | |
| | | TGTTATTGCT ATTAATGAAG CTATTGACCG TAGAATTCCA | |
| | | GCCGACACAT TTGCAGCTTT GAAAAATCCG AATGCCATGC | |
| | | TTGTAAATCT TGAAGAGCCC TTGGCATCCA CTTACCAGGA | |
| | | TATACTTTAC CAGGCTAAGC AGGACAAAAT GACAAATGCT | |
| | | AAAAACAGGA CAGAAAACTC AGAGAGAGAA AGAGATGTTT | |
| | | ATGAGGAGCT GCTCACGCAA GCTGAAATTC AAGGCAATAT | |
| | | AAACAAAGTC AATACATTTT CTGCATTAGC AAATATCGAC | |
| | | CTGGCTTTAG AACAAGGAGA TGCACTGGCC TTGTTCAGGG | |
| | | CTCTGCAGTC ACCAGCCCTG GGGCTTCGAG GACTGCAGCA | |
| | | ACAGAATAGC GACTGGTACT TGAAGCAGCT CCTGAGTGAT | |
| | | AAACAGCAGA AGAGACAGAG TGGTCAGACT GACCCCCTGC | |
| | | AGAAGGAGGA GCTGCAGTCT GGAGTGGATG CTGCAAACAG | |
| | | TGCTGCCCAG CAATATCAGA GAAGATTGGC AGCAGTAGCA | |
| | | CTGATTAATG CTGCAATCCA GAAGGGTGTT GCTGAGAAGA | |
| | | CTGTTTTGGA ACTGATGAAT CCCGAAGCCC AGCTGCCCCA | |
| | | GGTGTATCCA TTTGCCGCCG ATCTCTATCA GAAGGAGCTG | |
| | | GCTACCCTGC AGCGACAAAG TCCTGAACAT AATCTCACCC | |
| | | ACCCAGAGCT CTCTGTCGCA GTGGAGATGT TGTCATCGGT | |
| | | GGCCCTGATC AACAGGGCAT TGGAATCAGG AGATGTGAAT | |
| | | ACAGTGTGGA AGCAATTGAG CAGTTCAGTT ACTGGTCTTA | |
| | | CCAATATTGA GGAAGAAAAC TGTCAGAGGT ATCTCGATGA | |
| | | GTTGATGAAA CTGAAGGCTC AGGCACATGC AGAGAATAAT | |
| | | GAATTCATTA CATGGAATGA TATCCAAGCT TGCGTGGACC | |
| | | ATGTGAACCT GGTGGTGCAA GAGGAACATG AGAGGATTTT | |
| | | AGCCATTGGT TTAATTAATG AAGCCCTGGA TGAAGGTGAT | |
| | | GCCCAAAAGA CTCTGCAGGC CCTACAGATT CCTGCAGCTA | |
| | | AACTTGAGGG AGTCCTTGCA GAAGTGGCCC AGCATTACCA | |
| | | AGACACGCTG ATTAGAGCGA AGAGAGAGAA AGCCCAGGAA | |
| | | ATCCAGGATG AGTCAGCTGT GTTATGGTTG GATGAAATTC | |
| | | AAGGTGGAAT CTGGCAGTCC AACAAAGACA CCCAAGAAGC | |
| | | ACAGAAGTTT GCCTTAGGAA TCTTTGCCAT TAATGAGGCA | |
| | | GTAGAAAGTG GTGATGTTGG CAAAACACTG AGTGCCCTTC | |
| | | GCTCCCCTGA TGTTGGCTTG TATGGAGTCA TCCCTGAGTG | |
| | | TGGTGAAACT TACCACAGTG ATCTTGCTGA AGCCAAGAAG | |
| | | AAAAAACTGG CAGTAGGAGA TAATAACAGC AAGTGGGTGA | |
| | | AGCACTGGGT AAAAGGTGGA TATTATTATT ACCACAATCT | |
| | | GGAGACCCAG GAAGGAGGAT GGGATGAACC TCCAAATTTT | |
| | | GTGCAAAATT CTATGCAGCT TTCTCGGGAG GAGATCCAGA | |
| | | GTTCTATCTC TGGGGTGACT GCCGCATATA ACCGAGAACA | |
| | | GCTGTGGCTG GCCAATGAAG GCCTGATCAC CAGGCTGCAG | |
| | | GCTCGCTGCC GTGGATACTT AGTTCGACAG GAATTCCGAT | |
| | | CCAGGATGAA TTTCCTGAAG AAACAAATCC CTGCCATCAC | |
| | | CTGCATTCAG TCACAGTGGA GAGGATACAA GCAGAAGAAG | |
| | | GCATATCAAG ATCGGTTAGC TTACCTGCGC TCCCACAAAG | |
| | | ATGAAGTTGT AAAGATTCAG TCCCTGGCAA GGATGCACCA | |
| | | AGCTCGAAAG CGCTATCGAG ATCGCCTGCA GTACTTCCGG | |
| | | GACCATATAA ATGACATTAT CAAAATCCAG GCTTTTATTC | |
| | | GGGCAAACAA AGCTCGGGAT GACTACAAGA CTCTCATCAA | |
| | | TGCTGAGGAT CCTCCTATGG TTGTGGTCCG AAAATTTGTC | |
| | | CACCTGCTGG ACCAAAGTGA CCAGGATTTT CAGGAGGAGC | |
| | | TTGACCTTAT GAAGATGCGG GAAGAGGTTA TCACCCTCAT | |
| | | TCGTTCTAAC CAGCAGCTGG AGAATGACCT CAATCTCATG | |
| | | GATATCAAAA TTGGACTGCT AGTGAAAAAT AAGATTACGT | |
| | | TGCAGGATGT GGTTTCCCAC AGTAAAAAAC TTACCAAAAA | |
| | | AAATAAGGAA CAGTTGTCTG ATATGATGAT GATAAATAAA | |
| | | CAGAAGGGAG GTCTCAAGGC TTTGAGCAAG GAGAAGAGAG | |
| | | AGAAGTTGGA AGCTTACCAG CACCTGTTTT ATTTATTGCA | |
| | | AACCAATCCC ACCTATCTGG CCAAGCTCAT TTTTCAGATG | |
| | | CCCCAGAACA AGTCCACCAA GTTCATGGAC TCTGTAATCT | |
| | | TCACACTCTA CAACTACGCG TCCAACCAGC GAGAGGAGTA | |
| | | CCTGCTCCTG CGGCTCTTTA AGACAGCACT CCAAGAGGAA | |
| | | ATCAAGTCGA AGGTAGATCA GATTCAAGAG ATTGTGACAG | |
| | | GAAATCCTAC GGTTATTAAA ATGGTTGTAA GTTTCAACCG | |
| | | TGGTGCCCGT GGCCAGAATG CCCTGAGACA GATCTTGGCC | |
| | | CCAGTCGTGA AGGAAATTAT GGATGACAAA TCTCTCAACA | |
| | | TCAAAACTGA CCCTGTGGAT ATTTACAAAT CTTGGGTTAA | |
| | | TCAGATGGAG TCTCAGACAG GAGAGGCAAG CAAACTGCCC | |
| | | TATGATGTGA CCCCTGAGCA GGCGCTAGCT CATGAAGAAG | |
| | | TGAAGACACG GCTAGACAGC TCCATCAGGA ACATGCGGGC | |
| | | TGTGACAGAC AAGTTTCTCT CAGCCATTGT CAGCTCTGTG | |
| | | GACAAAATCC CTTATGGGAT GCGCTTCATT GCCAAAGTGC | |
| | | TGAAGGACTC GTTGCATGAG AAGTTCCCTG ATGCTGGTGA | |
| | | GGATGAGCTG CTGAAGATTA TTGGTAACTT GCTTTATTAT | |
| | | CGATACATGA ATCCAGCCAT TGTTGCTCCT GATGCCTTTG | |
| | | ACATCATTGA CCTGTCAGCA GGAGGCCAGC TTACCACAGA | |
| | | CCAACGCCGA AATCTGGGCT CCATTGCAAA AATGCTTCAG | |
| | | CATGCTGCTT CCAATAAGAT GTTTCTGGGA GATAATGCCC | |
| | | ACTTAAGCAT CATTAATGAA TATCTTTCCC AGTCCTACCA | |
| | | GAAATTCAGA CGGTTTTTCC AAACTGCTTG TGATGTCCCA | |
| | | GAGCTTCAGG ATAAATTTAA TGTGGATGAG TACTCTGATT | |
| | | TAGTAACCCT CACCAAACCA GTAATCTACA TTTCCATTGG | |
| | | TGAAATCATC AACACCCACA CTCTCCTGTT GGATCACCAG | |
| | | GATGCCATTG CTCCGGAGCA CAATGATCCA ATCCACGAAC | |
| | | TGCTGGACGA CCTCGGCGAG GTGCCCACCA TCGAGTCCCT | |
| | | GATAGGGGAA AGCTCTGGCA ATTTAAATGA CCCAAATAAG | |
| | | GAGGCACTGG CTAAGACGGA AGTGTCTCTC ACCCTGACCA | |
| | | ACAAGTTCGA CGTGCCTGGA GATGAGAATG CAGAAATGGA | |
| | | TGCTCGAACC ATCTTACTGA ATACAAAACG TTTAATTGTG | |
| | | GATGTCATCC GGTTCCAGCC AGGAGAGACC TTGACTGAAA | |
| | | TCCTAGAAAC ACCAGCCACC AGTGAACAGG AAGCAGAACA | |
| | | TCAGAGAGCC ATGCAGAGAC GTGCTATCCG TGATGCCAAA | |
| | | ACACCTGACA AGATGAAAAA GTCAAAATCT GTAAAGGAAG | |
| | | ACAGCAACCT CACTCTTCAA GAGAAGAAAG AGAAGATCCA | |
| | | GACAGGTTTA AAGAAGCTAA CAGAGCTTGG AACCGTGGAC | |
| | | CCAAAGAACA AATACCAGGA ACTGATCAAC GACATTGCCA | |
| | | GGGATATTCG GAATCAGCGG AGGTACCGAC AGAGGAGAAA | |
| | | GGCCGAACTA GTGAAACTGC AACAGACATA CGCTGCTCTG | |
| | | AACTCTAAGG CCACCTTTTA TGGGGAGCAG GTGGATTACT | |
| | | ATAAAAGCTA TATCAAAACC TGCTTGGATA ACTTAGCCAG | |
| | | CAAGGGCAAA GTCTCCAAAA AGCCTAGGGA AATGAAAGGA | |
| | | AAGAAAAGCA AAAAGATTTC TCTGAAATAT ACAGCAGCAA | |
| | | GACTACATGA AAAAGGAGTT CTTCTGGAAA TTGAGGACCT | |
| | | GCAAGTGAAT CAGTTTAAAA ATGTTATATT TGAAATCAGT | |
| | | CCAACAGAAG AAGTTGGAGA CTTCGAAGTG AAAGCCAAAT | |
| | | TCATGGGAGT TCAAATGGAG ACTTTTATGT TACATTATCA | |
| | | GGACCTGCTG CAGCTACAGT ATGAAGGAGT TGCAGTCATG | |
| | | AAATTATTTG ATAGAGCTAA AGTAAATGTC AACCTCCTGA | |
| | | TCTTCCTTCT CAACAAAAAG TTCTACGGGA AGTAATTGAT | |
| | | CGTTTGCTGC CAGCCCAGAA GGATGAAGGA AAGAAGCACC | |
| | | TCACAGCTCC TTTCTAGGTC CTTCTTTCCT CATTGGAAGC | |
| | | AAAGACCTAG CCAACAACAG CACCTCAATC TGATACACTC | |
| | | CCGATGCCAC ATTTTTAACT CCTCTCGCTC TGATGGGACA | |
| | | TTTGTTACCC TTTTTTCATA GTGAAATTGT GTTTCAGGCT | |
| | | TAGTCTGACC TTTCTGGTTT CTTCATTTTC TTCCATTACT | |
| | | TAGGAAAGAG TGGAAACTCC ACTAAAATTT CTCTGTGTTG | |
| | | TTACAGTCTT AGAGGTTGCA GTACTATATT GTAAGCTTTG | |
| | | GTGTTTGTTT AATTAGCAAT AGGGATGGTA GGATTCAAAT | |
| | | GTGTGTCATT TAGAAGTGGA AGCTATTAGC ACCAATGACA | |
| | | TAAATACATA CAAGACACAC AACTAAAATG TCATGTTATT | |
| | | AACAGTTATT AGGTTGTCAT TTAAAAATAA AGTTCCTTTA | |
| | | TATTTCTGTC CCATCAGGAA AACTGAAGGA TATGGGGAAT | |
| | | CATTGGTTAT CTTCCATTGT GTTTTTCTTT ATGGACAGGA | |
| | | GCTAATGGAA GTGACAGTCA TGTTCAAAGG AAGCATTTCT | |
| | | AGAAAAAAGG AGATAATGTT TTTAAATTTC ATTATCAAAC | |
| | | TTGGGCAATT CTGTTTGTGT AACTCCCCGA CTAGTGGATG | |
| | | GGAGAGTCCC ATTGCTAAAA TTCAGCTACT CAGATAAATT | |
| | | CAGAATGGGT CAAGGCACCT GCCTGTTTTT GTTGGTGCAC | |
| | | AGAGATTGAC TTGATTCAGA GAGACAATTC ACTCCATCCC | |
| | | TATGGCAGAG GAATGGGTTA GCCCTAATGT AGAATGTCAT | |
| | | TGTTTTTAAA ACTGTTTTAT ATCTTAAGAG TGCCTTATTA | |
| | | AAGTATAGAT GTATGTCTTA AAATGTGGGT GATAGGAATT | |
| | | TTAAAGATTT ATATAATGCA TCAAAAGCCT TAGAATAAGA | |
| | | AAAGCTTTTT TTAAATTGCT TTATCTGTAT ATCTGAACTC | |
| | | TTGAAACTTA TAGCTAAAAC ACTAGGATTT ATCTGCAGTG | |
| | | TTCAGGGAGA TAATTCTGCC TTTAATTGTC TAAAACAAAA | |
| | | ACAAAACCAG CCAACCTATG TTACACGTGA GATTAAAACC | |
| | | AATTTTTTCC CCATTTTTTC TCCTTTTTTC TCTTGCTGCC | |
| | | CACATTGTGC CTTTATTTTA TGAGCCCCAG TTTTCTGGGC | |
| | | TTAGTTTAAA AAAAAAATCA AGTCTAAACA TTGCATTTAG | |
| | | AAAGCTTTTG TTCTTGGATA AAAAGTCATA CACTTTAAAA | |
| | | AAAAAAAAAA CTTTTTCCAG GAAAATATAT TGAAATCATG | |
| | | CTGCTGAGCC TCTATTTTCT TTCTTTGATG TTTTGATTCA | |
| | | GTATTCTTTT ATCATAAATT TTTAGCATTT AAAAATTCAC | |
| | | TGATGTACAT TAAGCCAATA AACTGCTTTA ATGAATAACA | |
| | | AACTATGTAG TGTGTCCCTA TTATAAATGC ATTGGAGAAG | |
| | | TATTTTTATG AGACTCTTTA CTCAGGTGCA TGGTTACAGC | |
| | | CCACAGGGAG GCATGGAGTG CCATGGAAGG ATTCGCCACT | |
| | | ACCCAGACCT TGTTTTTTGT TGTATTTTGG AAGACAGGTT | |
| | | TTTTAAAGAA ACATTTTCCT CAGATTAAAA GATGATGCTA | |
| | | TTACAACTAG CATTGCCTCA AAAACTGGGA CCAACCAAAG | |
| | | TGTGTCAACC CTGTTTCCTT AAAAGAGGCT ATGAATCCCA | |
| | | AAGGCCACAT CCAAGACAGG CAATAATGAG CAGAGTTTAC | |
| | | AGCTCCTTTA ATAAAATGTG TCAGTAATTT TAAGGTTTAT | |
| | | AGTTCCCTCA ACACAATTGC TAATGCAGAA TAGTGTAAAA | |
| | | TGCGCTTCAA GAATGTTGAT GATGATGATA TAGAATTGTG | |
| | | GCTTTAGTAG CACAGAGGAT GCCCCAACAA ACTCATGGCG | |
| | | TTGAAACCAC ACAGTTCTCA TTACTGTTAT TTATTAGCTG | |
| | | TAGCATTCTC TGTCTCCTCT CTCTCCTCCT TTGACCTTCT | |
| | | CCTCGACCAG CCATCATGAC ATTTACCATG AATTTACTTC | |
| | | CTCCCAAGAG TTTGGACTGC CCGTCAGATT GTTGCTGCAC | |
| | | ATAGTTGCCT TTGTATCTCT GTATGAAATA AAAGGTCATT | |
| | | TGTTCATGTT AAAAAAAAA | |
| LOC494127 | NR_036691.1 | GCAGTGCCCG ACTCCGCAGG AGCGCCAGGG CGGCTCCTGC | 18 |
| | | TCTTCCTGGA CTCCCTGAAG AGGCGTTTGT CGAAATGTCC | |
| | | ACAGAAGGAG GATTTGGTGG TACTAGCCGC AGTGATGCCC | |
| | | AGCAAAGCCT AAAGTCCTTC TGGCTTCGGG TCATGGAAGA | |
| | | AATCTGGAAT TTAGCAGTGA AAGATTTCTG AATGCAGGAA | |
| | | CTCCCACTGG CTCGTATTAA GAAGATTATG AAACTGGATG | |
| | | AAGATGTGAA GATGATCAGT GCAGAGGCCC CTGTGCTCTT | |
| | | TGCCAGGGCA GCCCAGATTT TTTATCACAG AGTTGACTCT | |
| | | TCGAGCCTGG ATTCACACAG AGGATAACAA CTGCCGGACT | |
| | | TATGTCGCCA TGGCAATTAC GAAATTTGAT CAATTGGATT | |
| | | TTCTCATCGA TATTGTTCTA AGAGATGAAC TGAAACCTCC | |
| | | AAAGTGTCAG GAGGAGGTGC TGCAGTCTGT AACTCCTGCT | |
| | | GAGCCAGTCC AATACTATTT CACGCTGGCT CAGCAGCCCA | |
| | | CCGCCCGTCC AAGTCCAGGG ACAGCAGCAA GGCCAGACCA | |
| | | CCGCCAGCTC CATGACCACC ATGCAGCCTG GGCAGATCAT | |
| | | CATCGCACAG CTTCAGCAGG GCCAGACCAC GCCCGTGACG | |
| | | ACGCAGGTTG GAGAAGGTCA GCAGGTGCAG ATTGTCCAGG | |
| | | CCCAGCCACA GGGTCAAGCC CAGTAGGCCC AGAGTGGTAC | |
| | | TGGATGGACC GTGCAGGTGA TGTAGCAGAT CCTCACTAAC | |
| | | ACAGGAGAGA TCCAGCAGAT CCCGGTGCAG CTGAAGGCTG | |
| | | GCCAGCTGCA GTGTATCCGC TTAGCCCAGT CTGTATCAGG | |
| | | CACCCACGTT CTGCAGGGAC AGATCCAGAC ACTTGCCACC | |
| | | AGCGCTCAAC CGATTACACA GACAGAGGTC CAGCAAAGAC | |
| | | AGCAGTAGTT CAGCCAGTTC ACAGATGGAC AGCAGCTCTA | |
| | | CCAGATCCAG CAAGTATCCA TACCTGCGGG CCAGGACCTG | |
| | | CCCAGCCCAT GTTTTTCCAG TCAGTCAACC AGCCCTCTGA | |
| | | TGGGCAGGCC CCCCGGGTGA CTGGCGGCTG AGGGCCGGAG | |
| | | CTGGCAAGGC CGAGGACACT CAACACAATT TTTGCCGTAC | |
| | | AGCCCCAGGT CATGAACACA GCCTTCTTCC CCAGAGGACC | |
| | | CGGCCGACCT CAGCTCCTCC TGCAGGCTAG GACAATGGCG | |
| | | CACTAGGCCT CATGCCTGGG GGCCGAGATT CTCCAACAGA | |
| | | AAGATGCAAT ATTTTTTGTT TCCTTTTTTC TCCAAGGAAT | |
| | | CAATATTTCA ATATGTTGAG CTGTGTGTCC AATGCTATGA | |
| | | AATTAAAATA TTAAATCACA AAAAAAAAAA AAAA | |
| LOC646471 | NR_024498.1 | AATGAATCCA ACTACTGTCC TTGTCCTCTC CTCTCTAGGT | 19 |
| | | TTGTGGAGAC TGAATTCCAC AAACCTAGGG ACAGGGCACT | |
| | | CTCTAGGGGC AAGTCAACTC TCAATTATAG TGAGGGCAGG | |
| | | TTCCCCAGTT GCCAGCCTAC ACCCTGGCCA GCCACCCAAG | |
| | | GGAATACCTG CTGCTGCTAA GGCAGTCAAT GTTGGGAGGG | |
| | | TCAGGGAAGG GGAGAGGAAG TAGCTGAGTG TAGAGATTAT | |
| | | CCAGGCTTTC CTTCCCGTCC TCTGTACAGA AAGGCAGACA | |
| | | TACACTGACT CCTGAAGTGC CCCAGGATCA TAGTTGGTTC | |
| | | TTCTTAGGGG GAGGGAGTGA AACGGTGGCC TTGCTCCAAT | |
| | | TCCAGGCCTC TGGAGAAAGG AGTGCTCAAC TGGAGAGTCT | |
| | | CAGAACCTTT TACAGTACTT CAGCAGGCCC AGCACCCAAC | |
| | | CTTCCCCACC ATACCCCTGC CCTGGGAATG CCCCTTACCA | |
| | | CAGTGGCTTC CATTCCTGAC ATTTGAACCA GCTCCTGCAC | |
| | | TTGAAGGACA AGGCAGCCAC TGGGCAGCTC CGGGAGGCGG | |
| | | GAGGCGTGAC TCCTTCATAT CAGGCCATCT GGAGGAGCAC | |
| | | CAGATTTCCC TCTTGTGAAA CAACCCCCCA CAGTGGGGAT | |
| | | TTAATGGTCA CCACACAAGC CCCAAAAAAC CAGGCCCTGC | |
| | | AGAGCCATGA CTTCACAGGG GAGAAAACCA GCCCAAGAGG | |
| | | CTGCCCTCAC CCAACACTTA GCTCTAAGTC CAGACTCTTA | |
| | | AAGGTTCTGT TAAAGATCTG CGGCCAGGCG CAGTGGCTCA | |
| | | CATCTGTAAT CCCAGCACTT TGGGAGGCCG AGGTGGGCGG | |
| | | ATCACGAGGT CAGGAGAGCG AGACCATCCT GGCTAACACA | |
| | | GTGAAGCCCT GTCTCTACTA AAAATACAAA AAATTAGCCA | |
| | | GGTGTGGTGG CGGGCACCTG TAGTACCAGC TACTTGGGAG | |
| | | GCTGAGGCAG GAGAATGGCG TGAACCCAGG AGGCAGAGCT | |
| | | TGCAGTGAGC CGAGATCACG CCACTGCACT CCAACCTGGG | |
| | | TGACAGAGCG AGACTCCGTC TCAAAAAAAA AAAAAAAAAA | |
| | | AAAAGATTTG CCTAGTGAGC TTGGCCAACC CAAGAGCCTT | |
| | | CCTGGGCTCT CAGGTTTCTT GAGGCAAAAC TTCTAAAGAT | |
| | | GCAAATAGAA TCCTTGGTAG CTACAAAGCT CATCTAGCCA | |
| | | AATTATAAGT ACTATCCAAG CCCAGGATTC ATTCTTGAAG | |
| | | ATGAGATGTA AAAGCACTGC TGGGCCCTTG CAAAGGGAGA | |
| | | GATCAGAGAA AATAGGAGAA CGAACACCAG CTCATGCCAA | |
| | | TCTCAGTCTC ATGAGCTACA GAGAAAAAGA GCCAAAAAAG | |
| | | TTTATCCTGG CTCAGCCACC TTCTGGCTTT GTAGCTACAG | |
| | | GCAAGTTATT TAACTTTTCT GAATGTTGGT TTTTCCATCT | |
| | | GGAAAAAGGA GACAGGAAAA GCTGCCACTG GAGGCTACCT | |
| | | TAAGGGTCAA TGAGTGAAAT GGGGTTGAGT GCTTAATGAA | |
| | | CTGTTAAAAC ATGACACAGA TGGGTGAGGT GACATCTGAC | |
| | | TGCTCTTCAC AGCAGTCCTT GCATGGTGAG CAGATGCAAT | |
| | | CAGTGTGCTC GTTCATTCAC TTGTTCATCA AACACCAACA | |
| | | GTACCCACTT GTGCCAGGCC TTGTGCTGGG CACCGTAGGG | |
| | | GCTATGGAAG GAGTAAGACA CAGTCATGCT CTCAAGGAAC | |
| | | TCGCTGTCCA GCAGAGTGAT AGGACATGAA TACATACTTC | |
| | | TAGCAAAGGC TGAGTAGCAT TAGGCAGGTG GCCAGATGAG | |
| | | GTCAGAGCTC TGGCCCTAGT GCCCTGTAGT AAGTTGCGAG | |
| | | GGAGTAGGAA ATGAAGACAA AACAGCAAGA GCACAGGTCC | |
| | | AGCACAAGAA GGTGAAGGAC ATGGAAAGAG GAGCAGAGCA | |
| | | TGATAAAGCT ACAAAAGTAG GGGGCAAGTC ATGAGGTCCG | |
| | | GGTCACTGGG GAGCCCTGGA CAGGCTCTGA ATGGGAGATA | |
| | | GACTAGACTG ACAAAGCAGC TCTTTCTGGA GACCTCCTGT | |
| | | GGAGGGTCCC CTCTTCTACC CTTGGGAGAC GAGGTGTCAT | |
| | | TCACTTCACG ATACACATGG GGAAAGGAAG TTGGACAGGG | |
| | | GAAGGCACTT GTCTAAGGTC ACTCAGTGGT CATGCCAGTG | |
| | | CAAAGCACCC AAAGCCTGGT CCAGGTCCTG CCACCTCCTG | |
| | | CGTGGGCTCC ATCTTCTCGG CTCAGTTCAA GCCTTTATTA | |
| | | TCAGAGTCCC TCTGTGTGCC AGGCTCTGTG CTGAATGTCC | |
| | | GTGTCGGCCA CAAATTGCAC TAACGGTGCT TACTGCCACT | |
| | | TCCTCTCCGA CCTTACCTCC AAAGACCCAG CCGGGCCTAA | |
| | | CCCTCACACA CAGCCAGAGG AATCCTTCCC AAACACAGTT | |
| | | AATCACATCG CTCATCCGTT TAAAACTGGC TTACTAGAGC | |
| | | CAACACAGGA TAAAATCCAC AATCAGTCCC AAACCCATCT | |
| | | TTCTCTGCCG TCTCTAGACC ACATTCACTG AGCACCAAGG | |
| | | CCTGCACGCT TCGCAGCCAG CCCTCCTGCT CTCCCCCTTG | |
| | | CCCTCCAGCC ACACTGGCCC TCAACCCCCA ACACTGGGCC | |
| | | CACCTCTGAG CCCTGCATCA AACACCAGCA CCCCCTTCGC | |
| | | CGAGCTCATT CTTCATCATT CCTCAGAGCT CAGCTTAAGC | |
| | | ATCACCTTCC TTCCCTGCGA CAGGGTCAGG TCCTTTCCTG | |
| | | TGCGCTCCGC AGCACCCTGC CTGTCCCTCG GAGTCCTCGC | |
| | | GAGAGGAACT AGATAACTGT GATCCCCGCG CACAGCACGG | |
| | | TGTCTGCCTA GGACACAGTG AGCGCTTCAA CGCGCTGAAC | |
| | | TAAACGAATG AAGGGGAACA CCAACTCAGC TGTCACCCCC | |
| | | GCCCCCAAGT GTCACTGGCT AGAAGATCTT CCCCCGGTTC | |
| | | GCCGGCAGCA CCGGTGGGTC TTGTCACCTC TGCGCCCCGC | |
| | | AGCCCTCCGG AGGCTGCAGT TGCTGTCTGC TGACAGGCGT | |
| | | CCCCTCAGGA GAACGGGAGC TCCTCCAGCG CAGGGCCCTG | |
| | | CTCAGCACCC CTGGTCCCGG GGGCCCCCAC GGAGGCCGCC | |
| | | GCGCTCAGTG AAACTTTGCC GCGCGCAGCA GTGGCCGGAG | |
| | | ACCCGCGCGG ACCCTCTCCC CGCGGGGACC CGCCAGGGCG | |
| | | AGTCGCCCCT CCCCCGCGGC AGACGACCCG ACCTGGGCCA | |
| | | AGGTCGGCCA AGTGCTCGCC GCCTGCCCAG CGGCCCTTGG | |
| | | ACCCACAGGA CCCGGACCCG AGTGCGCGGA AGCCTCCGAG | |
| | | CCGCGGCCAG GTCTCAGAAT GCGCGGCGGG GCAGCCCGGC | |
| | | CCGTCAAGCA GCGAAGCCGA AACTAGAAGC CTGAGGTCTG | |
| | | GCTTGGTCAC TGTTTGCTGG GGAACTCCGA CGAGCAACTA | |
| | | CCCTTCGGGG ACTCAGTTTC CCCCGGGCCA AGTGAGACGG | |
| | | ACAGCTGCGG CTCCCGCGAG AACTCGCGCG GGAACAGGAG | |
| | | CTGCGGGCTC CGCACAGCGC CCGGCACGTA GACCCAGTCT | |
| | | ACTAACGGGC TGGAAGGTCG CGCTCCCGCC TCCGCGCCTC | |
| | | CCTAGAACCT TTCTCACCTC AAGCTCGGGC TCCCGGACTC | |
| | | TCTCACCTCA CGGCCCCTCC TTCCACCTTG GGCCGCCCAG | |
| | | CCTCAACCGC CCT | |
| LOH12CR | NR_024061.1 | GGCTACGCAA GACCTTCAGT TCCGGATTAG GAGGCCCCGC | 20 |
| | | CCCCCGGCCC GAGGGAGGGG CGGAGAGACC CGCTCCTGCG | |
| | | ACTTAGGGCG ATGCCACCTT AAAGGGCTTG ACCTCCTCGA | |
| | | AGCCAGAACT GCGGAAGAGG ATGGAGAAAG AAACGCTAGA | |
| | | TAGACCCAGG ATTCGAACCC ACAACAGCTT GGATGCAAAG | |
| | | CTCATTTTGA ATTCTGAAGA GCTGGGAGTT TAGCAGTGGA | |
| | | CGACCAAACA AAAAAATACC AGAAGACGGT GAAAGCAGCA | |
| | | GCTGGAAATG AGAGAAAAAT TAAGAAATAG AAAATGCTGT | |
| | | TTCATGTATT AGGGAGTGCA CATCTTTAAA GAGAAGTGAA | |
| | | GAACCTTTTT GGCTAGGCGC TTTCTGGGGA CCTTGGCAGT | |
| | | TATCAACAAG TCACTCTGAA ACTAACAGAA GGTGAGGAGG | |
| | | GAGTAATCCA GAAAGGAAAC TGCCATTTTT GCTAGGGCAA | |
| | | GAAAGTAGAC CTAGCAATGC AATGCCATCA AGGAGTTAGC | |
| | | TGGCTCTAGC GTTCTCCGAA CTTTGCAACT CATTTTATAT | |
| | | TACATTGTCT GCGTCAAGAA ATTTCAAGTA AATGCCTTGA | |
| | | GATTTTATAT GTATAAAATG TAGTCTCTGG CCAGGCGGGG | |
| | | TGGCTCACGC CTGTAATCCC AGCACTTTGG GAGGCTGAGG | |
| | | CGAGTAGATC ACGATGTCAG GAGATCAAGA CCATCCTGGC | |
| | | TAACATGGTG AAACCCCGTC TCTACTAAAA AATACAAAAA | |
| | | ATTAGCCGGG CATGATGGTG GGCGCCTGTA GTCCCAGCTA | |
| | | CTCGGGAGGC TGAGACAGGA GAATGGTGTG AACCCGGGAG | |
| | | GCAGAGCTTG CAGTGAGCCG AGATCGCGCC ACTGCACTCC | |
| | | AGCCTGGGCG ACAGAGCGAG ACTCCGTCTC AAAAAAAAAA | |
| | | AAAAAAAAAA AAGTCTCAGA AGAAGTGTCA AGTTGACTAA | |
| | | ATGATTTTTA AGTCCCTTCC ACTCTACTAT ATCACAATAA | |
| | | TTTCCCCTAT TTTACCTTAT TTAATTTTCA CAACAACCCT | |
| | | GCAGCCTGTA AGTAGGTGAC TCCTCCATGG TCAGCAGGTG | |
| | | GTCAGTGGTC GAACTATTTA GGACTATCTG ATGCTCTTTC | |
| | | CTCTGCTCAC CATTGCAATA CAATGAGAAA AATGCAAATG | |
| | | AATCTAAGTA CAGTGTTCTC TGGGAATATG AAGATAGAAC | |
| | | TAAAAACTGC CTGAGAGATC AGGGCAGGCT TCTAGAAAGT | |
| | | TGTGTCTAAC TAGTCTTGAG GTTTACATAA ACCACACCAG | |
| | | GCTGATGGGA CTGGGAAGGG CCCACTAAGC AGTGAGACCA | |
| | | TTCCCTTTTG GAGAGTCCTT GCATCCCCTC CCAGATTTCC | |
| | | TCTTTGAGGG GAAGGTGAGA GAGGAGGTAA AAGGGGTGAG | |
| | | GAATGGAGAA TAACTCATTC TGGTTCTTGT TTCCCCTTTT | |
| | | CCACATAAAA GTATATTTGT CTTGTGTTCC ATACACCAGT | |
| | | CCATACTGAT GTGATGGTGT TTTTTATGCT TCCTTTTGAA | |
| | | TAAACATTTC ATTCTTAA | |
| PBXIP1 | NM_020524.3 | GGTCAGTTTC TGGTCACATG ATTTTCTTCT CGGGCTGCAA | 21 |
| | | ACAAAGGGAA GCCTGCAACA AGTTAAGCTG AAGACCGAAG | |
| | | CAAGAGCTGG TTCAGGTGGC AGCCACAGCA GCCTCAGGGA | |
| | | CCTCAGCAAC TATGGCCTCC TGCCCAGACT CTGATAATAG | |
| | | CTGGGTGCTT GCTGGCTCCG AGAGCCTGCC AGTGGAGACA | |
| | | CTGGGCCCGG CATCCAGGAT GGACCCAGAA TCTGAGAGAG | |
| | | CCCTGCAGGC CCCTCACAGC CCCTCCAAGA CAGATGGGAA | |
| | | AGAATTAGCT GGGACCATGG ATGGAGAAGG GACGCTCTTC | |
| | | CAGACTGAAA GCCCTCAGTC TGGCAGCATT CTAACAGAGG | |
| | | AGACTGAGGT CAAGGGCACC CTGGAAGGTG ATGTTTGTGG | |
| | | TGTGGAGCCT CCTGGCCCAG GAGACACAGT AGTCCAGGGA | |
| | | GACCTGCAGG AGACCACCGT GGTGACAGGC CTGGGACCAG | |
| | | ACACACAGGA CCTGGAAGGC CAGAGCCCTC CACAGAGCCT | |
| | | GCCTTCAACC CCCAAAGCAG CTTGGATCAG GGAGGAGGGC | |
| | | CGCTGCTCCA GCAGTGACGA TGACACCGAC GTGGACATGG | |
| | | AGGGTCTGCG GAGACGGCGG GGCCGGGAGG CCGGCCCACC | |
| | | TCAGCCCATG GTGCCCCTGG CTGTGGAGAA CCAGGCTGGG | |
| | | GGTGAGGGTG CAGGCGGGGA GCTGGGCATC TCCCTCAACA | |
| | | TGTGCCTCCT TGGGGCCCTG GTTCTGCTTG GCCTGGGGGT | |
| | | CCTCCTCTTC TCAGGTGGCC TCTCAGAGTC TGAGACTGGG | |
| | | CCCATGGAGG AAGTGGAGCG GCAGGTCCTC CCAGACCCCG | |
| | | AGGTGCTGGA AGCTGTGGGG GACAGGCAGG ATGGGCTAAG | |
| | | GGAACAGCTG CAGGCCCCAG TGCCTCCTGA CAGTGTCCCC | |
| | | AGCCTGCAAA ACATGGGTCT TCTGCTGGAC AAGCTGGCCA | |
| | | AGGAGAACCA GGACATCCGG CTGCTGCAGG CCCAGCTGCA | |
| | | GGCCCAAAAG GAAGAGCTTC AGAGCCTGAT GCACCAGCCC | |
| | | AAAGGGCTAG AGGAGGAGAA TGCCCAGCTC CGGGGGGCTC | |
| | | TGCAGCAGGG CGAAGCCTTC CAGCGGGCTC TGGAGTCAGA | |
| | | GCTGCAGCAG CTGCGGGCCC GGCTCCAGGG GCTGGAGGCC | |
| | | GACTGTGTCC GGGGCCCAGA TGGGGTGTGC CTCAGTGGGG | |
| | | GTAGAGGCCC ACAGGGTGAC AAGGCCATCA GGGAGCAAGG | |
| | | CCCCAGGGAG CAGGAGCCAG AACTCAGCTT CCTGAAGCAG | |
| | | AAGGAACAGC TGGAGGCTGA GGCACAGGCA TTAAGGCAAG | |
| | | AGTTAGAGAG GCAGCGACGG CTGCTGGGGT CTGTACAGCA | |
| | | GGATCTGGAG AGGAGCTTGC AGGATGCCAG CCGCGGGGAC | |
| | | CCAGCTCATG CTGGCTTGGC TGAGCTGGGC CACAGATTGG | |
| | | CCCAGAAACT GCAGGGCCTG GAGAACTGGG GCCAGGACCC | |
| | | TGGGGTCTCT GCCAATGCCT CAAAGGCCTG GCACCAGAAG | |
| | | TCCCACTTCC AGAATTCTAG GGAGTGGAGT GGAAAGGAAA | |
| | | AGTGGTGGGA TGGGCAGAGA GACCGGAAGG CTGAGCACTG | |
| | | GAAACATAAG AAGGAAGAAT CTGGCCGGGA AAGGAAGAAG | |
| | | AACTGGGGAG GTCAGGAGGA CAGGGAGCCA GCAGGAAGGT | |
| | | GGAAGGAGGG CAGGCCAAGG GTGGAGGAGT CGGGGAGCAA | |
| | | GAAGGAGGGC AAGCGACAGG GCCCGAAGGA ACCCCCAAGG | |
| | | AAAAGTGGTA GCTTCCACTC CTCTGGAGAA AAGCAGAAGC | |
| | | AACCTCGGTG GAGGGAAGGG ACTAAGGACA GCCATGACCC | |
| | | CCTGCCATCC TGGGCAGAGC TGTTGAGGCC CAAGTACCGG | |
| | | GCACCCCAGG GCTGCTCAGG TGTGGACGAG TGTGCCCGGC | |
| | | AGGAGGGCCT GACTTTCTTT GGCACAGAGC TAGCCCCAGT | |
| | | GCGGCAACAG GAGCTGGCCT CTCTGCTAAG AACATACTTG | |
| | | GCACGGCTGC CCTGGGCTGG GCAGCTGACC AAGGAGCTAC | |
| | | CCCTCTCACC TGCTTTCTTT GGTGAGGATG GCATCTTCCG | |
| | | TCATGACCGC CTCCGCTTCC GGGATTTTGT GGATGCCCTG | |
| | | GAGGACAGCT TGGAGGAGGT GGCTGTGCAA CAGACAGGTG | |
| | | ATGATGATGA AGTAGATGAC TTTGAGGACT TCATCTTCAG | |
| | | CCACTTCTTT GGAGACAAAG CACTGAAGAA GAGGTCAGGG | |
| | | AAGAAGGACA AGCACTCACA GAGCCCAAGA GCTGCGGGGC | |
| | | CCAGGGAGGG GCACAGCCAT AGCCACCACC ACCACCACCG | |
| | | GGGCTGACAC CCTGCCCCAC AGGGAATGGC CTTGGCCTGG | |
| | | CCCAGCCCAA GATCCCAGCG TTATCTAACT CCTGGAGGGT | |
| | | GGACTCTGTC CTGGCTTGTT TGGTGTCCTC AGATATCTTT | |
| | | CACACAGTAG AGCAAAATCA CCAGCCCTGC ACTGATGTCA | |
| | | CTTTATGTAG AAAAAGGCCT TAGCTGGACC TGCGTTGCCG | |
| | | TCTATGCAAA TGCATGCAAA TACTCCAGGC CCTGGGATGT | |
| | | GGGCTTGTGT TTTGTCACTG TGAAGGGGGA GATGGGAGAG | |
| | | GAGCCTGTTT TGGGGTGGGG TCTGGGGAAG GCAATCTGAT | |
| | | TCTGAAGCTA AAGAGCTTTC ATCCTCTTGA GTGTATGTCC | |
| | | CCATAGTGGG CCCCTTGACC CACATGCTGA CCGGTGCCTT | |
| | | GGGATTTGAC TAGAGTTGCT GGCTCGAGGC CCAGCACGAG | |
| | | GACTTACCCT GGGGTTTTGT TAGGTTTGGA AGCAGCTGTC | |
| | | CCTAGGGGGT GAAGTCCCCC CCCTTTTTTT TTTTACCCCT | |
| | | GCTTCTCCCA CGGCTTCACC TCCCTATGTG AACTGTAGAC | |
| | | TCAGATCCCA ATAAAGTGCT GTTGCAGCTA TGATGCTAGG | |
| | | TGGTTTCTAA GCACAGGGGA CACCCCACAC CCCCTGCCTG | |
| | | AATGGATGGG TCCATCCCAG GCACTGGTAC TTGCCCCCTT | |
| | | GTTCTGTATC CCCCTTTGCC CTTGCCTTGC CCTTCCAACA | |
| | | AACCCTAGGC CCTTGAGAAG CTGATACTTC TCCTTTTGCT | |
| | | CACAGCTGCC TTGGCCCCAC CCCTGGGAGA TGTAGCAAAT | |
| | | TGAGTGTGGG TTTTGGAGTC TGAGCCTCAG GCTCAAATCC | |
| | | AGGCCAAGTG ATCTTGGGCA AGTTAATCTC TGGGAACTTT | |
| | | GGGTTTCTTA TCCTCAAAAA AGGCGATGGA AGGGCTGGGG | |
| | | AAGTGATTAA ATAAAAGCAA CGCAAGAAAA AAAAAAAAAA | |
| | | AAAAA | |
| RNF5 | NM_006913.3 | AATAGTGATT AGGAAACCTT GAAGCCTGCC CAACGATCGT | 22 |
| | | GGGCAGGAGG TGGTTTCTGG TTTGTTGGGG CGTGTGTATG | |
| | | TGTATTTGGG GGGACTGAAG GGTACGTGGG GCGAAACAAA | |
| | | ACCGGCCATG GCAGCAGCGG AGGAGGAGGA CGGGGGCCCC | |
| | | GAAGGGCCAA ATCGCGAGCG GGGCGGGGCG GGCGCGACCT | |
| | | TCGAATGTAA TATATGTTTG GAGACTGCTC GGGAAGCTGT | |
| | | GGTCAGTGTG TGTGGCCACC TGTACTGTTG GCCATGTCTT | |
| | | CATCAGTGGC TGGAGACACG GCCAGAACGG CAAGAGTGTC | |
| | | CAGTATGTAA AGCTGGGATC AGCAGAGAGA AGGTTGTCCC | |
| | | GCTTTATGGG CGAGGGAGCC AGAAGCCCCA GGATCCCAGA | |
| | | TTAAAAACTC CACCCCGCCC CCAGGGCCAG AGACCAGCTC | |
| | | CGGAGAGCAG AGGGGGATTC CAGCCATTTG GTGATACCGG | |
| | | GGGCTTCCAC TTCTCATTTG GTGTTGGTGC TTTTCCCTTT | |
| | | GGCTTTTTCA CCACCGTCTT CAATGCCCAT GAGCCTTTCC | |
| | | GCCGGGGTAC AGGTGTGGAT CTGGGACAGG GTCACCCAGC | |
| | | CTCCAGCTGG CAGGATTCCC TCTTCCTGTT TCTCGCCATC | |
| | | TTCTTCTTTT TTTGGCTGCT CAGTATTTGA GCTATGTCTG | |
| | | CTTCCTGCCC ACCTCCAGCC AGAGAAGAAT CAGTATTGAG | |
| | | GGTCCCTGCT GACCCTTCCG TACTCCTGGA CCCCCTTGAC | |
| | | CCCTCTATTT CTGTTGGCTA AGGCCAGCCC TGGACATTGT | |
| | | CCAGGAAGGC CTGGGGAGGA GGAGTGAAGT CTGTGCATAG | |
| | | ATGGGAGAGC CTTCTGCTCA GAGGCTCACT CAGTAACGTT | |
| | | GTTTAATTCT CTGCCCTGGG GAAGGAGGAT GGATTGAGAG | |
| | | AATGTCTTTC TCCTCTCCTA AGTCTTTGCT TTCCCTGATT | |
| | | TCTTGATTTG ATCTTCAAAG GTGGGCAAAG TTCCCTCTGA | |
| | | CTCTTCCCCC ACTCCCCATC TTACTGATTT AATTTAATTT | |
| | | TTCACTCCCC AGAGTCTAAT ATGGATTCTG ACTCTTAAGT | |
| | | GCTTCCGCCC CCTCACTACC TCCTTTAATA CAAATTCAAT | |
| | | AAAAAAGGTG AAATATAAAA AAAAAAAAAA AAAAAAAAAA | |
| | | AAAAAAAAAA AAAAAA | |
| SERTAD2 | NM_014755.2 | CTCCTGCACG GCGAGTGCTG GAGCACGACG TACCGCTCGC | 23 |
| | | TCGGTCAGGG CGCCCCCTCC GCCCGCCTCC TGCTTCCTCC | |
| | | TCCGCTGCCT GCCGCCGCCG CCTCCACCAT TGTATAATGC | |
| | | TCGGGGCGCG CAGGCAGAGA ACGGCGGAGT CTTAGCTTCA | |
| | | GCCTCGCCTG CTGCCCGCTC CCCGGCGCCA CCCTCGGGCC | |
| | | CCTGGAGCGG GGCACTCCGC ATGGAGCGGG AGTAGCTGAG | |
| | | GAGTGGGCGG AAACCCCTCC TGATGCGTTA GTTCCCAGGT | |
| | | GGAGCTGCAT GTGATATATG TTGGGTAAAG GAGGAAAACG | |
| | | GAAGTTTGAT GAGCATGAAG ATGGGCTGGA AGGCAAAATC | |
| | | GTGTCTCCCT GTGACGGTCC ATCCAAGGTG TCTTACACCT | |
| | | TACAGCGCCA GACTATCTTC AACATTTCCC TTATGAAACT | |
| | | CTATAACCAC AGGCCCCTGA CAGAGCCCAG CTTGCAAAAG | |
| | | ACCGTTTTAA TTAACAACAT GTTGAGGCGG ATCCAGGAGG | |
| | | AACTCAAACA GGAAGGCAGC CTGAGGCCCA TGTTCACCCC | |
| | | CTCCTCCCAG CCCACCACCG AGCCCAGCGA CAGCTACCGA | |
| | | GAGGCCCCGC CGGCCTTCAG CCACCTGGCG TCCCCGTCCT | |
| | | CCCACCCCTG CGACCTCGGA AGCACTACGC CCCTGGAGGC | |
| | | CTGCCTCACC CCGGCCTCAC TGCTCGAGGA CGACGATGAC | |
| | | ACGTTTTGCA CCTCCCAGGC CATGCAGCCC ACGGCTCCCA | |
| | | CCAAACTGTC ACCTCCAGCC CTCTTGCCAG AAAAGGACAG | |
| | | TTTCTCCTCT GCCTTGGACG AGATCGAGGA GCTCTGTCCC | |
| | | ACATCTACCT CCACAGAGGC GGCCACGGCT GCGACTGACA | |
| | | GTGTGAAAGG GACCTCCAGC GAGGCTGGCA CCCAGAAACT | |
| | | CGACGGTCCT CAAGAGAGCC GCGCAGATGA CTCAAAACTG | |
| | | ATGGACTCTC TGCCTGGGAA TTTTGAAATA ACGACGTCCA | |
| | | CGGGTTTCCT GACAGACTTG ACCCTGGATG ACATCCTGTT | |
| | | TGCTGACATT GATACGTCCA TGTATGATTT TGACCCCTGC | |
| | | ACTTCCTCAT CAGGGACAGC CTCAAAAATG GCCCCTGTGT | |
| | | CTGCCGACGA CCTCCTCAAA ACTCTGGCTC CTTACAGCAG | |
| | | TCAGCCTGTC ACCCCAAGTC AGCCTTTCAA AATGGACCTC | |
| | | ACAGAGCTGG ACCACATCAT GGAGGTGCTT GTTGGGTCCT | |
| | | AAGACCCAGG GACCCAGCGA CTATGCCCAC CCAGACCCCA | |
| | | GAGCGTTCCC ATAACCCTGA CAGTTCTCCA CACTGTGCAT | |
| | | GCACCCTTGC TTGCCTTTTT CAGAGAAAAA GAAAATTTTA | |
| | | CAACAGGATC ACACTAGTTT TTGCTTTGAG CAGAGTTGGA | |
| | | GTGCCTTCAT CCAAGTATGA CCACTTTTAA TACACTTTTT | |
| | | TGAGTGGTTC CTCAGAGACC TACTACCCTG GTATAGGAAA | |
| | | GAATCCATTT GAAGACAATG TTGCAATGTT GAATGACAAA | |
| | | AATAAACAGT TCAAGTGAAG CACAAGGATT AAGTTGGAAA | |
| | | AGCTGTAAAT TGCATGTGCA TATTTGTCTA TTTTTTCTAT | |
| | | AAGTTTTATT GCAAGAGGTA AAGAAGAAAA CTATATATAT | |
| | | ATATCTTATT TAGATAATCT CAGTACCTTT TCTGGCATTT | |
| | | TTGCCCTGTA TAGGTTGACT TGGCAATTCG GCCTTTTTAG | |
| | | AGGCATTAAC TACTCCTCGT AAGTGTTGCA TTTACATGGC | |
| | | TGTTTAGAAA ACTGCTGCCC AAATTTATTT TATATTTTTG | |
| | | TACAGATTCT GCAGTTTATG ATATTGTTTT TCTAAAAACA | |
| | | AATGCTGTTT ATACATATGA GATAGCTATT TTGATAGGAT | |
| | | TTGCTCACAT AGTTCCTGCA AACTTCAGAT GTACAAGTTG | |
| | | CACTTGTACT TTTATAGAGT TGTAATGTTT TATATGTGTA | |
| | | TGGTGCAAGA GAAAATTGGA TCAAATCAAT CTGCAGTTGA | |
| | | TGTCCCCAAA TGCAAACACA GGCACACACA TGCACACACC | |
| | | CATAAACACA CACACAGTGC TTTAAGAAAG GGCCAGGTGA | |
| | | TATCACACCC AAATTTCACA AGCACTGACC CCCTGGCACC | |
| | | AACACCCGCC AGTACTGTGA CTTCCAAAGC CAGAGCCACA | |
| | | TGTGCTCATC AAACTTGCAT TAAGCAGTTG GCGGGAGATG | |
| | | GCTGTGGAGC TGGGGGTTTA AGTGATGGTT CTCTTTTGCT | |
| | | CCCTCTTTTG AGGGTAAAGC TACTGTCTTT CTTAAGAGTG | |
| | | TATTTATGCC AAGTTTGCGC TTTTAATTGT TTTTATTTTG | |
| | | TTTTTTAATG AAAACCCAGA TCTTTCCTTT TTGGCATAAT | |
| | | TTTTATGATG ACCTGAAATT TTACATCCGA ACAAAATTTT | |
| | | ACATCCGAAA AGCAACCAAC TTCTTCATGG AACTCAGCCC | |
| | | TGTTGCAATG CTTAGGGCCC TTAAAGAAGA AAATCTCCCC | |
| | | AGAAGGCATC CATCATGTTG CTTAATTGTC TTCTGCAGCT | |
| | | TCCTTTCCCT AGAGCTTTCC CTGTGTTGCT AAGAGCTGAA | |
| | | AATGGCATCT TCGTGATCAC CACAGTGAGC TTGGCTCGCC | |
| | | TCGGCCGGCC CGGGATGCAC TCTTACAACA TGTGTGACTC | |
| | | TTGAACCTGG AGTTCATCAC ATTACGTCAC AGCTTCCCAT | |
| | | CTGGTTGCTT TCCTGAGTCA GCTACTTCAC ACTTGTCAAG | |
| | | GCTGTTTTAC CCCAAAACTC AGACAGGACT TTCTATGCAT | |
| | | GTTTTCCCTC CTCCCCCCAA TTCCCCCCCC CATCACCTTA | |
| | | TCTCCCAGGA CACACTTGAG AAGTAGCTTT TTATTCCTAG | |
| | | TGGTGTACAT TTAATTTTAA AAAGGTTGCA ATGTATCATG | |
| | | CTTGTTGCCG AAACTGTTTA TGGCCTTCTT GTTTCAGTTT | |
| | | TTTCTTTTCT TCCAATGGTA CTTTAGCTGT TGAGTGCAGG | |
| | | TTACAACCTA TATTGTTATG CAGATGGCTT CTTTAGGAAT | |
| | | AACTTTTATA TTTATTTAAA AATTTTTAAA TTATGGGATG | |
| | | TTTTGTTGTT GTTGTTGTCT TTGTTGTTGG TCATTTGTCA | |
| | | ATATTCAGTC ACCAATTCTG CTCACTTCTT GCCATGGATA | |
| | | AAATTGGGTC TTTCTGGCTA ATTAAAAAAG ACAACTTTAT | |
| | | AAAATGGCAC TTTAAGCAAG CCATAGTTAG TTTTATTTTT | |
| | | GTAATGCACA TGGCAAAGCA AAGACGTTTG TGATGAAGGA | |
| | | ACTGCTCATC TAAGCAAAAG ATTTGAGTAT GATATGATAA | |
| | | AGGCTTTCTA CATTCTAATT TACTTTTTCC CCCCACTTGA | |
| | | ATGTGTTTTA AAGGCTAATT ATCAGCTCAG TAGAGCAGTG | |
| | | AGAAACTGAT CAAATTGCAC TTGTTCTCCT ACAAGCAACC | |
| | | TCCACGCAGA CACCTCGTAC TGCTACAGGT GTGTCATTTC | |
| | | CTTTAATAGG ACCAGGGACC ATGTAACTGA GGTGAGGGTT | |
| | | GTAGTAGATG CTTCCAGTGT CAGTATGCCT GTTAATTTTA | |
| | | AGAGCTTCCC TTTCTTGCAG AGAACAAGTC TGCCCAGATT | |
| | | CCATGCTTTC TATAACTGGA GGACCTGGCA AACCTGCCGC | |
| | | ATGCTGCACA CATCTACCTA CGTACACATA TACAATAGTA | |
| | | TTGATGATTC TGAACAATAA CAGGGTAAAA CAGTTGGTTT | |
| | | GCCATTGTTA AAAACTGATT TACAGTAACT TACAACAACT | |
| | | GTACTTTTGT TGGATTAGCA AATCATGTGT TTAAACAAAT | |
| | | CCCATATGTT GGGCAACAGT TCAAATAAGC ACGGAGAAGT | |
| | | GTTGCCCAAA CTTGGTTCTC TGACTCTTAT GTATTTGTAA | |
| | | GGCTGGGCTT CAAAATCAAA ACAAAAACCC CAAAAACAGC | |
| | | AGGCAAATGC TTTTTAACTC TGACACCGTT GCCATAAATC | |
| | | CCTGATACTC AAAGTCTAAC AAGAAAGACA TGGAAAATTA | |
| | | GCAGCCCATT TTCAGAAAGA TCAAAATGAT CTAGGGTTCT | |
| | | AATTGCTTTT GCATCCTATT CTTACAAAGT GATGTCCCAA | |
| | | CAGGGAACAG TAGGAGCTGG AGTGGGATCT CCAAGTCCCA | |
| | | GTTTGAGTGT GGGATGTGCT TCCAGCAGTG CCTTCCCTTT | |
| | | ATGAAAGACA TCACATGGCA TCCAGGGCCA GGCAGGCAGC | |
| | | TTGAGGTGCC TTTACGAGAA AACCGAGCTG GGGCTGGGAG | |
| | | AGGACAGTTA TTGACACTGA TGTGCAATGA AGTGACAAGA | |
| | | TGAGAGCAGA ATCGTAAGAG CTTTGAATTT GAAGTGAGTT | |
| | | TTTTTCCCCC CATAAGTTAT TTATTCCTTT TTTCTGTGTA | |
| | | AATATATTTA TTTTACTGTG GAGCGCTAAC ATCTGGATCG | |
| | | TAACATGTGC AGAATGTATG GTAGGAATGT ATTCTCTTGT | |
| | | AGGAATGTAA ATCTGTATTA AAAGGGGGTC CAAGCCAGGC | |
| | | CCCCAGGTCT TCTCATTGTA TGCACAGTCC GCATTCATTT | |
| | | TTACTCTTCT CTAATATGGG TCTATTTGAA ATATGCAAAA | |
| | | GGTATGAGGA ATGTTTTAAT ACCTCCAAAT TTTTAAGAAA | |
| | | AGCATCAAAG GGTTGATATT TTTTAAAGTT TTTTTAGTAG | |
| | | CACTTTCTCT GGATGACAGA AGGAGCAACC ACATGGGCAC | |
| | | CCTTGTTCAT ACCAAAGGGT GAGCAGTGGC CAGAGCCTCC | |
| | | TCTGCACCTC TCGAGTGTCT TTACCAATTG AGCTTTTTAT | |
| | | CGCCATAGCC CCTTGGAGTG CCCCAGCTGC CCTGAGGTCA | |
| | | ATCAAGGAAA ATTTCTTAAT GAAATAAGCT CCAAAGAGCC | |
| | | AAAGTATCAA CTTACAGATC GTTTTTAAAG CTTAAATTTA | |
| | | TGAACCACCT TTGTGGTAAA CAATGAATTA TGAATACCGC | |
| | | AGGGCAGCCT TCTTAAATGA CAAATGTAAA AAAAAAAAAA | |
| | | AAAAAGACTC TACTTCGTGC AGCAATTGCT ACTCTATACG | |
| | | AATTGTCTTA ATTTGAAAAC CTTGCTGTTA CAAATTGGAC | |
| | | CTTTATACAT TTTCTGAAAA CAATGAAAAG AGTATATTTA | |
| | | ACCTTTTCTG GCTGTAAATG GTTACCTTCC TGTAACTGCC | |
| | | CCGCACCTGG AGGCATGGAG TTGTGTGCAT CCTGCTTATG | |
| | | TACAATTGTT TTCAGTGTTT CTAAGAATGA GTCTGAATGG | |
| | | TTCTTGAAAA TTAGCCAGGA TCAAATGCTA TTGCAGACAA | |
| | | AGCCAATAAA AAGTTGGACT TCTTTTGGGG ATAACAAGTT | |
| | | TTGGAAGAGA AATGCAGGCC ATATGTGCGC ATGACCGAGA | |
| | | TTTTGAAAAA AGATGTACAT AGTGACATGT TTGGTGCATG | |
| | | GTTTTTGAGG AGGGCTTTTG TCAAAAAGGA GGTATAACCT | |
| | | TTCCCCCACA GACCTGAGAG CTGTGCCTTT TCTATGCAAT | |
| | | ATTACAGACG TTACATCGGA ACCCAGATGG CTGTATTCAC | |
| | | ATGTAGGTTT GGGCTGTAAT CTAAACAATT GGACAGATTA | |
| | | AATGTACATG GAAATGAGCA GTCTTACTTT TGTAGTTTTA | |
| | | TATTATACAA TAAACAGTTA AAAGATGAAA AAAAAAAAAA | |
| | | AAAAA | |
| SLC35G5 | NM_054028.1 | TACCACCTCA GGAGAGTTCC AGGGAAGAAC CCCACCCGCA | 24 |
| | | CTCCAATGAG GTCACAATGG CTGGAGCTCT GAGGGGCCCA | |
| | | GGCTCCCTGA GCCAGGAGGA GAGGAGAAAG TCCAAGGAAA | |
| | | GATGGCTGGC AGTCACCCCT ACTTCAACCT GCCTGACTCC | |
| | | ACACACCCAT CGCCGCCCTC CGCTCCACCC AGCCTCCGCT | |
| | | GGCACCAGCG CTGCCAGCCC TCTGGTGCCA CCAATGGCCT | |
| | | GCTGGTGGCC CTGCTGGGTG GGGGCCTGCC TGCTGGCTTC | |
| | | GTGGGCCCCC TTTCTCGTAT GGCTTACCAG GGTTCCAACC | |
| | | TGCCCTCGCT GGAGCTGCTC ATCTGTCGAT GCCTCTTCCA | |
| | | CCTCCCTATT GCCCTGCTAC TTAAACTGCG TGGCGACCCC | |
| | | CTTCTGGGAC CTCCTGACAT CCGAGGCTGG GCCTGCTTCT | |
| | | GTGCCCTGCT CAACGTCCTC AGCATTGGAT GTGCCTACAG | |
| | | TGCAGTTCAG GTGGTGCCCG CTGGCAACGC TGCCACTGTT | |
| | | CGCAAAGGTT CTTCCACCGT ATGCTCCGCT GTCCTCACCC | |
| | | TCTGCCTTGA GAGCCAGGGT CTCGGTGGCT ACGAGTGGTG | |
| | | TGGACTGTTG GGCAGCATCC TAGGACTAAT CATCATTCTG | |
| | | GGACCTGGAC TCTGGACACT ACAGGAGGGG ACCACAGGTG | |
| | | TCTACACCAC CCTGGGCTAT GTGCAGGCTT TCCTGGGAGG | |
| | | CCTGGCGCTG TCCCTGGGGC TTCTGGTCTA TCGTTCTCTG | |
| | | CACTTTCCCT CCTGCCTCCC AACAGTGGCC TTCCTATCTG | |
| | | GCTTGGTGGG GCTGCTGGGC TGTGTGCCAG GCCTCTTTGT | |
| | | GCTGCAGACC CCCGTGTTGC CCAGTGACCT CCTGAGTTGG | |
| | | AGTTGTGTGG GGGCAGAGGG GATCCTCGCC TTGGTCTCCT | |
| | | TCACATGTGT GGGCTATGCG GTCACCAAGG CCCACCCTGC | |
| | | CCTGGTGTGC GCTGTCCTGC ATTCCGAGGT GGTTGTGGCC | |
| | | CTTATACTGC AGTATTATAT GCTCCATGAG ACTGTGGCAC | |
| | | TTTCTGACAT CATGGGGGCA GGGGTTGTGC TGGGCAGCAT | |
| | | TGCCATCATT ACAGCCCGGA ACCTCAGCTG TGAGAGGACA | |
| | | GGGAAGGTGG AGGAGTGAGA TAGAACTTGG GAGCCCGGGG | |
| | | GTTGGGAGGG ACAGGGATAA ATAAAGACAA AGACTGAAGA C | |
| SPATS2L | NM_001100422.1 | AGTGCTGAGG GAGCAAAGTT CATTTTCTCG GGTAGGAGAA | 25 |
| | | GATGATTCTC TTGCAACACG TGCGGATTGT GACAAAATCT | |
| | | TTCATTAACA AGGGGAGTTT CGGTGAAGTG GAGGTTTGGG | |
| | | GAAAGGCGAG GAAGTCGGTC TGGAGCAAGC AAGCAAAGTG | |
| | | CGGAAGCTGT ACTGGGATTC TTCTAGAAAG TGGGGTGGGA | |
| | | AAGGAGCTAG GGAGGGCGTG TGGAGGGACG AGATCTGTGT | |
| | | CAGAACGTGC GTGTGAGCGG ATACAAAACC CGAGAGAGGC | |
| | | GTGAGCAGCG CTGTGTTTGC GAGCGGGAGC GAGGGGCGCC | |
| | | GGCTGGGGTG TGTGCTCCTG AGCTCTTCAG AAACCAGGCT | |
| | | GCTTTCAGGA ACATTGCTGT GGATTCCCAG GGCCTATTCC | |
| | | ACTAGAAGCA AGATGGCTGA ACTCAATACT CATGTGAATG | |
| | | TCAAGGAAAA GATCTATGCA GTTAGATCAG TTGTTCCCAA | |
| | | CAAAAGCAAT AATGAAATAG TCCTGGTGCT CCAACAGTTT | |
| | | GATTTTAATG TGGATAAAGC CGTGCAAGCC TTTGTGGATG | |
| | | GCAGTGCAAT TCAAGTTCTA AAAGAATGGA ATATGACAGG | |
| | | AAAAAAGAAG AACAATAAAA GAAAAAGAAG CAAGTCCAAG | |
| | | CAGCATCAAG GCAACAAAGA TGCTAAAGAC AAGGTGGAGA | |
| | | GGCCTGAGGC AGGGCCCCTG CAGCCGCAGC CACCACAGAT | |
| | | TCAAAACGGC CCCATGAATG GCTGCGAGAA GGACAGCTCG | |
| | | TCCACAGATT CTGCTAACGA AAAACCAGCC CTTATCCCTC | |
| | | GTGAGAAAAA GATCTCGATA CTTGAGGAAC CTTCAAAGGC | |
| | | ACTTCGTGGG GTCACAGAAG GCAACAGACT ACTGCAACAG | |
| | | AAACTATCCT TAGATGGGAA CCCCAAACCT ATACATGGAA | |
| | | CAACAGAGAG GTCAGATGGC CTACAGTGGT CAGCTGAGCA | |
| | | GCCTTGTAAC CCAAGCAAGC CTAAGGCAAA AACATCTCCT | |
| | | GTTAAGTCCA ATACCCCTGC AGCTCATCTT GAAATAAAGC | |
| | | CAGATGAGTT GGCAAAGAAA AGAGGCCCAA ATATTGAGAA | |
| | | ATCAGTGAAG GATTTGCAAC GCTGCACCGT TTCTCTAACT | |
| | | AGATATCGCG TCATGATTAA GGAAGAAGTG GATAGTTCCG | |
| | | TGAAGAAGAT CAAAGCTGCC TTTGCTGAAT TACACAACTG | |
| | | CATCATTGAC AAAGAAGTTT CATTAATGGC AGAAATGGAT | |
| | | AAAGTTAAAG AAGAAGCCAT GGAAATCCTG ACTGCTCGTC | |
| | | AGAAGAAAGC AGAAGAACTA AAGAGACTCA CTGACCTTGC | |
| | | CAGTCAGATG GCAGAGATGC AGCTGGCCGA ACTCAGGGCA | |
| | | GAAATTAAGC ACTTTGTCAG CGAGCGTAAA TATGACGAGG | |
| | | AGCTCGGGAA AGCTGCCCGG TTTTCCTGTG ACATCGAACA | |
| | | GCTGAAGGCC CAAATCATGC TCTGCGGAGA AATTACACAT | |
| | | CCAAAGAACA ACTATTCCTC AAGAACTCCC TGCAGCTCCC | |
| | | TGCTGCCTCT GCTGAATGCG CACGCAGCAA CCTCTGGGAA | |
| | | ACAGAGTAAC TTTTCCCGAA AATCATCCAC TCACAATAAG | |
| | | CCCTCTGAAG GCAAAGCGGC AAACCCCAAA ATGGTGAGCA | |
| | | GTCTCCCCAG CACCGCCGAC CCCTCTCACC AGACCATGCC | |
| | | GGCCAACAAG CAGAATGGAT CTTCTAACCA AAGACGGAGA | |
| | | TTTAATCCAC AGTATCATAA CAACAGGCTA AATGGGCCTG | |
| | | CCAAGTCGCA GGGCAGTGGG AATGAAGCCG AGCCACTGGG | |
| | | AAAGGGCAAC AGCCGCCACG AACACAGAAG ACAGCCGCAC | |
| | | AACGGCTTCC GGCCCAAAAA CAAAGGCGGT GCCAAAAATC | |
| | | AAGAGGCTTC CTTGGGGATG AAGACCCCCG AGGCCCCGGC | |
| | | CCATTCTGAA AAGCCCCGGC GAAGGCAGCA CGCTGCAGAC | |
| | | ACCTCGGAGG CCAGGCCCTT CCGGGGTAGT GTCGGTAGGG | |
| | | TTTCACAGTG CAATCTCTGC CCCACGAGAA TAGAAGTTTC | |
| | | CACAGATGCA GCAGTTCTCT CAGTCCCGGC TGTGACGTTG | |
| | | GTGGCCTGAG CTAGGAGGAA AAAGAGCAGT TTTCACTCAG | |
| | | TTTTGGTTCC CTGCCCGAGG TGCTGACCCA ATTCGCTGCC | |
| | | AAAAGAGTGT CAATCAGAAT ATACAAATCC CGTATGGTTG | |
| | | TGTCATCCTC TCTTAATCAT TTTTACTAAT TCTAATAATC | |
| | | AGCTCTAGCT TGCTTCATAA TTTTCATGGC TTTGCTTGAT | |
| | | CTGTTGATGC TTTCTCTCAT CAAGACTTTG CAGCATTTTA | |
| | | GCCAGGCAGT ATTTACTCAT TATTAGGAAA ATCAAGATGT | |
| | | GGCTGAAGAT CAGAGGCTCA GTTAGCAACC TGTGTTGTAG | |
| | | CAGTGATGTC AGTCCATTGA TTGTCTTTAG AGAGTTAATG | |
| | | TTACAAAAAA GAATTCTTAA TAATCAGACA AACATGATCT | |
| | | GCTGAGGACA CATGCGCTTT TGTAGAATTT AACATCTGGT | |
| | | GTTTTTCTGA AAAAATATAT ATACATATAT TGCTTTATTT | |
| | | GAAACAAATT AAAATATGCT GCATTTGACA CCTGGCTAGT | |
| | | TTCTTTTATT GATACCCACC TAGTTATTGA ATGTACTGTT | |
| | | TAGTGCTTTC AAAAAAAACT TTAGAGACTA GAGGTTGTGG | |
| | | TGCAAAGCTG TGTACAATAA ATACTGTTTC TGTTGAGACA | |
| | | AGTACTCTTT CAGGAAAATA TATATATGCC CTTTCAATTA | |
| | | GATTACACAA ATAGATGGAT ATGCACCCTG ATCATCTTAG | |
| | | ACACACCATG TGGCAGTTGG GCAGTTGGAA ACATTGGTTG | |
| | | CAAGTACTAC AAACCTCAGC TGAGCCTAGC TTCAACAATA | |
| | | AGAATTTATT GGTTCAGTGA GTGAAAAGTC CAGTTGGTCC | |
| | | AATTTGATCA GGATTTCAGC TCCCATCTCT TTTTTTTTTT | |
| | | TCTTTTTTCT TTTTCTTTTT TTGAGACAGT CTTGCTTTGT | |
| | | CACCCAGGTG GAGTGCAATG GCATGATCTC GGCTCACTGC | |
| | | AACCTCCGCC TCCTGGGTTC AAGCCATTCT CCTGCCCGGC | |
| | | CTTCCGAGTA GCTGGGATTA CAGGCATGTG CCACCACGCC | |
| | | CTGCTAATTT TTATATTATT AGTACAGACA GGATTTCACC | |
| | | ATGTTGTCCA GGCTGGTCTC AAACCCCTGA CCTCAGGTGA | |
| | | TCCACCTGCT TTGGCCTCCG AAACTGCCGG AATTACAGGC | |
| | | ATGAGCCACC GTACCCGGCC AGCTCCCATC TCTTAATTAT | |
| | | CTTAGCTCTC CTTTCCTCCT TGGGTTGACA TTGCATTCAG | |
| | | AATTGTGGCA AAGGGCCACC ATGCTCTTAA GACTCAAGTC | |
| | | TATTTTCCAC ACTGTCCAGA GGAGAGAAGT TATCCTAGTA | |
| | | GCTTCTACAC AGAGCAAGTG TCTTTCTCAG AAATCCCAGC | |
| | | AAAGGTCTTG CATACCATTG CTGGTAGGCC TGTCTCTTAA | |
| | | ACCAATCATG AAAGGGGGGA GAGAAGTGAA TGGGATGAAC | |
| | | TAATTTGCAT AGACTAATTA GAGCCCACCC CTGGAGCCTG | |
| | | GGTGTGGCCA TCTTCCCAGA GTTCCTGAGC TAGATGGAGA | |
| | | AGGGGTACTT CTCAGAAAGG GAAAATGGAT ACCCAATGGC | |
| | | CCAAACCCAA AATAGCCCCA GTTCCCCTAA CTTTGACTAC | |
| | | AGGGCAGTCC AGTTTGGGTG CCGCTTCCGT TGCACTCACA | |
| | | TGTCCTACAT ATCTGTTGAC TACTCACAAG TGCAAATGCT | |
| | | TATTCTCAAC TCAACATTAA CATTTTTTCT GGCAACCCAG | |
| | | GTTCACTGGT TCTCTTCCAC AGAGCGGCCC TGAGCAGCTG | |
| | | AGCCTGCAAG CCACGCAAGC ATCTGTTTCT TCTTTTGCCA | |
| | | AGTACAGGAG GATGTTTGCT CTCTCTGTAG AGAGCTTTCT | |
| | | GAGGTCTCTG GGTGTACCCA GAGATTTAAT AGAAATTCTT | |
| | | AACGTTAAGT CACATTCCAG GAAGGAAGGA AGAGTTGTTC | |
| | | GTTCAAATAA GAAAGATAAA TGTTCGGCAC TGTAGGCCCT | |
| | | GTTTACCCCA TCTGAGGCCC TGAATTCATA TATTACAAGA | |
| | | CGGAAGGATT TTGCACAGTT TTTTATGTAG CAAGATTTTG | |
| | | CTCACCACTG AAAAATGTCA GTGTAAATGT GACCGCTTTA | |
| | | AAGATGAGTC AAGTAATTCT TGGAACAGGG AAAAAAATGA | |
| | | ATTTGCCAGG TCAGGAGTTC ACCTGCCTTT GTCAGAGTTG | |
| | | AACCCAACCA CTCTTGACCT CGACTCACTC CCTTAGGGTT | |
| | | AAGAAAGCCC AAACACATTC CTGAGCACAG AGCAAACACT | |
| | | CCCATGTCAC TGAAAAGAAA CAAAAGAATG CTAAAAAGTG | |
| | | CTCAGACCTG ATCACATTTT TTCCAATATT TTTTCCTTTT | |
| | | TTTTTTTTTT TTTGAGACAG GGTCTTGCTC TATCACCTAG | |
| | | GCTGGAGTGC ATGTTTTTGA GACAGGGTCT TGCTCTATCA | |
| | | CCTAGGCTGG AGTGCATGAT CATGGCTCAC TATAGCCTTG | |
| | | AACTCCTGGG CTCAAGCCAT CCTCAGCCTC CCAAGTTGCG | |
| | | AGGACTACAG GTGTGCACCA CAACGTCCCA GCTACTTTTT | |
| | | AAATTTTTAG TAGAAACGAG GTCTCACTAT GGTGCCCAGA | |
| | | CTGGTCTCGA ACTCCGGAGC TCAAGTGATC TTCCTGCTTT | |
| | | GGCCTCCCAA AGTGTTAGAA TTACAGGCAT GAGCCACCTC | |
| | | GCCTGGCTGG TTTTTGCCTT TCTTATAGAC CCTGGGCATG | |
| | | TAAGCATTTA TTAGTTTGCA TTTTTGAAAC AGTAATTTCA | |
| | | ATATTTTAGT GCCAATGTCA GGCCGCTTAA ACACTGTATT | |
| | | ACATATCTTC ATCTGTCTGG TGGAACTATT GGTGTGATCC | |
| | | TAGAGAACTG AGTCCTATTC TGCCATTCAT TTAAAGTGTT | |
| | | TTAAACTCTA ATCTCTCTAC TTAATGCACA GTAGTCAGAT | |
| | | TATTCTCTTA AACATTTGCC TAGTAGAGGT TAAAATAGTT | |
| | | TAATCCTTAT GAAGATGGAA TAACTTCAAA CTCACATTGT | |
| | | GGCACTTAGA TCTTCCACCA AGACTTCATC CGTGAAATCC | |
| | | ACACCTCCCT GTTGGGTTCC CAATTACATT CCAAATTTAC | |
| | | ATTTCTTTTG AGAATCTCTG CATACTCCAG CTCTGTCCTG | |
| | | TTGATCCTAT TCTAGAAGTG CTTAATGCAG CAAGACACAG | |
| | | AAAGTTAAAC GCAAATTGCT GCAAAATTCA CCCTCAGTGG | |
| | | AGGACTAGAA ACACAACATG TCCAATTTAA AGCTCAGTTC | |
| | | ACAAGCAGTT CAATTCTGCT GGCATCAGAA AAGGAGATTC | |
| | | TAATTAAACA TTCTTAGGGA AGGACATCAA ATGAGGTTAA | |
| | | TGGGAAACGT TACCAGATTA AAAGCAGTTT TTTGACAAAG | |
| | | TAACAGATTT GGAAATTCTG ACTCTCTGAA AGCCTTGATT | |
| | | TGAACCTCAA ACTTGATTTC ACCATGAGAA GTGGGGATCA | |
| | | AGGGCCTGCG CAGTTCTTTT CCTAAATCGA TTCGGTGCTC | |
| | | CCCACCCCGA CGCAGGCACA GGTCCGCAAC CAGATAGGGA | |
| | | GATGCCTGAA TTTCAGGCTA CCTTTGACAA AGCTTTCTTC | |
| | | CTCCCTCCCT CCCTTTGCAC GCTGCATCCC ACGCTGCCTG | |
| | | CTTAAAGCGC CCTCATGTGT CTACAGATGG TAAAACGTTT | |
| | | ATTTCTCAAC AGACATTCCA GTGATAGCAT CCAATGACCT | |
| | | ATGTAACGGC ACCCTTTTTT GCTGTACGCG TTTTTTGAGA | |
| | | TGGAGTCTCG CTCTGTCGCC CAGGCTGGAG TGCAGTGGCG | |
| | | CGGTCTCGGC TCACTGCAAG CTCCGCCTCC CGGGTTCACG | |
| | | CCATTCTCCT GCCTCAGCCT CCCGAGTAGC TGGGACTACA | |
| | | GGCACCCGCC ACCACACCTG GCTAATTTTT TGTATTTTTA | |
| | | GTAGATACAG GGTTTCACCG TGTTAGCCAG GATGGTTTTG | |
| | | ATCTCCTGAC CTCGTGATCC ACCCCCCTCG GCCTCCCAAA | |
| | | GTGCTGGGAT TACAGGGGTG AGCCACTGCG CCCGGCCCCA | |
| | | GTCACTTGTT CTTAAGTTTC TTAAGCAAAC TATAAAATAG | |
| | | CAAAAGACCA AAAAAAAAGG AAAAAAAGCA GTTCGCCTAA | |
| | | TACATTGTTC CAGCATTTCC TTGAAAGTAC TGAGCCATCT | |
| | | CAATTGCTCT GATTTTGTGA GAAAATTATG AAGAGTTGCA | |
| | | AAGTCCCAGT GATTCTCTTG TTACTTAGCT AAGAATTTGA | |
| | | AATGTAATTT AAATACTATT CTTTACAATC CATTATAAGG | |
| | | ATTTTAAAAT CTTTTTGCTT CTTTAATAAA TTCTAACAAA | |
| | | GAAAAAAAAA AAAAA | |
| TDRD7 | NM_001302884.1 | AGAGCCGAGG CCAGGCTGCC CTCGAAGCGG GGCGGGGCGA | 26 |
| | | AGCGGGGCGG GGCCGAGCAG GGCGGGGCGG GGGCTTGAGG | |
| | | TGATTCCCAA GCCGCGGGGC GGCTCCGGTG GTGCGGGGAA | |
| | | ACCGAAAGTG GGCGGCGGCC GCGGCGGGGC CCCTGGCGGA | |
| | | GACGGCGGCA GGAGCTGGGC CCAGAGACGC GGGGACGGGC | |
| | | CGTGGGCCCC CGGAACGAGA TTACCTGCTA TGCCATGGCC | |
| | | TGCACAGAAA CTGCAAGAAT TGCTCAGCTT GTGGCTCGTC | |
| | | AAAGGAGTTC TAAAAGGAAA ACCGGGCGTC AAGTTAATTG | |
| | | TCAGATGAGA GTGAAGAAAA CCATGCCATT TTTTCTAGAA | |
| | | GGAAAACCAA AAGCAACCCT CAGACAACCA GGATTTGCTT | |
| | | CAAATTTTTC TGTTGGCAAA AAACCTAATC CAGCACCGTT | |
| | | AAGAGACAAA GGAAACTCTG TTGGAGTTAA GCCTGATGCT | |
| | | GAAATGTCTC CTTATATGCT ACACACAACT CTTGGAAATG | |
| | | AAGCATTCAA AGACATTCCA GTGCAAAGGC ATGTGACCAT | |
| | | GTCCACCAAC AACAGGTTTA GCCCAAAGGC GTCCCTTCAA | |
| | | CCACCTTTGC AGATGCATCT CTCAAGAACC TCTACTAAGG | |
| | | AAATGAGTGA TAATTTAAAT CAGACTGTTG AAAAACCCAA | |
| | | TGTCAAGCCT CCTGCCTCTT ACACTTATAA AATGGATGAG | |
| | | GTTCAAAATC GCATAAAGGA AATACTAAAC AAGCATAACA | |
| | | ATGGCATTTG GATATCTAAG CTTCCACATT TTTACAAAGA | |
| | | GTTATATAAA GAAGACCTTA ATCAAGGAAT TTTACAACAG | |
| | | TTTGAACACT GGCCTCATAT TTGCACGGTG GAGAAACCTT | |
| | | GCAGTGGTGG CCAAGATTTA CTTCTTTATC CAGCTAAGAG | |
| | | AAAGCAGCTT TTGAGAAGTG AACTGGATAC TGAGAAAGTA | |
| | | CCTCTATCCC CACTACCTGG TCCCAAACAA ACACCACCGT | |
| | | TGAAAGGGTG TCCAACAGTT ATGGCAGGAG ACTTTAAAGA | |
| | | AAAAGTGGCA GACCTGCTGG TGAAATACAC AAGTGGCCTT | |
| | | TGGGCCAGTG CACTTCCGAA AGCATTTGAG GAAATGTACA | |
| | | AAGTGAAATT CCCTGAGGAT GCCTTAAAAA ATCTTGCCTC | |
| | | ACTTTCTGAT GTATGCAGCA TAGACTACAT TTCTGGAAAT | |
| | | CCCCAGAAGG CCATTCTCTA TGCTAAACTT CCATTGCCCA | |
| | | CTGACAAAAT CCAAAAGGAT GCAGGGCAAG CACATGGTGA | |
| | | TAATGATATC AAGGCTATGG TTGAACAAGA GTATTTGCAG | |
| | | GTAGAAGAAA GCATTGCTGA AAGTGCTAAT ACCTTTATGG | |
| | | AGGACATAAC AGTTCCTCCT TTAATGATTC CAACTGAAGC | |
| | | ATCACCATCT GTATTGGTGG TTGAACTGAG CAACACAAAT | |
| | | GAAGTGGTTA TCAGGTATGT GGGCAAAGAC TATTCTGCTG | |
| | | CTCAGGAATT AATGGAAGAT GAGATGAAGG AATATTACAG | |
| | | TAAGAATCCT AAGATCACAC CAGTCCAGGC TGTGAATGTT | |
| | | GGGCAGTTGC TGGCCGTAAA TGCCGAGGAG GACGCCTGGT | |
| | | TACGGGCACA GGTCATCTCA ACAGAAGAGA ACAAAATAAA | |
| | | GGTATGCTAT GTTGACTATG GTTTTAGTGA AAATGTTGAA | |
| | | AAAAGCAAAG CATACAAATT AAACCCGAAG TTTTGTTCAC | |
| | | TCTCATTTCA AGCTACAAAA TGTAAGCTTG CAGGCTTGGA | |
| | | AGTCCTAAGC GATGACCCTG ATCTAGTGAA GGTGGTTGAA | |
| | | TCTTTAACTT GTGGAAAGAT CTTTGCAGTG GAAATACTTG | |
| | | ACAAAGCTGA CATTCCACTT GTTGTTCTGT ACGATACCTC | |
| | | AGGAGAAGAT GATATCAATA TCAATGCCAC CTGCTTGAAG | |
| | | GCTATATGTG ACAAGTCACT AGAGGTTCAC CTGCAGGTTG | |
| | | ACGCCATGTA CACAAATGTC AAAGTAACTA ATATTTGCTC | |
| | | TGATGGGACA CTCTACTGCC AGGTGCCTTG TAAGGGTCTG | |
| | | AACAAGCTCA GTGACCTTCT ACGTAAGATA GAGGACTACT | |
| | | TCCATTGCAA GCACATGACC TCTGAGTGCT TTGTTTCATT | |
| | | ACCCTTCTGT GGGAAAATCT GCCTCTTCCA TTGCAAAGGA | |
| | | AAATGGTTAC GAGTAGAGAT CACAAATGTT CACAGCAGCC | |
| | | GGGCTCTTGA TGTTCAGTTC CTGGACTCTG GCACTGTGAC | |
| | | ATCTGTAAAA GTGTCAGAGC TCAGGGAAAT TCCACCTCGG | |
| | | TTTCTACAAG AAATGATTGC AATACCACCT CAGGCCATTA | |
| | | AGTGCTGTTT AGCAGATCTT CCACAATCTA TTGGCATGTG | |
| | | GACACCAGAT GCAGTGCTGT GGTTAAGAGA TTCTGTTTTG | |
| | | AATTGCTCGG ACTGTAGCAT TAAGGTTACA AAAGTGGATG | |
| | | AAACCAGAGG GATCGCACAT GTTTATTTAT TTACCCCTAA | |
| | | GAACTTCCCT GACCCTCATC GCAGTATTAA TCGCCAGATT | |
| | | ACAAATGCAG ACTTGTGGAA GCATCAGAAG GATGTGTTTT | |
| | | TGAGTGCCAT ATCCAGTGGA GCTGACTCTC CCAACAGCAA | |
| | | AAATGGCAAC ATGCCCATGT CGGGCAACAC TGGAGAGAAT | |
| | | TTCAGAAAGA ACCTCACAGA TGTCATCAAA AAGTCCATGG | |
| | | TGGACCATAC GAGCGCTTTC TCCACAGAGG AACTGCCACC | |
| | | TCCTGTCCAC TTATCAAAGC CAGGGGAACA CATGGATGTG | |
| | | TATGTGCCTG TGGCCTGTCA CCCAGGCTAC TTCGTCATCC | |
| | | AGCCTTGGCA GGAGATACAT AAGTTGGAAG TTCTGATGGA | |
| | | AGAGATGATT CTATATTACA GCGTGTCTGA AGAGCGCCAC | |
| | | ATAGCAGTGG AGAAAGACCA AGTGTATGCT GCAAAAGTGG | |
| | | AAAATAAGTG GCACAGGGTG CTTTTAAAAG GAATCCTGAC | |
| | | CAATGGACTG GTATCTGTGT ATGAGCTGGA TTATGGCAAA | |
| | | CACGAATTAG TCAACATAAG AAAAGTACAG CCCCTAGTGG | |
| | | ACATGTTCCG AAAGCTGCCC TTCCAAGCAG TCACAGCTCA | |
| | | ACTTGCAGGA GTGAAGTGCA ACCAGTGGTC TGAGGAGGCT | |
| | | TCTATGGTGT TTCGAAATCA TGTGGAGAAG AAACCTCTGG | |
| | | TGGCACTGGT GCAGACAGTC ATTGAAAATG CTAACCCTTG | |
| | | GGACCGGAAA GTAGTGGTCT ACTTAGTGGA CACATCGTTG | |
| | | CCAGACACCG ATACCTGGAT TCATGATTTT ATGTCAGAGT | |
| | | ATCTGATAGA GCTTTCAAAA GTTAATTAAT GACTGCCTCT | |
| | | GAAACCTTGA CAACTAATTC AGATTTTTTA GCAATAACAA | |
| | | AATGTAGTAG GCTTAAAAAA AATCTTAACT CTGCTACATG | |
| | | GCTCTGACTG CTGTGGGGGA TTGAAAAGAA TATGCTTATG | |
| | | TTTGATGAAA GATATTTAAC AAGTTTTGTT TTAACAGAGT | |
| | | TGACTTTTCA AAGAAAATTG TACTTGAATT ATTACTATAA | |
| | | TATTAGAATA AAAATGTTTA TCAATATAAA AAAAAAAAAA | |
| | | AAAAAAAAAA AA | |
| TOX4 | NM_001303523.1 | AGCAGAGAGA ACACACGTCC TTGCGGAAGT GACGGCAGTT | 27 |
| (house- | | CCGAGTCCAG TGGGGGCGGT GGGAGCGATG AGGGTCTGAG | |
| keeping | | ACGGTGGGAG CGGTTGTGTG AAGATGGAGA CATTCCATAC | |
| gene) | | ACCAAGCTTG GGTGATGAGG AATTTGAAAT CCCACCTATC | |
| | | TCCTTGGATT CTGATCCCTC ATTGGCTGTC TCAGATGTGG | |
| | | TTGGCCACTT TGATGACCTG GCAGACCCTT CCTCTTCACA | |
| | | GGATGGCAGT TTTTCAGCCC AGTATGGGGT CCAGACATTG | |
| | | GACATGCCTG TGGGCATGAC CCATGGCTTG ATGGAGCAGG | |
| | | GCGGGGGGCT CCTGAGTGGG GGCTTGACCA TGGACTTGGA | |
| | | CCACTCTATA GGAACTCAGT ATAGTGCCAA CCCACCTGTT | |
| | | ACAATTGATG TACCAATGAC AGACATGACA TCTGGCTTGA | |
| | | TGGGGCATAG CCAGTTGACC ACCATTGATC AGTCAGAACT | |
| | | GAGTTCCCAG CTGGGTTTGA GCCTAGGGGG TGGCACCATC | |
| | | CTGCCACCTG CCCAGTCACC TGAAGATCGT CTTTCAACCA | |
| | | CCCCTTCACC TACTAGTTCA CTTCACGAGG ATGGTGTTGA | |
| | | GGATTTCCGG AGGCAACTTC CCAGCCAGAA GACAGTCGTG | |
| | | GTGGAAGCAG GGAAAAAGCA GAAGGCCCCA AAGAAGAGAA | |
| | | AAAAGAAAGA TCCTAATGAA CCTCAGAAAC CAGTTTCAGC | |
| | | ATATGCTTTA TTCTTTCGTG ATACACAGGC TGCCATCAAG | |
| | | GGACAGAATC CTAATGCCAC TTTTGGTGAG GTTTCAAAAA | |
| | | TTGTGGCCTC CATGTGGGAT AGTCTTGGAG AGGAGCAAAA | |
| | | ACAGGTATAT AAGAGGAAAA CTGAGGCTGC CAAGAAAGAG | |
| | | TATCTGAAGG CACTGGCTGC TTACAAAGAC AACCAGGAGT | |
| | | GTCAGGCCAC TGTGGAAACA GTGGAATTGG ATCCAGCACC | |
| | | ACCATCACAA ACTCCTTCTC CACCTCCTAT GGCTACTGTT | |
| | | GACCCAGCAT CTCCAGCACC AGCTTCAATA GAGCCCCCTG | |
| | | CCCTGTCCCC ATCCATTGTT GTTAACTCCA CCCTTTCATC | |
| | | CTATGTGGCA AACCAGGCAT CTTCTGGAGC TGGGGGTCAG | |
| | | CCCAATATCA CCAAGTTGAT TATTACCAAA CAAATGTTGC | |
| | | CCTCTTCTAT TACTATGTCT CAAGGAGGGA TGGTTACTGT | |
| | | TATCCCAGCC ACAGTGGTGA CCTCCCGGGG GCTCCAACTA | |
| | | GGCCAAACCA GTACAGCTAC TATCCAGCCC AGTCAACAAG | |
| | | CCCAGATTGT CACTCGGTCA GTGTTGCAGG CAGCAGCAGC | |
| | | TGCTGCTGCT GCTGCTTCTA TGCAACTGCC TCCACCCCGA | |
| | | CTACAGCCCC CTCCATTACA ACAGATGCCA CAGCCCCCGA | |
| | | CTCAGCAGCA AGTTACCATT CTGCAGCAGC CTCCTCCACT | |
| | | CCAGGCCATG CAACAGCCTC CACCTCAGAA AGTTCGAATC | |
| | | AATTTACAGC AACAGCCTCC TCCTCTGCAG ATCAAGAGTG | |
| | | TGCCTCTACC CACTTTGAAA ATGCAGACTA CCTTAGTCCC | |
| | | ACCAACTGTG GAAAGTAGTC CTGAGCGGCC TATGAACAAC | |
| | | AGCCCTGAGG CCCATACAGT GGAGGCACCT TCTCCTGAGA | |
| | | CTATCTGTGA GATGATCACA GATGTAGTTC CTGAGGTTGA | |
| | | GTCTCCTTCT CAGATGGATG TTGAATTGGT GAGTGGGTCT | |
| | | CCTGTGGCAC TCTCACCCCA GCCTCGATGT GTGAGGTCTG | |
| | | GTTGTGAGAA CCCTCCCATT GTGAGTAAGG ACTGGGACAA | |
| | | TGAATACTGC AGCAATGAGT GTGTGGTGAA GCACTGCAGG | |
| | | GATGTATTCT TGGCCTGGGT AGCCTCTAGA AATTCAAACA | |
| | | CAGTGGTGTT TGTGAAATAG TCCTTCCTGT TCTCCAAGCC | |
| | | AGTGAAGAGT TATCTGCTGG GAAAGTGTCC AAGAGCCTGT | |
| | | TTTTGAAACA CAAGCTGGGC TTCTGGTAGT GCCTCATCAC | |
| | | AACCCATGAT GGCTGTTCAT GTTTCACCCC TTTTCTTCCT | |
| | | TCAGCAGAGG CCAGGCTATG GAGCAGGGCC ACTGAATTTG | |
| | | CTGTAATCTG GAGATGCTTT TTACTTTCAA CCATAAGCGG | |
| | | TAATAGCAGA GGAAAGGGTG AAGGGAGTCT GGGCAAGCAA | |
| | | AGCATAGAGA TGGTGGGGTG GTGGTGGGGT TGAAGAAACT | |
| | | TGTTGGTATA ATTGTCATAG GACTTGCCTA AAATATTATT | |
| | | AAAATTACGG GAGTGTACTC AGCTTTGAGC CTAGGAGAAA | |
| | | ATGCCACTGT GTGCATCCAT TTTAAAGGGT TCCCTCATAA | |
| | | AAAAATGTTA TTCCCCATTA TCACATCAGT ACACTGCTTT | |
| | | GAAAACAAAA CTTTTCAACA TGGGCATACT GGGCTACATG | |
| | | GAAAATGACA TCACCCAGGA GTGATTTCTC TTTATATATA | |
| | | TTATTTCTGC AGTTACCATC CTTATCTGAG TTATCACAGT | |
| | | TCATGAATCT AAGAGGCGGA ACTCTACATC ATTAGTAAGA | |
| | | GGTTCCACCA AAGTCTAAAG TTGTATTCAC TTGTGTTTGA | |
| | | TGAACTATCT TTAAAAGACC ATAGGTCTAT CATTATTTCT | |
| | | TAGACATAAT CTAAAGAAAA ACAGACTAGA GAAGCCACCT | |
| | | GGTTGTAACA GAATAAGCAG AAGTTTACAG CATGATAGTC | |
| | | CAAGTGGTGA TAACTTTAAA TAAAACTCAA ATTTTTACTG | |
| | | TTTGTAGACA GGAATGCTGT CCTAGAGAAC CTCCTCCTCA | |
| | | ACCAGCTACG TACATAGTTT TATCCTATGC ATTCCTGTTT | |
| | | TCTGTGTGTT TTTTGTTTTT TTTTTTTTTT TTTTTTTTTG | |
| | | AGACAGAGTC TCGCTCTGTC ACCCAGGCTG GAGTGCAGTG | |
| | | GTGCGACCTC AGCTCACTGA AACCTCTGCC TCCCGGGTTC | |
| | | AAGCGATTCT CCTGCATCAG CCTCCCGAGT AGCTAGGATT | |
| | | ACAGGCGCCC GCCACTACGC CCAGCTAATT TGTGGTATTT | |
| | | TTAGTAGAGA CAGGGTTTCA CCATGTTGGC CAGGCTGGTC | |
| | | TCGAACTCCT GACCTCATGA TCCGCCCGCC TTGACCTCCC | |
| | | AAAGTGCTGG GATTACAGGC ATGAGCCACC GCACCCAGCC | |
| | | TGCATTCCTG TTTTTTTAAT GGTTTTGGAG GGTAGCAGTA | |
| | | GAGATGGGGT CTCACTATGT TGCCCAGTCT AGTCTTGAAC | |
| | | TCCTGGGCTA CAGTTACCCT CCTACCTCGG CTTCCCAAAG | |
| | | TGCTCGGATT ACAGGTGTGA GCCACTGTGC CTAGCCTATA | |
| | | ATGATCATTT TAATGTTTCC CATGCACTCA TTTAGTTTGA | |
| | | ACCTTCACAG CAACCCAATG AGGTAATACT CCCATTTCAC | |
| | | ATATAATACT GAGAGATGAG TTGCACAAGA TTATACACTG | |
| | | TTAAGTAGCA GAGCCAGAAT GGACTTCAGA ATCCCAACTA | |
| | | CAATACAAAT GTTTATTTAA ATAAAGAAGA AAGCTATTGT | |
| | | ACAAATATCA CTCTTCAGGT TTAGCTTACA GAGCCATGGC | |
| | | TATGGATTCT TAGCTCTGTA AGGAAGTGCT TCTATAAATT | |
| | | CTTAGGTTTA GAGATGATAC CATCTGGGTA CCTTTGCTTG | |
| | | AACCGTGCAA CCACATCTGG GTCTAGTAGG TGGATCCCAT | |
| | | CCAGTTGGTT TCCAAGGGTG ATCCTGAAAC AGTGTAAAAG | |
| | | GAGGGGCAAA CCAGAAATCC TGGAATTAGA GGGTTTAATA | |
| | | TTGTTAAAAA ATGCATACCA AATGAAGACT GCCTATCATC | |
| | | ATATCAAATA TGCCAATTCT AAAAAGAGCT TAACATTAGA | |
| | | ATAGTATATG GTAGAATTAC TAGTTCAGAA TTGGCATAGA | |
| | | TTCTGGTGTT AAAATAGACT GGATCTGTAT TATCTGAGGG | |
| | | TTAGTAACTA ATGCTTAGCC AGGCCTGCTT CACAGAGTTG | |
| | | CTACCAGGGA GTATTCTTTG GATAAGCAAA ATGCTAGCAG | |
| | | CATGTGTTTT AAGCTCTGTT AAGGGGTGAA AGATGTAATT | |
| | | ATTGACAGAT TAAATAGATA ACTTCGTAAC CACCAGGGGG | |
| | | CAGATTCAAT ACATCACAGA ATGGCTGAGG AAGATCCTTG | |
| | | GGTTGTGAAG AGAGTAGAAA CCCTAGGGAG CAGTGCTTTT | |
| | | GGGTCCTAGA ACCTGTTGAG TTTCTAATGA ATATTTGTAG | |
| | | AATCTCATAA AACAGTTTAA ATACAAGCTT AAGTGGCTTA | |
| | | TGAATCCTGT GAAGCTCATT TATGGACTAG TGTAAAACAA | |
| | | TGTGAAGCTC TACTAAGTTC TGTCCTTAAT CATAAATAAT | |
| | | AGCCCCTTGA GGACTAGCCT GTTCTCTGGT CACCTTACCA | |
| | | GTTGGGTTGC ACATTGTGTG GTCGTCCAAA TAACTCAATC | |
| | | TTGCGAGTGC CAGGAGATAG TCTTTCAATC ATGCCATAGA | |
| | | TTTCATCTGG TTTATGACTG GTGGAACGAA CCTAGGAAAT | |
| | | AAAAACTAGC TGCTTTTTAA GTTACACAAG AAAAAA | |
| TPT1 | NM_001286272.1 | CTTCGTGCCA CGTCACCGCC TGCGTCGCTT CCGGAGGCGC | 28 |
| (house- | | AGCGGGCGAT GACGTAGAGG GACGTGCCCT CTATATGAGG | |
| keeping | | TTGGGGAGCG GCTGAGTCGG CCTTTTCCGC CCGCTCCCCC | |
| gene) | | CTCCCCCCGA GCGCCGCTCC GGCTGCACCG CGCTCGCTCC | |
| | | GAGTTTCAGG CTCGTGCTAA GCTAGCGCCG TCGTCGTCTC | |
| | | CCTTCAGTCG CCATCATGAT TATCTACCGG GACCTCATCA | |
| | | GCCACGATGA GATGTTCTCC GACATCTACA AGATCCGGGA | |
| | | GATCGCGGAC GGGTTGTGCC TGGAGGTGGA GGGGAAGATG | |
| | | GTCAGTAGGA CAGAAGGTAA CATTGATGAC TCGCTCATTG | |
| | | GTGGAAATGC CTCCGCTGAA GGCCCCGAGG GCGAAGGTAC | |
| | | CGAAAGCACA GTAATCACTG GTGTCGATAT TGTCATGAAC | |
| | | CATCACCTGC AGGAAACAAG TTTCACAAAA GAAGCCTACA | |
| | | AGAAGTACAT CAAAGATTAC ATGAAATCAA TCAAAGGGAA | |
| | | ACTTGAAGAA CAGAGACCAG AAAGAGTAAA ACCTTTTATG | |
| | | ACAGGGGCTG CAGAACAAAT CAAGCACATC CTTGCTAATT | |
| | | TCAAAAACTA CCAGTTCTTT ATTGGTGAAA ACATGAATCC | |
| | | AGATGGCATG GTTGCTCTAT TGGACTACCG TGAGGATGGT | |
| | | GTGACCCCAT ATATGATTTT CTTTAAGGAT GGTTTAGAAA | |
| | | TGGAAAAATG TGATGCAAAA GAAAGAAATC CCTGCGCTTT | |
| | | CTGTCTGTCT TTGTGGCGGC CCAGATTGAA TTGGGGAATA | |
| | | CATCTTTAGC CTGGAAATGT AGGCTGCATG TTAATGGTAA | |
| | | TGTAACTTTT GCAGTGTAAT GTTTGAAAAA TATTAATGTA | |
| | | GTTTTTGCTT TTACAGTAAC AAATGTGGCA ATTATTTTGG | |
| | | ATCTATCACC TGTCATCATA ACTGGCTTCT GCTTGTCATC | |
| | | CACACAACAC CAGGACTTAA GACAAATGGG ACTGATGTCA | |
| | | TCTTGAGCTC TTCATTTATT TTGACTGTGA TTTATTTGGA | |
| | | GTGGAGGCAT TGTTTTTAAG AAAAACATGT CATGTAGGTT | |
| | | GTCTAAAAAT AAAATGCATT TAAACTCATT TGAGAGAATG | |
| | | CCTTTTAGTT TAATGCATAT TTAAACTAAA TTGATCCTGT | |
| | | AGTGTTCCTG GAGAAGCTAG AGCCTGATTG TAGGCTACTA | |
| | | CTCATCAATT AACTTCTACA GTGGAGACTA CTTCTGGGAC | |
| | | TGGAATATAA AAAAGAATCA AAGGTTCTGA TTTTGAGTTG | |
| | | CAATAAAGGG AAAGACCATG CTCATAGCAG TGCCAACATC | |
| | | TGAAGTGTGG AGCCTTACCC ATTTCATCAC CTACAACGGA | |
| | | AGTAGTTAAC TGGAAGAGAT TACCAAGAGA ATAAAAAGAG | |
| | | ACTCATTCAG TGGAAGCAAC TTTGTCTCAG CTTATTTCAC | |
| | | ATAAAGAGAG CGAAGTCTTT TGGGATGAAT GTTAATTAAA | |
| | | CTCCCTGGTA ACTAGAACAG GGACTGGCAA ACTAGCCTAT | |
| | | CTGACCACCT GTTTTGTACA CTTTAAGGTG GTTGGTTGCC | |
| | | TTTTTAAATG GTTGAGGGGA AAAGAATACC TTGTGGGATA | |
| | | TGGAATTTAA GTTCGAGTCC AGTTTTATTG GAACGTGGCT | |
| | | ATGCTTATTC ATTTATGGAT TGACTGTGGC TGTTGTCAGT | |
| | | GCATGAGCAG AGTTGTGTCT AACAGACTAG AGCCTGCAAG | |
| | | TTTGCCAGCC CCTGATTTAA AAGATGAAGG TACACAGAAT | |
| | | GTGGGCTGGC TGGTGGGCAA AGGGGTAAAA ATGTTCTCTA | |
| | | TATTGTATCT GAAAAGATGG GGTGTCTGAA TAAGAAAATG | |
| | | CATCTATTTG ACAGACCTGG AGCAGTTGCT ATCTGCTGCT | |
| | | ATGGTTTCCA CCACAGATGC AAGAAGAACA TGTCCTTGCG | |
| | | CTTTCCGTCT GTCTAATTGT GGCAGCTGAG ATTGAATAGA | |
| | | GGAATACAGG AGGAAAAAAA GCGGGAAGAG TTTTTGAGGC | |
| | | AGGTCGGTCA CCCAGGCTTG TAGTGCAGTG GCACAAGCAA | |
| | | CTCACTGCAT TCTCTGCATC CTGTGCTCAA GCCATTTTCC | |
| | | CACCTCAGTC TCACTAGTTG CTGGGACTGC AGGCATGCAC | |
| | | CCCTATGCCC AGCTAATTTT TGTAGAGACC GAGTATCGCT | |
| | | TAGTTGCCCA GGGTGGTCTC AACTCCTGGG CTCAAGGAGA | |
| | | TCTGCCCACC TCAGCCTCCC AAAGTGCAGG CCTAGCCTGG | |
| | | GAGGGGAATT TTCAAAACGT GAGTTTTGGG AAATAGTCTA | |
| | | TCAGCCTTAC CTGGTTGATT ACACTTGTAA AAGAAAGATT | |
| | | AAAAGCAGGC CAGTGACTCT GGTCTGCTTG AACATGTGAA | |
| | | TGTAGTGGTT TGAGCAATCT GGAGTTTGCC CTAGTGTCAA | |
| | | ATTCCAGACT GTCCATAGTG TCCAAAACCT GAGGCAGATA | |
| | | CTAATGTTAA CCCCCAGCAC CCCGTGATTG GAAACAAACC | |
| | | TAAATACGTA TTGGGAACTT AATAGCAATT TTAAGCATTC | |
| | | TGATAGATTT TTTGTAGGGA TGGGGTCATG CCATGTGGCC | |
| | | CAGGCTGGTC TGAAAACTCT GGCCTCAAGT GATCTCAAGC | |
| | | TTTGGCCTTC TAAAGTGTTG GGATTACAGG TGTGAGGCAT | |
| | | TGCACCTGGC TTAGCGTTCT GATTTGACAT TGTAATGAAA | |
| | | AGTGTGAGTC TCATCTACAG GGCCTTTTGT CCTCTGAAAT | |
| | | GATAGCAGGA AGGGAATTTT CAGGCAGTGG TCAAAGCTGG | |
| | | GGAAACCAGG ATAGTGAAGA AGGCCTTGAG GTGAGAGATG | |
| | | GAAGCTAATT GGTGAACTAG CCTTGGAAGC CTGAAACAGA | |
| | | CAAGTAGCAA TTCAGAGACT TTGTGGGCTC CACTGCTCCA | |
| | | ACTTGTTTTG AAGATTTTCA GTTCTGCAGA AGAGGTATTT | |
| | | CCCCAGTTGT CCTTTCAGTG CTCTTAGCTG TTTTCCCAAC | |
| | | ATCCAGATCC AATCAAGGCT GGGACATAGC ATTTTATCAT | |
| | | GTCTATTTAA GTCAGAAGTG ATGAACCCCA GCTGTTTACC | |
| | | TCATGGTAAA CCTTTGAAGA TTCCAGGTAG AATCTTCTCA | |
| | | GACTTTGAAG ACTGTCTCAT TTTATATCTT TTTCTCGTTA | |
| | | TTCCTAGGGT CAAGACGTTT TGGGCAAGAA TAAGGATGTG | |
| | | AACATCAGAA AGCTCATAAC ATTTTGTTTT TGATGCTAAG | |
| | | TTTAACAAAG GCATGCTTTA GTAGCCTGTG GGCCCTAGGG | |
| | | TTTGTTAAAG TGTGGAGAAC AACTGAGTGG AGCAAGAGGA | |
| | | CTTTTCTAGG AAGGTCCTTG TAATGTGACA TTTGAAAACA | |
| | | AATGAAGGTG TGGAAGTAGG CCATGTGGAT ATCAGGACAA | |
| | | ACCATTCCAG GCCAAGACAA CAGCAGTTAG TCTGGAGTGT | |
| | | GATGTGTTCT GGGAAAAAAG TGGCCACTTT GCTAACCCAA | |
| | | GAAGACAGGA AGGGTTGTAA AGCAGTGGGA GTGTGCAAGG | |
| | | AAGGAAGACC AGACCTCAAG GAAACCACAG GCGCTCTGAG | |
| | | CAGAAGAGTT ACATGATATG ACTCAAATTT TTAAAGGATC | |
| | | ACTTTGGCTG CCAGGTGGCA GGGTAAAAGC ATAGAATAAT | |
| | | TGTGTATAAT GTGTTTTTAA GGCAAAGATA GTGGCTTAGT | |
| | | CTAGGGTAGT AGACTGAGGT GGTAGGAAAT GAAGATAGAG | |
| | | ACAACAGGAT ATGCTGGTGG GTGAGGATGG ATTTAATGTT | |
| | | GATACAAGTA TTTTGGTCTG AGCGTTTGGA AGAAAGTTGG | |
| | | CACTGAGGTG GGAAGTCGAG TTTAGTTTTG TTAGTTTTGG | |
| | | ATGTGTTAAG TTTGAGATGC TGATTCTTCA GAGAAGTCTA | |
| | | AGCTGGAGAA CTATATAGAG AGTGGAAAGA TAACAATAGA | |
| | | CATTGAAAGC CATGATACAG GATAAGGTCA TTTGGAGAGA | |
| | | GGATAGACTG CATTCCAACA TGAGATTGGT TGACAAAGAG | |
| | | AAACCAACAA AGGTAATTAA GAGGTGCTCC CACTGCACTT | |
| | | GTACTCAGAA GGCTGAGGTA GGATTGTTAG AGGCCAGCCT | |
| | | GGGCACCACA GGGAGACCCC ATCTCTAAAA TTTAGCCAGG | |
| | | AACCATGGCT CATGCCTGTA GCCCCAGGAA TTTGGGAGGC | |
| | | TGAGTGGGGA GGATCGCTTG AGGTCAGGAG TTTGAGACCA | |
| | | GCCTGGGCAA CATAGGGAGA CCTAAAAAAA TTAATTGGGC | |
| | | ATCTGTAGTC CCAGCTACTC AGGCGGCTGA GCTGAGAGGA | |
| | | TGGCTTGAGT CCGAGAGATT GAGGGTGCAG TGAGCTGTGA | |
| | | TCATACCACT GCACTCCAGC CTGGGCGGCA GTGAGACACT | |
| | | ATCTGAAAAA AGTTTAAAAA TTTTAAAAAA GAAGGAACTG | |
| | | CCCCTGAGGT AAGAACCAAG GGAGGGCCTC CCAGAGGTCA | |
| | | GGTGGAAAAA GTTTTAGGAA GGAGGAAGTA GTCAACAGGG | |
| | | TTACCTGTTG CAAAGTACTT AAGTAATATG AGGCCTGATA | |
| | | GTGGTAAACT TGACTACCGT TGGATTTCAC TAGTGGGAAA | |
| | | GGAAGTCTAA TTAAAATGCA CTCAAGAGAC TAACAGTCGC | |
| | | AGGCATGAAA TACAATACAG GTACATGGTT TTTTATTATG | |
| | | TGTGCATCTG CTTCAGTAAT AGGTGTGAAT TACTCATTTG | |
| | | GATCATTAGG AGTTTCAAAA TCTAGTTAAA TGACTAGATT | |
| | | TTTGTTGATG TAAATTCTGT CATTCTGAAC TGCAGGGATT | |
| | | GTCAGTAACT TAACTGCAAA CTAAACTGGT GATAATTATG | |
| | | GTAAAATTGC AAGACGAGCA ATAAATCTCA ACCAACTTGA | |
| | | GAGAACACTG ATAA | |
| TXK | NM_003328.2 | GATTTCAGTT GAAAGATGTG TTTTTGTGAG TAGAGCACCG | 29 |
| | | CAGAAGAACT GAAGACTGTT GTGTGCTCCC CGCAGAAGGG | |
| | | GCTACCATGA TCCTTTCCTC CTATAACACC ATCCAGTCGG | |
| | | TTTTCTGTTG CTGCTGTTGC TGTTCAGTGC AGAAGCGACA | |
| | | AATGAGAACA CAGATAAGCC TGAGCACAGA TGAAGAGCTT | |
| | | CCAGAAAAAT ACACCCAGCG TCGCAGGCCG TGGCTCAGCC | |
| | | AATTGTCAAA TAAGAAGCAA TCCAACACGG GCCGTGTGCA | |
| | | GCCGTCAAAA CGAAAGCCAC TGCCTCCCCT CCCACCCTCT | |
| | | GAGGTTGCTG AAGAGAAGAT CCAAGTCAAG GCACTTTATG | |
| | | ATTTTCTGCC CAGAGAACCC TGTAATTTAG CCTTAAGGAG | |
| | | AGCAGAAGAA TACCTGATAC TGGAGAAATA CAATCCTCAC | |
| | | TGGTGGAAGG CAAGAGACCG TTTGGGGAAT GAAGGCTTAA | |
| | | TCCCAAGCAA CTATGTGACT GAAAACAAAA TAACTAATTT | |
| | | AGAAATATAT GAGTGGTACC ATAGAAACAT TACCAGAAAT | |
| | | CAGGCAGAAC ATCTATTGAG ACAAGAGTCT AAAGAAGGTG | |
| | | CATTTATTGT CAGAGATTCA AGACATTTAG GATCCTACAC | |
| | | AATTTCCGTA TTTATGGGAG CTAGAAGAAG TACGGAGGCT | |
| | | GCCATAAAAC ATTATCAGAT AAAAAAGAAT GACTCAGGAC | |
| | | AGTGGTATGT GGCTGAAAGA CACGCCTTTC AATCAATCCC | |
| | | TGAGTTAATC TGGTATCACC AGCACAATGC AGCCGGTCTC | |
| | | ATGACTCGTC TCCGATATCC AGTTGGGCTG ATGGGCAGTT | |
| | | GTTTACCAGC CACAGCTGGG TTTAGCTACG AAAAGTGGGA | |
| | | GATAGATCCA TCTGAGTTGG CTTTTATAAA GGAGATTGGA | |
| | | AGCGGTCAGT TTGGAGTGGT CCATTTAGGT GAATGGCGGT | |
| | | CACATATCCA GGTAGCTATC AAGGCCATCA ATGAAGGCTC | |
| | | CATGTCTGAA GAGGATTTCA TTGAAGAGGC CAAAGTGATG | |
| | | ATGAAATTAT CTCATTCAAA GCTAGTGCAA CTTTATGGAG | |
| | | TCTGTATACA GCGGAAGCCC CTTTACATTG TGACAGAGTT | |
| | | CATGGAAAAT GGCTGCCTGC TTAACTATCT CAGGGAGAAT | |
| | | AAAGGAAAGC TTAGGAAGGA AATGCTACTG AGTGTATGCC | |
| | | AGGATATATG TGAAGGAATG GAATATCTGG AGAGGAATGG | |
| | | CTATATTCAT AGGGATTTGG CGGCAAGGAA TTGTTTGGTC | |
| | | AGTTCAACAT GCATAGTAAA AATTTCAGAC TTTGGAATGA | |
| | | CAAGGTACGT TTTGGATGAT GAGTATGTCA GTTCTTTTGG | |
| | | AGCCAAGTTC CCAATCAAGT GGTCCCCTCC TGAAGTTTTT | |
| | | CTTTTCAATA AGTACAGCAG TAAATCTGAT GTCTGGTCAT | |
| | | TTGGAGTTTT AATGTGGGAA GTTTTTACAG AAGGAAAAAT | |
| | | GCCTTTTGAA AATAAGTCAA ATTTGCAAGT CGTGGAAGCT | |
| | | ATTTCTGAAG GCTTCAGGCT ATATCGCCCT CACCTGGCAC | |
| | | CAATGTCCAT ATATGAAGTC ATGTACAGCT GCTGGCATGA | |
| | | GAAACCTGAA GGCCGCCCTA CATTTGCCGA GCTGCTGCGG | |
| | | GCTGTCACAG AGATTGCGGA AACCTGGTGA CCGGAAACAG | |
| | | AATGCCAACC CAAAGAGTCA TCTTGCAAAA CTGTCATTTA | |
| | | TTGTGAATAT CTTCACCATA TGGGGTCACT TATGGTGAAT | |
| | | ATCTTTCTTC AGAGTTGCTG ACTCTTGAAA ACAGTGCAAA | |
| | | GATCACAGTT TTTAAAAGTT TTAAAAATTT AAGAATATTC | |
| | | ACACAATCGT TTTTCTATGT GTGAGAGGGA TTTGCACACT | |
| | | CTTATTTTTC TGTAAAATAT TTCACATCCC AAATGTGAAG | |
| | | AAGTGAAAAA GACTTCGCAG CAGTCTTCAT TGTGGTGCTC | |
| | | TTCATGATCA TAGCCCCAGG AACCCTTGAG GTTCTTCTTC | |
| | | ACAAGGCTGA GAGTGCTTCC TTCTTGAAGA CGAGTGACAT | |
| | | TCATCACTTC AGTGATCCAT GCATAGAATA TGAAAATAAA | |
| | | TTCTTCCAAC TCATGGGATA AAGGGGACTC CCTTGAAGAA | |
| | | TTTCATGTTT TTGGGCTGTA TAGCTCTTTA CAGAAAATGC | |
| | | ACCTTTATAA ATCACATGAA TGTTAGTATT CTGGAAATGT | |
| | | CTTTTGTTAA TATAATCTTC CCATGTTATT TAACAAATTG | |
| | | TTTTTGCACA TATCTGATTA TATTGAAAGC AGTTTTTTGC | |
| | | ATTCGAGTTT TAAACACTGT TATAAAATGT AGCCAAAGCT | |
| | | CACCTTTGAA CAGATCCCGG TGACATTCTA TTTCCAGGAA | |
| | | AATCCGGAAC CTGATTTTAG TTCTGTGATT TTACACTTTT | |
| | | TACATGTGAG ATTGGACAGT TTCAGAGGCC TTATTTTGTC | |
| | | ATACTAAGTG TCTCCTGTAA TTTTCAGGAA GATGATTTGT | |
| | | TCTTTCCAGA AGAGGAGACA AAAGCAAGAT AGCCAAATGT | |
| | | GACATCAAGC TCCATTGTTT CGGAAATCCA GGATTTTGAA | |
| | | TTCGAGATGA AACAACCAGC AATCACAGTT AAATCTTAAC | |
| | | TTTGCCTGCA CTCTTTGTAG GAATGATCAG AAATTTATCT | |
| | | TTATCATTCT GAGTGCTTCA GGAGTACAAT AGGAAGAAAG | |
| | | ATACTGGAGA AAGCACTAAT GTAATCACCA TGAAGTCTGA | |
| | | CAACAGGAGC CCATTATTTG CGTACTGTCC CACCCTGTAT | |
| | | CATGGTTCTC TGGGAACAAG CTTTATGATT CTCATTAGAG | |
| | | TTTATTTGTT GATTGTCAGT AGTTGCGACT TTTAAATTAT | |
| | | ATTTCCCCCA CTCAAAGAAT GGTATCTTTA TATATCAATG | |
| | | ACATTCAATA AATGTGTATT ATTTCTAATG AGAA | |
| YY2 | NM_206923.3 | ATTAAATAAG AGAAAAAAGC ATAAAAGGAG TAGGCTTTTT | 30 |
| | | TGGCAGATAC TGTTATATGG GATGGACAGG AGGGGAAGAC | |
| | | ATGAAAATCA ACTAAAATGA AATACATAAA AAATCAGTAA | |
| | | AGAGCCTGCT TACGAAAGTG GAAAAACAAC AGCTGGGCGG | |
| | | GGTCGCAGGG TGGCAAACGT ACGCGGGCAC GTGCACGTGC | |
| | | TTTTGGGGCC GACAGACGCG CCAGTTGCCT AGGCGCATGC | |
| | | GTCTGGCTAT CCCAGAAGCA CCTGCGCCTT CCGGCTCGTG | |
| | | CTTTCCTCAG TCTCGCGCCT TTCTCTGCAG CTCGCGCCTT | |
| | | TCTCTGCAGC TCGCGCCTTT CTCTGCAGCT CGCGCCTTTC | |
| | | TCTGCAGCTC GCGCCTTTCT CTGCAGCTCG CCCCTTCCTC | |
| | | TGCAGCTCGC CCCTTCCTCT GCAGCTCCCA CCTCACTCCC | |
| | | CTCAGCGTTC TTTTTCCCAC GGTCTTCCCG TTGCCGCTAA | |
| | | CCTAACTAAC TCTCAGCCAT GGCCTCCAAC GAAGATTTCT | |
| | | CCATCACACA AGACCTGGAG ATCCCGGCAG ATATTGTGGA | |
| | | GCTCCACGAC ATCAATGTGG AGCCCCTTCC TATGGAGGAC | |
| | | ATTCCGACGG AAAGCGTCCA GTACGAGGAT GTGGATGGCA | |
| | | ATTGGATCTA CGGTGGCCAC AACCATCCGC CATTGATGGT | |
| | | GTTGCAGCCG CTCTTCACGA ACACGGGCTA TGGCGACCAC | |
| | | GACCAGGAAA TGCTTATGTT GCAGACACAA GAGGAAGTGG | |
| | | TGGGCTATTG CGACTCAGAC AACCAGCTAG GCAACGACTT | |
| | | GGAGGACCAG TTGGCCCTCC CGGATAGCAT TGAAGACGAG | |
| | | CACTTCCAGA TGACCCTGGC CTCTCTGTCG GCCTCGGCGG | |
| | | CATCAACATC AACATCAACC CAGAGCCGCA GCAAAAAGCC | |
| | | CAGCAAAAAG CCCAGCGGCA AGAGTGCCAC CAGCACTGAG | |
| | | GCCAACCCGG CAGGCAGCAG CTCCAGCCTG GGCACGAGGA | |
| | | AGTGGGAGCA GAAGCAAATG CAGGTCAAAA CGCTGGAGGG | |
| | | TGAGTTTTCC GTGACTATGT GGTCCCCTAA CGATAACAAT | |
| | | GACCAAGGGG CAGTGGGTGA AGGCCAGGCT GAAAACCCAC | |
| | | CTGATTATTC CGAGTACTTG AAAGGGAAGA AACTTCCTCC | |
| | | TGGGGGGTTA CCAGGCATTG ATCTCTCAGA TCCTAAACAG | |
| | | CTGGCAGAAT TTACTAAAGT GAAGCCCAAA AGGTCCAAAG | |
| | | GAGAACCTCC CAAAACAGTC CCTTGCTCTT ATAGCGGCTG | |
| | | CGAAAAGATG TTCCGGGATT ACGCCGCCAT GAGAAAACAT | |
| | | CTCCACATCC ACGGGCCCAG AGTCCACGTA TGTGCAGAAT | |
| | | GTGGCAAAGC TTTTCTTGAG AGCTCAAAGC TGAGACGACA | |
| | | CCAGCTGGTC CACACCGGCG AGAAGCCCTT TCAGTGCACA | |
| | | TTCGAAGGCT GCGGGAAACG CTTTTCCCTT GATTTCAATT | |
| | | TGCGCACACA CTTGCGCATC CACACCGGCG ATAAGCCCTT | |
| | | CGTGTGCCCC TTCGATGTTT GCAACAGGAA GTTCGCTCAG | |
| | | TCAACCAACC TGAAAACCCA CATATTAACG CATGTGAAGA | |
| | | CCAAAAACAA CCCGTGAAAA GGAGAAGACC CCTCTCAGAC | |
| | | TTGGGAATTA TCTTCCAGGA CTGCGGTAGG GAATAAATAT | |
| | | GCCTCTCAAA GCTTTGTATG TTGTTTCTAA GAGTTTTAAA | |
| | | AAAAAATGAA TCCTGCACAT TTAAGGTTCG TGTTTTGTTA | |
| | | GAGTAGTAAA AATAGAATTT AAACGTTTTT AAAAAGGTAA | |
| | | ACCTTGACAT AAGATAATAG TGCTAAGATG CCATAGCTTG | |
| | | TTCTGTAACT ATTTTTGTAA AGTTTGGTCC CAACAGGAGA | |
| | | AAAATTCGTA GACTTCACAT CAAGAGACGG TTCTTACAAA | |
| | | CTGTTTAAAA TGGGACTTTT CACATTCTTA GAAATAGGAA | |
| | | GTTCATTTAT TGTTTACAAT GTTTTTAAAA AACTTGTTAA | |
| | | AAAATTCAAA GTGTTCATGT TTATACTTTT AGGAATATGC | |
| | | TTAATAAGTC TATGTATGGT TTTTCTGGAG GTTGATAACT | |
| | | TTGGGAAAGA TTTACTTTAA AAGAGTGAAC AATTATATGC | |
| | | ATACGTGAAG TATTTTCCTG CTTAAAAAAG TTATATAGGT | |
| | | GTTATTTGTT TTAATCTTGG TTGTAGTCTT GGATGTTAAC | |
| | | ACATCTTGCA TTTTAGCTGT ATTAGGTCAT GTAGTATTGA | |
| | | TATTAGGTGA TTTAATAGTA CTAGTTTAAA CCTATTTTAG | |
| | | TCATTTTATT TTCCCCAAAA TACTACCAGA TGCTGTTGTT | |
| | | TAGTGTAATT TCTTTGCCTG TTCAGTTAAA GTAGTGCTTG | |
| | | CTTGTAGAAT ATATTGTGTA TATGTTGACT TTAACACTTA | |
| | | AGAAGTACAT CCTGTGTAAT AGAAAAAGCA AAATAAAACA | |
| | | CCTCTTCTAA AGAAGGAAAA AAGTAGTTTG CCTATATCAG | |
| | | TACAGAAGTT AGAACTAAAG AAAAGGGGGA GGGTGCTACT | |
| | | GGCATCTGGT GAGTGGAGGG AGATCAGGAA TGCCACTAAA | |
| | | CATCCTACAA TGCACAGACA GCCCCACGAA ACACATAATT | |
| | | ATTTGGCTAA AATGCCAATA GTGTCAGGTG CAGGGGCTCA | |
| | | GGCCTGTAAT GTCAACACTT TGGGAGGCCG AGGTGGGTGG | |
| | | ATCGTTTGAG CTCAGGAATT GAACATCAGC CTGGGCAACA | |
| | | TGGCATAACT CGGTCTCTAC C | |
| ALG9 | NM_001077690.1 | GTCTTTTGTC CCTCGGCGGA CACCGTTTGC CAGCCAAAGC | 31 |
| | | TATGTCTGCG CGCTCACCGA CTTCATAGGG TGCCGAATTC | |
| | | TTTTTTCCCC AGGCTTGCCA TGGCTAGTCG AGGGGCTCGG | |
| | | CAGCGCCTGA AGGGCAGCGG GGCCAGCAGT GGGGATACGG | |
| | | CCCCGGCTGC GGACAAGCTG CGGGAGCTGC TGGGCAGCCG | |
| | | AGAGGCGGGC GGCGCGGAGC ACCGGACCGA GTTATCTGGG | |
| | | AACAAAGCAG GACAAGTCTG GGCACCTGAA GGATCTACTG | |
| | | CTTTCAAGTG TCTGCTTTCA GCAAGGTTAT GTGCTGCTCT | |
| | | CCTGAGCAAC ATCTCTGACT GTGATGAAAC ATTCAACTAC | |
| | | TGGGAGCCAA CACACTACCT CATCTATGGG GAAGGGTTTC | |
| | | AGACTTGGGA ATATTCCCCA GCATATGCCA TTCGCTCCTA | |
| | | TGCTTACCTG TTGCTTCATG CCTGGCCAGC TGCATTTCAT | |
| | | GCAAGAATTC TACAAACTAA TAAGATTCTT GTGTTTTACT | |
| | | TTTTGCGATG TCTTCTGGCT TTTGTGAGCT GTATTTGTGA | |
| | | ACTTTACTTT TACAAGGCTG TGTGCAAGAA GTTTGGGTTG | |
| | | CACGTGAGTC GAATGATGCT AGCCTTCTTG GTTCTCAGCA | |
| | | CTGGCATGTT TTGCTCATCA TCAGCATTCC TTCCTAGTAG | |
| | | CTTCTGTATG TACACTACGT TGATAGCCAT GACTGGATGG | |
| | | TATATGGACA AGACTTCCAT TGCTGTGCTG GGAGTAGCAG | |
| | | CTGGGGCTAT CTTAGGCTGG CCATTCAGTG CAGCTCTTGG | |
| | | TTTACCCATT GCCTTTGATT TGCTGGTCAT GAAACACAGG | |
| | | TGGAAGAGTT TCTTTCATTG GTCGCTGATG GCCCTCATAC | |
| | | TATTTCTGGT GCCTGTGGTG GTCATTGACA GCTACTATTA | |
| | | TGGGAAGTTG GTGATTGCAC CACTCAACAT TGTTTTGTAT | |
| | | AATGTCTTTA CTCCTCATGG ACCTGATCTT TATGGTACAG | |
| | | AACCCTGGTA TTTCTATTTA ATTAATGGAT TTCTGAATTT | |
| | | CAATGTAGCC TTTGCTTTGG CTCTCCTAGT CCTACCACTG | |
| | | ACTTCTCTTA TGGAATACCT GCTGCAGAGA TTTCATGTTC | |
| | | AGAATTTAGG CCACCCGTAT TGGCTTACCT TGGCTCCAAT | |
| | | GTATATTTGG TTTATAATTT TCTTCATCCA GCCTCACAAA | |
| | | GAGGAGAGAT TTCTTTTCCC TGTGTATCCA CTTATATGTC | |
| | | TCTGTGGCGC TGTGGCTCTC TCTGCACTTC AGAAATGTTA | |
| | | CCACTTTGTG TTTCAACGAT ATCGCCTGGA GCACTATACT | |
| | | GTGACATCGA ATTGGCTGGC ATTAGGAACT GTCTTCCTGT | |
| | | TTGGGCTCTT GTCATTTTCT CGCTCTGTGG CACTGTTCAG | |
| | | AGGATATCAC GGGCCCCTTG ATTTGTATCC AGAATTTTAC | |
| | | CGAATTGCTA CAGACCCAAC CATCCACACT GTCCCAGAAG | |
| | | GCAGACCTGT GAATGTCTGT GTGGGAAAAG AGTGGTATCG | |
| | | ATTTCCCAGC AGCTTCCTTC TTCCTGACAA TTGGCAGCTT | |
| | | CAGTTCATTC CATCAGAGTT CAGAGGTCAG TTACCAAAAC | |
| | | CTTTTGCAGA AGGACCTCTG GCCACCCGGA TTGTTCCTAC | |
| | | TGACATGAAT GACCAGAATC TAGAAGAGCC ATCCAGATAT | |
| | | ATTGATATCA GTAAATGCCA TTATTTAGTG GATTTGGACA | |
| | | CCATGAGAGA AACACCCCGG GAGCCAAAAT ATTCATCCAA | |
| | | TAAAGAAGAA TGGATCAGCT TGGCCTATAG ACCATTCCTT | |
| | | GATGCTTCTA GATCTTCAAA GCTGCTGCGG GCATTCTATG | |
| | | TCCCCTTCCT GTCAGATCAG TATACAGTGT ACGTAAACTA | |
| | | CACCATCCTC AAACCCCGGA AAGCAAAGCA AATCAGGAAG | |
| | | AAAAGTGGAG GTTAGCAACA CACCTGTGGC CCCAAAGGAC | |
| | | AACCATCTTG TTAACTATTG ATTCCAGTGA CCTGACTCCC | |
| | | TGCAAGTCAT CGCCTGTAAC ATTTGTAATA AAGGTCTTCT | |
| | | GACATGAATA CTGGAATCTG GGTGCTCTGG GCTAGTCAAA | |
| | | GTCTATTTCA AAGTCTAATC AAAGTCACAT TTGCTCCCTG | |
| | | TGTGTGTCTC TGTTCTGCAT GTAAACTTTT TGCAGCTAGG | |
| | | CAGAGAAAGG CCCTAAAGCA CAGATAGATA TATTGCTCCA | |
| | | CATCTCATTG TTTTTCCTCT GTTCAATTAT TTACTAGACC | |
| | | GGAGAAGAGC AGAACCAACT TACAGGAAGA ATTGAAAATC | |
| | | CTGGTACTGG ATGGCTGTGA TAAGCTGTTC TCCACACTCT | |
| | | GGCCTGGCAT CTGAGAACTA GCAAGCCTCT CTTAGGCCAT | |
| | | ATGGGCTTCT CCACCAAAGC TGTTTGGCAG CTCCTAGCAG | |
| | | ACCTTCTTAT TGAAATCCTC ATGCTGAAAA TGAACACAGC | |
| | | CTAGTTGCCA ACCCACATGT CCTTTTCACC TCCAGCAAGA | |
| | | CTAAGCTTCT TTAAAGCACT TCACAGGACT AGGACCCTGT | |
| | | CCTGGAGCTA TCTCAGGAAA AAGGTGACCA TTTGAGGAAC | |
| | | TGTGACCTAA TTTTATTATA ATGATGCCTC TAATTTTCAT | |
| | | TTCCTTTACA ACCAACTGTA ACTATAAGGT TGTATTGCTT | |
| | | TTTTGTTCAG TTTTAGCATG CTATTTTTTG AATTCTAGAC | |
| | | TCCTCCATGT GAAGATATCA ACAGACAAAA CTACAACTGT | |
| | | ATAGGACATA TTTGGAGAAA ATTCTATCAA TTGATACATT | |
| | | TGGATGACAT CACATTTTTA AGTAATGTAA TCTGAGGCCA | |
| | | TTGCTGAGGA AATTAAGAAT TTTCCTTTTT TTTTAACCAC | |
| | | CCCCAGTGAA AAGGATCAGT GTATATTTAT AGCACCTATT | |
| | | TTTTAGTTCT GTCTGTTGTG AGGCACATCC TGCATGGGGC | |
| | | ACTTCTAGTC AAATAGGCAA TGATAAGGAC CTAATTAAAA | |
| | | TGTGATAAGT GTATACTATT ACTTTAAAAG CCTTTACAGT | |
| | | CAGTACTTCA GTTTACAAGG CACTTTCACA GCATCTCGTT | |
| | | TGATCCTCAC AGTCACAACA TGTGGTAGAC AAGGCAGGTG | |
| | | ATTTTTATCC CCATTTTACA GATAAGGAAA CAGGCTGCGG | |
| | | GTGGGGAGTG AGGGGAGGTA AAGATAGTTA GTTGCCTAAG | |
| | | GTCACACAGC CAGTAAGTAA TAGAGCTGGG ACTGGAACCC | |
| | | AGGTTTCCTT ACTCTCATCT ATTGCTCCTC CATATTCCTC | |
| | | ACTCAACCAT GAAAACATTA CTTGAAAGGA CTGATGAGGT | |
| | | TAACCAGAGA CCTAACTGAT ATTGTAACTT TCTATTTTAA | |
| | | GGAAGAATTG TGTCTGTATT TGAGTTCTTT GGAGCCTCCA | |
| | | GTCTGCCTGT GTGTTAGACC AGCACAGCAG TGCTGTGTGA | |
| | | TGCAGCCTGA CCTGTGGCAG GAAAGTAGTG CTTCTGTTTG | |
| | | GAAGTCATGT TCTTTTGCAG CCACACAGGA TCCAAATATC | |
| | | AGTACTATTC CTGTAGTCAA TCTGGGGTCA CATTATAGGT | |
| | | GCCTTATTTC CCTAAGGGTA ACTGATCTGA ATATCTGCAA | |
| | | ATAGGATGAA TCTATTTTTC AGAAGTTCCA TCTTTCATTT | |
| | | TTCTTTTTTT TTTTGAGACA GAGTCTCATT CTGTCGCCCA | |
| | | TGCTGGAGTG CAGTGGCGCG ATCTCGGCTC GCTGCAACCT | |
| | | CTGCCTCCCA GGTTGAAGCA ATTCTCATGC CTCAGCCACC | |
| | | CGAGTAGCTG GGATTACAGG CATGCGCCAT CATGCCCAGC | |
| | | TAATTTATGT ATTTTTAGTA GAGTTGGAGT TTCACCATGT | |
| | | TGGCCAGGCT GGTCTTGGAC TCCTGACCTC AGGTCATCCA | |
| | | CCCGCCTCAG CCTCCCAAAG TGCTGGTATT ACAGGCGTGA | |
| | | GCCACCGCAC CCAGCCCCAT CTTTCATTTT CAAAGAGAAG | |
| | | GGCATTCTAA TAGGAACTGG TGCCAAGAGA GAAGAAAAGA | |
| | | AGTGATAACA GAAGAAATGG CTAGTTACAA TATTAAAAAG | |
| | | CTCCTCTTTG AGATCTCCTC TGCAGGAATA TCAGAGACGG | |
| | | AGTTGAAGCG CTGGAGAGGT AATAGGTCTA GACAGTACAG | |
| | | AACAATAACT GGGGAGTGTG TGAGGATAGA CTGGGCTCCC | |
| | | CCTTGCTTGA AAGATCTCTG GCATTTAATT CTCAATTCTT | |
| | | GATTACTATT TTCCAGTGTA AAACTAGCAC ATATGATCTG | |
| | | ACTACAGGAC AGAGAATTTT AAGTGAAACA TTTGCCTTAC | |
| | | TTGCAGTAAT AATGTGCTGT TCTTCACAGT AGCTAAGGCC | |
| | | CTCTATGTTT CCCAGAGGTA AATAAGAATC CAGGAATGGA | |
| | | GGTCCATCTG TGATGAATGG CTTTTTTCTA ATCAAAGTAG | |
| | | TATAATGCTG TTTTATCTGT TTTGTCATCT TGTTTTTTTT | |
| | | TTTTTTTAAA AAAACAAAAC CTTAATTATA ATATAGCGCA | |
| | | AAGAAAGGCC AGGACTGATG CAGGGATTCC TTGGAAATAT | |
| | | CAGTTCCTAT CACTTTTAAA ACCTGATTTT GGATCTCTCT | |
| | | GTTCTATGTA TGTCTTTAGT GAGAGCACAA TACATGGCAG | |
| | | AACGCTGTGC CAAATGTTAT AGGTAAGGAA TATAGAAATG | |
| | | AATGTTTTTT GTTGTGAAGG TGTTTTCATG TGATATTTTA | |
| | | TAAACACATT TTAAAAAATC TCCATCACTT TTTAGTATAG | |
| | | GAAGGATAGC TTTGCCTGGG AAAAACAGTT TCAACACACC | |
| | | TGCTCAGAGT AGCAGTTCTC CCTCAAAAAA GCAGTGTTCA | |
| | | GCCTGCACTG ACTGTTCTGC TTGCCAAAAG GAGGAAGCAT | |
| | | GCAAGATACT TATTTCTCCA TAGATTGTGG AGTATAGAGG | |
| | | GATGTGGGAC TACAGATTAT TATTTTTTTT CCCCGAGACA | |
| | | GAGTCTTGCT CTGTCGCCCA GGTTGGAACA CAATGGCACG | |
| | | ACCTCAGCTC ACTGCAACCT CTGTCTCCCG GGTTCAAGCA | |
| | | ATTCTCCTGC TTCAGCCTCC TGAGTAGCTG GGATTACAGG | |
| | | CACACACCAC CACCGCACTC AGCTAATTTT TGTATTTTTA | |
| | | GTAGAGGTGG GGTTTTACCA TGTTGGCCAG GCTGGTCTTA | |
| | | AACTCCTGAC CTTGTAATCA TCCCGCCTCG GCCTCCTAAA | |
| | | GTGCTAGGAT TACAGGCATG AGCCACCGCA CCCGGCCCAG | |
| | | ATAATTTTTA ATAGCCTTTG ATCATGGGGT GAGTGAGGGA | |
| | | GTAGGTATAC TTGGCAAATG CATGGTTCTC TGATTTCTAG | |
| | | CTCTAAAGCA GCCTTATCTG AATCCCCAAA TCTTGTGATG | |
| | | CTGAGTACCA TTACTGAACC AGTCTGCACG GTAGGCATCT | |
| | | GCTACCAAAA TTTACCTCCT ACCTGGTAGG TGTCATCTGA | |
| | | TAAGAAAGAA GACAGGTTAT TTTAATTTTT TGAGATAATC | |
| | | ACAGAAAATT GCAGCCCATA CTCTTTATTA CCGAATTCAA | |
| | | GTTTGGAAAT AGACCCTTTG TTTTAAATCA TGATGGGTCT | |
| | | TTATCCCAAT CATTTATCTG GGTCATTTTT CCAACTTTGG | |
| | | AGTTCTAGGA AAGAACCTTG AAAACCTGAT ATGATTCTGC | |
| | | AGCATGAGGT CTACGGTGAC CATTTGGGCA AAGCTCCAGT | |
| | | GGCAATCATT TATTGTGTTT TGCATTTCCT GGGATTTATT | |
| | | GAAATAAGAA TTCACTGTGA TTATGTAGTC TTCTGGCTAG | |
| | | TATCAGGCAG CTCTGCTTTT AATTTGGTTA ATTTTATTTT | |
| | | CTCTGAAGAG GGAGAAGAGG TACAATTTAA TCTTGGCCTC | |
| | | CACAAGCATA TTAAAGCTCA CGTGTTAATC AGTGCATTCT | |
| | | TATGCTCCTA CATTAAATGC CTTGGGTAAA TGGATAAATG | |
| | | GACATGTGCC CAGCTTTAAT TTTTTTTGCA ACAGAAAGAT | |
| | | CAGACTTCCG TATGGCATCG TTGGATTTCA GAGGCTTTCT | |
| | | GGTGTATCTG TAAATCTGAA TGTTGCCTTC TGCCAGTCTG | |
| | | TATAACCAGG TGATTCATGC TGCAAATGAA ATCAGGAAGC | |
| | | AGTAAAGTGT TAAAGCAAGA GTATTGTCCA ATTCACTTGT | |
| | | CTTCCTGATC CTTGTACTTT ATTTCACGTG TCGGTGTTTA | |
| | | CATTACATAC TTATATTTCC TGTGAAAGAA AGAGTTAAAT | |
| | | AAATTGTAGC AGTTTGA | |
| SEPN | NM_031475.2 | AGCGGAGCGC CAGGCAGCGC GGAGCGGAGG CCAGGCCCAC | 32 |
| | | AGCCGCTCCG CCTCCCGGCC CGCAGATCCC CGACGGCCGC | |
| | | ACCGCGGGCT CCTCTGGCCC GCAAGAACAC GTGCATGGCG | |
| | | TCCTGGGGAA GGCGCTGAGT GCGGAGTCGC GGCGCCGCAC | |
| | | GCGGCACCAT GGCCCTGGAG CAGGCGCTGC AGGCGGCGCG | |
| | | GCAGGGCGAG CTGGACGTGC TGAGGTCGCT GCACGCCGCA | |
| | | GGCCTCCTGG GGCCCTCGCT GCGCGACCCG CTGGACGCGC | |
| | | TGCCCGTGCA CCACGCGGCC CGCGCTGGGA AGCTGCACTG | |
| | | TCTGCGCTTC CTGGTGGAGG AAGCCGCCCT CCCCGCCGCG | |
| | | GCCCGCGCCC GCAACGGCGC CACACCGGCC CACGACGCCT | |
| | | CCGCCACCGG CCACCTCGCC TGCCTGCAGT GGCTGCTGTC | |
| | | GCAGGGCGGC TGCAGAGTGC AGGACAAAGA CAATTCTGGT | |
| | | GCCACAGTCT TGCATCTGGC TGCCCGCTTC GGCCACCCCG | |
| | | AGGTGGTGAA CTGGCTCTTG CATCATGGCG GTGGGGACCC | |
| | | CACCGCGGCC ACAGACATGG GCGCCCTGCC TATCCACTAC | |
| | | GCTGCCGCCA AAGGAGACTT CCCCTCCCTG AGGCTTCTCG | |
| | | TCGAGCACTA CCCTGAGGGA GTGAATGCCC AAACCAAGAA | |
| | | CGGTGCCACG CCCCTGTACC TGGCGTGCCA GGAGGGCCAC | |
| | | CTGGAGGTGA CCCAGTACCT GGTGCAGGAA TGCGGCGCAG | |
| | | ACCCGCACGC GCGCGCCCAC GACGGCATGA CCCCGCTGCA | |
| | | CGCCGCGGCG CAGATGGGCC ACAGCCCAGT CATCGTGTGG | |
| | | TTGGTGAGCT GCACCGACGT GAGCCTGTCC GAGCAGGACA | |
| | | AAGACGGCGC CACCGCCATG CACTTCGCGG CGAGCCGCGG | |
| | | CCACACCAAG GTGCTCAGCT GGCTGCTGCT GCACGGCGGG | |
| | | GAGATCTCGG CTGACCTGTG GGGCGGGACC CCGCTGCACG | |
| | | ACGCCGCCGA GAACGGGGAG CTAGAGTGCT GCCAGATCCT | |
| | | GGTAGTGAAC GGCGCGGAGC TGGACGTCCG CGACCGCGAC | |
| | | GGGTACACGG CCGCCGACCT GTCGGACTTC AACGGCCACA | |
| | | GCCACTGCAC CCGCTACCTG CGCACGGTGG AGAACCTGAG | |
| | | CGTGGAGCAC CGCGTGCTTT CCCGGGATCC ATCCGCAGAG | |
| | | CTGGAGGCTA AGCAGCCGGA TTCAGGCATG TCCTCACCCA | |
| | | ATACCACGGT GTCGGTCCAG CCGCTGAACT TTGACCTCAG | |
| | | CTCGCCTACC AGCACCCTCT CCAACTACGA CTCCTGCTCC | |
| | | TCCAGCCACT CCAGCATCAA GGGCCAGCAC CCTCCATGTG | |
| | | GGCTTTCCAG CGCTAGAGCT GCAGACATAC AGAGCTACAT | |
| | | GGACATGCTG AACCCGGAGC TGGGCCTGCC TCGGGGCACG | |
| | | ATTGGGAAGC CCACACCCCC ACCACCCCCA CCCAGCTTCC | |
| | | CCCCGCCACC CCCGCCCCCA GGCACCCAAC TGCCCCCACC | |
| | | CCCACCTGGC TACCCAGCTC CCAAGCCTCC TGTAGGACCA | |
| | | CAGGCAGCTG ACATCTACAT GCAGACCAAG AACAAACTCC | |
| | | GCCACGTGGA GACAGAGGCC CTCAAGAAGG AGCTGAGCTC | |
| | | CTGTGACGGC CACGACGGGC TGCGGAGGCA GGACTCCAGC | |
| | | CGCAAGCCCC GCGCCTTCAG CAAGCAGCCC AGCACGGGGG | |
| | | ACTACTACCG GCAGCTGGGC CGCTGCCCCG GCGAGACGCT | |
| | | GGCCGCACGC CCGGGCATGG CGCACAGCGA GGAGGTGCGT | |
| | | GCCCGCCAGC CCGCGCGCGC CGGCTGCCCG CGCCTCGGCC | |
| | | CTGCCGCCCG CGGCTCACTC GAAGGCCCCT CCGCTCCCCC | |
| | | GCAGGCGGCG CTGCTTCCTG GGAACCATGT TCCTAACGGC | |
| | | TGCGCCGCGG ACCCCAAGGC GTCCAGGGAG CTGCCACCGC | |
| | | CGCCCCCACC GCCGCCGCCG CCCCTGCCGG AGGCCGCGAG | |
| | | TTCGCCACCG CCGGCCCCGC CTCTGCCCCT CGAGAGCGCT | |
| | | GGCCCTGGCT GCGGGCAGCG CCGCTCCTCC TCGTCCACCG | |
| | | GCAGCACCAA GTCTTTCAAC ATGATGTCCC CGACGGGCGA | |
| | | CAACTCGGAG CTACTGGCTG AGATTAAGGC AGGCAAGAGC | |
| | | CTGAAGCCGA CGCCCCAGAG CAAGGGGCTG ACCACAGTGT | |
| | | TCTCAGGCAT CGGGCAGCCG GCCTTCCAGC CCGATTCGCC | |
| | | GCTGCCTTCT GTGTCACCTG CACTGTCACC AGTCCGGAGC | |
| | | CCCACACCGC CAGCTGCGGG GTTTCAGCCG CTGCTCAATG | |
| | | GAAGCTTGGT TCCCGTGCCG CCCACTACTC CTGCGCCGGG | |
| | | AGTGCAGCTG GACGTGGAGG CTCTCATCCC CACGCACGAT | |
| | | GAGCAGGGCC GGCCCATCCC CGAGTGGAAG CGCCAGGTGA | |
| | | TGGTGCGCAA GATGCAGCTG AAGATGCAGG AGGAGGAGGA | |
| | | GCAGAGGCGG AAGGAGGAGG AGGAGGAGGC CCGGCTGGCC | |
| | | AGCATGCCCG CCTGGAGGCG GGACCTCCTG CGGAAGAAGC | |
| | | TGGAAGAAGA GAGGGAGCAG AAGCGGAAAG AGGAGGAGCG | |
| | | ACAGAAGCAG GAGGAGCTGC GGCGGGAGAA GGAACAGTCA | |
| | | GAGAAGCTGC GGACGCTGGG CTACGATGAG AGCAAGCTGG | |
| | | CGCCCTGGCA GCGACAGGTC ATCCTGAAGA AGGGGGACAT | |
| | | CGCTAAGTAC TAGAGGCCGC AGACTCCTGT CCGCAGCCTC | |
| | | GCAGCTCCGT GGGGCCCTCC GCCCCAGCCC CAGCCAGCCA | |
| | | GGCCCTGGTG GAAAGGCTGG GAGCCGCACA GCCCTCCCCT | |
| | | CCTGCGCTGG AAACCCTCCC TGACCCCCAC CCTGGCCCCC | |
| | | CGTATCCCCA GCCCTTGGCA ACACTGGAGT GCACACGCCG | |
| | | CCACGGTTGC CCAGAAAAAG TGCCCAAGCT GCTGACGCAA | |
| | | ACAACAACAA ATGCTGCTTA TTTGCATGCC GACTTACATA | |
| | | TATTTGCATG TTCGTTGACT ATCAAAGAGT GCAGAGCTCT | |
| | | CCCCAGCCCC GTGGGTGGTG ACTTTGTTTT CCTGCGGGGC | |
| | | TCAGCCCCCT CCAGGATGCA GCCCCCTCCC CCGCACCCCG | |
| | | GAACCGGCGT CGCTGGCGCA TCCTGGGTGG AGGCAGGCCC | |
| | | CGAGCTCGGG GAAGGGGTTT TCCCTTCCTC TCTGACCCAG | |
| | | ATCTGCGCGC GGCCTAGCCC GGGCCTCATT TCTTATCCCC | |
| | | GCCAAGGGTT TCCTCTCAGT CATTTGTTTA CCAGAAACAT | |
| | | GAAAACTGCC TGTCTGGCCG GGCCGCACTT GTGGCCCCCG | |
| | | GGACCCCACC TCTGGCCCCA CCTCCCTCAA GTCTGCGCCC | |
| | | CGTCCCCAGC CAGACCCACT CGCTGCCGGG ACCCTTTCAC | |
| | | TGCCCCGGTG GAGTGAATAG AGGATGAGGG GCCCTGACCC | |
| | | TGTGTCTCCA ACTGCTGCAC CCCATCCCGA CCCTGTCTCC | |
| | | GCCACCTCGC AGCCCCATTA AAGCGCTCTC ATCTGGGCTC | |
| | | CGGTTCACTC A | |
| YWHAQ | NM_006826.3 | TTGGGCGGTG GACCGCCCCT CGGCCCCGGG GTAGGCTGAC | 33 |
| | | ACGGGAGGGT CCTCAGCTAA AGCCAAAAGC AGATCAAAGT | |
| | | GGTGGGACTC GCGTCGCGGC CGCGGAGACG TGAAGCTCTC | |
| | | GAGGCTCCTC CCGCTGCGGG TCGGCGCTCG CCCTCGCTCT | |
| | | CCTCGCCCTC CGCCCCGGCC CCGGCCCCGC GCCCGCCATG | |
| | | GAGAAGACTG AGCTGATCCA GAAGGCCAAG CTGGCCGAGC | |
| | | AGGCCGAGCG CTACGACGAC ATGGCCACCT GCATGAAGGC | |
| | | AGTGACCGAG CAGGGCGCCG AGCTGTCCAA CGAGGAGCGC | |
| | | AACCTGCTCT CCGTGGCCTA CAAGAACGTG GTCGGGGGCC | |
| | | GCAGGTCCGC CTGGAGGGTC ATCTCTAGCA TCGAGCAGAA | |
| | | GACCGACACC TCCGACAAGA AGTTGCAGCT GATTAAGGAC | |
| | | TATCGGGAGA AAGTGGAGTC CGAGCTGAGA TCCATCTGCA | |
| | | CCACGGTGCT GGAATTGTTG GATAAATATT TAATAGCCAA | |
| | | TGCAACTAAT CCAGAGAGTA AGGTCTTCTA TCTGAAAATG | |
| | | AAGGGTGATT ACTTCCGGTA CCTTGCTGAA GTTGCGTGTG | |
| | | GTGATGATCG AAAACAAACG ATAGATAATT CCCAAGGAGC | |
| | | TTACCAAGAG GCATTTGATA TAAGCAAGAA AGAGATGCAA | |
| | | CCCACACACC CAATCCGCCT GGGGCTTGCT CTTAACTTTT | |
| | | CTGTATTTTA CTATGAGATT CTTAATAACC CAGAGCTTGC | |
| | | CTGCACGCTG GCTAAAACGG CTTTTGATGA GGCCATTGCT | |
| | | GAACTTGATA CACTGAATGA AGACTCATAC AAAGACAGCA | |
| | | CCCTCATCAT GCAGTTGCTT AGAGACAACC TAACACTTTG | |
| | | GACATCAGAC AGTGCAGGAG AAGAATGTGA TGCGGCAGAA | |
| | | GGGGCTGAAA ACTAAATCCA TACAGGGTGT CATCCTTCTT | |
| | | TCCTTCAAGA AACCTTTTTA CACATCTCCA TTCCTTATTC | |
| | | CACTTGGATT TCCTATAGCA AAGAAACCCA TTCATGTGTA | |
| | | TGGAATCAAC TGTTTATAGT CTTTTCACAC TGCAGCTTTG | |
| | | GGAAAACTTC ATTCCTTGAT TTGTGTTTGT CTTGGCCTTC | |
| | | CTGGTGTGCA GTACTGCTGT AGAAAAGTAT TAATAGCTTC | |
| | | ATTTCATATA AACATAAGTA ACTCCCAAAC ACTTATGTAG | |
| | | AGGACTAAAA ATGTATCTGG TATTTAAGTA ATCTGAACCA | |
| | | GTTCTGCAAG TGACTGTGTT TTGTATTACT GTGAAAATAA | |
| | | GAAAATGTAG TTAATTACAA TTTAAAGAGT ATTCCACATA | |
| | | ACTTCTTAAT TTCTACATTC CCTCCCTTAC TCTTCGGGGG | |
| | | TTTCCTTTCA GTAAGCAACT TTTCCATGCT CTTAATGTAT | |
| | | TCCTTTTTAG TAGGAATCCG GAAGTATTAG ATTGAATGGA | |
| | | AAAGCACTTG CCATCTCTGT CTAGGGGTCA CAAATTGAAA | |
| | | TGGCTCCTGT ATCACATACG GAGGTCTTGT GTATCTGTGG | |
| | | CAACAGGGAG TTTCCTTATT CACTCTTTAT TTGCTGCTGT | |
| | | TTAAGTTGCC AACCTCCCCT CCCAATAAAA ATTCACTTAC | |
| | | ACCTCCTGCC TTTGTAGTTC TGGTATTCAC TTTACTATGT | |
| | | GATAGAAGTA GCATGTTGCT GCCAGAATAC AAGCATTGCT | |
| | | TTTGGCAAAT TAAAGTGCAT GTCATTTCTT AATACACTAG | |
| | | AAAGGGGAAA TAAATTAAAG TACACAAGTC CAAGTCTAAA | |
| | | ACTTTAGTAC TTTTCCATGC AGATTTGTGC ACATGTGAGA | |
| | | GGGTGTCCAG TTTGTCTAGT GATTGTTATT TAGAGAGTTG | |
| | | GACCACTATT GTGTGTTGCT AATCATTGAC TGTAGTCCCA | |
| | | AAAAAGCCTT GTGAAAATGT TATGCCCTAT GTAACAGCAG | |
| | | AGTAACATAA AATAAAAGTA CATTTTATAA ACCATTTACT | |
| | | ATGGCTTTGT AACAATTGCA TACCCATATT TTAAGGGACA | |
| | | GGTGAATTTA CTACTTTCTA AAGTTTATTG ATACTTCCCT | |
| | | TTTATGTAAA ATGTAGTAGT GATACCTATA TTTCCACATT | |
| | | GTGCATTGTG ACACACTTGT CTAGGGATGC CTGGAAGTGT | |
| | | ATAAAATTGG ACTGCATTTC TTAGAGTGTT TTACTATAGA | |
| | | TCAGTCTCAT GGGCCATCTC TTCCTCAGAT GTAAATGATA | |
| | | TCTGGTTAAG TGTTATATGG AATAAAGTGG ACATTTTAAA | |
| | | ACTAGCAAAG TTAAAAAAAA AAAAAAAAAA AA | |
| VPS37A | NM_001145152.1 | GCAGAGGGGG CGGAGAGCGC CCCCGGGGGC GGGGCACGCA | 34 |
| | | AGTGACGGCG GCGCGGGTGG TGGAGCGCTG GGCGGCCAGG | |
| | | CTCCCTGGCT GGCCGGTTTG GGCGTCTGGG CCGTGAAGGT | |
| | | GGGACCTCCT GTTCCGGGCC GCAAGTTTCC CTCTCCAGCC | |
| | | GCCCGCCGTT CGTAGCATGT CCCCCAGAAC TCGGGGAGCG | |
| | | CAGGCAGGAC AGGCTTAGAG AAGACGCGGT CCCCAGCGCT | |
| | | TGGGCCACGG ACGTCCCACC CCGCTCCTCT GTCGCTGGAG | |
| | | AACCGCCGGG CCGAGCCACT GGGAGAAGCA GGCCAGAGCC | |
| | | TTCCAGGGCC TCCGGCCCGT GGACCCGAGG AGGATGAGCT | |
| | | GGCTTTTTCC CCTGACCAAG AGCGCCTCCT CCTCCGCGGC | |
| | | TGGGTCCCCC GGTGGCCTCA CCAGCCTCCA GCAGCAGAAG | |
| | | CAGCGCCTGA TCGAGTCCCT CCGGAACTCA CACTCCAGAT | |
| | | TGCTTCCTCC ACAGTTTCCT CAGGAAAAAC CAGTGATCAG | |
| | | TGTTTATCCA CCAATACGAC ATCACTTAAT GGATAAACAA | |
| | | GGAGTGTATG TTACCTCTCC ATTAGTAAAC AATTTTACAA | |
| | | TGCACTCAGA TCTTGGAAAA ATTATTCAGA GTCTGTTGGA | |
| | | TGAGTTTTGG AAGAATCCTC CAGTTTTAGC TCCTACTTCA | |
| | | ACAGCATTTC CTTATCTATA CAGTAACCCA AGTGGGATGT | |
| | | CTCCTTATGC TTCTCAGGGT TTTCCATTTC TTCCTCCATA | |
| | | TCCTCCACAA GAAGCAAACA GGAGTATCAC TTCTTTATCT | |
| | | GTTGCTGACA CTGTTTCTTC TTCAACAACA AGTCATACCA | |
| | | CAGCCAAGCC TGCCGCTCCT TCATTTGGTG TCCTTTCAAA | |
| | | TCTGCCATTA CCCATTCCCA CAGTGGATGC TTCAATACCG | |
| | | ACAAGCCAAA ATGGTTTTGG GTACAAGATG CCAGATGTCC | |
| | | CTGATGCATT TCCAGAACTC TCAGAACTAA GTGTGTCACA | |
| | | ACTCACAGAT ATGAATGAAC AAGAGGAGGT ATTACTAGAA | |
| | | CAGTTTCTGA CTTTGCCTCA ACTAAAACAA ATTATTACCG | |
| | | ACAAAGATGA CTTAGTAAAA AGTATTGAGG AACTAGCAAG | |
| | | AAAAAATCTC CTTTTGGAGC CCAGCTTGGA AGCCAAAAGA | |
| | | CAAACTGTTT TAGATAAGTA TGAATTACTT ACACAGATGA | |
| | | AGTCCACTTT CGAAAAGAAG ATGCAAAGGC AGCATGAACT | |
| | | TAGTGAGAGC TGTAGTGCAA GTGCCCTTCA GGCAAGATTG | |
| | | AAAGTAGCTG CACATGAAGC TGAGGAAGAA TCTGATAATA | |
| | | TTGCAGAAGA CTTCTTGGAG GGAAAGATGG AAATAGATGA | |
| | | TTTTCTCAGT AGCTTCATGG AAAAGAGAAC AATTTGCCAC | |
| | | TGTAGAAGAG CCAAGGAAGA GAAACTTCAG CAGGCGATAG | |
| | | CAATGCACAG CCAATTTCAT GCTCCACTAT AGATTTTCCT | |
| | | GGAAACATGA ACTGCCAAGA GAGGAATGGG ACACAAAACC | |
| | | AAACACTGTT TTATATTTAT GGTTTGCAAA CTGGCATTTC | |
| | | ATCAGTGGCT AAATTCACAG ATATCCTATA TAGATTGTAT | |
| | | ACAGAACTGA GACTGATTTT GTACCGATTA GAATGATTGC | |
| | | TATGATCTTT GAGAAATTTT TCTGCACTAT TTGCACTGAA | |
| | | ATGTTTATTT ATTGTTGATA AATTGTATCA TATTTAAGTT | |
| | | CCACTGCTGT TCCTCTTACC TTGATTAAAT GCCTATGCAT | |
| | | GTACTTTTAG CTAGTTTTTA ATATTTTATA AAACTTCATT | |
| | | TAAATTTGTA TTTTTAACTT GAAGTTCCAT TTCTTTATCA | |
| | | AGGATGGTAT TTAGATTTTT TTCCTCTTAA CCTTTTTTCA | |
| | | AAAACTATTT TCAACTGTGA GGAAACCCTT ATTTTTCTTT | |
| | | CTTTGTGGAT AAAACTTTCA AAAGCAATTT AAGATATTCA | |
| | | TAGTGTTAGG AAACACCAAA CCTGCCTATG TGCCATCTCA | |
| | | CAAAAGAAAC TTTTAATACC TACAATAAAT CAAAAGAATA | |
| | | AACCAGCTGT TCTTATATAT TGTTTCATTT TTAAAACTAA | |
| | | AGATGCATTT AAGAAGCAAT ACAAGTAAAT ATTTTACCTA | |
| | | ATAGGAAAAA AAAAAGTTGC CTTTCATTTA AACCATTCCA | |
| | | ACAGAAATTC TTATGCTAAT TTAAAACATA TATATATCTG | |
| | | GTAGGTTTGT GGTTGGATAG GTTTTCTAAA TTCCTAATGT | |
| | | TAAAAACAAT CTTTATGTTA ATATACACTA AATCTATACA | |
| | | CAAAAAAAGT CAGTGAACTT TTCTGACCTT TACTGTGAGT | |
| | | TACCTTTTCC TAAGAGGAAA GCTATAGTAA TAAGTAAAAT | |
| | | TTAATTTTTA GGCAATCCTG ATTTTTAATG AATTTAATTG | |
| | | AGTGTTCTTG TATACTACAT TGAGCAGTTT GCTTCTATAC | |
| | | CGTGTCACAA AATTCATGTA TTTCTTGAGA AGCCCTAAAA | |
| | | GCTCATAAAG GAAAATGCCG TGAACTATGT AGCTCAGGCT | |
| | | TGGTAAGGTG CCATCTAAAT TACAAAACAA ACTAATGCAT | |
| | | AATTTTGCTT AAATTTCATC CCAGTATGAT TGTCTTCCCA | |
| | | ACACCAGCAT ATAGTATAGA TTGTCTGTCT TTTTTATATT | |
| | | TTTTAGTTCT TCCTGTACAT GTTTTTGGCA ATAAAGTTAT | |
| | | AGGAAGAACA AAATTATTTT GTTAGAATTA AAACATGCTT | |
| | | AATATTTAGT CTGTTTGTGG AGGGCAGGTA TTCACGTGGA | |
| | | CTGAGATACA ATGTTGGATA CAGAAAATAA CTTTCATTGT | |
| | | CTTCCTGACA CTGTGCTAAG GACATGCTGT TAAAGCTTCA | |
| | | AAGTGACCAG ATGAGGAAGG AATAATTAAT TATTACTCCT | |
| | | GATTTGTAGA TAACTGAGGT AAGAGTGTTT CAAATTTATG | |
| | | ATAGTCTTTT GGGTATTCAG AAACCTTTCC TTATACTGCA | |
| | | CTGGCCACCA GAGCTTAATT TTCCCAGCAG TTACAGCAAT | |
| | | GGGAGATAGA ACAGTCTCAA TCTTTTGCCA ACCATCAGGT | |
| | | TCCTAGAAAC CAGGTAGGTG TATCCCATAA CAAGGGAGGA | |
| | | GCATACCACA GCCCCTCATT TGATTAATTC ATTTGATCTA | |
| | | TCTATGTTAT TAAGTACCTA CTAGGAATAA GGCATTGTGG | |
| | | AAATACTATA CAAAGATAAA CATTGTTTAG ATGCTTATCT | |
| | | ACTTTCCTTT TCACCAGAAA AACAGAAAAA AAAGAAACAT | |
| | | TTTCTTACAG AGTAAAAATG TTCTACATAA TCACATGAGT | |
| | | AGTTCATCTC AGTGTTTTTT ATTCTTTAAA GTTGAACTAT | |
| | | CCCAGTTTCA TTCTATACCA TTCATTGGAT AACCTTGTTA | |
| | | CAACCCAGTC ATGAAACAGA GCAGTGTGAT CAGTTATCTG | |
| | | CATTTAACAA ATAGACAAAT CAGTTTACAT AAAGGTTATG | |
| | | TATGTCACCC ACGATGAAAA GAATCTGCAT TTGAATATGC | |
| | | CCGTATGAAT GTGGGTTCTG TTTTTGCAAC AGAGATTAAG | |
| | | TGACCATTTT TTCTAATTTT ATGGCTATAT ATTTTCTTCA | |
| | | TAAAAATTGG TCACATCGGA GAAGCAGTGC CACAGGAAAA | |
| | | ATGAAATGCA TGTGAAAGTT TGTATTCTGA TTTTACAAGA | |
| | | TGAGATAGAA ATCAGAATTA AAGAGGAATA CTTAGGAGTT | |
| | | ACTAGGCTAA TCAGTGTACG AATTTGTCAT AGGTAGAGAT | |
| | | TTAAAGGTTA ATATCTTAAA ATAGAAGAAA ATTCTAAATC | |
| | | AATCAATCAG TGAGATATAA ACTAAACAGA CCCACTTCAA | |
| | | AGTTGAAAGA AATTTCTAGG CATAAATTGA GACTAGGAAA | |
| | | TTTATATCAG AATAGAGGGT GCTTGACACA TATATATGCT | |
| | | TAAATTGAAG GACAGCTCAG ATTCATTTTT AGGAGAAGAA | |
| | | AGTAAACTAA TGTGCTCTTA AAGAATAAAA ATTTATTCTA | |
| | | TGGTTTCTGT CTCTGATCAT CACCTTCCAT TCTATAAAAA | |
| | | GCTCAGTTAC TGATTTGCTG GGTCATGGTC AAAATTCTTA | |
| | | CCTATTTATT TCATATCAAC TTTAAAAAAT AAATTACTTG | |
| | | CATTCTATAT ATTACTAATT GGGAAGTAAT ATGCCTCAAA | |
| | | TCAGTTTTAT ACTGGATTAT TCCCTATGCT TTAAACCACT | |
| | | GCTCTCAATA AAACACTTCC TGATTAATGT TTGATTATTA | |
| | | GATATTTTAG TCTTGTTGGG GATATTTTAG TCTTGTTGGG | |
| | | TTAGCCATGC TCTGAAGAAT CTGTGAAAGT ACAGTAAAGT | |
| | | TTTAATAAGC AATAAATGTA ACCTTTTATA TAAATCTCAG | |
| | | TGCTAGGTTA ACTTCTAATA AGCAGACGAA CATGTTACAT | |
| | | AAATTATAAT GTCTGTCTTG TAAAAAAGTT GAGGGGACTA | |
| | | AAAGTTTATG ACTCTGATAT GGAAGTTGTC ATATTAAAAA | |
| | | ACTACATTTT AAAACATCAA ATATTTATAC TATTTGCTTT | |
| | | TCAAATAAAA GCATAGTGCT GTTTGGCATA | |
| PRRC2B | NM_013318.3 | GCAGATCGGG AGCGGTGCCG AGAAAAATTT CCTTACTAGA | 35 |
| | | TGACATTTCA TCGCAATGTC CGATCGTTTG GGGCAAATTA | |
| | | CCAAGGGCAA GGATGGGAAA AGCAAGTACT CGACTCTCAG | |
| | | CCTGTTTGAT AAGTATAAAG GAAAATCAGT AGACGCGATT | |
| | | AGATCCTCAG TTATTCCTAG ACATGGCTTA CAGAGTCTTG | |
| | | GGAAAGTTGC TGCAGCCCGG CGCATGCCAC CGCCTGCAAA | |
| | | CCTGCCAAGC TTGAAGTCTG AAAACAAAGG AAACGACCCC | |
| | | AACATCGTGA TAGTACCCAA GGACGGGACG GGATGGGCAA | |
| | | ACAAGCAGGA TCAGCAAGAC CCAAAGAGTT CCAGTGCGAC | |
| | | GGCCTCTCAG CCGCCGGAGT CGCTGCCGCA GCCGGGTTTG | |
| | | CAGAAATCTG TCTCCAATTT GCAGAAACCG ACACAGTCAA | |
| | | TCAGTCAGGA GAATACAAAT TCAGTGCCAG GTGGACCAAA | |
| | | GTCATGGGCA CAGCTGAATG GAAAGCCAGT AGGACACGAA | |
| | | GGTGGTTTAA GGGGCTCAAG CCGACTGTTA TCCTTCTCTC | |
| | | CCGAGGAATT TCCGACGCTG AAAGCAGCTG GAGGGCAGGA | |
| | | CAAGGCTGGC AAAGAAAAGG GCGTCTTAGA TCTGTCGTAT | |
| | | GGGCCAGGAC CAAGCCTCCG CCCTCAGAAT GTGACAAGCT | |
| | | GGAGGGAGGG CGGTGGGCGA CACATAATTT CTGCCACGTC | |
| | | TCTGAGCACC TCCCCAACTG AGCTGGGCAG CAGGAACTCG | |
| | | AGTACGGGAG ATGGAGCCCC CTCCTCGGCA TGTACCAGCG | |
| | | ATTCTAAGGA CCCCTCTCTC CGCCCGGCTC AGCCTGTCCG | |
| | | AAAAGGGGCT TCACAGTTCA TGGGAAATGT ATACCACCCA | |
| | | CCTACATACC ATGACATGCT TCCTGCTTTT ATGTGTTCGC | |
| | | CGAAGTCATC AGAAAACCAG GGTACAGTGG AACGAGGCTC | |
| | | TTTTCCCCTT CCTCAGCTCC GCCTTGAACC TCGAGTTCCT | |
| | | TTTAGACAGT TCCAGATGAA TGACCAAGAC GGAAAAGAAA | |
| | | ACAGGCTGGG ATTGTCTCGC CCACTCCGCC CACTAAGGCA | |
| | | GCTGGTGGAG CGGGCACCAC GGCCCACCAT TATCAATGCG | |
| | | GAAAACCTGA AGGGCCTTGA CGATCTGGAC GCCGATGCCG | |
| | | ATGATGGCTG GGCAGGCCTC CATGAAGAAG TGGACTATTC | |
| | | TGAGAAACTG AAGTTCAGTG ATGATGAAGA GGAGGAAGAA | |
| | | GTTGTGAAGG ACGGCAGGCC AAAGTGGAAC AGTTGGGACC | |
| | | CTAGGAGGCA GCGGCAGTTG TCAATGAGCT CTGCAGACAG | |
| | | TGCGGACGCT AAGCGGACTC GAGAGGAAGG GAAGGACTGG | |
| | | GCTGAAGCAG TGGGTGCGTC CCGTGTGGTC CGAAAGGCGC | |
| | | CAGACCCTCA GCCACCGCCC AGGAAGCTTC ATGGCTGGGC | |
| | | ACCAGGCCCT GACTACCAGA AGTCATCAAT GGGCAGCATG | |
| | | TTCCGGCAAC AGTCCATCGA GGACAAGGAG GACAAGCCCC | |
| | | CACCAAGGCA GAAGTTCATT CAGTCAGAGA TGTCCGAGGC | |
| | | GGTGGAGCGA GCCCGAAAGC GCCGGGAAGA AGAGGAGCGC | |
| | | CGAGCCCGGG AGGAGAGGCT GGCCGCCTGT GCTGCCAAAC | |
| | | TCAAGCAGCT GGACCAGAAG TGTAAGCAGG CACGAAAGGC | |
| | | AGGTGAGGCC CGGAAGCAGG CAGAGAAGGA AGTGCCCTGG | |
| | | TCTCCAAGTG CTGAGAAGGC ATCTCCCCAG GAAAACGGCC | |
| | | CTGCTGTCCA CAAAGGCTCC CCAGAATTCC CTGCCCAAGA | |
| | | GACCCCCACC ACATTCCCAG AAGAGGCACC CACAGTGTCC | |
| | | CCAGCAGTGG CACAGAGCAA CAGCAGTGAG GAAGAGGCCA | |
| | | GAGAGGCTGG GTCCCCTGCA CAGGAGTTCA AGTATCAGAA | |
| | | GTCCCTTCCT CCCCGATTCC AGCGCCAGCA GCAGCAACAA | |
| | | CAGCAGGAGC AGCTGTACAA GATGCAGCAC TGGCAGCCGG | |
| | | TGTACCCCCC GCCGTCCCAC CCCCAGCGCA CCTTTTACCC | |
| | | ACACCACCCC CAGATGTTGG GCTTCGATCC CAGGTGGATG | |
| | | ATGATGCCTT CCTACATGGA CCCACGTATC ACGCCCACTC | |
| | | GGACCCCGGT GGACTTCTAC CCCTCCGCCC TGCATCCCTC | |
| | | AGGACTGATG AAGCCCATGA TGCCCCAGGA GTCCCTCAAT | |
| | | GGGACAGGCT GTCGCTCTGA GGATCAGAAC TGTGTGCCCC | |
| | | CACTCCAAGA AAGAAAAGTG ACCCCCATCG ACTCACCCCC | |
| | | TGTGTGGAGC CCAGAGGGCT ACATGGCACT GCAGAGCAAG | |
| | | GGCTACCCGC TCCCGCACCC GAAGTCGAGT GACACCTTGG | |
| | | CTATGGACAT GCGTGTCAGG AATGAAAGCT CTTTCTCTGC | |
| | | CTCACTCGGA AGGGCAGGGG GCGTAAGTGC TCAGCGCGAT | |
| | | CTCTTTGAGG AGAGAGGGGA GGAGTACTTG AGTGCTTTTG | |
| | | ACAAGAAGGC CCAAGCAGAC TTTGACAGCT GTATCTCTTC | |
| | | TCAAAGAATA GGCCAGGAGC TTTTGTTTCC ACCCCAAGAA | |
| | | AATGTTCAGG ATGCAGGTGC TCCTGGGGGT CACACCCAAA | |
| | | ACCTCAGGTG TTCCCCATTG GAGCCTGACT TTGTCCCAGA | |
| | | TGAGAAAAAG CCAGAGTGTG GCAGTTGGGA TGTTAGCCAC | |
| | | CAGCCAGAGA CCGCTGACAC AGCCCATGGT GTTGAGCGGG | |
| | | AGACACCCCG GGAGGGGACG GCCTTTAACA TCTCCTCCTG | |
| | | GGACAAGAAC GGGAGCCCCA ACAAACAGCC ATCCTCGGAG | |
| | | CCTGAATGGA CTCCCGAGCC CCGGAGCTCC AGCAGCCAGC | |
| | | ACCCGGAGCA GACGGGCAGG ACCCGGAGGT CGGGACCCAT | |
| | | CAAGAAACCA GTCCTGAAAG CCCTCAAGGT GGAAGACAAG | |
| | | GAGAAGGAGC TTGAGAAGAT TAAGCAGGAG CTAGGGGAGG | |
| | | AGAGTACCCG GCTGGCCAAG GAGAAGGAGC AGAGCCCCAC | |
| | | GGCAGAAAAG GATGAGGACG AAGAGAACGA TGCCTCTCTG | |
| | | GCCAACTCCT CCACCACCAC TTTGGAGGAC AAAGGCCCTG | |
| | | GCCATGCCAC TTTTGGCCGC GAGGCCACCA AATTTGAAGA | |
| | | GGAGGAGAAA CCTGACAAGG CCTGGGAAGC CAGACCCCCA | |
| | | CGAGAGTCCA GCGATGTTCC CCCCATGAAG AGAAATAACT | |
| | | GGATCTTTAT TGATGAGGAG CAAGCCTTTG GGGTCAGAGG | |
| | | ACAGGCCCGG GGCCGGGGCC GTGGTTTCAG AGAGTTCACT | |
| | | TTTCGTGGTC GGCCTGCTGG CGGAAATGGG AGCGGCCTCT | |
| | | GTGGTGGGGG GGTCCTGGGG GCCCGCAGCA TCTACTGCAG | |
| | | CAGTCAGCGC AGCGGCCGTG GCCGGGGCCT GCGAGAGTTT | |
| | | GCGCGGCCAG AGGACTGCCC CAGAGCCAAG CCCCGACGGA | |
| | | GAGTTGCCAG TGAGACCCAT AGCGAGGGCT CAGAGTATGA | |
| | | AGAACTTCCC AAGCGCCGCC GGCAGAGGGG CTCCGAGAAC | |
| | | GGGAATGAAG GCTCGCTCCT GGAGAGGGAG GAGAGCACCT | |
| | | TGAAGAAGGG CGACTGCAGA GATTCTTGGC GGTCCAACAA | |
| | | GGGGTGCTCT GAGGACCACA GCGGTCTAGA TGCCAAGAGC | |
| | | CGAGGCCCTC GGGCCTTTGG GCGAGCCCTC CCTCCCCGGC | |
| | | TGAGCAATTG CGGGTATGGA CGGAGAACCT TCGTCTCCAA | |
| | | AGAGTCACCC CACTGGCAGA GCAAAAGTCC AGGCAGCTCT | |
| | | TGGCAGGAAT ATGGCCCTTC CGACACATGC GGATCCCGGC | |
| | | GACCTACAGA CAGAGACTAT GTCCCAGATT CCTACAGACA | |
| | | CCCTGACGCA TTTGGTGGCC GGGGCTTTGA GGACAGCCGC | |
| | | GCGGAGGACA AGAGATCCTT CTTCCAAGAT GAACACGTGG | |
| | | CAGATTCTGA AAATGCAGAG AACCGGCCCT TCAGGAGAAG | |
| | | GCGCCCCCCA CGCCAAGATA AGCCCCCTCG ATTCCGGCGC | |
| | | CTCCGGCAAG AGCGGGAGTC CCTGGGCCTG TGGGGACCCG | |
| | | AGGAGGAGCC CCACCTGCTG GCAGGTCAGT GGCCAGGCAG | |
| | | GCCCAAACTG TGTTCTGGGG ACAAGAGTGG CACTGTGGGC | |
| | | CGCAGGTCCC CTGAGCTCTC CTACCAGAAC TCCTCCGATC | |
| | | ACGCCAATGA GGAGTGGGAG ACGGCCTCCG AAAGCAGCGA | |
| | | CTTCAGCGAG CGGCGGGAGC GGCGGGAAGG CCCTGGGTCC | |
| | | GAGCCCGACT CCCAGGTGGA TGGTGGCCTG TCGGGGGCTA | |
| | | GTTTGGGTGA GAAGAAGGAG CTGGCCAAGA GGAGCTTCTC | |
| | | CAGTCAGAGA CCCGTGGTTG ACAGACAGAG CCGAAAGCTG | |
| | | GAGCCGGGAG GGTTTGGGGA GAAGCCCGTT AGGCCAGGTG | |
| | | GTGGTGACAC CTCCCCTCGC TATGAGAGCC AACAGAATGG | |
| | | GACGCCTTTG AAAGTGAAAA GATCCCCAGA CGAGGCCTTG | |
| | | CCTGGAGGTC TTAGTGGCTG CAGCAGTGGG AGTGGCCACT | |
| | | CCCCCTATGC CCTGGAGCGG GCAGCCCATG CCAGTGCTGA | |
| | | CCTTCCCGAA GCCTCCAGTA AAAAGGCAGA GAAGGAGGCC | |
| | | AAGTTGGCTG CTCCGAGGGC AGGTGAACAG GGAGAGGCCA | |
| | | TGAAACAGTT TGACCTGAAC TATGGAAGTG CCATCATTGA | |
| | | AAATTGCGGG TCCAGCCCCG GGGAGGAGAG TGAGGTGGGT | |
| | | TCTATGGTGG GCGAAGGCTT CATCGAAGTC CTGACCAAGA | |
| | | AGCAGCGCCG CCTGCTGGAG GAAGAGAGAA GAAAGAAGGA | |
| | | GCAGGCCGTG CAGGTGCCTG TCAAAGGTCG AGGCCTTTCC | |
| | | TCCCGTATTC CTCCTCGATT TGCAAAAAAG CAGAACAACT | |
| | | TATGTCTGGA GCAAGGTGAC GTGACCGTGC CTGGCAGCAG | |
| | | CCTGGGCACT GAGATCTGGG AGAGCAGCAG CCAGGCTCTC | |
| | | CCTGTGCAGG CCCCAGCCAA CGACTCCTGG AGGAAAGCTG | |
| | | TCACTGCCTT CAGCAGCACC GAGACTGGCT CTGCGGAGCA | |
| | | GGGTTTTAAG AGCAGCCAGG GAGATAGTGG CGTTGACTTG | |
| | | AGTGCCGAGT CTCGGGAGTC GTCTGCGACC TCCTCGCAGC | |
| | | GCAGCTCCCC ATATGGGACT CTGAAGCCAG AGGAGATGAG | |
| | | CGGGCCCGGC CTGGCGGAAC CCAAGGCCGA CAGCCACAAG | |
| | | GAGCAGGCTC CAAAGCCATC TGAGCAGAAG GATTCAGAAC | |
| | | AAGGCTCTGG ACAGAGCAAG GAGCACAGAC CAGGACCCAT | |
| | | CGGCAACGAG CGTTCTCTGA AAAACAGAAA GGGCTCGGAG | |
| | | GGGGCCGAGC GGCTGCAAGG GGCTGTCGTC CCGCCTGTTA | |
| | | ACGGGGTGGA GATTCACGTG GACTCCGTGC TGCCTGTGCC | |
| | | ACCCATTGAA TTTGGAGTCA GTCCAAAAGA CTCCGATTTC | |
| | | AGCTTGCCAC CTGGTTCTGC CTCTGGTCCT ACTGGGAGTC | |
| | | CAGTTGTTAA ACTTCAGGAT GCCTTGGCCA GTAATGCAGG | |
| | | GTTAACACAG AGTATCCCCA TCCTGCGGCG GGACCATCAC | |
| | | ATCCAGAGGG CCATCGGTCT CTCCCCAATG TCCTTCCCCA | |
| | | CCGCCGACCT TACTCTGAAG ATGGAGTCTG CGCGCAAGGC | |
| | | TTGGGAAAAC TCCCCCAGTT TGCCGGAGCA GAGCTCTCCA | |
| | | GGCGGCGCTG GCTCAGGCAT CCAGCCTCCA TCCTCTGTGG | |
| | | GTGCCTCCAG CGGGGTCAAC TACAGCTCCT TCGGTGGAGT | |
| | | GTCCATGCCA CCCATGCCTG TGGCCTCTGT AGCACCTTCT | |
| | | GCTTCTATGC CAGGCAGCCA CCTCCCGCCC CTGTACCTGG | |
| | | ATGGCCATGT GTTTGCAAGT CAGCCCCGGC TGGTTCCTCA | |
| | | AACGATACCT CAGCAGCAGA GTTACCAACA GGCCGCCGCT | |
| | | GCCCAGCAGA TCCCGATCTC CCTTCACACA TCTCTGCAGG | |
| | | CACAAGCTCA GCTTGGACTG AGGGGTGGGC TTCCTGTGTC | |
| | | CCAGTCCCAG GAGATCTTCA GCTCCTTGCA GCCCTTCAGA | |
| | | TCTCAGGTGT ACATGCACCC CAGCCTGTCA CCGCCCAGCA | |
| | | CCATGATCCT CTCTGGGGGC ACAGCCTTGA AGCCTCCATA | |
| | | CTCGGCGTTC CCAGGCATGC AGCCCTTGGA GATGGTGAAG | |
| | | CCGCAGTCTG GCTCACCCTA CCAGCCCATG AGCGGGAACC | |
| | | AAGCCCTGGT CTACGAGGGC CAGCTCAGCC AGGCTGCTGG | |
| | | CCTGGGTGCC TCCCAGATGT TGGACTCCCA GCTCCCACAG | |
| | | CTGACCATGC CACTGCCTCG GTACGGCTCC GGGCAGCAGC | |
| | | CACTGATCCT GCCCCAGTCT ATTCAGCTGC CACCTGGGCA | |
| | | GAGCCTCTCC GTTGGGGCCC CCCGAAGGAT TCCTCCGCCC | |
| | | GGGTCCCAGC CGCCAGTCCT GAACACCAGC AGAGAGCCCT | |
| | | CTCAGATGGA GATGAAAGGC TTCCACTTTG CCGACAGTAA | |
| | | ACAGAATGTC CCTTCAGGAG GCCCCGTGCC ATCGCCACAG | |
| | | ACCTACAGGC CTAGCTCTGC TAGCCCCAGT GGGAAGCCCT | |
| | | CTGGATCAGC AGTTAACATG GGCTCTGTGC AGGGACACTA | |
| | | CGTGCAACAG GCAAAACAAC GAGTGGATGA GAAACCCAGC | |
| | | CTGGGAGCCG TGAAGCTGCA GGAGGCCCCC TCGGCTGCCT | |
| | | CCCAGATGAA GCGAACCGGA GCGATCAAGC CTCGGGCTGT | |
| | | CAAAGTGGAG GAGAGTAAGG CCTGACAGTG CCTGGCTGCC | |
| | | ACCTCGCCTC TCCCTACTGA GGACGGTGCC GCCATGCGGC | |
| | | CTCGACACAG CCGACACTCG GGAGCCTCAC CAGATCCACC | |
| | | GTCCAAATGC GTGGCCCAGA CTGAGAGACC TCCCTCCTCT | |
| | | CCACTCCCGA AAGCTCCGTT GTCAACCAGC TTGCACCCGT | |
| | | GGATATATGG CATTGACCCG CTTGCTTTGA TACGAAACAA | |
| | | AAAAGCAGAC GACTCCTTCA TCCCATCTGC TCCTACCGTG | |
| | | ACTGTGGAGT GACGCCTCCT GTGCAGTGCA GATTTGCCCT | |
| | | CCCTGCCTCC TCCCTGTCCT GCCGCGCAGC CAGGGCGCCT | |
| | | TCTCAGCAGT GCTTCCGGCC CAGCCGCCCA TCCCTAGGCA | |
| | | CAGTGATTTG GCAGCAGGGT CATTTTACTT TGAGGCTTTT | |
| | | TGTTTTAAAA TGTAGCCAAG GTTTTTACAA AGGGGAAAGG | |
| | | AAAAGAAAAC AAAAACGCAA GCTCCATGTG TATAGCTGAA | |
| | | CTTTTATATG TTTCTTGCCA GCCCCTCCGC TCCCTTCCAT | |
| | | CTCTAGCCTC TGTCCTGTTT AGTTTGATAC GTCACTGCAG | |
| | | TACCTTAAGA GGTGACTCTT AAGAATGCAT CCCCTCCTGA | |
| | | TTCCTCAGCT GGTTCACCCT TGAGGTTATT TGCAAAAAGA | |
| | | AAAGGAGGTT CTTGAGGGCA CCGATTGCGA GCATTCTGGT | |
| | | GCCTGGCTCC CCGCCTGGGA AGCGATGGGG TGCTCAGAGC | |
| | | AGCAGGCAGG TTGGGGGAGG GGGGGGGTCA TAGTTGGGTT | |
| | | CCAGCTCCTG GCTTGATGAG CCCAGGGCGC TTACAGGCAG | |
| | | CCCATGAAGT TGATGACAGT TTTAGCATGA GAATCACACA | |
| | | GGGTCCCTGT CCTGGGCTCC TCTAAAGCCA GTGGATGTGC | |
| | | TGGGCACCAG AGACAAATCA TGGAGATGGC TGCTGGTGGC | |
| | | TCCCAGGTTG GCCCAGATGG GGTGAGCTGA CATACCACAG | |
| | | GCCCATCCCA GGCCCCGTGG GCTCTGCTTC TGGGGCTCCA | |
| | | TACCCTGCCC TGCAGGGGTG CTGTGTTTTT CACACATTTC | |
| | | TTTCCCTGAA GCCTTCTGTA ACCTGTCATT TTCCTTCCTT | |
| | | CCTCTTCCGG AGCCTGCTGC TTTCTCTGGA CCTGTCTCCA | |
| | | CCTCCCACAC AGCTCATCGT GAACACCACT TGGTGATGGA | |
| | | GGGAGTGGAC CCGTGTGTGG TCCCCAAGTG AGGCCACTGG | |
| | | GAGTTTGTCC TTTTCCTCCT TTGCTTCACT CCCAGCAGCA | |
| | | GACCCAGGTT GTCAGGACAG GAGGGCCTGA GCTAAGCAGT | |
| | | AGGCATCAGT CTCGTTTGTC TTCAGACGGC GGGGGCAGGT | |
| | | CCAGGGTGAG GCTGGGTGGA GGGCTGACCA AGGTCCAAAG | |
| | | GGCCTGCGCA GCCTCCGGGA GGGCAGCTTC TCCAGCCAGA | |
| | | GGCTTGTGTG AGCCATCGTG TGCTGGGCTT GTTTTTAAGT | |
| | | AAGAAACAAG GAAATCACTC CAGATTCTGT CATTCCAAGG | |
| | | AAAGGGAAGG GGACAGTTCA GGTTTCTCAG CTGTTCTTAG | |
| | | GGGTCACTGA GCGTCTACCT CCTCCTCCAG AGGAGGCTGG | |
| | | CTCAGAACAC CTAGAGGAGG GGGCCGGGGA TGCACCCCCC | |
| | | ACCAGAGGCT GCCTTCAGCG TCTCACGGGT GCAGGACAGC | |
| | | GCTCAGGCTT GGGCTCTAAG CTCTGTGTCT AGTGTAGAAC | |
| | | ATGGGGAAGG AGCATCTTAG GAACTGCTGA AGTAACTTCT | |
| | | TACTGCTCTC ACAATTCTAA GGAAGCGGGA GAACGGCCTC | |
| | | CTACCAACAG CGCCCACCCC AGAGCTGCCT GGGAAAGGGC | |
| | | AGTTTTACTG AAAGGTGCTT TACTGTTCAC CTGCATCTTT | |
| | | CAGCAGCTCC CCTCCTGCCC TCACCTGGTC TTTTCCCTCT | |
| | | TTATCCCAAG CCTTTATGCT TGAGTCCCTT CCCCAGGGGC | |
| | | TGCCCACCCG ACAGTTCCAG GCATTCCCTA CCTGAGCTTC | |
| | | TTGTCTGCTT TTCCTTCTCC CACTGCAAGC GGCTGCTTGT | |
| | | GGGGCCTGGG ATGAGCCCTC TCTGTCCCCA CCGGCCCTCC | |
| | | TTGCCAAGCC ATTCCTGGGT GAGTTCAGGC CTGCGGGAGC | |
| | | CACACATTCA TCTCCACCTG GACACTTGAG CCGCATGGCC | |
| | | AGACCCCTCC CACCTGATGC GGTGGTGCGT GTGATTTGTC | |
| | | AAAAGAAAGC CTTCTGGATG CTGTTAAGAT GTACCCTTCA | |
| | | GGTGAACCTG GTATCAGACC CACAGTACTT GCTGTTTGAG | |
| | | AAAAAATAAA AACAAAAAGG TCACCTGTTC TCCAGCCCTT | |
| | | TTCTCTTACC TGGTATTTCC TTCCTTTCTC CTCCCCCACC | |
| | | CCAAATAAAA AAACAAAAAA CACTAGAATT TATTTATATG | |
| | | TATTGATGTT GTAGGTCTAG GTGAAAAAAA AAGAAGTAAA | |
| | | TGTTTCACTG CTCTATTTAT ATATAATGTC TGAATTAATT | |
| | | CTGTGCAGGA AAGGCCAGGA AATTGCATGT GAAGTTCGGT | |
| | | GCAGTCACCA CCTGTGTGTG ACCTGAGCTG CAGTCTCTTC | |
| | | GCTGAGATGC AGGTTTTAAA TGAGACTTGG GGGGCTGAGG | |
| | | GCAGGCCTCA GGCCTCCCAG CGCCCCAACC CCTCCTTGGT | |
| | | CTAATGAAAT GCAGTTCTTA GTGCAGAGAT GTTTTAAGGT | |
| | | GCAATATATC TCTTCCTTTC CCGTGGTTTT AGAGCCAAGC | |
| | | TCAAGGTAGT AGGACGTAGG GTCTTATTTT GTTTTCAAAC | |
| | | CCCCATCCTC AGAGCGCAGA TACATGCAGA GGCTTCTGCC | |
| | | AGGATACCAC GGGGCCTTAG TGGGAACAGG TGGAGACCAG | |
| | | CACTTCCCTT TCCTGCTGCT GAGGTAGGGA TTGGGGGGTC | |
| | | AGAACCCACT CACTTTTGCC TGTTAAAGTT GCCCTCCTGA | |
| | | CGCTGGCAGC TCTGCCTTGG TCACTGGGGA TGCGGCTCGT | |
| | | TGCTCAGCCA CCAGTGGCCT TGCGGTATTG TCCACCATCC | |
| | | ACTAGAGTGG GATGAAGTCC AGAGTGTGGG TATACATCTC | |
| | | AGATGCCCAT CTACCCACTG GGGACTTCAA TGCCAGCTGC | |
| | | ATTTGGTTTG GTTTTCTTAA CTGTTGGCTT CTCCCCACAG | |
| | | CGTTTTTTGT TTTTTTTTAA ACATTCATAT TGTTTTCAAA | |
| | | CTTGGAATTC ATAGACACTC TGGCTCTAGG TTCCTTAAGG | |
| | | GGGAAAACAA AAGATGACTT TATTTCACAT TCAAGAAAAT | |
| | | CAGTTCAGTT CCAAAGCTGT GGTCCTTCCA GCCACTTCTA | |
| | | GGGACACTGG GGAACCTTGT TAAACGTTGA CATCAGTGCT | |
| | | CTCCAGCCGT GCTGTCACCC TCCTATCTTC TGGATCTGCC | |
| | | TTCGCGATGG TCAGTGACAG CTTCTGGAAG CTGAGCACAC | |
| | | ACAGGTGCAC AGCCATGCTG TGGTCTGGCC TGCTACGGCA | |
| | | GCATGGCAGC TCTGGTGGAG CCTTCTCCCT TGCCATTTGG | |
| | | TTCCCCTGTG CCAAGTAGCT GCAGGCTGCC CCTCAAATCT | |
| | | TCATTTGTCC CTTTTCACTT CCTGCAGAAC AAGCCTGGGT | |
| | | TAGAGGGTCT GCTGGAAATG GCCTTTGAAG ACCAAGGATA | |
| | | CCAGGATGTG TGCACTCTGT CGTGTTCTGT GATGAATGGG | |
| | | AAACGTAGGC TTCCAGAAAG CCAGCTCTCT TCTGAAATGT | |
| | | GACGGACCTA AGCAGGAAGT CATCCAGGAC AGGAGTGGCT | |
| | | CAGTGTTGGG GATGGACGCT GTCGCCCAGC CATGCTCCAC | |
| | | CAGGGCCACC AATGTGTAGT TGGCTGGTGG TCTTCGGGCA | |
| | | TGTGAGACCT GCTCTTCACT GTTTCCACCC CACTTGGTGG | |
| | | CCTCCAGGAT GGTAGTGGCA CCCTCAGAGC CCCATCTTCA | |
| | | GCATGTTCTG AAGCCTCAGA GTGGAAATTC CTGCTAAGGC | |
| | | TCTGTGTGGA CGCCTTTCTC CCGTGATCTA AAGGGGACAC | |
| | | TGTACTCAAG CTTTTGACCT CATGCCTTGT GTAGTAAAAA | |
| | | AGGATTTGGG GGTTTTGTTT GGTTCCTGAG AGGGTTGTGT | |
| | | TTTGTTTTTG TTTCCTTTTG TTTATGTTTT GGCCTTTCCT | |
| | | CTTTGTCTTT CCATGTAGAC CAGATATTTG AAAGGGCAGA | |
| | | CGATGGCTAG AGGTGTAATG TGCAGCTTGT TTATACGGTA | |
| | | TTTTGGGAAA CTTACCTTGG ATGGGAAATC GAATCGTGGA | |
| | | TTCACCAGGC CGGTGCTGGC ACACTCACCC TCGCCCTTTC | |
| | | CCTCCGGTTC AGTACCTATT GTTTCTCCTT TCAAATATGT | |
| | | GATTGTACTA GCTCTTTCCA TATGAAAGAA TTCTCCTTAT | |
| | | TTAAATAAAA AAAGTTTAAA AA | |
| DOPEY2 | NM_005128.3 | TCCCACAGTG CCTGGCCCAG AAGCCTTGCT AAATATTTGA | 36 |
| | | ACAGGATTGC CCAATACTTT TCTGCTGTGA GAATGTAAGA | |
| | | TGGATCCAGA AGAGCAGGAG CTCTTAAATG ATTACAGATA | |
| | | CAGAAGCTAC TCTTCAGTGA TTGAAAAGGC TTTGAGAAAT | |
| | | TTTGAGTCCT CGAGTGAATG GGCGGATCTC ATATCTTCAC | |
| | | TTGGCAAACT CAACAAGGCT CTTCAGAGTA ACCTGAGGTA | |
| | | CTCCTTGTTG CCAAGACGGC TCCTCATCAG CAAAAGATTA | |
| | | GCTCAGTGTT TGCACCCTGC CCTGCCCAGT GGTGTCCACT | |
| | | TAAAAGCTCT GGAAACCTAC GAGATTATCT TTAAAATCGT | |
| | | GGGGACCAAA TGGCTGGCCA AGGACTTGTT TCTGTACAGC | |
| | | TGCGGGTTAT TTCCTCTCCT GGCACACGCG GCGGTGTCGG | |
| | | TGAGGCCGGT GCTGCTCACC CTGTACGAGA AGTACTTCCT | |
| | | CCCACTGCAG AAGCTGCTCC TGCCCAGTCT GCAGGCCTTC | |
| | | ATCGTGGGCC TGCTGCCCGG CCTTGAAGAG GGCTCCGAGA | |
| | | TCTCCGACAG AACGGATGCT CTGCTCCTGA GACTGTCGCT | |
| | | GGTGGTTGGC AAAGAGGTGT TTTACACCGC CCTCTGGGGG | |
| | | AGCGTCCTGG CCAGCCCGTC CATCCGCCTC CCTGCCTCAG | |
| | | TCTTCGTGGT GGGCCACATC AACAGGGATG CCCCCGGCCG | |
| | | GGAGCAGAAG TACATGCTGG GGACCAATCA CCAACTCACG | |
| | | GTGAAGTCTT TGCGTGCCTC CCTGTTGGAC TCAAATGTTC | |
| | | TTGTGCAAAG AAATAATCTG GAAATCGTTC TGTTTTTCTT | |
| | | CCCATTTTAT ACCTGTCTGG ATTCCAATGA GAGAGCCATC | |
| | | CCCCTCCTCA GATCTGACAT CGTGCGCATT CTCTCAGCCG | |
| | | CCACCCAGAC CCTACTGAGA AGGGACATGT CCCTGAACAG | |
| | | AAGACTGTAT GCATGGTTAC TAGGCTCAGA CATAAAAGGA | |
| | | AATACCGTTG TGCCAGAATC TGAAATCTCA AATTCTTATG | |
| | | AAGACCAGTC GTCTTATTTT TTTGAAAAAT ACTCCAAGGA | |
| | | TCTTTTAGTT GAGGGTTTGG CTGAGATATT GCATCAGAAG | |
| | | TTCATAGATG CTGACGTGGA GGAACGCCAT CATGCATACC | |
| | | TGAAGCCTTT TCGCGTCCTC ATCAGTCTGC TTGACAAGCC | |
| | | AGAAATAGGG CCTCAAGTGG TTGGGAATTT GTTTCTCGAA | |
| | | GTCATCAGGG CCTTTTATTC TTACTGCAGA GATGCCCTTG | |
| | | GCTCTGATCT TAAACTTAGC TACACCCAGA GTGGAAATTC | |
| | | GCTGATAAGT GCAATCAAGG AAAACAGAAA TGCCTCTGAG | |
| | | ATTGTCAAAA CGGTAAATTT GCTGATAACT TCTCTAAGCA | |
| | | CAGACTTTCT CTGGGATTAT ATGACAAGGT GTTTTGAGGA | |
| | | ATGCTTTAGA CCAGTGAAGC AGCGTTACAG CGTGAGGAAC | |
| | | AGCGTCAGCC CTCCCCCCAC GGTCTCGGAG CTCTGCGCCC | |
| | | TCCTGGTCTT CCTGCTGGAT GTCATTCCTT TGGAACTTTA | |
| | | CTCTGAGGTG CAAACCCAGT ATCTCCCTCA GGTGCTCGGC | |
| | | TGCCTGGTGC AGCCTCTTGC TGAGGACATG GAGGCCTTAA | |
| | | GTTTACCTGA ACTCACGCAT GCCTTGAAGA CGTGTTTCAA | |
| | | GGTGCTCAGC AAAGTCCAGA TGCCTCCTTC CTACCTCGAC | |
| | | ACGGAGTCCA CCAGCGGAAC CTCGAGTCCA GTAAAAGGTG | |
| | | AAAACGGCAA AATAATTTTG GAAACAAAGG CAGTGATTCC | |
| | | CGGTGACGAA GATGCTTCGT TTCCCCCTCT GAAGTCTGAG | |
| | | GACAGTGGGA TCGGGCTCAG TGCCTCGTCA CCGGAGCTCT | |
| | | CTGAGCACTT GAGGGTTCCT CGAGTTTCTC TGGAAAGGGA | |
| | | CGACGTTTGG AAGAAGGGCG GGAGCATGCA GAGGACGTTT | |
| | | CTTTGCATCC AAGAGCTAAT CGCCAACTTT GCCAGCAAGA | |
| | | ACATTTTTGG AGTACAGCTG ACAGCGTCAG GAGAAGAAAG | |
| | | CAAGTCCGAG GAGCCTGCAG GGAAGAGGGA CAGGGATGGG | |
| | | ACGCAGAGCC TGGCAGCCAA TGATTCCAGC AGGAAGAACT | |
| | | CTTGGGAGCC CAAGCCCATC ACTGTGCCTC AGTTCAAGCA | |
| | | GATGCTGTCA GACTTGTTCA CAGCACGAGG GTCTCCATTC | |
| | | AAGACAAAAA GTTCAGAGTC ACCATCGTCT TCGCCCAGCA | |
| | | GCCCTGCCAG GAAAAACGGG GGAGAATGGG ATGTTGAGAA | |
| | | GGTGGTCATT GACCTGGGGG GTTCCAGGGA GGAACGCAGG | |
| | | GAGGCCTTTG CCGCCGCCTG CCACCTGCTG CTGGATTGTG | |
| | | CCACTTTCCC TGTCTACCTG TCCGAGGAAG AGACCGAGCA | |
| | | GCTCTGTGCA ACGCTCTTCC AGCTGCCAGG AGCCGGTGAT | |
| | | TCCAGTTTTC CATCTTGGCT GAAGTCCCTC ATGACTATTT | |
| | | GCTGCTGTGT GACTGACTGC TACCTCCAGA ACGTGGCCAT | |
| | | TTCCACTCTG CTGGAAGTGA TAAACCATTC CCAGTCCCTG | |
| | | GCGCTTGTCA TTGAAGACAA GATGAAACGC TATAAGAGCT | |
| | | CTGGACACAA CCCTTTTTTT GGCAAGCTGC AGATGGTGAC | |
| | | GGTTCCTCCC ATTGCTCCAG GGATATTGAA AGTCATTGCA | |
| | | GAGAAAACAG ATTTCTATCA GAGGGTGGCT CGTGTGCTTT | |
| | | GGAATCAGCT GAACAAAGAG ACCCGGGAGC ATCACGTCAC | |
| | | CTGCGTAGAA TTGTTCTACC GGCTGCACTG CCTGGCCCCT | |
| | | ACGGCCAACA TCTGCGAGGA CATCATCTGC CATGCCCTCC | |
| | | TGGACCCTGA CAAGGGAACA AGGCTGGAAG CTCTGTTTAG | |
| | | ATTTTCCGTG ATCTGGCATC TGACAAGAGA GATCCAAGGC | |
| | | AGTCGAGTAA CATCTCACAA TCGCTCCTTT GATAGGTCCT | |
| | | TGTTTGTCGT GCTGGACAGC CTGGCCTGCA CGGATGGTGC | |
| | | CATCGGTGCG GCAGCCCAGG GCTGGCTGGT GCGTGCGCTC | |
| | | TCCCTCGGGG ACGTGGCTCG CATCCTCGAA CCCGTGCTCC | |
| | | TGCTGCTGCT GCAGCCAAAA ACCCAGAGAA CCTCCATCCA | |
| | | CTGCCTCAAG CAGGAGAACT CGGCCGATGA CTTGCACCGT | |
| | | TGGTTTAACA GGAAGAAAAC CTCTTTCAGA GAGGCATGCG | |
| | | CAGTGCCCGA GCCTCAGGAG AGCGGCTCTG AAGAGCACCT | |
| | | GCCTCTGAGC CAGTTCACCA CAGTGGACCG TGAAGCCATT | |
| | | TGGGCCGAAG TGGAGAAGGA GCCCGAGAAG TACCCGCTGC | |
| | | GAGGCGAGCT GAGCGAGGAA GAGCTGCCCT ACTACGTGGA | |
| | | GCTTCCAGAC AGGACGGCCC ACGGCGCCCC GGACAGCAGC | |
| | | GAGCACACCG AGTCTGCAGA TACAAGCTCC TGCCACACGG | |
| | | ACAGCGAGAA CACGTCCTCC TTCTCCTCCC CTTCCCACGA | |
| | | CCTGCAGGAG CTGAGCAACG AAGAGAACTG CTGTGCACCC | |
| | | ATCCCCATGG GGGGCAGGGC GTACCCCAAG CGCTCGGCCC | |
| | | TGCTGGCGGC CTTCCAGTCA GAAAGCTTCA AGGCTGGGGC | |
| | | CAAGTTAAGC CTGGTGCGGG TGGACTCGGA CAAGACGCAG | |
| | | GCTTCTGAGT CGTTCTCCAG CGACGAGGAG GCGGACTTGG | |
| | | AGCTCCAGGC CCTCACCACA TCCAGGCTGC TAAAGCAGCA | |
| | | GCGGGAAAGG CAGGAGGCCG TCGAGGCCTT GTTCAAGCAC | |
| | | ATCCTGCTCT ACCTGCAGCC CTACGACTCT CGGCGGGTCC | |
| | | TCTATGCCTT CTCGGTGCTG GAGGCTGTGC TCAAAACCAA | |
| | | CCCTAAGGAA TTCATCGAGG CTGTGTCCAG GACTAGCATG | |
| | | GATACCAGCT CCACCGCGCA CCTCAACCTC ATCTCCAACC | |
| | | TCCTCGCTCG CCACCAGGAG GCCCTCATTG GCCAGAGTTT | |
| | | CTACGGAAAG CTCCAGACCC AGGTCCCCAA CGTGTGCCCC | |
| | | CACTCTCTGC TCCTGGAGCT GCTCACCTAC CTCTGCCTGA | |
| | | GCTTCCTGCG CTCCTACTAC CCTTGCTATT TGAAGGTCTC | |
| | | GCACCGAGAC ATTCTCGGCA ACCGGGACGT GCAGGTCAAA | |
| | | AGTGTCGAGG TTTTGATCAG GATAATGATG CAGCTGGTCT | |
| | | CAGTGGCCAA GTCTTCGGAA GGGAAGAACG TGGAGTTCAT | |
| | | CCACAGCTTG CTGCAGAGGT GCAAAGTTCA GGAGTTTGTC | |
| | | CTGCTCTCCC TGTCGGCGTC CATGTACACG AGCCAGAAGC | |
| | | GCTACGGGCT GGCCACCGCC CACCACGGCA GGGCCCTGCC | |
| | | AGAGGACAGC CTCTTTGAGG AGAGTCTCAT TAACTTGGGT | |
| | | CAGGACCAGA TCTGGAGTGA GCACCCGCTG CAGATTGAGC | |
| | | TGCTGAAGCT GCTGCAGGTG CTGATTGTCT TGGAACACCA | |
| | | CCTGGGTCGG GCCCATGAGG AGGCGGAAAA CCAGCCCGAC | |
| | | CTGTCCCGGG AGTGGCAGAG AGCCCTGAAC TTCCAGCAGG | |
| | | CCATCAGCGC CCTGCAGTAC GTGCAGCCCC ACCCCCTCAC | |
| | | CTCCCAGGGT CTTCTGGTCT CTGCGGTGGT GAGGGGTCTG | |
| | | CAGCCCGCCT ACGGTTACGG CATGCATCCG GCCTGGGTGA | |
| | | GCTTGGTCAC GCATTCCTTG CCCTACTTCG GAAAGTCCCT | |
| | | GGGCTGGACG GTGACACCCT TTGTTGTCCA GATTTGCAAA | |
| | | AACTTGGATG ACTTGGTCAA GCAGTATGAA AGCGAATCTG | |
| | | TGAAGCTCTC TGTCAGCACA ACCTCCAAGA GGGAAAACAT | |
| | | TTCTCCAGAT TATCCACTCA CCCTTCTAGA AGGTCTAACG | |
| | | ACCATTAGTC ATTTTTGTCT TTTGGAACAA GCCAACCAAA | |
| | | ACAAAAAGAC CATGGCTGCA GGTGATCCTG CCAACTTGAG | |
| | | GAATGCCAGA AATGCCATTT TGGAAGAGCT GCCTCGAACT | |
| | | GTTAACACCA TGGCCCTTCT CTGGAATGTT CTCAGAAAGG | |
| | | AGGAGACTCA AAAGAGACCT GTCGATCTCC TAGGGGCCAC | |
| | | GAAGGGATCC TCTTCCGTTT ACTTTAAAAC CACCAAAACC | |
| | | ATAAGACAAA AAATTTTAGA CTTCTTAAAC CCCTTGACGG | |
| | | CCCATCTTGG GGTTCAGTTG ACAGCGGCTG TTGCGGCAGT | |
| | | GTGGAGCAGA AAGAAAGCCC AGCGTCACAG TAAGATGAAG | |
| | | ATTATCCCAA CGGCAAGTGC ATCCCAGCTA ACCCTTGTCG | |
| | | ACTTGGTGTG TGCACTCAGC ACCCTGCAGA CTGACACGCT | |
| | | GCTGCACCTG GTGAAGGAGG TGGTGAAGAG GCCACCCCAA | |
| | | GTCAAAGGGG GTGATGAGAA ATCGCCCCTA GTGGACATTC | |
| | | CTGTGTTGCA GTTTTGCTAT GCTTTTCTCC AAAGGCTCCC | |
| | | AGTACCAGCC TTGCAAGAGA ACTTTTCTTC ACTGTTGGGA | |
| | | GTATTGAAAG AGTCTGTACA GTTGAATCTA GCCCCACCTG | |
| | | GGTATTTTCT GCTTCTCAGC ATGCTGAATG ACTTTGTAAC | |
| | | AAGAACTCCC AACCTGGAAA ACAAGAAGGA CCAAAAAGAC | |
| | | CTGCAGGAAA TCACTCAGAA AATCCTAGAA GCTGTGGGGA | |
| | | ACATTGCCGG CTCTTCCTTG GAGCAAACCA GCTGGCTAAG | |
| | | CAGAAACCTG GAAGTGAAGG CCCAACCTCA GGCCTCTCTA | |
| | | GAAGAATCTG ATGCTGAGGA GGACCTGTAT GATGCTGCTG | |
| | | CAGCTTCAGC AATGGTGTCT TCATCCGCCC CGTCGGTGTA | |
| | | CAGCGTGCAA GCCCTCTCTC TCCTGGCAGA GGTACTGGCT | |
| | | TCCCTCCTGG ACATGGTTTA TCGAAGTGAT GAGAAGGAGA | |
| | | AAGCTGTGCC GTTAATCTCC CGTCTGCTTT ACTATGTTTT | |
| | | TCCATACTTA CGCAACCACA GTGCCTACAA TGCTCCCAGC | |
| | | TTCCGGGCTG GCGCTCAGCT GCTGAGCTCC CTGAGTGGCT | |
| | | ATGCCTACAC AAAGCGAGCC TGGAGGAAGG AGGTCCTGGA | |
| | | GCTGTTTCTC GACCCCGCTT TCTTTCAGAT GGATACTTCC | |
| | | TGTGTTCATT GGAAGTCCAT TATTGACCAT CTTTTGACTC | |
| | | ATGAGAAAAC AATGTTTAAG GATTTAATGA ACATGCAGAG | |
| | | CAGTTCTTTG AAACTATTCT CAAGTTTTGA ACAGAAAGCC | |
| | | ATGCTGTTAA AGCGCCAGGC TTTTGCTGTC TTCAGTGGAG | |
| | | AACTTGATCA ATACCACCTT TACCTTCCAC TGATACAAGA | |
| | | ACGCCTGACA GACAATCTCA GAGTTGGACA GACATCCATA | |
| | | GTTGCTGCTC AGATGTTTCT TTTTTTCAGA GTTTTGCTGC | |
| | | TAAGAATATC TCCTCAACAT TTGACTTCAT TGTGGCCAAT | |
| | | AATGGTCTCT GAATTGATTC AGACATTCAC ACAGCTTGAA | |
| | | GAAGATCTAA AAGATGAAGA TGAGTCATTG AGAAGCACCA | |
| | | ACAAAGTAAA CAGAACGAAA GTTTCAGTCC CGGATGCAAA | |
| | | TGGACCCTCA GTGGGGGAGA TACCCCAGAG TGAACTCATC | |
| | | TTGTATTTAT CAGCTTGCAA ATTCTTGGAC ACAGCGCTTT | |
| | | CTTTTCCACC TGACAAGATG CCATTATTTC AAATTTATAG | |
| | | GTGGGCATTT ATTCCAGAAG TGGACACAGA GGGCCCTGCC | |
| | | TTCCTGTCGG ATGTAGAGGA GAATCACCAA GAATGCAAAC | |
| | | CCCACACTGT CAGGATTCTA GAACTTCTAA AATTAAAGTT | |
| | | TGGGGAAATC AGTAGCTCTG ATGAGATCAC CATGAAGAGT | |
| | | GAATTCCCGC TTCTGCGCCA ACATTCTGTT TCCAGCATCA | |
| | | GGCAGTTGAT GCCATTCTTC ATGACTCTAA ATGGTGCATT | |
| | | TAAGACCCAG AGACAGCTGC CTGCTGATAG CCCAGGAACT | |
| | | CCATTCTTGG ACTTTCCTGT CACAGATAGC CCAAGGATCT | |
| | | TAAAACAACT GGAAGAATGC ATCGAATATG ATTTTCTGGA | |
| | | ACATCCAGAA TGTTAACCAT GTGAGAGAGA ATATGTTTAA | |
| | | TCCATGTATT GGTACTTTAC TGAAAACCAG GTTATATTCT | |
| | | AAAGAAGAAA GAAGGCAGGA TAGTGCTTTT GAACAAGCCT | |
| | | ATTTCCATTT TGAAAGTAGA TTTCAGGCTA GGTGCGGTGG | |
| | | CTCACACCTG TAATCTCAGC ACTTTGGGAG GCCAAGGCAG | |
| | | GCAGATCACT TGAGGTCAGG AGTTCGAGAC CAGCCTGACC | |
| | | AACATGGTGA GACCCTGTCT CTACTAAAAA TACAAAAATT | |
| | | AGCTGGGTGT GGTGGCGGCG CCTGTAATCC CAGCTACTTG | |
| | | GGAGGCTAAG GCATGAGAAT TGCTTGAACC CAGGAGGTGG | |
| | | AGGCTGCAGT GAGCCGAGAT CACGACACTG CACTCCAGCT | |
| | | GTGTGACAGA ATGAGACCAT CTCCAAAAAA AAAAAAAAGT | |
| | | AGATTTCAGA TAATTTACTG TTCAGCAACA GGACACACCT | |
| | | CCCTAAATGC CTTGTAATAT ATTTGAATCT GATTCTGCAT | |
| | | TTCTTCCTCA ATTTATGTAA TGAAAATAAA ATTAATATAT | |
| | | CATCTAACAG TAGCACAAAA TTTGTAATAT GAAGTAAAGT | |
| | | ATGAAGATAA TGAAGAAGTT GTTTTCTTTG TTGAAGCAGT | |
| | | TATATGGGTC TTTCTCAGTA TATTTCTCTT TTCTCTAAAA | |
| | | GTTTAAACTT ATTAAAAGAA TGTTATTTTT AACCTTTCAA | |
| | | AAAAAAAAA | |
| NDUFB11 | NM_019056.6 | GCTCTGGCCG GCCCCGGCGA TTGGTCACCG CCCGCTAGGG | 37 |
| | | GACAGCCCTG GCCTCCTCTG ATTGGCAAGC GCTGGCCACC | |
| | | TCCCCACACC CCTTGCGAAC GCTCCCCTAG TGGAGAAAAG | |
| | | GAGTAGCTAT TAGCCAATTC GGCAGGGCCC GCTTTTTAGA | |
| | | AGCTTGATTT CCTTTGAAGA TGAAAGACTA GCGGAAGCTC | |
| | | TGCCTCTTTC CCCAGTGGGC GAGGGAACTC GGGGCGATTG | |
| | | GCTGGGAACT GTATCCACCC AAATGTCACC GATTTCTTCC | |
| | | TATGCAGGAA ATGAGCAGAC CCATCAATAA GAAATTTCTC | |
| | | AGCCTGGCCG AAAATGGTTG GCCCCACGAA GCCACGACAA | |
| | | CTGGAGGCAA AGAGGGTTGC TCAACGCCCC GCCTCATTGG | |
| | | AAAACCAAAT CAGATCTGGG ACCTATATAG CGTGGCGGAG | |
| | | GCGGGGCGAT GATTGTCGCG CTCGCACCCA CTGCAGCTGC | |
| | | GCACAGTCGC ATTTCTTTCC CCGCCCCTGA GACCCTGCAG | |
| | | CACCATCTGT CATGGCGGCT GGGCTGTTTG GTTTGAGCGC | |
| | | TCGCCGTCTT TTGGCGGCAG CGGCGACGCG AGGGCTCCCG | |
| | | GCCGCCCGCG TCCGCTGGGA ATCTAGCTTC TCCAGGACTG | |
| | | TGGTCGCCCC GTCCGCTGTG GCGGGAAAGC GGCCCCCAGA | |
| | | ACCGACCACA CCGTGGCAAG AGGACCCAGA ACCCGAGGAC | |
| | | GAAAACTTGT ATGAGAAGAA CCCAGACTCC CATGGTTATG | |
| | | ACAAGGACCC CGTTTTGGAC GTCTGGAACA TGCGACTTGT | |
| | | CTTCTTCTTT GGCGTCTCCA TCATCCTGGT CCTTGGCAGC | |
| | | ACCTTTGTGG CCTATCTGCC TGACTACAGG TGCACAGGGT | |
| | | GTCCAAGAGC GTGGGATGGG ATGAAAGAGT GGTCCCGCCG | |
| | | CGAAGCTGAG AGGCTTGTGA AATACCGAGA GGCCAATGGC | |
| | | CTTCCCATCA TGGAATCCAA CTGCTTCGAC CCCAGCAAGA | |
| | | TCCAGCTGCC AGAGGATGAG TGACCAGTTG CTAAGTGGGG | |
| | | CTCAAGAAGC ACCGCCTTCC CCACCCCCTG CCTGCCATTC | |
| | | TGACCTCTTC TCAGAGCACC TAATTAAAGG GGCTGAAAGT | |
| | | CTGAAAAAAA AAAAAAAA | |
| ND4 | NC_012920.1 | ATGCTAAAACTAATCGTCCCAACAATTATATTACTACCACTGAC | 38 |
| | | ATGACTTTCCAAAAAACACATAATTTGAATCAACACAACCACCC | |
| | | ACAGCCTAATTATTAGCATCATCCCTCTACTATTTTTTAACCAAA | |
| | | TCAACAACAACCTATTTAGCTGTTCCCCAACCTTTTCCTCCGACC | |
| | | CCCTAACAACCCCCCTCCTAATACTAACTACCTGACTCCTACCC | |
| | | CTCACAATCATGGCAAGCCAACGCCACTTATCCAGTGAACCACT | |
| | | ATCACGAAAAAAACTCTACCTCTCTATACTAATCTCCCTACAAA | |
| | | TCTCCTTAATTATAACATTCACAGCCACAGAACTAATCATATTT | |
| | | TATATCTTCTTCGAAACCACACTTATCCCCACCTTGGCTATCATC | |
| | | ACCCGATGAGGCAACCAGCCAGAACGCCTGAACGCAGGCACAT | |
| | | ACTTCCTATTCTACACCCTAGTAGGCTCCCTTCCCCTACTCATCG | |
| | | CACTAATTTACACTCACAACACCCTAGGCTCACTAAACATTCTA | |
| | | CTACTCACTCTCACTGCCCAAGAACTATCAAACTCCTGAGCCAA | |
| | | CAACTTAATATGACTAGCTTACACAATAGCTTTTATAGTAAAGA | |
| | | TACCTCTTTACGGACTCCACTTATGACTCCCTAAAGCCCATGTC | |
| | | GAAGCCCCCATCGCTGGGTCAATAGTACTTGCCGCAGTACTCTT | |
| | | AAAACTAGGCGGCTATGGTATAATACGCCTCACACTCATTCTCA | |
| | | ACCCCCTGACAAAACACATAGCCTACCCCTTCCTTGTACTATCC | |
| | | CTATGAGGCATAATTATAACAAGCTCCATCTGCCTACGACAAAC | |
| | | AGACCTAAAATCGCTCATTGCATACTCTTCAATCAGCCACATAG | |
| | | CCCTCGTAGTAACAGCCATTCTCATCCAAACCCCCTGAAGCTTC | |
| | | ACCGGCGCAGTCATTCTCATAATCGCCCACGGGCTTACATCCTC | |
| | | ATTACTATTCTGCCTAGCAAACTCAAACTACGAACGCACTCACA | |
| | | GTCGCATCATAATCCTCTCTCAAGGACTTCAAACTTACTCCCAC | |
| | | TAATAGCTTTTTGATGACTTCTAGCAAGCCTCGCTAACCTCGCC | |
| | | TTACCCCCCACTATTAACCTACTGGGAGAACTCTCTGTGCTAGT | |
| | | AACCACGTTCTCCTGATCAAATATCACTCTCCTACTTACAGGAC | |
| | | TCAACATACTAGTCACAGCCCTATACTCCCTCTACATATTTACC | |
| | | ACAACACAATGGGGCTCACTCACCCACCACATTAACAACATAA | |
| | | AACCCTCATTCACACGAGAAAACACCCTCATGTTCATACACCTA | |
| | | TCCCCCATTCTCCTCCTATCCCTCAACCCCGACATCATTACCGGG | |
| | | TTTTCCTCTT | |
| MORF4L1 | NM_001265603.1 | CGGCGTGCCC TGGGGCGGCG CGGGCGCAGG GGCGCGTGCG | 39 |
| | | CGGCGGGCTG TCGTTGGCTG GAGCAGCGGC TGCGCGGGTC | |
| | | GCGGTGCTGT GAGGTCTGCG GGCGCTGGCA AATCCGGCCC | |
| | | AGGATGTAGA GCTGGCAGTG CCTGACGGCG CGTCTGACGC | |
| | | GGAGTTGGGT GGGGTAGAGA GTAGGGGGCG GTAGTCGGGG | |
| | | GTGGTGGGAG AAGGAGGAGG CGGCGAATCA CTTATAAATG | |
| | | GCGCCGAAGC AGGACCCGAA GCCTAAATTC CAGGAGGTTG | |
| | | GGATGAATGG GTTCCGGAGA GCAGAGTACT CAAATACGTG | |
| | | GACACCAATT TGCAGAAACA GCGAGAACTT CAAAAAGCCA | |
| | | ATCAGGAGCA GTATGCAGAG GGGAAGATGA GAGGGGCTGC | |
| | | CCCAGGAAAG AAGACATCTG GTCTGCAACA GAAAAATGTT | |
| | | GAAGTGAAAA CGAAAAAGAA CAAACAGAAA ACACCTGGAA | |
| | | ATGGAGATGG TGGCAGTACC AGTGAGACCC CTCAGCCTCC | |
| | | TCGGAAGAAA AGGGCCCGGG TAGATCCTAC TGTTGAAAAT | |
| | | GAGGAAACAT TCATGAACAG AGTTGAAGTT AAAGTAAAGA | |
| | | TTCCTGAAGA GCTAAAACCG TGGCTTGTTG ATGACTGGGA | |
| | | CTTAATTACC AGGCAAAAAC AGCTCTTTTA TCTTCCTGCC | |
| | | AAGAAGAATG TGGATTCCAT TCTTGAGGAT TATGCAAATT | |
| | | ACAAGAAATC TCGTGGAAAC ACAGATAATA AGGAGTATGC | |
| | | GGTTAATGAA GTTGTGGCAG GGATAAAAGA ATACTTCAAC | |
| | | GTAATGTTGG GTACCCAGCT ACTCTATAAA TTTGAGAGAC | |
| | | CACAGTATGC TGAAATTCTT GCAGATCATC CCGATGCACC | |
| | | CATGTCCCAG GTGTATGGAG CGCCACATCT CCTGAGATTA | |
| | | TTTGTACGAA TTGGAGCAAT GTTGGCTTAT ACACCTCTGG | |
| | | ATGAGAAGAG CCTTGCTTTA TTACTCAATT ATCTTCACGA | |
| | | TTTCCTAAAG TACCTGGCAA AGAATTCTGC AACTTTGTTC | |
| | | AGTGCCAGCG ATTATGAAGT GGCTCCTCCT GAGTACCATC | |
| | | GGAAAGCTGT GTGAGAGGCA CTCTCACTCA CTTATGTTTG | |
| | | GATCTCCGTA AACACATTTT TGTTCTTAGT CTATCTCTTG | |
| | | TACAAACGAT GTGCTTTGAA GATGTTAGTG TATAACAATT | |
| | | GATGTTTGTT TTCTGTTTGA TTTTAAACAG AGAAAAAATA | |
| | | AAAGGGGGTA ATAGCTCCTT TTTTCTTCTT TCTTTTTTTT | |
| | | TTTCATTTCA AAATTGCTGC CAGTGTTTTC AATGATGGAC | |
| | | AACAGAGGGA TATGCTGTAG AGTGTTTTAT TGCCTAGTTG | |
| | | ACAAAGCTGC TTTTGAATGC TGGTGGTTCT ATTCCTTTGA | |
| | | CACTACGCAC TTTTATAATA CATGTTAATG CTATATGACA | |
| | | AAATGCTCTG ATTCCTAGTG CCAAAGGTTC AATTCAGTGT | |
| | | ATATAACTGA ACACACTCAT CCATTTGTGC TTTTGTTTTT | |
| | | TTTTATGGTG CTTAAAGTAA AGAGCCCATC CTTTGCAAGT | |
| | | CATCCATGTT GTTACTTAGG CATTTTATCT TGGCTCAAAT | |
| | | TGTTGAAGAA TGGTGGCTTG TTTCATGGTT TTTGTATTTG | |
| | | TGTCTAATGC ACGTTTTAAC ATGATAGACG CAATGCATTG | |
| | | TGTAGCTAGT TTTCTGGAAA AGTCAATCTT TTAGGAATTG | |
| | | TTTTTCAGAT CTTCAATAAA TTTTTTCTTT AAATTTCAAA | |
| | | GAACAAAAAA AAAAAAAAA | |
| MRPL19 | NM_014763.3 | GTAGTCTTGA CGTGAGCTAG CTGGCATGGC GGCCTGCATT | 40 |
| | | GCAGCGGGGC ACTGGGCTGC AATGGGCCTA GGCCGGAGTT | |
| | | TCCAAGCCGC CAGGACTCTG CTCCCCCCGC CGGCCTCTAT | |
| | | CGCCTGCAGG GTCCACGCGG GGCCTGTCCG GCAGCAGAGC | |
| | | ACTGGGCCTT CCGAGCCCGG TGCGTTCCAA CCGCCGCCGA | |
| | | AACCGGTCAT CGTGGACAAG CACCGCCCCG TGGAACCGGA | |
| | | ACGCAGGTTC TTGAGTCCTG AATTCATTCC TCGAAGGGGA | |
| | | AGAACAGATC CTCTGAAATT TCAAATAGAA AGAAAAGATA | |
| | | TGTTAGAAAG GAGAAAAGTA CTCCACATTC CAGAGTTCTA | |
| | | TGTTGGAAGT ATTCTTCGTG TTACTACAGC TGACCCATAT | |
| | | GCCAGTGGAA AAATCAGCCA GTTTCTGGGG ATTTGCATTC | |
| | | AGAGATCAGG AAGAGGACTT GGAGCTACTT TCATCCTTAG | |
| | | GAATGTTATC GAAGGACAAG GTGTCGAGAT TTGCTTTGAA | |
| | | CTTTATAATC CTCGGGTCCA GGAGATTCAG GTGGTCAAAT | |
| | | TAGAGAAACG GCTGGATGAT AGCTTGCTAT ACTTACGAGA | |
| | | TGCCCTTCCT GAATATAGCA CTTTTGATGT GAATATGAAG | |
| | | CCAGTAGTAC AAGAGCCTAA CCAAAAAGTT CCTGTTAATG | |
| | | AGCTGAAAGT AAAAATGAAG CCTAAGCCCT GGTCTAAACG | |
| | | CTGGGAACGT CCAAATTTTA ATATTAAAGG AATCAGATTT | |
| | | GATCTTTGTT TAACTGAACA GCAAATGAAA GAAGCTCAGA | |
| | | AGTGGAATCA GCCATGGCTT GAATTTGATA TGATGAGGGA | |
| | | ATATGATACT TCAAAAATTG AAGCTGCAAT ATGGAAGGAA | |
| | | ATTGAAGCGT CGAAAAGGTC TTGATTCTGA GAATGAATTT | |
| | | GGTTAGTTGC AGAAGATACA TTGGCTCTAA GAGGATATAT | |
| | | TTTGAGACCA ATTTAATTTC ATTTATAAGA ACATAGTAAT | |
| | | TAAGTGAACT AAGCATTCAT TGTTTTATTA ATACTTTTTT | |
| | | TCTAAAATAA AACTTGTACA CCAGTTTATT ACTCTAAAAA | |
| | | GAGAATTACA CATGCCAAAT GGACCAATGT CCATTTGCTT | |
| | | ATTGGAGGCA AAGCTACAAT AGAAGTCAGA GCATCACCAG | |
| | | AATGGTCTTT AATGAGCATG GAACCTGAGC AAAGGGAATA | |
| | | GGTGGGATGA ATTTTTTTTT TAATTGTGAA ACAATTCATA | |
| | | AGCACAATAT GATTTACAGA ATAATAAACA TTCATGTACC | |
| | | CACTATCAGG TTAAGAAATA GAACATTTAT TAATATGTAG | |
| | | GAATGTTAAG AAATAAAACA TTTAATAAGA TCTCAGAAGA | |
| | | CTCCAGTAAA TCTGCAATTG TATCTCTCTC CTTTTTAAAT | |
| | | GTAAATATCA TCTTGACTTG TTAATTATTC CCTTGCATTT | |
| | | CTTTTAGTTT ACTGCCAACA CATATATTCT TCAACAATAT | |
| | | ATTTAATTTT GAAAAACCTG AAAAAAAAAA CCTGTTAGCA | |
| | | AGTATAAAGG GGCAGTATTA CTATTATTGC ATGAAGGCTT | |
| | | CAAGGGAAAC GTTACAGTCT TTGGGTCATA GTCTGGCTTC | |
| | | AGCTTCCTCT GAGAGTTTAC AGAGGCCAAT TTTGAGCAAA | |
| | | TTCATGGCTA AGGTTATGAG TGAGTTCTGC TAAACAGAAG | |
| | | GCTCACCACA AGGTATCTGG CAGGATTATA CTGGGTAGCT | |
| | | GGATGTTGCA GAAATGTGGT TAGAGGAAGT AAACTGTTTT | |
| | | TTGATGCTCA CAGCATGATG AATCAAACTC TGTATCTTAG | |
| | | GATTAGGTTA AAACAATACC TTTGGTATGA TATGAGTGTT | |
| | | GTTGCTGATC CATGCAGCAT GGATTGGAAA GCTGGGGTAT | |
| | | AAGCACACAT GCTAAAGAAA AACATGTAAT TTGGTCCATA | |
| | | CTCACCTGGA TATACTGTTC CTCAGGTTAA AAAATACAGT | |
| | | ACTATCCTAA ATCTTGAAGG CAACTCTCAG CCTATCCATT | |
| | | GAGTTACCTT CAGATCTGCC CTCTGGTTCC TAGCTGTCTT | |
| | | GGGACTAACT TCTTTCCTGC GCTCAGCTGT TTTCTGGATT | |
| | | CCATGTTTTC CATTTTATTG AGTACTAACT TGTTTTGCTG | |
| | | CAGCACATCC TTTGGTAGCT TCTAGAGGAA GTTTGTGTGG | |
| | | AGGTAAAATT TTTGAGACCT TGCATGTCTC ATGTTTGATT | |
| | | GATACTTTAT ACGTTTAGGT AGGAGGTAAT TTTCCTTCAG | |
| | | GACTTTAAAA ATATTGTTGC TCCATTTTCT TTGTTTCTAT | |
| | | TGTTGTATTG AGAAATCCAA TGCCATTTTG ATTTCCCCAT | |
| | | CATAAATTTC ATGATGATGT GTCTTGGTGT GGGTCTATAT | |
| | | TTATCCATTG TATTGGGTTT TAGGTGAACC CTTCCAGATA | |
| | | GTAACTCATT TCTGTCAGTT CTGGGAAACA CTTAGCATTG | |
| | | GTTGATGATT TATTCTCTGC TGCTTTGTTC TCCCAACTAT | |
| | | TATTTGGATG TTGGATATCC AGCACTGGGT ATCTATTTTC | |
| | | TTACCTCCCT CCCTTGACCC CAGTCTCTGT TTTTTAGCTC | |
| | | TTTAGCTCAA TCTTCCAACT CTTTGCTATT GTATTTTAAA | |
| | | ATCTTAAGAC CCCTTCTTGA TTTGTAGAAG TTCCTTTTCT | |
| | | TACAACCAAA AAGCCTTTAT CTATGGATTT GTTCACAGAT | |
| | | AAGGGGTATT CAATATAGTG TATTTTTTTT TCATTTAAAA | |
| | | TTGTTTGCGC ATCTATTTCC TCCAAATTTC TTTCTGTATT | |
| | | TATTTTTTGT TGTCTATATT TCAGACTTTT CCAGGATATC | |
| | | TGATAATCTT TGGCTGTCTT CTTATGGTTG AAAGAGGGAC | |
| | | TAAAAAGCTT GGAAAGCCTT TGGGTTGTGG GAAGGGGCTG | |
| | | TCTTTAGGAT TATCTGAATG GGCTTTTTTG GGAGTCCCCT | |
| | | CCTCCACATG AATATTTTGG TTTTGTCAGA TTCCCTAGAA | |
| | | TAGAGGCTTC CAATCTCCTT CCTGGAGGGG TCTGTCCAGG | |
| | | AAGGAGATTG TCTAGGGGTC TGTCAGACAG CAGCTTTCAG | |
| | | CTACTTCCTT GATCTTTTTC ACTAATGATT ATATAGTCAT | |
| | | CTAACTACTG TCAACAAGTA ATAGATATCC TATCCTTCAC | |
| | | TTGTTTAGAT TATTTGCTGA GATAACCTCT CAAAAGAACC | |
| | | TCTCAAAATA AAAGGTTAAC AAGAGCCTAT ATCTTATATT | |
| | | TTTCTTCTCT TTATCTTGTT AGAAGATAGC TATTAAAACC | |
| | | TGTTCTTTTT CTGTCTTGAT AAACACACTT CAATCTTGGT | |
| | | AGAATGGTAG ATGGGACAGT ATATTTTAGG ACCTAAAGCT | |
| | | CTGCAAATGT ATGATCAGCT TGTAAGTACA GGTGCTCAAA | |
| | | AACATGTAAA CAATCATGCT TTTTACTCTG TAGGAATATC | |
| | | TTTAAAATTC TTGTGAATTT TTCCCCAGAA GTAAAGCAAA | |
| | | TCTTCCCCCA GAAATAAAAT TAAATGTGCA TAATCTAAAG | |
| | | CTTTTTTTTT TTATTGTGGT AGGATATATA TATAAAACAT | |
| | | AATTTGCCAT TGTAAACATT TTAAATTTAC AAGTCAGAGG | |
| | | CATTAATTAC ATCACAATGT TGTGAAATTA TTACTACTAT | |
| | | TTCCAAAATT TTCTCATCAC CCCAAACTGA AACTCTGTAA | |
| | | CTGTTGAGCA ATAACCTCAT TCCTGTATCT CTCCCAACCC | |
| | | CAGGTAACCT CAAATCTTTC TTTTTATCTT TGAGACAAGG | |
| | | TCTCATTCTA TCACTCAGGT AGGAGTGCAG TGGTGTGATC | |
| | | ATAGCTCATT GCAGCCTCAA AATCCTGGGC TCAAGCAATC | |
| | | CTCCTTGAGT AGCTAAGACT ATAGGCACAC ATTAACTGCG | |
| | | CCTGGCTGAT TTTGTTTTTT GTAGAGATGT GGTCTTGCTA | |
| | | TGTTTCCCAT GCTGGTCTTG AGTTCCTGGC CTCAAGCAGT | |
| | | CCTTAAGATT CATCCATGTT GTGGCATGTG TCAGAATTTC | |
| | | ATTTGTTTTT ATGACTAAAT AATATTCCAT TGTATGTATA | |
| | | TACATTTTGT TCATCCATCT TCTGATGAAC ACTGGGATAT | |
| | | GTCTACCTTT TGGCTATTGT GAATAATGCT GCAGTAAACA | |
| | | TTGACATAAC AAGTATGTAT TTGATTGCCT GTTTCTAAGT | |
| | | TCTTTTGGGT ATACATCTTG AGTAGAATTG CTAGATAATG | |
| | | TCATGTTTTA TTTCTCTTGT GATTTCTTCT TCGATCCCCT | |
| | | GGTTGAGTGT GTTAATTTCT ACATGTTTAT GAATTTCCCA | |
| | | CTGTTTTTTT GTTATTGATT TCCAAGTTCA TTCCATTGTG | |
| | | ATTAGAGAAG ATACTTAGTA TGATTTTAAT GTTTTTGAGA | |
| | | ATTGGTGTGT GGCCTGATAG ATGGTCTGTC CTGGAGAATG | |
| | | TTCCTCATAC ACTTGAGCAA AATATTTATC ATGCTATTGT | |
| | | TGACTGTAGT TTTCTATATG TCTCTTAGGT CAAGGTGGTT | |
| | | TACAATGTGT TAAGGTTCTC TTTTTTTAAA AAAATTTTTG | |
| | | CACAGAGTAT CTTTTTCTAT GTGTTCCATG TATTTGTGTC | |
| | | TTTGGAGCTA TAGTCTCTTG TAGACAGCAT ATCACTATCT | |
| | | TGTTTTGTTT TGTTTTTTCT GTCCATTCTG CCAATTTCTG | |
| | | CCTTTTGATT GGAAAATTTA ATCCATTTGC ATTTAAAGTA | |
| | | ATTAAGGAAG GACTTTCTTC TACCATTTAA CACTTCTTCT | |
| | | ATATGTCATA TACTTTTTTG GCCCCTCATT TCCTCTTTAT | |
| | | GGCCTTCTTT TCTGTTTTTT TGTAGTGAAC TAGTCTGATT | |
| | | CTCTTTCCAC TCCCCTTTGT GTATATTTGT TAGATGTTTT | |
| | | ATTTGTGGTT GCTATGGGGA TTATAGTTAA CATCCTACAC | |
| | | TTAAAACAAT CTAATTTAAA CTGATACCAA TTTACCTTCA | |
| | | ATAGCATACA AAATCTCTAC TCCTGTAAAG CTCTGCCCCT | |
| | | GCCCCCCTTA TGTTATTGAT GGCACAAATT GCCTAATAAA | |
| | | TAATTTATAG TTATTTGTAT GAGTTTGTCT TTTAAATCAT | |
| | | TTAGGAAATA AAAAGTGGAG TTAGAAAACA GTATGATAGT | |
| | | AATACTGACT TTTATATTTG TCAATATATT TATCTTATTT | |
| | | TGGATCCTTA TTTCATTATA TAGATTTGAG TTACTGTCTA | |
| | | GTGCCCTTCC ATTTCGGCCC AAAGGATTCC CTTATGCATT | |
| | | TCTTGCAGGG CAAGTCTAAT TGTAATAAAC TCCCTCAGCT | |
| | | TTTGTTTTAT CTGAGAATGT CTTGATTTCT CCCTTATTTT | |
| | | TGATGGATAA TTTTGCCAGA TACATGAATT TTTGGTAACA | |
| | | GTATTTTTCT TTCAGCACTT TAAATATGTC ATCCCACTAC | |
| | | CTTCTGACTT CATGGTTTCT CATGAGATAT TAGATGTTAT | |
| | | AAAATTTGAG GATTCCTCAT TCTTGATGAG TCAGTTCTGT | |
| | | CTTATTGCTT TTCGGATTTG CTCAGCTTTT GTCTTTTGAC | |
| | | AGTTTGATTA TAACGCGGCT CAGTGTGGGT CTCTGAGTTT | |
| | | ATCCCACTTA GAGTTTGTTG AGTTTCTTGG AGTCATAGAT | |
| | | TTATGTCTTT TATCAAATTT TGGACATATT TGGCTATTAT | |
| | | TTCTTCAATT TTTTTCACTG CTTCTTTCTT TTCCTTCTGA | |
| | | AATATTCTTA ATGTATATGT TGGTCTGTTT GATGCTGTCT | |
| | | CACCAGTTTC TTAGGCTGTG TTCTCTTTTG TTCCTCAGAC | |
| | | TTGATTATTG CAGTTGCCCT TCTTTTTATT TTTTTCAAGT | |
| | | TTGTTGATTC TTCTCCCTGT TCAGATCAAC TGTTGAACTC | |
| | | CTCTAGTGAA TTTATTTCAG TTACTGTACT TTTCAGCTCC | |
| | | AAGATTTATC TTTGGTTCCT TTTTATAACG TCTGTGTCTT | |
| | | TATTGATATT CTCATTTTGT TCATATGTCT CTTTCTTCCT | |
| | | TTAGTTCTTT GTCCATGTTT TCCTTTAGCT CTTTGGGCTT | |
| | | ATTTAAGACA ATTGTTTAAA GTCTTTGCAT AGTAAGTCCA | |
| | | ATGTCTGTGT TTCTTCAGGG ATGGTTTTCA TTATTTTGTT | |
| | | TTCAATGAGC CATACTTTCC TGTGTCTTTG TATGCTGTCT | |
| | | TTTTGTTGTT GAAAACTGTA TGTTTGAACA TCATAACGTG | |
| | | GTGGCCCTGA AAATCAGATA TTCCCCCCTT CCTGAGAGTT | |
| | | AGTTTTATTT TTATTATTGA AGATTGTAGC AGTCTATTGC | |
| | | TACATGTGCA GTCATTTCCA AACTATTTTT GCAAAGACTG | |
| | | TATTCCTTCT GTGTGTCATC ACTGAAGTCT CTGTTCCTTA | |
| | | GTTTGTGTTT AATAGTTTGA CATAGATTTC CTTGAAAGGA | |
| | | GTTAAAACTA GCAGAAAAAT CTCTCTCCCA GTCTTTCCAG | |
| | | TCTTTGTAGA TTGGTTCTGT GCTGGGCTTT TCCATTAATA | |
| | | CTTAGCCAGG CTTGTACTGA GCCTAACAAT CAGGCCCAAA | |
| | | AGCGTAGGGT CTTTGCAGAT CTTGTCTGAG CATGCTTCTT | |
| | | GCTGTGTATG CACGTAGTTT TCTAAATCTC CCTGTATGTG | |
| | | CTGTTGAATA TTCTAATTTC CCAAAGAAAC TCCTTTGCAG | |
| | | CTTTTTCTCA CAGAACATAG ATGGTTTTTT GGATATCTTG | |
| | | ACCATAGTCT TTCGACCCAG GTGTTTGCGG TTGTTAGTTC | |
| | | ACCTTACACT TTTTTCAAGC ATTGCCTACT GCTTACGATG | |
| | | AGTGCTCTGT CAATCCTTTA AGTAGCCCCA GACAGGCTAC | |
| | | CAGAGACTTA AACAAGAATT TGTAAGTTCT GCTCAGCTTC | |
| | | CTCTAGAAAT GGGGATCAGG GTCCAAGACA GAATGCAGTT | |
| | | GCTGATTTCA AGACTGCTGC AACACCAGGG AGCTTGTGGG | |
| | | GGAAGGGCAA GCAGAAATGT CACAAAGCTT TCTTGCCATT | |
| | | TTAAAGTTGC CTGTTCTTGA CTCAGCATTT GCTTCATTGC | |
| | | TATAAACTTT TTACTGTTTT TCAGAGTTCT GATAAAATTG | |
| | | GCTATGCCTG TTCCTGCTTT AAAAAATATA TATATATTTT | |
| | | TTAGGGATTG GGGTCTCACT ATACTGACCA GGCTGGTCTT | |
| | | GAACTTCTGG CCTCAAGCCA TCCTCTCATT TCAGCTTCCC | |
| | | AAAGTGCTGC AATTACACGC GTGAACCACC ACACCCAGCC | |
| | | CCTGCTTGTT TTTCAATGTG CCTACTCCAC CATGTTGCTC | |
| | | AAGTATGTAT ATTTTCTAAA CTACCTTGTA GTGTTGTGAT | |
| | | GGGAAATAAA TCCCTGAGCC TTTTGAATAA CTCAGAGAGA | |
| | | TCAAAAACTT AGTTTATCCT ATTCGAAGGA TTAGAAAAAT | |
| | | GATATATCTT TCACTTTTTC AGGGATAGGC TCCTCATTAG | |
| | | AAGGCTCCTA TGTGCCGATG CTGTACAAGA CATTTCATTT | |
| | | CTCTTAATGT TTACAACAAG CTTGTTGCCA AGGCTGATCT | |
| | | TGAACTCCTG GCCTCAAACG ATCCTCCCAG CTCAGTCTCA | |
| | | CAAAGTGTTG GGATGTCTGG CCAACTAATG ACTATCTTAA | |
| | | CTCTTGTGTT TCAATGTTTA TGCCTTCTTT TATCTTGACT | |
| | | GATTGTATGA CTATGTCTTC TAGAACAATG TTGAACAGAA | |
| | | ATGGTGAGAG CAGACATCCT TGCTTTAATA TTTCACCATT | |
| | | ATATATGATG TTAGGTATAG ATTTTTCTCA CAGATGCCTT | |
| | | TTATCAGATT GAGGAATTTA TATTCCTACT TTGCCGAAAG | |
| | | GTTTTTGTAG TATGAGGGGG TGCTGAATTT TGTCAAACAC | |
| | | TTTTTCGGTA ATAATTGAGA TGATTGGTTC TGCAGTCATC | |
| | | GAGATGTGGA TTTTCTCCTT TATTCTGTTC GTGAGTGATT | |
| | | ACACTGGTTG ACTAATGTTA AAACAACCTT ACTTTCCAGG | |
| | | AATAAACCCT ATTATCTTTT TTATACA | |
| PSMC4 | NM_153001.2 | TGCGGGTACG GACAGCGCAT GAGCTTATGT TGAGGGCGGA | 41 |
| | | GCCCAGACCA GCCCTTCGTC CTATCCTGCC CTTCCAGCAC | |
| | | CTCTCAGCCG TAACTTAAAC TACACTTCCC AGAAGCCTCC | |
| | | TCAGCCAGGG ACTTCCGTTG TCGTCAGCGG AAGCGGTGAC | |
| | | AGATCATCCC AGGCCACACA GAGGCCGGCT TGGTCACTAT | |
| | | GGAGGAGATA GGCATCTTGG TGGAGAAGGC TCAGGATGAG | |
| | | ATCCCAGCAC TGTCCGTGTC CCGGCCCCAG ACCGGCCTGT | |
| | | CCTTCCTGGG CCCTGAGCCT GAGGACCTGG AGGACCTGTA | |
| | | CAGCCGCTAC AAGGAGGAGG TGAAGCGAAT CCAAAGCATC | |
| | | CCGCTGGTCA TCGGACAATT TCTGGAGGCT GTGGATCAGA | |
| | | ATACAGCCAT CGTGGGCTCT ACCACAGGCT CCAACTATTA | |
| | | TGTGCGCATC CTGAGCACCA TCGATCGGGA GCTGCTCAAG | |
| | | CCCAACGCCT CAGTGGCCCT CCACAAGCAC AGCAATGCAC | |
| | | TGGTGGACGT GCTGCCCCCC GAAGCCGACA GCAGCATCAT | |
| | | GATGCTCACC TCAGACCAGA AGCCAGATGT GATGTACGCG | |
| | | GACATCGGAG GCATGGACAT CCAGAAGCAG GAGGTGCGGG | |
| | | AGGCCGTGGA GCTCCCGCTC ACGCATTTCG AGCTCTACAA | |
| | | GCAGATCGGC ATCGATCCCC CCCGAGGCGT CCTCATGTAT | |
| | | GGCCCACCTG GCTGTGGGAA GACCATGTTG GCAAAGGCGG | |
| | | TGGCACATCA CACAACAGCT GCATTCATCC GGGTCGTGGG | |
| | | CTCGGAGTTT GTACAGAAGT ATCTGGGTGA GGGCCCCCGC | |
| | | ATGGTCCGGG ATGTGTTCCG CCTGGCCAAG GAGAATGCAC | |
| | | CTGCCATCAT CTTCATAGAC GAGATTGATG CCATCGCCAC | |
| | | CAAGAGATTC GATGCTCAGA CAGGGGCCGA CAGGGAGGTT | |
| | | CAGAGGATCC TGCTGGAGCT GCTGAATCAG ATGGATGGAT | |
| | | TTGATCAGAA TGTCAATGTC AAGGTAATCA TGGCCACAAA | |
| | | CAGAGCAGAC ACCCTGGATC CGGCCCTGCT ACGGCCAGGA | |
| | | CGGCTGGACC GTAAAATTGA ATTTCCACTT CCTGACCGCC | |
| | | GCCAGAAGAG ATTGATTTTC TCCACTATCA CTAGCAAGAT | |
| | | GAACCTCTCT GAGGAGGTTG ACTTGGAAGA CTATGTGGCC | |
| | | CGGCCAGATA AGATTTCAGG AGCTGATATT AACTCCATCT | |
| | | GTCAGGAGAG TGGAATGTTG GCTGTCCGTG AAAACCGCTA | |
| | | CATTGTCCTG GCCAAGGACT TCGAGAAAGC ATACAAGACT | |
| | | GTCATCAAGA AGGACGAGCA GGAGCATGAG TTTTACAAGT | |
| | | GACCCTTCCC TTCCCTCCAC CACACCACTC AGGGGCTGGG | |
| | | GCTTCTCTCG CACCCCCAGC ACCTCTGTCC CAAAACCTCA | |
| | | TTCCCTTTTT TCTTTACCCA GGATTGGTTT CTTCAATAAA | |
| | | TAGATAAGAT CGAATCCATT TAATTTCTTC TTAGAAGTTT | |
| | | AACTCCTTTG GAGAATGTGG GCCTTGAATA GGATCCTCTG | |
| | | GGTCCCTCTT AATCTGACAG ATGAGCAGAC GAGGTGCATG | |
| | | GCCTGGGTTG CAGCTTGAGA GAACCAAAAT ATTCAAACCA | |
| | | GATGACTTCC AAAATGTGGG GAAAGGGATG GAAAATGAAC | |
| | | CTGAGATGGA GTCCTTAATC ACGGGATAAA GCCCTGTGCA | |
| | | TCTCCCTCAT TTCCTACAGG TAAAAGACAG TAAAGAAATT | |
| | | CAGGTCACAG GCCTTGGGAG TTCATAGGAA GGAGATGTCC | |
| | | AGTGCTGTCC AGTAGAACTT T | |
| SF3A1 | NM_005877.5 | GGTCCCGGAA GTGCGCCAGT CGTACCTTCG CGGCCGCAAC | 42 |
| | | TCGCTCGGCC GCCGCCATCT TGCGAGCTCG TCGTACTGAC | |
| | | CGAGCGGGGA GGCTGTCTTG AGGCGGCACC GCTCACCGAC | |
| | | ACCGAGGCGG ACTGGCAGCC CTGAGCGTCG CAGTCATGCC | |
| | | GGCCGGACCC GTGCAGGCGG TGCCCCCGCC GCCGCCCGTG | |
| | | CCCACGGAGC CCAAACAGCC CACAGAAGAA GAAGCATCTT | |
| | | CAAAGGAGGA TTCTGCACCT TCTAAGCCAG TTGTGGGGAT | |
| | | TATTTACCCT CCTCCAGAGG TCAGAAATAT TGTTGACAAG | |
| | | ACTGCCAGCT TTGTGGCCAG AAACGGGCCT GAATTTGAAG | |
| | | CTAGGATCCG ACAGAACGAG ATCAACAACC CCAAGTTCAA | |
| | | CTTTCTGAAC CCCAATGACC CTTACCATGC CTACTACCGC | |
| | | CACAAGGTCA GCGAGTTCAA GGAAGGGAAG GCTCAGGAGC | |
| | | CGTCCGCCGC CATCCCCAAG GTCATGCAGC AGCAGCAGCA | |
| | | GACCACCCAG CAGCAGCTGC CCCAGAAGGT CCAAGCCCAA | |
| | | GTAATCCAAG AGACCATCGT GCCCAAAGAG CCTCCTCCTG | |
| | | AGTTTGAGTT CATTGCTGAT CCTCCCTCTA TCTCAGCCTT | |
| | | CGACTTGGAT GTGGTGAAGC TGACGGCTCA GTTTGTGGCC | |
| | | AGGAATGGGC GCCAGTTTCT GACCCAGCTG ATGCAGAAAG | |
| | | AGCAGCGCAA CTACCAGTTT GACTTTCTCC GCCCACAGCA | |
| | | CAGCCTCTTC AACTACTTCA CGAAGCTAGT GGAACAGTAC | |
| | | ACCAAGATCT TGATTCCACC CAAAGGTTTA TTTTCAAAGC | |
| | | TCAAGAAAGA GGCTGAAAAC CCCCGAGAAG TTTTGGATCA | |
| | | GGTGTGTTAC CGAGTGGAAT GGGCCAAATT CCAGGAACGT | |
| | | GAGAGGAAGA AGGAAGAAGA GGAGAAGGAG AAGGAGCGGG | |
| | | TGGCCTATGC TCAGATCGAC TGGCATGATT TTGTGGTGGT | |
| | | GGAAACAGTG GACTTCCAAC CCAATGAGCA AGGGAACTTC | |
| | | CCTCCCCCCA CCACGCCAGA GGAGCTGGGG GCCCGAATCC | |
| | | TCATTCAGGA GCGCTATGAA AAGTTTGGGG AGAGTGAGGA | |
| | | AGTTGAGATG GAGGTCGAGT CTGATGAGGA GGATGACAAA | |
| | | CAGGAGAAGG CGGAGGAGCC TCCTTCCCAG CTGGACCAGG | |
| | | ACACCCAAGT ACAAGATATG GATGAGGGTT CAGATGATGA | |
| | | AGAAGAAGGG CAGAAAGTGC CCCCACCCCC AGAGACACCC | |
| | | ATGCCTCCAC CTCTGCCCCC AACTCCAGAC CAAGTCATTG | |
| | | TCCGCAAGGA TTATGATCCC AAAGCCTCCA AGCCCTTGCC | |
| | | TCCAGCCCCT GCTCCAGATG AGTATCTTGT GTCCCCCATT | |
| | | ACTGGGGAGA AGATCCCCGC CAGCAAAATG CAGGAACACA | |
| | | TGCGCATTGG ACTTCTTGAC CCTCGCTGGC TGGAGCAGCG | |
| | | GGATCGCTCC ATCCGTGAGA AGCAGAGCGA TGATGAGGTG | |
| | | TACGCACCAG GTCTGGATAT TGAGAGCAGC TTGAAGCAGT | |
| | | TGGCTGAGCG GCGTACTGAC ATCTTCGGTG TAGAGGAAAC | |
| | | AGCCATTGGT AAGAAGATCG GTGAGGAGGA GATCCAGAAG | |
| | | CCAGAGGAAA AGGTGACCTG GGATGGCCAC TCAGGCAGCA | |
| | | TGGCCCGGAC CCAGCAGGCT GCCCAGGCCA ACATCACCCT | |
| | | CCAGGAGCAG ATTGAGGCCA TTCACAAGGC CAAAGGCCTG | |
| | | GTGCCAGAGG ATGACACTAA AGAGAAGATT GGCCCCAGCA | |
| | | AGCCCAATGA AATCCCTCAA CAGCCACCGC CACCATCTTC | |
| | | AGCCACCAAC ATCCCCAGCT CGGCTCCACC CATCACTTCA | |
| | | GTGCCCCGAC CACCCACAAT GCCACCTCCA GTTCGTACTA | |
| | | CAGTTGTCTC CGCAGTACCC GTCATGCCCC GGCCCCCAAT | |
| | | GGCATCTGTG GTCCGGCTGC CCCCAGGCTC AGTGATCGCC | |
| | | CCCATGCCGC CCATCATCCA CGCGCCCAGA ATCAACGTGG | |
| | | TGCCCATGCC TCCCTCGGCC CCTCCTATTA TGGCCCCCCG | |
| | | CCCACCCCCC ATGATTGTGC CAACAGCCTT TGTGCCTGCT | |
| | | CCACCTGTGG CACCTGTCCC AGCTCCAGCC CCAATGCCCC | |
| | | CTGTGCATCC CCCACCTCCC ATGGAAGATG AGCCCACCTC | |
| | | CAAAAAACTG AAGACAGAGG ACAGCCTCAT GCCAGAGGAG | |
| | | GAGTTCCTGC GCAGAAACAA GGGTCCAGTG TCCATCAAAG | |
| | | TCCAGGTGCC CAACATGCAG GATAAGACGG AATGGAAACT | |
| | | GAATGGGCAG GTGCTGGTCT TCACCCTCCC ACTCACGGAC | |
| | | CAGGTCTCTG TCATTAAGGT GAAGATTCAT GAAGCCACAG | |
| | | GCATGCCTGC AGGGAAACAG AAGCTACAGT ATGAGGGTAT | |
| | | CTTCATCAAA GATTCCAACT CACTGGCTTA CTACAACATG | |
| | | GCCAATGGCG CAGTCATCCA CCTGGCCCTC AAGGAGAGAG | |
| | | GCGGGAGGAA GAAGTAGACA AGAGGAACCT GCTGTCAAGT | |
| | | CCCTGCCATT TTGCCTCTCC TGTCTCCCAC CCCCTGCCCC | |
| | | AGACCCAGGA GCCCCCCTGA GGCTTTGCCT TGCCTGCATA | |
| | | TTTGTTTCGC TCTTACTCAG TTTGGGAATT CAAATTGTCC | |
| | | TGCAGAGGTT CATTCCCCTG ACCCTTTCCC CACATTGGTA | |
| | | AGAGTAGCTG GGTTTTCTAA GCCACTCTCT GGAATCTCTT | |
| | | TGTGTTAGGG TCTCGATTTG AGGACATTCA TTTCTTCAGC | |
| | | AGCCCATTAG CAACTGAGAG CCCAGGGATG TCCTACAGGA | |
| | | TAGTTTCATA GTGACAGGTG GCACTTGGCT AATAGAATAT | |
| | | GGCTGATATT GTCATTAATC ATTTTGTACC TTGACATGGG | |
| | | TTGTCTAATA AAACTCGGAC CCTTCTTGTG AAATCAGTTA | |
| | | AATAAGACTT GTCTCGGTCA CCTGTGCCCT GTCCAGACTC | |
| | | GAGGCAGTGG TAACACTGCA CAGTGCTATG TGGCTTCTCT | |
| | | TTGAGGATTT TTGGGTTTTG TAACTAAATT CTTGCTGCCC | |
| | | TCATACTTTT TATGTATTAG AATCATATTC GTATTGCCCT | |
| | | TTTAAAACAT TGGGATCCTC CAAAGGCCTG CCCCATGTAT | |
| | | TTAACAGTAA TACAGGAAGC ATGGCAGGCA CCATGCAAAC | |
| | | CAAGGATGGA TGGTGCAGTC CCTGTGTCAG TGGGCGGTGG | |
| | | TTTCCTGCTG GCCTGGAATC ACTCATCACC TGATTGATTG | |
| | | GCTCTGTGGT CCTGGGCAGG TGCCTCATAG GTGTGTGGAT | |
| | | ATGATGACGT TTCTTTAAAA TGTATGTATT TAACAAATAC | |
| | | TTAATTGTAT TAAGGTCATG TACCAAGGAT TTGATAAAGT | |
| | | TTAAATAATT TACTCTCTAC TTTTATCCAT TTTATCCATT | |
| | | TTAACTCATG TAATCCTCAT GTGAGTATTC CTGTTTAACA | |
| | | CTTGAGTAAA CTGAGGCACA GAGAACATAA GTTGCATGCC | |
| | | ATAGTCACAC ACTGTGAAAG TGAAAAGAGA ATGTGTGCAA | |
| | | AACACGTCAC AGTCCTGGTT TCTGAGTAAA GGCAGGCTGT | |
| | | TATCTTTAGA ATCAAGCTAT CACAGGGAGA TAGGCAATGC | |
| | | TGTGGGTGTT GGAGGAAGGT GAGAGCCTGT TGCTAACAAT | |
| | | TTCCTGGTTT TAAAGCTAAG GCTGATTTTA TTGGGAAGAT | |
| | | CTCACATGTG TGTGGCCCCT GAGAGTTCCC AGTGCCTTTT | |
| | | ATTTGCAGTC CTTCCATTTG GACCTCCTAG CTGCCCCATC | |
| | | AGGTCATCTC CAGGGCTCAG AGGGGTGAGA CCATTTCCCA | |
| | | AGGTCACAGA ACCAGCTCTC TAGTCACCAC CCTGCCTCTC | |
| | | CCTCTCACCC AGAGTCAGTA CCAGTTTTAT GGCTTTATTA | |
| | | CAAACTGCTG GGTCCCTCCC ATTTTCAACT TGATTGATGG | |
| | | GATGTCATCC CTTATCCTGT CTGACATTTG CCTCTGGCCT | |
| | | GGTTGCTAGA AGTTTGCCCC AGGGGCAAGA GTTGAAATTT | |
| | | GGCTTCCTGA GGTGGGCTTT GTGGTTTGCG TCCCTAAAGT | |
| | | GAGCCCACTA CTGGTTGCTT GTCCATGGCC AACACCAGAA | |
| | | ATCCCCTGAG CACTACCTGG GTCTCATTCC AAGAAGGAAG | |
| | | AGGGTCAGGA GACCTGGGGA GTCTCATATT CCAAGTTCTT | |
| | | CTTTCTTTCT GGGAGCAGTG GGCAGTTCAT GGTGTTAGGG | |
| | | CACTCACCCC CACAGACTGG CAAACCCTGC AGGACTTCCG | |
| | | TGGCTGAGGC TGTGACCGGA GGCCAGGAAT GCCGTTGGGT | |
| | | GGATTGTGAG TGAATGGGCC CTTTGAGCTG CCCTCTAGAG | |
| | | AGCAAATCCA GTTTCCTGGA GCTCCTGAAT GAATATCTGT | |
| | | ACTGGCTCGC TCAGATGCAG AAGCTCCATT GACCATGAGG | |
| | | CCTTGTGAAC ATCAGTGGCC ACAGGCCCAG TGTGCTGCTT | |
| | | GGCACTGCAC TAGTTTAGGA CCTGCAGCAT GTAGGTAGCG | |
| | | TCCTAGTGTT TATAATACAA AGCTGCTCTG CACAGCTTTT | |
| | | CTGATTCTTC TTGCAATCTC CTGAGGATTA TCTGCCCCAT | |
| | | TTTTAAAACG AGGTGGAATA CCCAAGGTCA TGTAGCCAGT | |
| | | GAGTGCTCTG GAAAGCCAAA GCAGCTCATC CCTTCCTGGG | |
| | | GACCACACTG CTCTGCTCCA CCAGACCACA CTATGAAATA | |
| | | GGAATAAGTG CTCCTGTTGC AGGACTGCTG GGAAAACAGG | |
| | | TGGTGTGGGA CTTAAGTCAC CATAATTTTG AAGACTTGCA | |
| | | TGCAGAGGGC TCCAGGAATT GTAGACATTA AGGAATTTCA | |
| | | CTTTCAGTTC TACCCACTAC TTAAGTACTT GTCATGTACT | |
| | | CTTAGAGGAG GCCAGTAATG ATCAGAACCA TTTTACTTTA | |
| | | AAATTAATAA TATTGTATTA GAGAATATAT TAAATGGTTA | |
| | | TATTGGGTTA TGTTAGGATA TATACTTGAA TGGAAATACA | |
| | | TGTACTATTA GCAATCATAT TTCATTTATC CCTGTAATTA | |
| | | GACAAGAAAG CATAATATAG CTCTACTCAT GGGTACACAT | |
| | | ACCAGTGTAT AAGATTTTTA GAAGTTTACT TTTTAAAAAT | |
| | | AAAAGCAAAA TGTAAGATCT TAAAAAAAAA AAAAAAAAA | |
| PUM1 | NM_001020658.1 | AGTGGGCCGC CATGTTGTCG GAGTGAAAGG TAAGGGGGAG | 43 |
| | | CGAGAGCGCC AGAGAGAGAA GATCGGGGGG CTGAAATCCA | |
| | | TCTTCATCCT ACCGCTCCGC CCGTGTTGGT GGAATGAGCG | |
| | | TTGCATGTGT CTTGAAGAGA AAAGCAGTGC TTTGGCAGGA | |
| | | CTCTTTCAGC CCCCACCTGA AACATCACCC TCAAGAACCA | |
| | | GCTAATCCCA ACATGCCTGT TGTTTTGACA TCTGGAACAG | |
| | | GGTCGCAAGC GCAGCCACAA CCAGCTGCAA ATCAGGCTCT | |
| | | TGCAGCTGGG ACTCACTCCA GCCCTGTCCC AGGATCTATA | |
| | | GGAGTTGCAG GCCGTTCCCA GGACGACGCT ATGGTGGACT | |
| | | ACTTCTTTCA GAGGCAGCAT GGTGAGCAGC TTGGGGGAGG | |
| | | AGGAAGTGGA GGAGGCGGCT ATAATAATAG CAAACATCGA | |
| | | TGGCCTACTG GGGATAACAT TCATGCAGAA CATCAGGTGC | |
| | | GTTCCATGGA TGAACTGAAT CATGATTTTC AAGCACTTGC | |
| | | TCTGGAGGGA AGAGCGATGG GAGAGCAGCT CTTGCCAGGT | |
| | | AAAAAGTTTT GGGAAACAGA TGAATCCAGC AAAGATGGAC | |
| | | CAAAAGGAAT ATTCCTGGGT GATCAATGGC GAGACAGTGC | |
| | | CTGGGGAACA TCAGATCATT CAGTTTCCCA GCCAATCATG | |
| | | GTGCAGAGAA GACCTGGTCA GAGTTTCCAT GTGAACAGTG | |
| | | AGGTCAATTC TGTACTGTCC CCACGATCGG AGAGTGGGGG | |
| | | ACTAGGCGTT AGCATGGTGG AGTATGTGTT GAGCTCATCC | |
| | | CCGGGCGATT CCTGTCTAAG AAAAGGAGGA TTTGGCCCAA | |
| | | GGGATGCAGA CAGTGATGAA AACGACAAAG GTGAAAAGAA | |
| | | GAACAAGGGT ACGTTTGATG GAGATAAGCT AGGAGATTTG | |
| | | AAGGAGGAGG GTGATGTGAT GGACAAGACC AATGGTTTAC | |
| | | CAGTGCAGAA TGGGATTGAT GCAGACGTCA AAGATTTTAG | |
| | | CCGTACCCCT GGTAATTGCC AGAACTCTGC TAATGAAGTG | |
| | | GATCTTCTGG GTCCAAACCA GAATGGTTCT GAGGGCTTAG | |
| | | CCCAGCTGAC CAGCACCAAT GGTGCCAAGC CTGTGGAGGA | |
| | | TTTCTCCAAC ATGGAGTCCC AGAGTGTCCC CTTGGACCCC | |
| | | ATGGAACATG TGGGCATGGA GCCTCTTCAG TTTGATTATT | |
| | | CAGGCACGCA GGTACCTGTG GACTCAGCAG CAGCAACTGT | |
| | | GGGACTTTTT GACTACAATT CTCAACAACA GCTGTTCCAA | |
| | | AGACCTAATG CGCTTGCTGT CCAGCAGTTG ACAGCTGCTC | |
| | | AGCAGCAGCA GTATGCACTG GCAGCTGCTC ATCAGCCGCA | |
| | | CATCGGTTTA GCTCCCGCTG CGTTTGTCCC CAATCCATAC | |
| | | ATCATCAGCG CTGCTCCCCC AGGGACGGAC CCCTACACAG | |
| | | CTGGATTGGC TGCAGCAGCG ACACTAGGCC CAGCTGTGGT | |
| | | CCCTCACCAG TATTATGGAG TTACTCCCTG GGGAGTCTAC | |
| | | CCTGCCAGTC TTTTCCAGCA GCAAGCTGCC GCTGCCGCTG | |
| | | CAGCAACTAA TTCAGCTAAT CAACAGACCA CCCCACAGGC | |
| | | TCAGCAAGGA CAGCAGCAGG TTCTCCGTGG AGGAGCCAGC | |
| | | CAACGTCCTT TGACCCCAAA CCAGAACCAG CAGGGACAGC | |
| | | AAACGGATCC CCTTGTGGCA GCTGCAGCAG TGAATTCTGC | |
| | | CCTTGCATTT GGACAAGGTC TGGCAGCAGG CATGCCAGGT | |
| | | TATCCGGTGT TGGCTCCTGC TGCTTACTAT GACCAAACTG | |
| | | GTGCCCTTGT AGTGAATGCA GGCGCGAGAA ATGGTCTTGG | |
| | | AGCTCCTGTT CGACTTGTAG CTCCTGCCCC AGTCATCATT | |
| | | AGTTCCTCAG CTGCACAAGC AGCTGTTGCA GCAGCCGCAG | |
| | | CTTCAGCAAA TGGAGCAGCT GGTGGTCTTG CTGGAACAAC | |
| | | AAATGGACCA TTTCGCCCTT TAGGAACACA GCAGCCTCAG | |
| | | CCCCAGCCCC AGCAGCAGCC CAATAACAAC CTGGCATCCA | |
| | | GTTCTTTCTA CGGCAACAAC TCTCTGAACA GCAATTCACA | |
| | | GAGCAGCTCC CTCTTCTCCC AGGGCTCTGC CCAGCCTGCC | |
| | | AACACATCCT TGGGATTCGG AAGTAGCAGT TCTCTCGGCG | |
| | | CCACCCTGGG ATCCGCCCTT GGAGGGTTTG GAACAGCAGT | |
| | | TGCAAACTCC AACACTGGCA GTGGCTCCCG CCGTGACTCC | |
| | | CTGACTGGCA GCAGTGACCT TTATAAGAGG ACATCGAGCA | |
| | | GCTTGACCCC CATTGGACAC AGTTTTTATA ACGGCCTTAG | |
| | | CTTTTCCTCC TCTCCTGGAC CCGTGGGCAT GCCTCTCCCT | |
| | | AGTCAGGGAC CAGGACATTC ACAGACACCA CCTCCTTCCC | |
| | | TCTCTTCACA TGGATCCTCT TCAAGCTTAA ACCTGGGAGG | |
| | | ACTCACGAAT GGCAGTGGAA GATACATCTC TGCTGCTCCA | |
| | | GGCGCTGAAG CCAAGTACCG CAGTGCAAGC AGCGCCTCCA | |
| | | GCCTCTTCAG CCCGAGCAGC ACTCTTTTCT CTTCCTCTCG | |
| | | TTTGCGATAT GGAATGTCTG ATGTCATGCC TTCTGGCAGG | |
| | | AGCAGGCTTT TGGAAGATTT TCGAAACAAC CGGTACCCCA | |
| | | ATTTACAACT GCGGGAGATT GCTGGACATA TAATGGAATT | |
| | | TTCCCAAGAC CAGCATGGGT CCAGATTCAT TCAGCTGAAA | |
| | | CTGGAGCGTG CCACACCAGC TGAGCGCCAG CTTGTCTTCA | |
| | | ATGAAATCCT CCAGGCTGCC TACCAACTCA TGGTGGATGT | |
| | | GTTTGGTAAT TACGTCATTC AGAAGTTCTT TGAATTTGGC | |
| | | AGTCTTGAAC AGAAGCTGGC TTTGGCAGAA CGGATTCGAG | |
| | | GCCACGTCCT GTCATTGGCA CTACAGATGT ATGGCTGCCG | |
| | | TGTTATCCAG AAAGCTCTTG AGTTTATTCC TTCAGACCAG | |
| | | CAGGTAATTA ATGAGATGGT TCGGGAACTA GATGGCCATG | |
| | | TCTTGAAGTG TGTGAAAGAT CAGAATGGCA ATCACGTGGT | |
| | | TCAGAAATGC ATTGAATGTG TACAGCCCCA GTCTTTGCAA | |
| | | TTTATCATCG ATGCGTTTAA GGGACAGGTA TTTGCCTTAT | |
| | | CCACACATCC TTATGGCTGC CGAGTGATTC AGAGAATCCT | |
| | | GGAGCACTGT CTCCCTGACC AGACACTCCC TATTTTAGAG | |
| | | GAGCTTCACC AGCACACAGA GCAGCTTGTA CAGGATCAAT | |
| | | ATGGAAATTA TGTAATCCAA CATGTACTGG AGCACGGTCG | |
| | | TCCTGAGGAT AAAAGCAAAA TTGTAGCAGA AATCCGAGGC | |
| | | AATGTACTTG TATTGAGTCA GCACAAATTT GCAAGCAATG | |
| | | TTGTGGAGAA GTGTGTTACT CACGCCTCAC GTACGGAGCG | |
| | | CGCTGTGCTC ATCGATGAGG TGTGCACCAT GAACGACGGT | |
| | | CCCCACAGTG CCTTATACAC CATGATGAAG GACCAGTATG | |
| | | CCAACTACGT GGTCCAGAAG ATGATTGACG TGGCGGAGCC | |
| | | AGGCCAGCGG AAGATCGTCA TGCATAAGAT CCGGCCCCAC | |
| | | ATCGCAACTC TTCGTAAGTA CACCTATGGC AAGCACATTC | |
| | | TGGCCAAGCT GGAGAAGTAC TACATGAAGA ACGGTGTTGA | |
| | | CTTAGGGCCC ATCTGTGGCC CCCCTAATGG TATCATCTGA | |
| | | GGCAGTGTCA CCCGCTGTTC CCTCATTCCC GCTGACCTCA | |
| | | CTGGCCCACT GGCAAATCCA ACCAGCAACC AGAAATGTTC | |
| | | TAGTGTAGAG TCTGAGACGG GCAAGTGGTT GCTCCAGGAT | |
| | | TACTCCCTCC TCCAAAAAAG GAATCAAATC CACGAGTGGA | |
| | | AAAGCCTTTG TAAATTTAAT TTTATTACAC ATAACATGTA | |
| | | CTATTTTTTT TAATTGACTA ATTGCCCTGC TGTTTTACTG | |
| | | GTGTATAGGA TACTTGTACA TAGGTAACCA ATGTACATGG | |
| | | GAGGCCACAT ATTTTGTTCA CTGTTGTATC TATATTTCAC | |
| | | ATGTGGAAAC TTTCAGGGTG GTTGGTTTAA CAAAAAAAAA | |
| | | AAGCTTTAAA AAAAAAAGAA AAAAAGGAAA AGGTTTTTAG | |
| | | CTCATTTGCC TGGCCGGCAA GTTTTGCAAA TAGCTCTTCC | |
| | | CCACCTCCTC ATTTTAGTAA AAAACAAACA AAAACAAAAA | |
| | | AACCTGAGAA GTTTGAATTG TAGTTAAATG ACCCCAAACT | |
| | | GGCATTTAAC ACTGTTTATA AAAAATATAT ATATATATAT | |
| | | ATATATATAT AATGAAAAAG GTTTCAGAGT TGCTAAAGCT | |
| | | TCAGTTTGTG ACATTAAGTT TATGAAATTC TAAAAAATGC | |
| | | CTTTTTTGGA GACTATATTA TGCTGAAGAA GGCTGTTCGT | |
| | | GAGGAGGAGA TGCGAGCACC CAGAACGTCT TTTGAGGCTG | |
| | | GGCGGGTGTG ATTGTTTACT GCCTACTGGA TTTTTTTCTA | |
| | | TTAACATTGA AAGGTAAAAT CTGATTATTT AGCATGAGAA | |
| | | AAAAAAATCC AACTCTGCTT TTGGTCTTGC TTCTATAAAT | |
| | | ATATAGTGTA TACTTGGTGT AGACTTTGCA TATATACAAA | |
| | | TTTGTAGTAT TTTCTTGTTT TGATGTCTAA TCTGTATCTA | |
| | | TAATGTACCC TAGTAGTCGA ACATACTTTT GATTGTACAA | |
| | | TTGTACATTT GTATACCTGT AATGTAAATG TGGAGAAGTT | |
| | | TGAATCAACA TAAACACGTT TTTTGGTAAG AAAAGAGAAT | |
| | | TAGCCAGCCC TGTGCATTCA GTGTATATTC TCACCTTTTA | |
| | | TGGTCGTAGC ATATAGTGTT GTATATTGTA AATTGTAATT | |
| | | TCAACCAGAA GTAAATTTTT TTCTTTTGAA GGAATAAATG | |
| | | TTCTTTATAC AGCCTAGTTA ATGTTTAAAA AGAAAAAAAT | |
| | | AGCTTGGTTT TATTTGTCAT CTAGTCTCAA GTATAGCGAG | |
| | | ATTCTTTCTA AATGTTATTC AAGATTGAGT TCTCACTAGT | |
| | | GTTTTTTTAA TCCTAAAAAA GTAATGTTTT GATTTTGTGA | |
| | | CAGTCAAAAG GACGTGCAAA AGTCTAGCCT TGCCCGAGCT | |
| | | TTCCTTACAA TCAGAGCCCC TCTCACCTTG TAAAGTGTGA | |
| | | ATCGCCCTTC CCTTTTGTAC AGAAGATGAA CTGTATTTTG | |
| | | CATTTTGTCT ACTTGTAAGT GAATGTAACA TACTGTCAAT | |
| | | TTTCCTTGTT TGAATATAGA ATTGTAACAC TACACGGTGT | |
| | | ACATTTCCAG AGCCTTGTGT ATATTTCCAA TGAACTTTTT | |
| | | TGCAAGCACA CTTGTAACCA TATGTGTATA ATTAACAAAC | |
| | | CTGTGTATGC TTATGCCTGG GCAACTATTT TTTGTAACTC | |
| | | TTGTGTAGAT TGTCTCTAAA CAATGTGTGA TCTTTATTTT | |
| | | GAAAAATACA GAACTTTGGA ATCTGAAAAA AAAAAAAAAA | |
| | | AAAAAAAAAA AAAAAA | |
| ACTB | NM_001101.4 | GAGTGAGCGG CGCGGGGCCA ATCAGCGTGC GCCGTTCCGA | 44 |
| | | AAGTTGCCTT TTATGGCTCG AGCGGCCGCG GCGGCGCCCT | |
| | | ATAAAACCCA GCGGCGCGAC GCGCCACCAC CGCCGAGACC | |
| | | GCGTCCGCCC CGCGAGCACA GAGCCTCGCC TTTGCCGATC | |
| | | CGCCGCCCGT CCACACCCGC CGCCAGCTCA CCATGGATGA | |
| | | TGATATCGCC GCGCTCGTCG TCGACAACGG CTCCGGCATG | |
| | | TGCAAGGCCG GCTTCGCGGG CGACGATGCC CCCCGGGCCG | |
| | | TCTTCCCCTC CATCGTGGGG CGCCCCAGGC ACCAGGGCGT | |
| | | GATGGTGGGC ATGGGTCAGA AGGATTCCTA TGTGGGCGAC | |
| | | GAGGCCCAGA GCAAGAGAGG CATCCTCACC CTGAAGTACC | |
| | | CCATCGAGCA CGGCATCGTC ACCAACTGGG ACGACATGGA | |
| | | GAAAATCTGG CACCACACCT TCTACAATGA GCTGCGTGTG | |
| | | GCTCCCGAGG AGCACCCCGT GCTGCTGACC GAGGCCCCCC | |
| | | TGAACCCCAA GGCCAACCGC GAGAAGATGA CCCAGATCAT | |
| | | GTTTGAGACC TTCAACACCC CAGCCATGTA CGTTGCTATC | |
| | | CAGGCTGTGC TATCCCTGTA CGCCTCTGGC CGTACCACTG | |
| | | GCATCGTGAT GGACTCCGGT GACGGGGTCA CCCACACTGT | |
| | | GCCCATCTAC GAGGGGTATG CCCTCCCCCA TGCCATCCTG | |
| | | CGTCTGGACC TGGCTGGCCG GGACCTGACT GACTACCTCA | |
| | | TGAAGATCCT CACCGAGCGC GGCTACAGCT TCACCACCAC | |
| | | GGCCGAGCGG GAAATCGTGC GTGACATTAA GGAGAAGCTG | |
| | | TGCTACGTCG CCCTGGACTT CGAGCAAGAG ATGGCCACGG | |
| | | CTGCTTCCAG CTCCTCCCTG GAGAAGAGCT ACGAGCTGCC | |
| | | TGACGGCCAG GTCATCACCA TTGGCAATGA GCGGTTCCGC | |
| | | TGCCCTGAGG CACTCTTCCA GCCTTCCTTC CTGGGCATGG | |
| | | AGTCCTGTGG CATCCACGAA ACTACCTTCA ACTCCATCAT | |
| | | GAAGTGTGAC GTGGACATCC GCAAAGACCT GTACGCCAAC | |
| | | ACAGTGCTGT CTGGCGGCAC CACCATGTAC CCTGGCATTG | |
| | | CCGACAGGAT GCAGAAGGAG ATCACTGCCC TGGCACCCAG | |
| | | CACAATGAAG ATCAAGATCA TTGCTCCTCC TGAGCGCAAG | |
| | | TACTCCGTGT GGATCGGCGG CTCCATCCTG GCCTCGCTGT | |
| | | CCACCTTCCA GCAGATGTGG ATCAGCAAGC AGGAGTATGA | |
| | | CGAGTCCGGC CCCTCCATCG TCCACCGCAA ATGCTTCTAG | |
| | | GCGGACTATG ACTTAGTTGC GTTACACCCT TTCTTGACAA | |
| | | AACCTAACTT GCGCAGAAAA CAAGATGAGA TTGGCATGGC | |
| | | TTTATTTGTT TTTTTTGTTT TGTTTTGGTT TTTTTTTTTT | |
| | | TTTTGGCTTG ACTCAGGATT TAAAAACTGG AACGGTGAAG | |
| | | GTGACAGCAG TCGGTTGGAG CGAGCATCCC CCAAAGTTCA | |
| | | CAATGTGGCC GAGGACTTTG ATTGCACATT GTTGTTTTTT | |
| | | TAATAGTCAT TCCAAATATG AGATGCGTTG TTACAGGAAG | |
| | | TCCCTTGCCA TCCTAAAAGC CACCCCACTT CTCTCTAAGG | |
| | | AGAATGGCCC AGTCCTCTCC CAAGTCCACA CAGGGGAGGT | |
| | | GATAGCATTG CTTTCGTGTA AATTATGTAA TGCAAAATTT | |
| | | TTTTAATCTT CGCCTTAATA CTTTTTTATT TTGTTTTATT | |
| | | TTGAATGATG AGCCTTCGTG CCCCCCCTTC CCCCTTTTTT | |
| | | GTCCCCCAAC TTGAGATGTA TGAAGGCTTT TGGTCTCCCT | |
| | | GGGAGTGGGT GGAGGCAGCC AGGGCTTACC TGTACACTGA | |
| | | CTTGAGACCA GTTGAATAAA AGTGCACACC TTAAAAATGA | |
| | | GGAAAAAAAA AAAAAAAAAA | |
| GAPD | NM_002046.6 | GCTCTCTGCT CCTCCTGTTC GACAGTCAGC CGCATCTTCT | 45 |
| | | TTTGCGTCGC CAGCCGAGCC ACATCGCTCA GACACCATGG | |
| | | GGAAGGTGAA GGTCGGAGTC AACGGATTTG GTCGTATTGG | |
| | | GCGCCTGGTC ACCAGGGCTG CTTTTAACTC TGGTAAAGTG | |
| | | GATATTGTTG CCATCAATGA CCCCTTCATT GACCTCAACT | |
| | | ACATGGTTTA CATGTTCCAA TATGATTCCA CCCATGGCAA | |
| | | ATTCCATGGC ACCGTCAAGG CTGAGAACGG GAAGCTTGTC | |
| | | ATCAATGGAA ATCCCATCAC CATCTTCCAG GAGCGAGATC | |
| | | CCTCCAAAAT CAAGTGGGGC GATGCTGGCG CTGAGTACGT | |
| | | CGTGGAGTCC ACTGGCGTCT TCACCACCAT GGAGAAGGCT | |
| | | GGGGCTCATT TGCAGGGGGG AGCCAAAAGG GTCATCATCT | |
| | | CTGCCCCCTC TGCTGATGCC CCCATGTTCG TCATGGGTGT | |
| | | GAACCATGAG AAGTATGACA ACAGCCTCAA GATCATCAGC | |
| | | AATGCCTCCT GCACCACCAA CTGCTTAGCA CCCCTGGCCA | |
| | | AGGTCATCCA TGACAACTTT GGTATCGTGG AAGGACTCAT | |
| | | GACCACAGTC CATGCCATCA CTGCCACCCA GAAGACTGTG | |
| | | GATGGCCCCT CCGGGAAACT GTGGCGTGAT GGCCGCGGGG | |
| | | CTCTCCAGAA CATCATCCCT GCCTCTACTG GCGCTGCCAA | |
| | | GGCTGTGGGC AAGGTCATCC CTGAGCTGAA CGGGAAGCTC | |
| | | ACTGGCATGG CCTTCCGTGT CCCCACTGCC AACGTGTCAG | |
| | | TGGTGGACCT GACCTGCCGT CTAGAAAAAC CTGCCAAATA | |
| | | TGATGACATC AAGAAGGTGG TGAAGCAGGC GTCGGAGGGC | |
| | | CCCCTCAAGG GCATCCTGGG CTACACTGAG CACCAGGTGG | |
| | | TCTCCTCTGA CTTCAACAGC GACACCCACT CCTCCACCTT | |
| | | TGACGCTGGG GCTGGCATTG CCCTCAACGA CCACTTTGTC | |
| | | AAGCTCATTT CCTGGTATGA CAACGAATTT GGCTACAGCA | |
| | | ACAGGGTGGT GGACCTCATG GCCCACATGG CCTCCAAGGA | |
| | | GTAAGACCCC TGGACCACCA GCCCCAGCAA GAGCACAAGA | |
| | | GGAAGAGAGA GACCCTCACT GCTGGGGAGT CCCTGCCACA | |
| | | CTCAGTCCCC CACCACACTG AATCTCCCCT CCTCACAGTT | |
| | | GCCATGTAGA CCCCTTGAAG AGGGGAGGGG CCTAGGGAGC | |
| | | CGCACCTTGT CATGTACCAT CAATAAAGTA CCCTGTGCTC | |
| | | AACCAGTTAA AAAAAAAAAA AAAAAAAAA | |
| GUSB | NM_000181.3 | GTCCTCAACC AAGATGGCGC GGATGGCTTC AGGCGCATCA | 46 |
| | | CGACACCGGC GCGTCACGCG ACCCGCCCTA CGGGCACCTC | |
| | | CCGCGCTTTT CTTAGCGCCG CAGACGGTGG CCGAGCGGGG | |
| | | GACCGGGAAG CATGGCCCGG GGGTCGGCGG TTGCCTGGGC | |
| | | GGCGCTCGGG CCGTTGTTGT GGGGCTGCGC GCTGGGGCTG | |
| | | CAGGGCGGGA TGCTGTACCC CCAGGAGAGC CCGTCGCGGG | |
| | | AGTGCAAGGA GCTGGACGGC CTCTGGAGCT TCCGCGCCGA | |
| | | CTTCTCTGAC AACCGACGCC GGGGCTTCGA GGAGCAGTGG | |
| | | TACCGGCGGC CGCTGTGGGA GTCAGGCCCC ACCGTGGACA | |
| | | TGCCAGTTCC CTCCAGCTTC AATGACATCA GCCAGGACTG | |
| | | GCGTCTGCGG CATTTTGTCG GCTGGGTGTG GTACGAACGG | |
| | | GAGGTGATCC TGCCGGAGCG ATGGACCCAG GACCTGCGCA | |
| | | CAAGAGTGGT GCTGAGGATT GGCAGTGCCC ATTCCTATGC | |
| | | CATCGTGTGG GTGAATGGGG TCGACACGCT AGAGCATGAG | |
| | | GGGGGCTACC TCCCCTTCGA GGCCGACATC AGCAACCTGG | |
| | | TCCAGGTGGG GCCCCTGCCC TCCCGGCTCC GAATCACTAT | |
| | | CGCCATCAAC AACACACTCA CCCCCACCAC CCTGCCACCA | |
| | | GGGACCATCC AATACCTGAC TGACACCTCC AAGTATCCCA | |
| | | AGGGTTACTT TGTCCAGAAC ACATATTTTG ACTTTTTCAA | |
| | | CTACGCTGGA CTGCAGCGGT CTGTACTTCT GTACACGACA | |
| | | CCCACCACCT ACATCGATGA CATCACCGTC ACCACCAGCG | |
| | | TGGAGCAAGA CAGTGGGCTG GTGAATTACC AGATCTCTGT | |
| | | CAAGGGCAGT AACCTGTTCA AGTTGGAAGT GCGTCTTTTG | |
| | | GATGCAGAAA ACAAAGTCGT GGCGAATGGG ACTGGGACCC | |
| | | AGGGCCAACT TAAGGTGCCA GGTGTCAGCC TCTGGTGGCC | |
| | | GTACCTGATG CACGAACGCC CTGCCTATCT GTATTCATTG | |
| | | GAGGTGCAGC TGACTGCACA GACGTCACTG GGGCCTGTGT | |
| | | CTGACTTCTA CACACTCCCT GTGGGGATCC GCACTGTGGC | |
| | | TGTCACCAAG AGCCAGTTCC TCATCAATGG GAAACCTTTC | |
| | | TATTTCCACG GTGTCAACAA GCATGAGGAT GCGGACATCC | |
| | | GAGGGAAGGG CTTCGACTGG CCGCTGCTGG TGAAGGACTT | |
| | | CAACCTGCTT CGCTGGCTTG GTGCCAACGC TTTCCGTACC | |
| | | AGCCACTACC CCTATGCAGA GGAAGTGATG CAGATGTGTG | |
| | | ACCGCTATGG GATTGTGGTC ATCGATGAGT GTCCCGGCGT | |
| | | GGGCCTGGCG CTGCCGCAGT TCTTCAACAA CGTTTCTCTG | |
| | | CATCACCACA TGCAGGTGAT GGAAGAAGTG GTGCGTAGGG | |
| | | ACAAGAACCA CCCCGCGGTC GTGATGTGGT CTGTGGCCAA | |
| | | CGAGCCTGCG TCCCACCTAG AATCTGCTGG CTACTACTTG | |
| | | AAGATGGTGA TCGCTCACAC CAAATCCTTG GACCCCTCCC | |
| | | GGCCTGTGAC CTTTGTGAGC AACTCTAACT ATGCAGCAGA | |
| | | CAAGGGGGCT CCGTATGTGG ATGTGATCTG TTTGAACAGC | |
| | | TACTACTCTT GGTATCACGA CTACGGGCAC CTGGAGTTGA | |
| | | TTCAGCTGCA GCTGGCCACC CAGTTTGAGA ACTGGTATAA | |
| | | GAAGTATCAG AAGCCCATTA TTCAGAGCGA GTATGGAGCA | |
| | | GAAACGATTG CAGGGTTTCA CCAGGATCCA CCTCTGATGT | |
| | | TCACTGAAGA GTACCAGAAA AGTCTGCTAG AGCAGTACCA | |
| | | TCTGGGTCTG GATCAAAAAC GCAGAAAATA CGTGGTTGGA | |
| | | GAGCTCATTT GGAATTTTGC CGATTTCATG ACTGAACAGT | |
| | | CACCGACGAG AGTGCTGGGG AATAAAAAGG GGATCTTCAC | |
| | | TCGGCAGAGA CAACCAAAAA GTGCAGCGTT CCTTTTGCGA | |
| | | GAGAGATACT GGAAGATTGC CAATGAAACC AGGTATCCCC | |
| | | ACTCAGTAGC CAAGTCACAA TGTTTGGAAA ACAGCCTGTT | |
| | | TACTTGAGCA AGACTGATAC CACCTGCGTG TCCCTTCCTC | |
| | | CCCGAGTCAG GGCGACTTCC ACAGCAGCAG AACAAGTGCC | |
| | | TCCTGGACTG TTCACGGCAG ACCAGAACGT TTCTGGCCTG | |
| | | GGTTTTGTGG TCATCTATTC TAGCAGGGAA CACTAAAGGT | |
| | | GGAAATAAAA GATTTTCTAT TATGGAAATA AAGAGTTGGC | |
| | | ATGAAAGTGG CTACTGAAAA AAAAAAAAAA AAAAAAAAAA A | |
| RPLP0 | NM_001002.3 | GTCTGACGGG CGATGGCGCA GCCAATAGAC AGGAGCGCTA | 47 |
| | | TCCGCGGTTT CTGATTGGCT ACTTTGTTCG CATTATAAAA | |
| | | GGCACGCGCG GGCGCGAGGC CCTTCTCTCG CCAGGCGTCC | |
| | | TCGTGGAAGT GACATCGTCT TTAAACCCTG CGTGGCAATC | |
| | | CCTGACGCAC CGCCGTGATG CCCAGGGAAG ACAGGGCGAC | |
| | | CTGGAAGTCC AACTACTTCC TTAAGATCAT CCAACTATTG | |
| | | GATGATTATC CGAAATGTTT CATTGTGGGA GCAGACAATG | |
| | | TGGGCTCCAA GCAGATGCAG CAGATCCGCA TGTCCCTTCG | |
| | | CGGGAAGGCT GTGGTGCTGA TGGGCAAGAA CACCATGATG | |
| | | CGCAAGGCCA TCCGAGGGCA CCTGGAAAAC AACCCAGCTC | |
| | | TGGAGAAACT GCTGCCTCAT ATCCGGGGGA ATGTGGGCTT | |
| | | TGTGTTCACC AAGGAGGACC TCACTGAGAT CAGGGACATG | |
| | | TTGCTGGCCA ATAAGGTGCC AGCTGCTGCC CGTGCTGGTG | |
| | | CCATTGCCCC ATGTGAAGTC ACTGTGCCAG CCCAGAACAC | |
| | | TGGTCTCGGG CCCGAGAAGA CCTCCTTTTT CCAGGCTTTA | |
| | | GGTATCACCA CTAAAATCTC CAGGGGCACC ATTGAAATCC | |
| | | TGAGTGATGT GCAGCTGATC AAGACTGGAG ACAAAGTGGG | |
| | | AGCCAGCGAA GCCACGCTGC TGAACATGCT CAACATCTCC | |
| | | CCCTTCTCCT TTGGGCTGGT CATCCAGCAG GTGTTCGACA | |
| | | ATGGCAGCAT CTACAACCCT GAAGTGCTTG ATATCACAGA | |
| | | GGAAACTCTG CATTCTCGCT TCCTGGAGGG TGTCCGCAAT | |
| | | GTTGCCAGTG TCTGTCTGCA GATTGGCTAC CCAACTGTTG | |
| | | CATCAGTACC CCATTCTATC ATCAACGGGT ACAAACGAGT | |
| | | CCTGGCCTTG TCTGTGGAGA CGGATTACAC CTTCCCACTT | |
| | | GCTGAAAAGG TCAAGGCCTT CTTGGCTGAT CCATCTGCCT | |
| | | TTGTGGCTGC TGCCCCTGTG GCTGCTGCCA CCACAGCTGC | |
| | | TCCTGCTGCT GCTGCAGCCC CAGCTAAGGT TGAAGCCAAG | |
| | | GAAGAGTCGG AGGAGTCGGA CGAGGATATG GGATTTGGTC | |
| | | TCTTTGACTA ATCACCAAAA AGCAACCAAC TTAGCCAGTT | |
| | | TTATTTGCAA AACAAGGAAA TAAAGGCTTA CTTCTTTAAA | |
| | | AAGTAAAAAA AAAAAAAAAA AAAAAAAAA | |
| TFRC | NM_003234.3 | AGAGCGTCGG GATATCGGGT GGCGGCTCGG GACGGAGGAC | 48 |
| | | GCGCTAGTGT GAGTGCGGGC TTCTAGAACT ACACCGACCC | |
| | | TCGTGTCCTC CCTTCATCCT GCGGGGCTGG CTGGAGCGGC | |
| | | CGCTCCGGTG CTGTCCAGCA GCCATAGGGA GCCGCACGGG | |
| | | GAGCGGGAAA GCGGTCGCGG CCCCAGGCGG GGCGGCCGGG | |
| | | ATGGAGCGGG GCCGCGAGCC TGTGGGGAAG GGGCTGTGGC | |
| | | GGCGCCTCGA GCGGCTGCAG GTTCTTCTGT GTGGCAGTTC | |
| | | AGAATGATGG ATCAAGCTAG ATCAGCATTC TCTAACTTGT | |
| | | TTGGTGGAGA ACCATTGTCA TATACCCGGT TCAGCCTGGC | |
| | | TCGGCAAGTA GATGGCGATA ACAGTCATGT GGAGATGAAA | |
| | | CTTGCTGTAG ATGAAGAAGA AAATGCTGAC AATAACACAA | |
| | | AGGCCAATGT CACAAAACCA AAAAGGTGTA GTGGAAGTAT | |
| | | CTGCTATGGG ACTATTGCTG TGATCGTCTT TTTCTTGATT | |
| | | GGATTTATGA TTGGCTACTT GGGCTATTGT AAAGGGGTAG | |
| | | AACCAAAAAC TGAGTGTGAG AGACTGGCAG GAACCGAGTC | |
| | | TCCAGTGAGG GAGGAGCCAG GAGAGGACTT CCCTGCAGCA | |
| | | CGTCGCTTAT ATTGGGATGA CCTGAAGAGA AAGTTGTCGG | |
| | | AGAAACTGGA CAGCACAGAC TTCACCGGCA CCATCAAGCT | |
| | | GCTGAATGAA AATTCATATG TCCCTCGTGA GGCTGGATCT | |
| | | CAAAAAGATG AAAATCTTGC GTTGTATGTT GAAAATCAAT | |
| | | TTCGTGAATT TAAACTCAGC AAAGTCTGGC GTGATCAACA | |
| | | TTTTGTTAAG ATTCAGGTCA AAGACAGCGC TCAAAACTCG | |
| | | GTGATCATAG TTGATAAGAA CGGTAGACTT GTTTACCTGG | |
| | | TGGAGAATCC TGGGGGTTAT GTGGCGTATA GTAAGGCTGC | |
| | | AACAGTTACT GGTAAACTGG TCCATGCTAA TTTTGGTACT | |
| | | AAAAAAGATT TTGAGGATTT ATACACTCCT GTGAATGGAT | |
| | | CTATAGTGAT TGTCAGAGCA GGGAAAATCA CCTTTGCAGA | |
| | | AAAGGTTGCA AATGCTGAAA GCTTAAATGC AATTGGTGTG | |
| | | TTGATATACA TGGACCAGAC TAAATTTCCC ATTGTTAACG | |
| | | CAGAACTTTC ATTCTTTGGA CATGCTCATC TGGGGACAGG | |
| | | TGACCCTTAC ACACCTGGAT TCCCTTCCTT CAATCACACT | |
| | | CAGTTTCCAC CATCTCGGTC ATCAGGATTG CCTAATATAC | |
| | | CTGTCCAGAC AATCTCCAGA GCTGCTGCAG AAAAGCTGTT | |
| | | TGGGAATATG GAAGGAGACT GTCCCTCTGA CTGGAAAACA | |
| | | GACTCTACAT GTAGGATGGT AACCTCAGAA AGCAAGAATG | |
| | | TGAAGCTCAC TGTGAGCAAT GTGCTGAAAG AGATAAAAAT | |
| | | TCTTAACATC TTTGGAGTTA TTAAAGGCTT TGTAGAACCA | |
| | | GATCACTATG TTGTAGTTGG GGCCCAGAGA GATGCATGGG | |
| | | GCCCTGGAGC TGCAAAATCC GGTGTAGGCA CAGCTCTCCT | |
| | | ATTGAAACTT GCCCAGATGT TCTCAGATAT GGTCTTAAAA | |
| | | GATGGGTTTC AGCCCAGCAG AAGCATTATC TTTGCCAGTT | |
| | | GGAGTGCTGG AGACTTTGGA TCGGTTGGTG CCACTGAATG | |
| | | GCTAGAGGGA TACCTTTCGT CCCTGCATTT AAAGGCTTTC | |
| | | ACTTATATTA ATCTGGATAA AGCGGTTCTT GGTACCAGCA | |
| | | ACTTCAAGGT TTCTGCCAGC CCACTGTTGT ATACGCTTAT | |
| | | TGAGAAAACA ATGCAAAATG TGAAGCATCC GGTTACTGGG | |
| | | CAATTTCTAT ATCAGGACAG CAACTGGGCC AGCAAAGTTG | |
| | | AGAAACTCAC TTTAGACAAT GCTGCTTTCC CTTTCCTTGC | |
| | | ATATTCTGGA ATCCCAGCAG TTTCTTTCTG TTTTTGCGAG | |
| | | GACACAGATT ATCCTTATTT GGGTACCACC ATGGACACCT | |
| | | ATAAGGAACT GATTGAGAGG ATTCCTGAGT TGAACAAAGT | |
| | | GGCACGAGCA GCTGCAGAGG TCGCTGGTCA GTTCGTGATT | |
| | | AAACTAACCC ATGATGTTGA ATTGAACCTG GACTATGAGA | |
| | | GGTACAACAG CCAACTGCTT TCATTTGTGA GGGATCTGAA | |
| | | CCAATACAGA GCAGACATAA AGGAAATGGG CCTGAGTTTA | |
| | | CAGTGGCTGT ATTCTGCTCG TGGAGACTTC TTCCGTGCTA | |
| | | CTTCCAGACT AACAACAGAT TTCGGGAATG CTGAGAAAAC | |
| | | AGACAGATTT GTCATGAAGA AACTCAATGA TCGTGTCATG | |
| | | AGAGTGGAGT ATCACTTCCT CTCTCCCTAC GTATCTCCAA | |
| | | AAGAGTCTCC TTTCCGACAT GTCTTCTGGG GCTCCGGCTC | |
| | | TCACACGCTG CCAGCTTTAC TGGAGAACTT GAAACTGCGT | |
| | | AAACAAAATA ACGGTGCTTT TAATGAAACG CTGTTCAGAA | |
| | | ACCAGTTGGC TCTAGCTACT TGGACTATTC AGGGAGCTGC | |
| | | AAATGCCCTC TCTGGTGACG TTTGGGACAT TGACAATGAG | |
| | | TTTTAAATGT GATACCCATA GCTTCCATGA GAACAGCAGG | |
| | | GTAGTCTGGT TTCTAGACTT GTGCTGATCG TGCTAAATTT | |
| | | TCAGTAGGGC TACAAAACCT GATGTTAAAA TTCCATCCCA | |
| | | TCATCTTGGT ACTACTAGAT GTCTTTAGGC AGCAGCTTTT | |
| | | AATACAGGGT AGATAACCTG TACTTCAAGT TAAAGTGAAT | |
| | | AACCACTTAA AAAATGTCCA TGATGGAATA TTCCCCTATC | |
| | | TCTAGAATTT TAAGTGCTTT GTAATGGGAA CTGCCTCTTT | |
| | | CCTGTTGTTG TTAATGAAAA TGTCAGAAAC CAGTTATGTG | |
| | | AATGATCTCT CTGAATCCTA AGGGCTGGTC TCTGCTGAAG | |
| | | GTTGTAAGTG GTCGCTTACT TTGAGTGATC CTCCAACTTC | |
| | | ATTTGATGCT AAATAGGAGA TACCAGGTTG AAAGACCTTC | |
| | | TCCAAATGAG ATCTAAGCCT TTCCATAAGG AATGTAGCTG | |
| | | GTTTCCTCAT TCCTGAAAGA AACAGTTAAC TTTCAGAAGA | |
| | | GATGGGCTTG TTTTCTTGCC AATGAGGTCT GAAATGGAGG | |
| | | TCCTTCTGCT GGATAAAATG AGGTTCAACT GTTGATTGCA | |
| | | GGAATAAGGC CTTAATATGT TAACCTCAGT GTCATTTATG | |
| | | AAAAGAGGGG ACCAGAAGCC AAAGACTTAG TATATTTTCT | |
| | | TTTCCTCTGT CCCTTCCCCC ATAAGCCTCC ATTTAGTTCT | |
| | | TTGTTATTTT TGTTTCTTCC AAAGCACATT GAAAGAGAAC | |
| | | CAGTTTCAGG TGTTTAGTTG CAGACTCAGT TTGTCAGACT | |
| | | TTAAAGAATA ATATGCTGCC AAATTTTGGC CAAAGTGTTA | |
| | | ATCTTAGGGG AGAGCTTTCT GTCCTTTTGG CACTGAGATA | |
| | | TTTATTGTTT ATTTATCAGT GACAGAGTTC ACTATAAATG | |
| | | GTGTTTTTTT AATAGAATAT AATTATCGGA AGCAGTGCCT | |
| | | TCCATAATTA TGACAGTTAT ACTGTCGGTT TTTTTTAAAT | |
| | | AAAAGCAGCA TCTGCTAATA AAACCCAACA GATACTGGAA | |
| | | GTTTTGCATT TATGGTCAAC ACTTAAGGGT TTTAGAAAAC | |
| | | AGCCGTCAGC CAAATGTAAT TGAATAAAGT TGAAGCTAAG | |
| | | ATTTAGAGAT GAATTAAATT TAATTAGGGG TTGCTAAGAA | |
| | | GCGAGCACTG ACCAGATAAG AATGCTGGTT TTCCTAAATG | |
| | | CAGTGAATTG TGACCAAGTT ATAAATCAAT GTCACTTAAA | |
| | | GGCTGTGGTA GTACTCCTGC AAAATTTTAT AGCTCAGTTT | |
| | | ATCCAAGGTG TAACTCTAAT TCCCATTTTG CAAAATTTCC | |
| | | AGTACCTTTG TCACAATCCT AACACATTAT CGGGAGCAGT | |
| | | GTCTTCCATA ATGTATAAAG AACAAGGTAG TTTTTACCTA | |
| | | CCACAGTGTC TGTATCGGAG ACAGTGATCT CCATATGTTA | |
| | | CACTAAGGGT GTAAGTAATT ATCGGGAACA GTGTTTCCCA | |
| | | TAATTTTCTT CATGCAATGA CATCTTCAAA GCTTGAAGAT | |
| | | CGTTAGTATC TAACATGTAT CCCAACTCCT ATAATTCCCT | |
| | | ATCTTTTAGT TTTAGTTGCA GAAACATTTT GTGGTCATTA | |
| | | AGCATTGGGT GGGTAAATTC AACCACTGTA AAATGAAATT | |
| | | ACTACAAAAT TTGAAATTTA GCTTGGGTTT TTGTTACCTT | |
| | | TATGGTTTCT CCAGGTCCTC TACTTAATGA GATAGTAGCA | |
| | | TACATTTATA ATGTTTGCTA TTGACAAGTC ATTTTAACTT | |
| | | TATCACATTA TTTGCATGTT ACCTCCTATA AACTTAGTGC | |
| | | GGACAAGTTT TAATCCAGAA TTGACCTTTT GACTTAAAGC | |
| | | AGAGGGACTT TGTATAGAAG GTTTGGGGGC TGTGGGGAAG | |
| | | GAGAGTCCCC TGAAGGTCTG ACACGTCTGC CTACCCATTC | |
| | | GTGGTGATCA ATTAAATGTA GGTATGAATA AGTTCGAAGC | |
| | | TCCGTGAGTG AACCATCATT ATAAACGTGA TGATCAGCTG | |
| | | TTTGTCATAG GGCAGTTGGA AACGGCCTCC TAGGGAAAAG | |
| | | TTCATAGGGT CTCTTCAGGT TCTTAGTGTC ACTTACCTAG | |
| | | ATTTACAGCC TCACTTGAAT GTGTCACTAC TCACAGTCTC | |
| | | TTTAATCTTC AGTTTTATCT TTAATCTCCT CTTTTATCTT | |
| | | GGACTGACAT TTAGCGTAGC TAAGTGAAAA GGTCATAGCT | |
| | | GAGATTCCTG GTTCGGGTGT TACGCACACG TACTTAAATG | |
| | | AAAGCATGTG GCATGTTCAT CGTATAACAC AATATGAATA | |
| | | CAGGGCATGC ATTTTGCAGC AGTGAGTCTC TTCAGAAAAC | |
| | | CCTTTTCTAC AGTTAGGGTT GAGTTACTTC CTATCAAGCC | |
| | | AGTACGTGCT AACAGGCTCA ATATTCCTGA ATGAAATATC | |
| | | AGACTAGTGA CAAGCTCCTG GTCTTGAGAT GTCTTCTCGT | |
| | | TAAGGAGATG GGCCTTTTGG AGGTAAAGGA TAAAATGAAT | |
| | | GAGTTCTGTC ATGATTCACT ATTCTAGAAC TTGCATGACC | |
| | | TTTACTGTGT TAGCTCTTTG AATGTTCTTG AAATTTTAGA | |
| | | CTTTCTTTGT AAACAAATGA TATGTCCTTA TCATTGTATA | |
| | | AAAGCTGTTA TGTGCAACAG TGTGGAGATT CCTTGTCTGA | |
| | | TTTAATAAAA TACTTAAACA CTGAAAAAAA AAAA | |
| 18S | X03205.1 | TACCTGGTTG ATCCTGCCAG TAGCATATGC TTGTCTCAAA | 49 |
| | | GATTAAGCCA TGCATGTCTA AGTACGCACG GCCGGTACAG | |
| | | TGAAACTGCG AATGGCTCAT TAAATCAGTT ATGGTTCCTT | |
| | | TGGTCGCTCG CTCCTCTCCC ACTTGGATAA CTGTGGTAAT | |
| | | TCTAGAGCTA ATACATGCCG ACGGGCGCTG ACCCCCTTCG | |
| | | CGGGGGGGAT GCGTGCATTT ATCAGATCAA AACCAACCCG | |
| | | GTCAGCCCCT CTCCGGCCCC GGCCGGGGGG CGGGCGCCGG | |
| | | CGGCTTTGGT GACTCTAGAT AACCTCGGGC CGATCGCACG | |
| | | CCCCCCGTGG CGGCGACGAC CCATTCGAAC GTCTGCCCTA | |
| | | TCAACTTTCG ATGGTAGTCG CCGTGCCTAC CATGGTGACC | |
| | | ACGGGTGACG GGGAATCAGG GTTCGATTCC GGAGAGGGAG | |
| | | CCTGAGAAAC GGCTACCACA TCCAAGGAAG GCAGCAGGCG | |
| | | CGCAAATTAC CCACTCCCGA CCCGGGGAGG TAGTGACGAA | |
| | | AAATAACAAT ACAGGACTCT TTCGAGGCCC TGTAATTGGA | |
| | | ATGAGTCCAC TTTAAATCCT TTAACGAGGA TCCATTGGAG | |
| | | GGCAAGTCTG GTGCCAGCAG CCGCGGTAAT TCCAGCTCCA | |
| | | ATAGCGTATA TTAAAGTTGC TGCAGTTAAA AAGCTCGTAG | |
| | | TTGGATCTTG GGAGCGGGCG GGCGGTCCGC CGCGAGGCGA | |
| | | GCCACCGCCC GTCCCCGCCC CTTGCCTCTC GGCGCCCCCT | |
| | | CGATGCTCTT AGCTGAGTGT CCCGCGGGGC CCGAAGCGTT | |
| | | TACTTTGAAA AAATTAGAGT GTTCAAAGCA GGCCCGAGCC | |
| | | GCCTGGATAC CGCAGCTAGG AATAATGGAA TAGGACCGCG | |
| | | GTTCTATTTT GTTGGTTTTC GGAACTGAGG CCATGATTAA | |
| | | GAGGGACGGC CGGGGGCATT CGTATTGCGC CGCTAGAGGT | |
| | | GAAATTCTTG GACCGGCGCA AGACGGACCA GAGCGAAAGC | |
| | | ATTTGCCAAG AATGTTTTCA TTAATCAAGA ACGAAAGTCG | |
| | | GAGGTTCGAA GACGATCAGA TACCGTCGTA GTTCCGACCA | |
| | | TAAACGATGC CGACCGGCGA TGCGGCGGCG TTATTCCCAT | |
| | | GACCCGCCGG GCAGCTTCCG GGAAACCAAA GTCTTTGGGT | |
| | | TCCGGGGGGA GTATGGTTGC AAAGCTGAAA CTTAAAGGAA | |
| | | TTGACGGAAG GGCACCACCA GGAGTGGAGC CTGCGGCTTA | |
| | | ATTTGACTCA ACACGGGAAA CCTCACCCGG CCCGGACACG | |
| | | GACAGGATTG ACAGATTGAT AGCTCTTTCT CGATTCCGTG | |
| | | GGTGGTGGTG CATGGCCGTT CTTAGTTGGT GGAGCGATTT | |
| | | GTCTGGTTAA TTCCGATAAC GAACGAGACT CTGGCATGCT | |
| | | AACTAGTTAC GCGACCCCCG AGCGGTCGGC GTCCCCCAAC | |
| | | TTCTTAGAGG GACAAGTGGC GTTCAGCCAC CCGAGATTGA | |
| | | GCAATAACAG GTCTGTGATG CCCTTAGATG TCCGGGGCTG | |
| | | CACGCGCGCT ACACTGACTG GCTCAGCGTG TGCCTACCCT | |
| | | ACGCCGGCAG GCGCGGGTAA CCCGTTGAAC CCCATTCGTG | |
| | | ATGGGGATCG GGGATTGCAA TTATTCCCCA TGAACGAGGA | |
| | | ATTCCCAGTA AGTGCGGGTC ATAAGCTTGC GTTGATTAAG | |
| | | TCCCTGCCCT TTGTACACAC CGCCCGTCGC TACTACCGAT | |
| | | TGGATGGTTT AGTGAGGCCC TCGGATCGGC CCCGCCGGGG | |
| | | TCGGCCCACG GCCCTGGCGG AGCGCTGAGA AGACGGTCGA | |
| | | ACTTGACTAT CTAGAGGAAG TAAAAGTCGT AACAAGGTTT | |
| | | CCGTAGGTGA ACCTGCGGAA GGATCATTA | |
| PPIA | NM_021130.4 | GGGGCCGAAC GTGGTATAAA AGGGGCGGGA GGCCAGGCTC | 50 |
| | | GTGCCGTTTT GCAGACGCCA CCGCCGAGGA AAACCGTGTA | |
| | | CTATTAGCCA TGGTCAACCC CACCGTGTTC TTCGACATTG | |
| | | CCGTCGACGG CGAGCCCTTG GGCCGCGTCT CCTTTGAGCT | |
| | | GTTTGCAGAC AAGGTCCCAA AGACAGCAGA AAATTTTCGT | |
| | | GCTCTGAGCA CTGGAGAGAA AGGATTTGGT TATAAGGGTT | |
| | | CCTGCTTTCA CAGAATTATT CCAGGGTTTA TGTGTCAGGG | |
| | | TGGTGACTTC ACACGCCATA ATGGCACTGG TGGCAAGTCC | |
| | | ATCTATGGGG AGAAATTTGA AGATGAGAAC TTCATCCTAA | |
| | | AGCATACGGG TCCTGGCATC TTGTCCATGG CAAATGCTGG | |
| | | ACCCAACACA AATGGTTCCC AGTTTTTCAT CTGCACTGCC | |
| | | AAGACTGAGT GGTTGGATGG CAAGCATGTG GTGTTTGGCA | |
| | | AAGTGAAAGA AGGCATGAAT ATTGTGGAGG CCATGGAGCG | |
| | | CTTTGGGTCC AGGAATGGCA AGACCAGCAA GAAGATCACC | |
| | | ATTGCTGACT GTGGACAACT CGAATAAGTT TGACTTGTGT | |
| | | TTTATCTTAA CCACCAGATC ATTCCTTCTG TAGCTCAGGA | |
| | | GAGCACCCCT CCACCCCATT TGCTCGCAGT ATCCTAGAAT | |
| | | CTTTGTGCTC TCGCTGCAGT TCCCTTTGGG TTCCATGTTT | |
| | | TCCTTGTTCC CTCCCATGCC TAGCTGGATT GCAGAGTTAA | |
| | | GTTTATGATT ATGAAATAAA AACTAAATAA CAATTGTCCT | |
| | | CGTTTGAGTT AAGAGTGTTG ATGTAGGCTT TATTTTAAGC | |
| | | AGTAATGGGT TACTTCTGAA ACATCACTTG TTTGCTTAAT | |
| | | TCTACACAGT ACTTAGATTT TTTTTACTTT CCAGTCCCAG | |
| | | GAAGTGTCAA TGTTTGTTGA GTGGAATATT GAAAATGTAG | |
| | | GCAGCAACTG GGCATGGTGG CTCACTGTCT GTAATGTATT | |
| | | ACCTGAGGCA GAAGACCACC TGAGGGTAGG AGTCAAGATC | |
| | | AGCCTGGGCA ACATAGTGAG ACGCTGTCTC TACAAAAAAT | |
| | | AATTAGCCTG GCCTGGTGGT GCATGCCTAG TCCTAGCTGA | |
| | | TCTGGAGGCT GACGTGGGAG GATTGCTTGA GCCTAGAGTG | |
| | | AGCTATTATC ATGCCACTGT ACAGCCTGGG TGTTCACAGA | |
| | | TCTTGTGTCT CAAAGGTAGG CAGAGGCAGG AAAAGCAAGG | |
| | | AGCCAGAATT AAGAGGTTGG GTCAGTCTGC AGTGAGTTCA | |
| | | TGCATTTAGA GGTGTTCTTC AAGATGACTA ATGTCAAAAA | |
| | | TTGAGACATC TGTTGCGGTT TTTTTTTTTT TTTTTTCCCC | |
| | | TGGAATGCAG TGGCGTGATC TCAGCTCACT GCAGCCTCCG | |
| | | CCTCCTGGGT TCAAGTGATT CTAGTGCCTC AGCCTCCTGA | |
| | | GTAGCTGGGA TAATGGGCGT GTGCCACCAT GCCCAGCTAA | |
| | | TTTTTGTATT TTTAGTATAG ATGGGGTTTC ATCATTTTGA | |
| | | CCAGGCTGGT CTCAAACTCT TGACCTCAGC TGATGCGCCT | |
| | | GCCTTGGCCT CCCAAACTGC TGAGATTACA GATGTGAGCC | |
| | | ACCGCACCCT ACCTCATTTT CTGTAACAAA GCTAAGCTTG | |
| | | AACACTGTTG ATGTTCTTGA GGGAAGCATA TTGGGCTTTA | |
| | | GGCTGTAGGT CAAGTTTATA CATCTTAATT ATGGTGGAAT | |
| | | TCCTATGTAG AGTCTAAAAA GCCAGGTACT TGGTGCTACA | |
| | | GTCAGTCTCC CTGCAGAGGG TTAAGGCGCA GACTACCTGC | |
| | | AGTGAGGAGG TACTGCTTGT AGCATATAGA GCCTCTCCCT | |
| | | AGCTTTGGTT ATGGAGGCTT TGAGGTTTTG CAAACCTGAC | |
| | | CAATTTAAGC CATAAGATCT GGTCAAAGGG ATACCCTTCC | |
| | | CACTAAGGAC TTGGTTTCTC AGGAAATTAT ATGTACAGTG | |
| | | CTTGCTGGCA GTTAGATGTC AGGACAATCT AAGCTGAGAA | |
| | | AACCCCTTCT CTGCCCACCT TAACAGACCT CTAGGGTTCT | |
| | | TAACCCAGCA ATCAAGTTTG CCTATCCTAG AGGTGGCGGA | |
| | | TTTGATCATT TGGTGTGTTG GGCAATTTTT GTTTTACTGT | |
| | | CTGGTTCCTT CTGCGTGAAT TACCACCACC ACCACTTGTG | |
| | | CATCTCAGTC TTGTGTGTTG TCTGGTTACG TATTCCCTGG | |
| | | GTGATACCAT TCAATGTCTT AATGTACTTG TGGCTCAGAC | |
| | | CTGAGTGCAA GGTGGAAATA AACATCAAAC ATCTTTTCAT | |
| | | TATCCCTA | |
| PGK1 | NM_000291.3 | GAGAGCAGCG GCCGGGAAGG GGCGGTGCGG GAGGCGGGGT | 51 |
| | | GTGGGGCGGT AGTGTGGGCC CTGTTCCTGC CCGCGCGGTG | |
| | | TTCCGCATTC TGCAAGCCTC CGGAGCGCAC GTCGGCAGTC | |
| | | GGCTCCCTCG TTGACCGAAT CACCGACCTC TCTCCCCAGC | |
| | | TGTATTTCCA AAATGTCGCT TTCTAACAAG CTGACGCTGG | |
| | | ACAAGCTGGA CGTTAAAGGG AAGCGGGTCG TTATGAGAGT | |
| | | CGACTTCAAT GTTCCTATGA AGAACAACCA GATAACAAAC | |
| | | AACCAGAGGA TTAAGGCTGC TGTCCCAAGC ATCAAATTCT | |
| | | GCTTGGACAA TGGAGCCAAG TCGGTAGTCC TTATGAGCCA | |
| | | CCTAGGCCGG CCTGATGGTG TGCCCATGCC TGACAAGTAC | |
| | | TCCTTAGAGC CAGTTGCTGT AGAACTCAAA TCTCTGCTGG | |
| | | GCAAGGATGT TCTGTTCTTG AAGGACTGTG TAGGCCCAGA | |
| | | AGTGGAGAAA GCCTGTGCCA ACCCAGCTGC TGGGTCTGTC | |
| | | ATCCTGCTGG AGAACCTCCG CTTTCATGTG GAGGAAGAAG | |
| | | GGAAGGGAAA AGATGCTTCT GGGAACAAGG TTAAAGCCGA | |
| | | GCCAGCCAAA ATAGAAGCTT TCCGAGCTTC ACTTTCCAAG | |
| | | CTAGGGGATG TCTATGTCAA TGATGCTTTT GGCACTGCTC | |
| | | ACAGAGCCCA CAGCTCCATG GTAGGAGTCA ATCTGCCACA | |
| | | GAAGGCTGGT GGGTTTTTGA TGAAGAAGGA GCTGAACTAC | |
| | | TTTGCAAAGG CCTTGGAGAG CCCAGAGCGA CCCTTCCTGG | |
| | | CCATCCTGGG CGGAGCTAAA GTTGCAGACA AGATCCAGCT | |
| | | CATCAATAAT ATGCTGGACA AAGTCAATGA GATGATTATT | |
| | | GGTGGTGGAA TGGCTTTTAC CTTCCTTAAG GTGCTCAACA | |
| | | ACATGGAGAT TGGCACTTCT CTGTTTGATG AAGAGGGAGC | |
| | | CAAGATTGTC AAAGACCTAA TGTCCAAAGC TGAGAAGAAT | |
| | | GGTGTGAAGA TTACCTTGCC TGTTGACTTT GTCACTGCTG | |
| | | ACAAGTTTGA TGAGAATGCC AAGACTGGCC AAGCCACTGT | |
| | | GGCTTCTGGC ATACCTGCTG GCTGGATGGG CTTGGACTGT | |
| | | GGTCCTGAAA GCAGCAAGAA GTATGCTGAG GCTGTCACTC | |
| | | GGGCTAAGCA GATTGTGTGG AATGGTCCTG TGGGGGTATT | |
| | | TGAATGGGAA GCTTTTGCCC GGGGAACCAA AGCTCTCATG | |
| | | GATGAGGTGG TGAAAGCCAC TTCTAGGGGC TGCATCACCA | |
| | | TCATAGGTGG TGGAGACACT GCCACTTGCT GTGCCAAATG | |
| | | GAACACGGAG GATAAAGTCA GCCATGTGAG CACTGGGGGT | |
| | | GGTGCCAGTT TGGAGCTCCT GGAAGGTAAA GTCCTTCCTG | |
| | | GGGTGGATGC TCTCAGCAAT ATTTAGTACT TTCCTGCCTT | |
| | | TTAGTTCCTG TGCACAGCCC CTAAGTCAAC TTAGCATTTT | |
| | | CTGCATCTCC ACTTGGCATT AGCTAAAACC TTCCATGTCA | |
| | | AGATTCAGCT AGTGGCCAAG AGATGCAGTG CCAGGAACCC | |
| | | TTAAACAGTT GCACAGCATC TCAGCTCATC TTCACTGCAC | |
| | | CCTGGATTTG CATACATTCT TCAAGATCCC ATTTGAATTT | |
| | | TTTAGTGACT AAACCATTGT GCATTCTAGA GTGCATATAT | |
| | | TTATATTTTG CCTGTTAAAA AGAAAGTGAG CAGTGTTAGC | |
| | | TTAGTTCTCT TTTGATGTAG GTTATTATGA TTAGCTTTGT | |
| | | CACTGTTTCA CTACTCAGCA TGGAAACAAG ATGAAATTCC | |
| | | ATTTGTAGGT AGTGAGACAA AATTGATGAT CCATTAAGTA | |
| | | AACAATAAAA GTGTCCATTG AAACCGTGAT TTTTTTTTTT | |
| | | TTCCTGTCAT ACTTTGTTAG GAAGGGTGAG AATAGAATCT | |
| | | TGAGGAACGG ATCAGATGTC TATATTGCTG AATGCAAGAA | |
| | | GTGGGGCAGC AGCAGTGGAG AGATGGGACA ATTAGATAAA | |
| | | TGTCCATTCT TTATCAAGGG CCTACTTTAT GGCAGACATT | |
| | | GTGCTAGTGC TTTTATTCTA ACTTTTATTT TTATCAGTTA | |
| | | CACATGATCA TAATTTAAAA AGTCAAGGCT TATAACAAAA | |
| | | AAGCCCCAGC CCATTCCTCC CATTCAAGAT TCCCACTCCC | |
| | | CAGAGGTGAC CACTTTCAAC TCTTGAGTTT TTCAGGTATA | |
| | | TACCTCCATG TTTCTAAGTA ATATGCTTAT ATTGTTCACT | |
| | | TCTTTTTTTT TTATTTTTTA AAGAAATCTA TTTCATACCA | |
| | | TGGAGGAAGG CTCTGTTCCA CATATATTTC CACTTCTTCA | |
| | | TTCTCTCGGT ATAGTTTTGT CACAATTATA GATTAGATCA | |
| | | AAAGTCTACA TAACTAATAC AGCTGAGCTA TGTAGTATGC | |
| | | TATGATTAAA TTTACTTATG TAAAAAAAAA AAAAAAAAA | |
| RPL13A | NM_012423.3 | CACTTCTGCC GCCCCTGTTT CAAGGGATAA GAAACCCTGC | 52 |
| | | GACAAAACCT CCTCCTTTTC CAAGCGGCTG CCGAAGATGG | |
| | | CGGAGGTGCA GGTCCTGGTG CTTGATGGTC GAGGCCATCT | |
| | | CCTGGGCCGC CTGGCGGCCA TCGTGGCTAA ACAGGTACTG | |
| | | CTGGGCCGGA AGGTGGTGGT CGTACGCTGT GAAGGCATCA | |
| | | ACATTTCTGG CAATTTCTAC AGAAACAAGT TGAAGTACCT | |
| | | GGCTTTCCTC CGCAAGCGGA TGAACACCAA CCCTTCCCGA | |
| | | GGCCCCTACC ACTTCCGGGC CCCCAGCCGC ATCTTCTGGC | |
| | | GGACCGTGCG AGGTATGCTG CCCCACAAAA CCAAGCGAGG | |
| | | CCAGGCCGCT CTGGACCGTC TCAAGGTGTT TGACGGCATC | |
| | | CCACCGCCCT ACGACAAGAA AAAGCGGATG GTGGTTCCTG | |
| | | CTGCCCTCAA GGTCGTGCGT CTGAAGCCTA CAAGAAAGTT | |
| | | TGCCTATCTG GGGCGCCTGG CTCACGAGGT TGGCTGGAAG | |
| | | TACCAGGCAG TGACAGCCAC CCTGGAGGAG AAGAGGAAAG | |
| | | AGAAAGCCAA GATCCACTAC CGGAAGAAGA AACAGCTCAT | |
| | | GAGGCTACGG AAACAGGCCG AGAAGAACGT GGAGAAGAAA | |
| | | ATTGACAAAT ACACAGAGGT CCTCAAGACC CACGGACTCC | |
| | | TGGTCTGAGC CCAATAAAGA CTGTTAATTC CTCATGCGTT | |
| | | GCCTGCCCTT CCTCCATTGT TGCCCTGGAA TGTACGGGAC | |
| | | CCAGGGGCAG CAGCAGTCCA GGTGCCACAG GCAGCCCTGG | |
| | | GACATAGGAA GCTGGGAGCA AGGAAAGGGT CTTAGTCACT | |
| | | GCCTCCCGAA GTTGCTTGAA AGCACTCGGA GAATTGTGCA | |
| | | GGTGTCATTT ATCTATGACC AATAGGAAGA GCAACCAGTT | |
| | | ACTATGAGTG AAAGGGAGCC AGAAGACTGA TTGGAGGGCC | |
| | | CTATCTTGTG AGTGGGGCAT CTGTTGGACT TTCCACCTGG | |
| | | TCATATACTC TGCAGCTGTT AGAATGTGCA AGCACTTGGG | |
| | | GACAGCATGA GCTTGCTGTT GTACACAGGG TATTTCTAGA | |
| | | AGCAGAAATA GACTGGGAAG ATGCACAACC AAGGGGTTAC | |
| | | AGGCATCGCC CATGCTCCTC ACCTGTATTT TGTAATCAGA | |
| | | AATAAATTGC TTTTAAAGAA AAAAAAAAAA AAAAAA | |
| B2M | NM_004048.2 | AATATAAGTG GAGGCGTCGC GCTGGCGGGC ATTCCTGAAG | 53 |
| | | CTGACAGCAT TCGGGCCGAG ATGTCTCGCT CCGTGGCCTT | |
| | | AGCTGTGCTC GCGCTACTCT CTCTTTCTGG CCTGGAGGCT | |
| | | ATCCAGCGTA CTCCAAAGAT TCAGGTTTAC TCACGTCATC | |
| | | CAGCAGAGAA TGGAAAGTCA AATTTCCTGA ATTGCTATGT | |
| | | GTCTGGGTTT CATCCATCCG ACATTGAAGT TGACTTACTG | |
| | | AAGAATGGAG AGAGAATTGA AAAAGTGGAG CATTCAGACT | |
| | | TGTCTTTCAG CAAGGACTGG TCTTTCTATC TCTTGTACTA | |
| | | CACTGAATTC ACCCCCACTG AAAAAGATGA GTATGCCTGC | |
| | | CGTGTGAACC ATGTGACTTT GTCACAGCCC AAGATAGTTA | |
| | | AGTGGGATCG AGACATGTAA GCAGCATCAT GGAGGTTTGA | |
| | | AGATGCCGCA TTTGGATTGG ATGAATTCCA AATTCTGCTT | |
| | | GCTTGCTTTT TAATATTGAT ATGCTTATAC ACTTACACTT | |
| | | TATGCACAAA ATGTAGGGTT ATAATAATGT TAACATGGAC | |
| | | ATGATCTTCT TTATAATTCT ACTTTGAGTG CTGTCTCCAT | |
| | | GTTTGATGTA TCTGAGCAGG TTGCTCCACA GGTAGCTCTA | |
| | | GGAGGGCTGG CAACTTAGAG GTGGGGAGCA GAGAATTCTC | |
| | | TTATCCAACA TCAACATCTT GGTCAGATTT GAACTCTTCA | |
| | | ATCTCTTGCA CTCAAAGCTT GTTAAGATAG TTAAGCGTGC | |
| | | ATAAGTTAAC TTCCAATTTA CATACTCTGC TTAGAATTTG | |
| | | GGGGAAAATT TAGAAATATA ATTGACAGGA TTATTGGAAA | |
| | | TTTGTTATAA TGAATGAAAC ATTTTGTCAT ATAAGATTCA | |
| | | TATTTACTTC TTATACATTT GATAAAGTAA GGCATGGTTG | |
| | | TGGTTAATCT GGTTTATTTT TGTTCCACAA GTTAAATAAA | |
| | | TCATAAAACT TGATGTGTTA TCTCTTA | |
| YWHAZ | NM_003406.3 | CTTTCTCCTT CCCCTTCTTC CGGGCTCCCG TCCCGGCTCA | 54 |
| | | TCACCCGGCC TGTGGCCCAC TCCCACCGCC AGCTGGAACC | |
| | | CTGGGGACTA CGACGTCCCT CAAACCTTGC TTCTAGGAGA | |
| | | TAAAAAGAAC ATCCAGTCAT GGATAAAAAT GAGCTGGTTC | |
| | | AGAAGGCCAA ACTGGCCGAG CAGGCTGAGC GATATGATGA | |
| | | CATGGCAGCC TGCATGAAGT CTGTAACTGA GCAAGGAGCT | |
| | | GAATTATCCA ATGAGGAGAG GAATCTTCTC TCAGTTGCTT | |
| | | ATAAAAATGT TGTAGGAGCC CGTAGGTCAT CTTGGAGGGT | |
| | | CGTCTCAAGT ATTGAACAAA AGACGGAAGG TGCTGAGAAA | |
| | | AAACAGCAGA TGGCTCGAGA ATACAGAGAG AAAATTGAGA | |
| | | CGGAGCTAAG AGATATCTGC AATGATGTAC TGTCTCTTTT | |
| | | GGAAAAGTTC TTGATCCCCA ATGCTTCACA AGCAGAGAGC | |
| | | AAAGTCTTCT ATTTGAAAAT GAAAGGAGAT TACTACCGTT | |
| | | ACTTGGCTGA GGTTGCCGCT GGTGATGACA AGAAAGGGAT | |
| | | TGTCGATCAG TCACAACAAG CATACCAAGA AGCTTTTGAA | |
| | | ATCAGCAAAA AGGAAATGCA ACCAACACAT CCTATCAGAC | |
| | | TGGGTCTGGC CCTTAACTTC TCTGTGTTCT ATTATGAGAT | |
| | | TCTGAACTCC CCAGAGAAAG CCTGCTCTCT TGCAAAGACA | |
| | | GCTTTTGATG AAGCCATTGC TGAACTTGAT ACATTAAGTG | |
| | | AAGAGTCATA CAAAGACAGC ACGCTAATAA TGCAATTACT | |
| | | GAGAGACAAC TTGACATTGT GGACATCGGA TACCCAAGGA | |
| | | GACGAAGCTG AAGCAGGAGA AGGAGGGGAA AATTAACCGG | |
| | | CCTTCCAACT TTTGTCTGCC TCATTCTAAA ATTTACACAG | |
| | | TAGACCATTT GTCATCCATG CTGTCCCACA AATAGTTTTT | |
| | | TGTTTACGAT TTATGACAGG TTTATGTTAC TTCTATTTGA | |
| | | ATTTCTATAT TTCCCATGTG GTTTTTATGT TTAATATTAG | |
| | | GGGAGTAGAG CCAGTTAACA TTTAGGGAGT TATCTGTTTT | |
| | | CATCTTGAGG TGGCCAATAT GGGGATGTGG AATTTTTATA | |
| | | CAAGTTATAA GTGTTTGGCA TAGTACTTTT GGTACATTGT | |
| | | GGCTTCAAAA GGGCCAGTGT AAAACTGCTT CCATGTCTAA | |
| | | GCAAAGAAAA CTGCCTACAT ACTGGTTTGT CCTGGCGGGG | |
| | | AATAAAAGGG ATCATTGGTT CCAGTCACAG GTGTAGTAAT | |
| | | TGTGGGTACT TTAAGGTTTG GAGCACTTAC AAGGCTGTGG | |
| | | TAGAATCATA CCCCATGGAT ACCACATATT AAACCATGTA | |
| | | TATCTGTGGA ATACTCAATG TGTACACCTT TGACTACAGC | |
| | | TGCAGAAGTG TTCCTTTAGA CAAAGTTGTG ACCCATTTTA | |
| | | CTCTGGATAA GGGCAGAAAC GGTTCACATT CCATTATTTG | |
| | | TAAAGTTACC TGCTGTTAGC TTTCATTATT TTTGCTACAC | |
| | | TCATTTTATT TGTATTTAAA TGTTTTAGGC AACCTAAGAA | |
| | | CAAATGTAAA AGTAAAGATG CAGGAAAAAT GAATTGCTTG | |
| | | GTATTCATTA CTTCATGTAT ATCAAGCACA GCAGTAAAAC | |
| | | AAAAACCCAT GTATTTAACT TTTTTTTAGG ATTTTTGCTT | |
| | | TTGTGATTTT TTTTTTTTTG ATACTTGCCT AACATGCATG | |
| | | TGCTGTAAAA ATAGTTAACA GGGAAATAAC TTGAGATGAT | |
| | | GGCTAGCTTT GTTTAATGTC TTATGAAATT TTCATGAACA | |
| | | ATCCAAGCAT AATTGTTAAG AACACGTGTA TTAAATTCAT | |
| | | GTAAGTGGAA TAAAAGTTTT ATGAATGGAC TTTTCAACTA | |
| | | CTTTCTCTAC AGCTTTTCAT GTAAATTAGT CTTGGTTCTG | |
| | | AAACTTCTCT AAAGGAAATT GTACATTTTT TGAAATTTAT | |
| | | TCCTTATTCC CTCTTGGCAG CTAATGGGCT CTTACCAAGT | |
| | | TTAAACACAA AATTTATCAT AACAAAAATA CTACTAATAT | |
| | | AACTACTGTT TCCATGTCCC ATGATCCCCT CTCTTCCTCC | |
| | | CCACCCTGAA AAAAATGAGT TCCTATTTTT TCTGGGAGAG | |
| | | GGGGGGATTG ATTAGAAAAA AATGTAGTGT GTTCCATTTA | |
| | | AAATTTTGGC ATATGGCATT TTCTAACTTA GGAAGCCACA | |
| | | ATGTTCTTGG CCCATCATGA CATTGGGTAG CATTAACTGT | |
| | | AAGTTTTGTG CTTCCAAATC ACTTTTTGGT TTTTAAGAAT | |
| | | TTCTTGATAC TCTTATAGCC TGCCTTCAAT TTTGATCCTT | |
| | | TATTCTTTCT ATTTGTCAGG TGCACAAGAT TACCTTCCTG | |
| | | TTTTAGCCTT CTGTCTTGTC ACCAACCATT CTTACTTGGT | |
| | | GGCCATGTAC TTGGAAAAAG GCCGCATGAT CTTTCTGGCT | |
| | | CCACTCAGTG TCTAAGGCAC CCTGCTTCCT TTGCTTGCAT | |
| | | CCCACAGACT ATTTCCCTCA TCCTATTTAC TGCAGCAAAT | |
| | | CTCTCCTTAG TTGATGAGAC TGTGTTTATC TCCCTTTAAA | |
| | | ACCCTACCTA TCCTGAATGG TCTGTCATTG TCTGCCTTTA | |
| | | AAATCCTTCC TCTTTCTTCC TCCTCTATTC TCTAAATAAT | |
| | | GATGGGGCTA AGTTATACCC AAAGCTCACT TTACAAAATA | |
| | | TTTCCTCAGT ACTTTGCAGA AAACACCAAA CAAAAATGCC | |
| | | ATTTTAAAAA AGGTGTATTT TTTCTTTTAG AATGTAAGCT | |
| | | CCTCAAGAGC AGGGACAATG TTTTCTGTAT GTTCTATTGT | |
| | | GCCTAGTACA CTGTAAATGC TCAATAAATA TTGATGATGG | |
| | | GAGGCAGTGA GTCTTGATGA TAAGGGTGAG AAACTGAAAT | |
| | | CCCAAACACT GTTTTGTTGC TTGTTTTATT ATGACCTCAG | |
| | | ATTAAATTGG GAAATATTGG CCCTTTTGAA TAATTGTCCC | |
| | | AAATATTACA TTCAAATAAA AGTGCAATGG AGAAAAAAAA | |
| | | AAA | |
| SDHA | NM_004168.3 | ACTGCAGCCC CGCTCGACTC CGGCGTGGTG CGCAGGCGCG | 55 |
| | | GTATCCCCCC TCCCCCGCCA GCTCGACCCC GGTGTGGTGC | |
| | | GCAGGCGCAG TCTGCGCAGG GACTGGCGGG ACTGCGCGGC | |
| | | GGCAACAGCA GACATGTCGG GGGTCCGGGG CCTGTCGCGG | |
| | | CTGCTGAGCG CTCGGCGCCT GGCGCTGGCC AAGGCGTGGC | |
| | | CAACAGTGTT GCAAACAGGA ACCCGAGGTT TTCACTTCAC | |
| | | TGTTGATGGG AACAAGAGGG CATCTGCTAA AGTTTCAGAT | |
| | | TCCATTTCTG CTCAGTATCC AGTAGTGGAT CATGAATTTG | |
| | | ATGCAGTGGT GGTAGGCGCT GGAGGGGCAG GCTTGCGAGC | |
| | | TGCATTTGGC CTTTCTGAGG CAGGGTTTAA TACAGCATGT | |
| | | GTTACCAAGC TGTTTCCTAC CAGGTCACAC ACTGTTGCAG | |
| | | CACAGGGAGG AATCAATGCT GCTCTGGGGA ACATGGAGGA | |
| | | GGACAACTGG AGGTGGCATT TCTACGACAC CGTGAAGGGC | |
| | | TCCGACTGGC TGGGGGACCA GGATGCCATC CACTACATGA | |
| | | CGGAGCAGGC CCCCGCCGCC GTGGTCGAGC TAGAAAATTA | |
| | | TGGCATGCCG TTTAGCAGAA CTGAAGATGG GAAGATTTAT | |
| | | CAGCGTGCAT TTGGTGGACA GAGCCTCAAG TTTGGAAAGG | |
| | | GCGGGCAGGC CCATCGGTGC TGCTGTGTGG CTGATCGGAC | |
| | | TGGCCACTCG CTATTGCACA CCTTATATGG AAGGTCTCTG | |
| | | CGATATGATA CCAGCTATTT TGTGGAGTAT TTTGCCTTGG | |
| | | ATCTCCTGAT GGAGAATGGG GAGTGCCGTG GTGTCATCGC | |
| | | ACTGTGCATA GAGGACGGGT CCATCCATCG CATAAGAGCA | |
| | | AAGAACACTG TTGTTGCCAC AGGAGGCTAC GGGCGCACCT | |
| | | ACTTCAGCTG CACGTCTGCC CACACCAGCA CTGGCGACGG | |
| | | CACGGCCATG ATCACCAGGG CAGGCCTTCC TTGCCAGGAC | |
| | | CTAGAGTTTG TTCAGTTCCA CCCTACAGGC ATATATGGTG | |
| | | CTGGTTGTCT CATTACGGAA GGATGTCGTG GAGAGGGAGG | |
| | | CATTCTCATT AACAGTCAAG GCGAAAGGTT TATGGAGCGA | |
| | | TACGCCCCTG TCGCGAAGGA CCTGGCGTCT AGAGATGTGG | |
| | | TGTCTCGGTC CATGACTCTG GAGATCCGAG AAGGAAGAGG | |
| | | CTGTGGCCCT GAGAAAGATC ACGTCTACCT GCAGCTGCAC | |
| | | CACCTACCTC CAGAGCAGCT GGCCACGCGC CTGCCTGGCA | |
| | | TTTCAGAGAC AGCCATGATC TTCGCTGGCG TGGACGTCAC | |
| | | GAAGGAGCCG ATCCCTGTCC TCCCCACCGT GCATTATAAC | |
| | | ATGGGCGGCA TTCCCACCAA CTACAAGGGG CAGGTCCTGA | |
| | | GGCACGTGAA TGGCCAGGAT CAGATTGTGC CCGGCCTGTA | |
| | | CGCCTGTGGG GAGGCCGCCT GTGCCTCGGT ACATGGTGCC | |
| | | AACCGCCTCG GGGCAAACTC GCTCTTGGAC CTGGTTGTCT | |
| | | TTGGTCGGGC ATGTGCCCTG AGCATCGAAG AGTCATGCAG | |
| | | GCCTGGAGAT AAAGTCCCTC CAATTAAACC AAACGCTGGG | |
| | | GAAGAATCTG TCATGAATCT TGACAAATTG AGATTTGCTG | |
| | | ATGGAAGCAT AAGAACATCG GAACTGCGAC TCAGCATGCA | |
| | | GAAGTCAATG CAAAATCATG CTGCCGTGTT CCGTGTGGGA | |
| | | AGCGTGTTGC AAGAAGGTTG TGGGAAAATC AGCAAGCTCT | |
| | | ATGGAGACCT AAAGCACCTG AAGACGTTCG ACCGGGGAAT | |
| | | GGTCTGGAAC ACGGACCTGG TGGAGACCCT GGAGCTGCAG | |
| | | AACCTGATGC TGTGTGCGCT GCAGACCATC TACGGAGCAG | |
| | | AGGCACGGAA GGAGTCACGG GGCGCGCATG CCAGGGAAGA | |
| | | CTACAAGGTG CGGATTGATG AGTACGATTA CTCCAAGCCC | |
| | | ATCCAGGGGC AACAGAAGAA GCCCTTTGAG GAGCACTGGA | |
| | | GGAAGCACAC CCTGTCCTAT GTGGACGTTG GCACTGGGAA | |
| | | GGTCACTCTG GAATATAGAC CCGTGATCGA CAAAACTTTG | |
| | | AACGAGGCTG ACTGTGCCAC CGTCCCGCCA GCCATTCGCT | |
| | | CCTACTGATG AGACAAGATG TGGTGATGAC AGAATCAGCT | |
| | | TTTGTAATTA TGTATAATAG CTCATGCATG TGTCCATGTC | |
| | | ATAACTGTCT TCATACGCTT CTGCACTCTG GGGAAGAAGG | |
| | | AGTACATTGA AGGGAGATTG GCACCTAGTG GCTGGGAGCT | |
| | | TGCCAGGAAC CCAGTGGCCA GGGAGCGTGG CACTTACCTT | |
| | | TGTCCCTTGC TTCATTCTTG TGAGATGATA AAACTGGGCA | |
| | | CAGCTCTTAA ATAAAATATA AATGAACAAA CTTTCTTTTA | |
| | | TTTCCAAATC CATTTGAAAT ATTTTACTGT TGTGACTTTA | |
| | | GTCATATTTG TTGACCTAAA AATCAAATGT AATCTTTGTA | |
| | | TTGTGTTACA TCAAAATCCA GATATTTTGT ATAGTTTCTT | |
| | | TTTTCTTTTT CTTTTCTTTT TTTTTTTTGA GACAGGATCG | |
| | | GTGCAGTAGT ACAATCACAG CTCACTGCAG CCTCAAACTC | |
| | | CTGGGCAGCT CAGGTGATCT TCCTGACTCA GCCTTCTGAG | |
| | | TAGTTGGGGC TACAGGTGTG CACCACCATG CCCAGCTCAT | |
| | | TTATTTTGTA ATTGTAGGGA CAGGGTCTCA CTGTGTTGCC | |
| | | TAGGCTGGTC TCAAGTGATC CTCCCTCCTT GGCCTCCCAA | |
| | | GGTGCTGGAA TTATAGGTGT GAACAAACCA AAAAAAAAAA | |
| | | AAA | |
| HPRT1 | NM_000194.2 | GGCGGGGCCT GCTTCTCCTC AGCTTCAGGC GGCTGCGACG | 56 |
| | | AGCCCTCAGG CGAACCTCTC GGCTTTCCCG CGCGGCGCCG | |
| | | CCTCTTGCTG CGCCTCCGCC TCCTCCTCTG CTCCGCCACC | |
| | | GGCTTCCTCC TCCTGAGCAG TCAGCCCGCG CGCCGGCCGG | |
| | | CTCCGTTATG GCGACCCGCA GCCCTGGCGT CGTGATTAGT | |
| | | GATGATGAAC CAGGTTATGA CCTTGATTTA TTTTGCATAC | |
| | | CTAATCATTA TGCTGAGGAT TTGGAAAGGG TGTTTATTCC | |
| | | TCATGGACTA ATTATGGACA GGACTGAACG TCTTGCTCGA | |
| | | GATGTGATGA AGGAGATGGG AGGCCATCAC ATTGTAGCCC | |
| | | TCTGTGTGCT CAAGGGGGGC TATAAATTCT TTGCTGACCT | |
| | | GCTGGATTAC ATCAAAGCAC TGAATAGAAA TAGTGATAGA | |
| | | TCCATTCCTA TGACTGTAGA TTTTATCAGA CTGAAGAGCT | |
| | | ATTGTAATGA CCAGTCAACA GGGGACATAA AAGTAATTGG | |
| | | TGGAGATGAT CTCTCAACTT TAACTGGAAA GAATGTCTTG | |
| | | ATTGTGGAAG ATATAATTGA CACTGGCAAA ACAATGCAGA | |
| | | CTTTGCTTTC CTTGGTCAGG CAGTATAATC CAAAGATGGT | |
| | | CAAGGTCGCA AGCTTGCTGG TGAAAAGGAC CCCACGAAGT | |
| | | GTTGGATATA AGCCAGACTT TGTTGGATTT GAAATTCCAG | |
| | | ACAAGTTTGT TGTAGGATAT GCCCTTGACT ATAATGAATA | |
| | | CTTCAGGGAT TTGAATCATG TTTGTGTCAT TAGTGAAACT | |
| | | GGAAAAGCAA AATACAAAGC CTAAGATGAG AGTTCAAGTT | |
| | | GAGTTTGGAA ACATCTGGAG TCCTATTGAC ATCGCCAGTA | |
| | | AAATTATCAA TGTTCTAGTT CTGTGGCCAT CTGCTTAGTA | |
| | | GAGCTTTTTG CATGTATCTT CTAAGAATTT TATCTGTTTT | |
| | | GTACTTTAGA AATGTCAGTT GCTGCATTCC TAAACTGTTT | |
| | | ATTTGCACTA TGAGCCTATA GACTATCAGT TCCCTTTGGG | |
| | | CGGATTGTTG TTTAACTTGT AAATGAAAAA ATTCTCTTAA | |
| | | ACCACAGCAC TATTGAGTGA AACATTGAAC TCATATCTGT | |
| | | AAGAAATAAA GAGAAGATAT ATTAGTTTTT TAATTGGTAT | |
| | | TTTAATTTTT ATATATGCAG GAAAGAATAG AAGTGATTGA | |
| | | ATATTGTTAA TTATACCACC GTGTGTTAGA AAAGTAAGAA | |
| | | GCAGTCAATT TTCACATCAA AGACAGCATC TAAGAAGTTT | |
| | | TGTTCTGTCC TGGAATTATT TTAGTAGTGT TTCAGTAATG | |
| | | TTGACTGTAT TTTCCAACTT GTTCAAATTA TTACCAGTGA | |
| | | ATCTTTGTCA GCAGTTCCCT TTTAAATGCA AATCAATAAA | |
| | | TTCCCAAAAA TTTAAAAAAA AAAAAAAAAA AAAAA | |

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In other embodiments, a nucleic acid molecule or nucleic acid sequence comprises other kinds of nucleic acid structures such a for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include tissue, organs, or bodily fluids such as whole blood, plasma, serum, tissue, lavage or any other specimen used for detection of disease.

The term “subject” as used herein refers to a mammal, preferably a human.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and five-fold. The change may be statistically significant with a p value of 0.1, 0.05, 0.001, or 0.0001.

The term “stable disease” refers to a diagnosis for the presence of a melanoma, however the melanoma has been treated and remains in a stable condition, i.e. one that that is not progressive, as determined by imaging data and/or best clinical judgment.

The term “progressive disease” refers to a diagnosis for the presence of a highly active state of a melanoma, i.e. one has not been treated and is not stable or has been treated and has not responded to therapy, or has been treated and active disease remains, as determined by imaging data and/or best clinical judgment.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Derivation of a 28-Marker Gene Panel

Raw probe intensities (n=6,892,960 features) from n=49 whole blood samples were used to identify genes that best discriminated between different types of melanoma samples e.g., treated versus untreated, simultaneously. A total of 28 transcripts were identified in an unbiased manner as potential markers of melanoma behavior (Table 2).

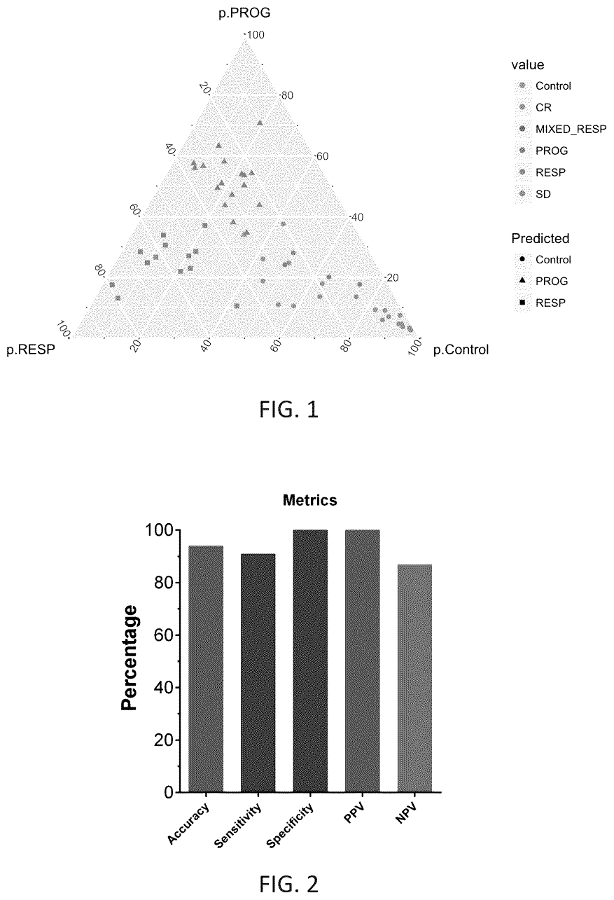

An artificial intelligence model of melanoma disease dynamics was built using normalized gene expression of these 28 markers in whole blood from Controls (n=90), Responders/Stable (n=68), and Progressive (n=66) samples. The dataset was randomly split into training (n=169) and testing (n=55) partitions for model creation and validation respectively. Five algorithms (XGB, RF, Treeflag, SVM, NNET) were identified that best predicted the training data. In the test set, each algorithm produced probability scores that predicted the sample. Each probability score reflects the “certainty” of an algorithm that an unknown sample belongs to Control, Responder/Stable or Progressive class. For example, an unknown sample S1 can have the following probability vector {Control=20, Responder=50, Progressive=30}. This sample would be considered a responder, given a score of 50, but there may be potential of progression (probability of 30) (FIG. 1).

TABLE 2

| | | | | | Amplicon | | |
|---|---|---|---|---|---|---|---|
| | | | | | produced | | |
| | | | | | using | | |
| | | | | | forward and | | |
| | | Chromosome | | | reverse | Assay | Exon |
| Melanoma Biomarker or Housekeeping Gene | | location | UniGene | | primers | Loca- | Bound- |
| Symbol | Name | [Cytogenetic band] | ID | RefSeq | Length (bp) | tion | ary |
| ATL1 | atlastin GTPase 1 | Chr.14: | Hs.584905 | NM_001127713.1 | 65 | 1544 | 12-13 |
| | | 50533082-50633068 | | | | | |
| | | [14q22.1] | | | | | |
| ATP6V0D | ATPase H+ transporting V0 | Chr.16: | Hs.106876 | NM_004691.4 | 72 | 224 | 1-2 |
| | subunit d1 | 67438014-67481186 | | | | | |
| | | [16q22.1] | | | | | |
| C1ORF21 | chromosome 1 open | Chr.1: | Hs.497159 | NM_030806.3 | 84 | 805 | 5-6 |
| | reading frame 21 | 184387016-184629021 | | | | | |
| | | [1q25.3] | | | | | |
| CFLAR | CASP8 and FADD like | Chr.2: | Hs.390736 | NM_001127183.2 | 59 | 874 | 3-4 |
| | apoptosis regulator | 201116015-201172688 | | | | | |
| | | [2q33.1] | | | | | |
| CFLAR-AS1 | CFLAR antisense RNA 1 | Chr.2: | Hs.664613 | NR_040030.1 | 103 | 883 | 6-7 |
| | | 201140289-201157792 | | | | | |
| | | [2q33.1] | | | | | |
| CHP1 | calcineurin like EF-hand | Chr.15: | Hs.406234 | NM_007236.4 | 120 | 212 | 1-2 |
| | protein 1 | 41231149-41281887 | | | | | |
| | | [15q15.1 ] | | | | | |
| DDX55 | DEAD-box helicase 55 | Chr.12: | Hs.286173 | NM_020936.2 | 74 | 183 | 1-2 |
| | | 123602077-123620943 | | | | | |
| | | [12q24.31] | | | | | |
| DMD | dystrophin | Chr.X: | Hs.495912 | NM_000109.3 | 76 | 9607 | 63-64 |
| | | 31119219-33339609 | | | | | |
| | | [Xp21.1] | | | | | |
| DNAJC9 | DnaJ heat shock protein | Chr.10: | Hs.408577 | NM_015190.4 | 102 | 198 | 1-1 |
| | family (Hsp40) member C9 | 73241954-73247331 | | | | | |
| | | [10q22.2] | | | | | |
| ENOSF1 | enolase superfamily | Chr.18: | Hs.658550 | NM_001126123.3 | 110 | X | 13-14 |
| | member 1 | 670012-712664 | | | | | |
| | | [18p11.32] | | | | | |
| FANCL | Fanconi anemia | Chr.2: | Hs.631890 | NM_001114636.1 | 138 | 540 | 6-7 |
| | complementation group L | 58159243-58241380 | | | | | |
| | | [2p16.1] | | | | | |
| HJURP | Holliday junction | Chr.2: | Hs.532968 | NM_018410.4 | 52 | 399 | 4-5 |
| | recognition protein | 233836701-233854566 | | | | | |
| | | [2q37.1] | | | | | |
| HLA-DOA | major histocompatibility | Chr.6: | Hs.631991 | NM_002119.3 | 124 | 160 | 1-2 |
| | complex, class II, DO alpha | 33004182-33009612 | | | | | |
| | | [6p21.32] | | | | | |
| HLA-DRA | major histocompatibility | Chr.6: | Hs.520048 | NM_019111.4 | 129 | 884 | 4-5 |
| | complex, class II, DR alpha | 32439842-32445046 | | | | | |
| | | [6p21.32] | | | | | |
| HNRNPA3P1 | heterogeneous nuclear | Chr.10: | Hs.632956 | NR_002726.2 | 99 | 2455 | 1-1 |
| | ribonucleoprotein A3 | 43787412-43790417 | | | | | |
| | pseudogene 1 | [10q11.21] | | | | | |
| IL23A | interleukin 23 subunit alpha | Chr.12: | Hs.382212 | NM_016584.2 | 107 | 323 | 1-2 |
| | | 56334159-56340410 | | | | | |
| | | [12q13.3] | | | | | |
| IQGAP1 | IQ motif containing | Chr.15: | Hs.430551 | NM_003870.3 | 69 | 4562 | 34-35 |
| | GTPase | 90388241-90502243 | | | | | |
| | activating protein 1 | [15q26.1] | | | | | |
| LOC494127 | NFYC pseudogene | Chr.9: | Hs.626316 | NR_036691.1 | 86 | 150 | 1-1 |
| | | 87083886-87085225 | | | | | |
| | | [9q21.33] | | | | | |
| LOC646471 | uncharacterized | Chr.1: | Hs.727271 | NR_024498.1 | 80 | 2858 | 1-1 |
| | LOC646471 | 25819954-25823606 | | | | | |
| | | [1p36.11] | | | | | |
| LOH12CR | loss of heterozygosity, 12, | Chr.12: | Hs.67553 | NR_024061.1 | 69 | 140 | 1-2 |
| | chromosomal region 2 | 12355408-12357067 | | | | | |
| | (non-protein coding) | [12p13.2] | | | | | |
| MTRNR2L2_MTO1 | CUSTOM PRIMER | | | | | | |
| PBXIP1 | PBX homeobox interacting | Chr.1: | Hs.505806 | NM_020524.3 | 60 | 189 | 2-3 |
| | protein 1 | 154944080-154956163 | | | | | |
| | | [1q21.3] | | | | | |
| RNF5 | ring finger protein 5 | Chr.6: | Hs.731774 | NM_006913.3 | 67 | 273 | 1-2 |
| | | 32178385-32180793 | | | | | |
| | | [6p21.32] | | | | | |
| SERTAD2 | SERTA domain containing | Chr.2: | Hs.591569 | NM_014755.2 | 91 | 288 | 1-2 |
| | 2 | 64631621-64751091 | | | | | |
| | | [2p14] | | | | | |
| SLC35G5 | solute carrier family 35 | Chr.8: | Hs.458397 | NM_054028.1 | 144 | 541 | 1-1 |
| | member G5 | 11330986-11332186 | | | | | |
| | | [8p23.1] | | | | | |
| SPATS2L | spermatogenesis associated | Chr.2: | Hs.120323 | NM_001100422.1 | 89 | 1364 | 10-11 |
| | serine rich 2 like | 200305881-200482263 | | | | | |
| | | [2q33.1] | | | | | |
| TDRD7 | tudor domain containing | Chr.9: | Hs.193842 | NM_001302884.1 | 59 | 3084 | 15-16 |
| | 7 | 97412020-97496125 | | | | | |
| | | [9q22.33] | | | | | |
| TOX4 | TOX high mobility group | Chr.14: | Hs.555910 | NM_001303523.1 | 145 | 447 | 3-3 |
| (housekeeping | box family member 4 | 21477176-21499177 | | | | | |
| gene) | | [14q11.2] | | | | | |
| TPT1 | tumor protein, | Chr.13: | Hs.374596 | NM_001286272.1 | 131 | 377 | 3-3 |
| (housekeeping | translationally-controlled 1 | 45333471-45341284 | | | | | |
| gene) | | [13q14.13] | | | | | |
| TXK | TXK tyrosine kinase | Chr.4: | Hs.479669 | NM_003328.2 | 113 | 1265 | 11-12 |
| | | 48066393-48135322 | | | | | |
| | | [4p12 ] | | | | | |
| YY2 | YY2 transcription factor | Chr.X: | Hs.673601 | NM_206923.3 | 110 | 552 | 1-1 |
| | | 21855987-21858727 | | | | | |
| | | [Xp22.12] | | | | | |

### Diagnosis: Identification of Samples as Melanoma

In the test set 1, the data for the utility of the test to differentiate melanoma from controls are included in Table 3. The metrics are included in FIG. 2. These are: sensitivity>90%, specificity 100%, PPV 100%, NPV 87%. The overall accuracy is 94%. The tool can therefore differentiate between controls and aggressive and stable melanoma disease.

TABLE 3
Confusion matrix showing classification accuracy of the
5-model algorithm that determines whether a sample
is a melanoma or a control in blood samples

| Predicted/Reference | Melanoma | Control |
|---|---|---|
| Control | 3 | 20 |
| Melanoma | 30 | 0 |

| | |
|---|---|
| Sensitivity | 91% |
| Specificity | 100% |
| Positive Predictive Value | 100% |
| Negative Predictive Value | 87% |
| Accuracy | 94% |

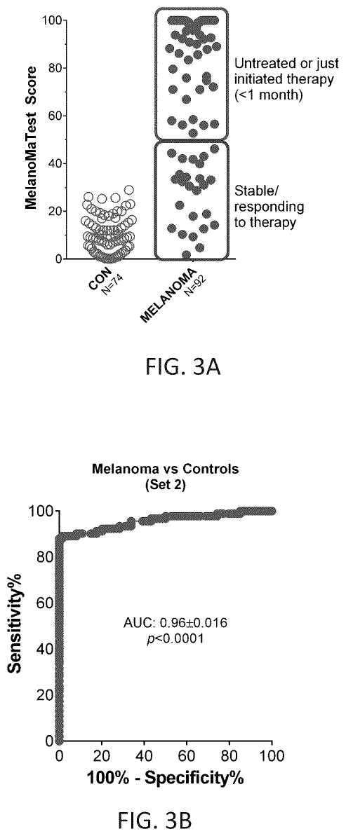

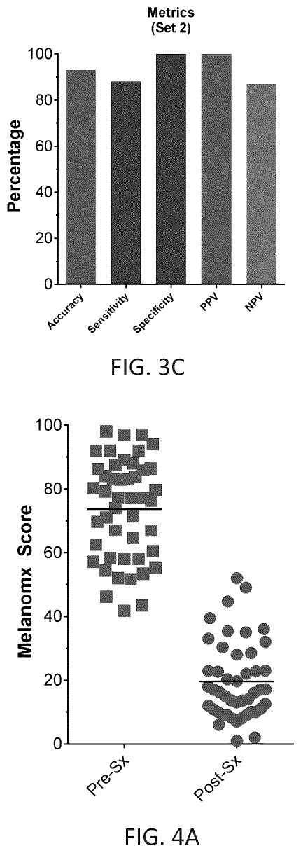

The test was evaluated in a second test set (test set 2) that included 74 controls and 92 melanoma patients. The mean Melanomx score in the melanoma group was 73±31 versus 10±8 in the control group (FIG. 3A). The receiver operator curve analysis demonstrated the score exhibited an area under the curve (AUC) of 0.96 (FIG. 3B) and the metrics were 88-100% (FIG. 3C).

Correlation with surgical removal of melanoma.

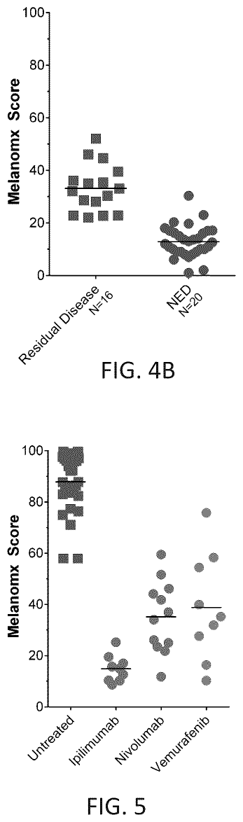

In the surgical series (n=46), removal of melanoma decreased the score from 74±16 to 20±12 (FIG. 4A). Evaluation of the post-surgical group identified the score was significantly different between patients with no evidence of disease 13±6 (not different to the control group) and those with residual disease (33±9) (FIG. 4B). The Melanomx score can therefore define the extent of surgery and identify those who are surgical cures.

Evaluation of immune-therapy and targeted (BRAF inhibitor) therapy.

In the therapy series (n=30), treatment with ipilimumab (immune therapy targeting CTLA-4), nivolumab (immune therapy targeting PD-1) or a BRAF inhibitor (vemurafenib) for 5 months significantly decreased the Melanomx scores from 88±12 to 15±5 (p<0.0001), 35±14 (p<0.001) and 38±21 (p<0.0001), respectively (FIG. 5). All patients in the treated groups were stable. This indicates that the Melanomx score can therefore be used to measure the efficacy of both immune and targeted-therapy in melanoma and that a decrease in score correlated with response to therapeutic intervention.

Confirmation that gene expression is melanoma derived.

We confirmed that melanoma was the source for the blood-based gene expression assay by evaluating expression in different melanoma cell lines, in tumor tissue and by comparing expression in blood with tumor tissue collected at the same time-point during surgery.

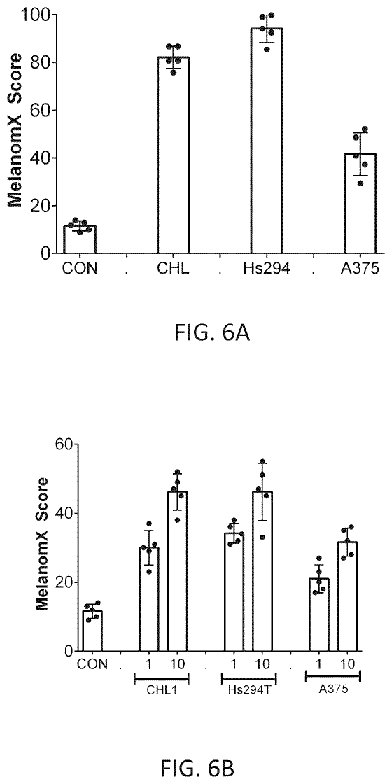

All 28 genes were highly expressed in all 3 melanoma cell lines. Scores ranged from 94±6 (Hs294T) to 82±5 (CHL) to 42±9 (A375) (FIG. 6A).

Spike-in experiments using these 3 cell lines and normal whole blood demonstrated that gene expression scores were detected when as few as 1 cell was spiked into 1 ml of blood. One single melanoma cell was detectable. Scores ranged from 21±4 (A375) to 30±5 (CHL) to 34±2 (H2294) (FIG. 6B). Scores were significantly elevated compared to control blood (no spike-in; p<0.0001).

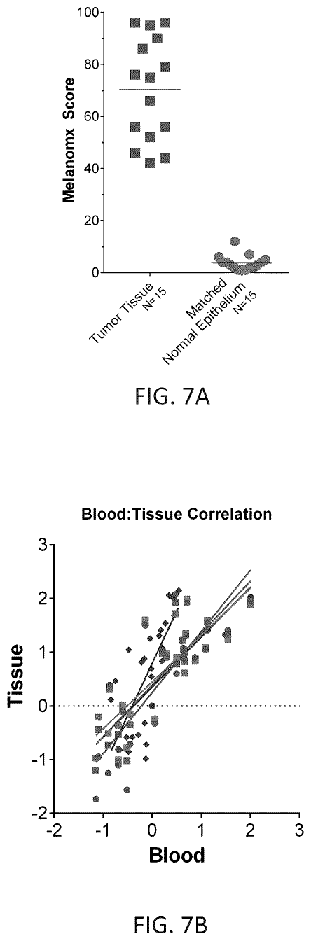

We then evaluated gene expression in tumor tissue. All 28 genes were highly expressed in melanoma compared to matched normal tissue and scores were significantly elevated 70±20 versus 4±3 (p<0.0001) (FIG. 7A).

We also compared gene expression in tumor tissue and blood collected at the same time point. Gene expression was highly concordant (Pearson r: 0.74-0.83, median: 0.819) identifying gene expression in tumor tissue and blood was concordant (FIG. 7B).

### REFERENCES

1. Schadendorf D, Fisher D E, Garbe C, et al. Melanoma. Nat Rev Dis Primers. 2015; 1:15003.: 10.1038/nrdp.2015.1033.
2. Mar V J, Wong S Q, Li J, et al. BRAF/NRAS wild-type melanomas have a high mutation load correlating with histologic and molecular signatures of UV damage. Clin Cancer Res. 2013; 19: 4589-4598. doi: 4510.1158/1078-0432.CCR-4513-0398. Epub 2013 July 4585.
3. Genomic Classification of Cutaneous Melanoma. Cell. 2015; 161: 1681-1696. doi: 1610.1016/j.cell.2015.1605.1044.
4. Wan P T, Garnett M J, Roe S M, et al. Mechanism of activation of the RAF-ERK signaling pathway by oncogenic mutations of B-RAF. Cell. 2004; 116: 855-867.
5. Krauthammer M, Kong Y, Bacchiocchi A, et al. Exome sequencing identifies recurrent mutations in NF1 and RASopathy genes in sun-exposed melanomas. Nat Genet. 2015; 47: 996-1002. doi: 1010.1038/ng.3361. Epub 2015 July 1027.
6. Svedman F C, Pillas D, Taylor A, Kaur M, Linder R, Hansson J. Stage-specific survival and recurrence in patients with cutaneous malignant melanoma in Europe—a systematic review of the literature. Clin Epidemiol. 2016; 8:109-22.: 10.2147/CLEP.S99021. eCollection 92016.
7. Gershenwald J E, Thompson W, Mansfield P F, et al. Multi-institutional melanoma lymphatic mapping experience: the prognostic value of sentinel lymph node status in 612 stage I or II melanoma patients. J Clin Oncol. 1999; 17: 976-983.
8. Minor D R, Moore D, Kim C, et al. Prognostic factors in metastatic melanoma patients treated with biochemotherapy and maintenance immunotherapy. Oncologist. 2009; 14: 995-1002. doi: 1010.1634/theoncologist.2009-0083. Epub 29 Sep. 1023.
9. Kaisaki P J, Cutts A, Popitsch N, et al. Targeted Next-Generation Sequencing of Plasma DNA from Cancer Patients: Factors Influencing Consistency with Tumour DNA and Prospective Investigation of Its Utility for Diagnosis. PLoS One. 2016; 11: e0162809. doi: 0162810.0161371/journal.pone.0162809. eCollection 0162016.
10. Knol A C, Vallee A, Herbreteau G, et al. Clinical significance of BRAF mutation status in circulating tumor DNA of metastatic melanoma patients at baseline. Exp Dermatol. 2016; 25: 783-788. doi: 710.1111/exd.13065.
11. Khoja L, Lorigan P, Dive C, Keilholz U, Fusi A. Circulating tumour cells as tumour biomarkers in melanoma: detection methods and clinical relevance. Ann Oncol. 2015; 26: 33-39. doi: 10.1093/annonc/mdu1207. Epub 2014 June 1096.
12. Stark M S, Klein K, Weide B, et al. The Prognostic and Predictive Value of Melanoma-related MicroRNAs Using Tissue and Serum: A MicroRNA Expression Analysis. EBioMedicine. 2015; 2: 671-680. doi: 610.1016/j.ebiom.2015.1005.1011. eCollection 2015 July
13. Gerami P, Cook R W, Wilkinson J, et al. Development of a prognostic genetic signature to predict the metastatic risk associated with cutaneous melanoma. Clin Cancer Res. 2015; 21: 175-183. doi: 110.1158/1078-0432.CCR-1113-3316.
14. Berger A C, Davidson R S, Poitras J K, et al. Clinical impact of a 31-gene expression profile test for cutaneous melanoma in 156 prospectively and consecutively tested patients. Curr Med Res Opin. 2016; 32: 1599-1604. doi: 1510.1080/03007995.03002016.01192997. Epub 0300216 Jun 03007993.
15. Hanahan D, Weinberg R A. The hallmarks of cancer. Cell. 2000; 100: 57-70.
16. Hanahan D, Weinberg R A. Hallmarks of cancer: the next generation. Cell. 2011; 144: 646-674. doi: 610.1016/j.cell.2011.1002.1013.
17. Kidd M, Drozdov I, Modlin I. Blood and tissue neuroendocrine tumor gene cluster analysis correlate, define hallmarks and predict disease status. Endocr Relat Cancer. 2015; 22: 561-575. doi: 510.1530/ERC-1515-0092. Epub 2015 June 1532.
18. Modlin I, Drozdov I, Kidd M. The Identification of gut neuroendocrine tumor disease by multiple synchronous transcript analysis in blood. Plos One. 2013; e63364.
19. Li S C, Essaghir A, Martijn C, et al. Global microRNA profiling of well-differentiated small intestinal neuroendocrine tumors. Mod Pathol. 2013; 26: 685-696. doi: 610.1038/modpathol.2012.1216. Epub 2013 January 1018.
20. Modlin I, Drozdov I, Alaimo D, et al. A multianalyte PCR blood test outperforms single analyte ELISAs for neuroendocrine tumor detection Endocr Relat Cancer. 2014; 21: 615-628.
21. Modlin I M, Frilling A, Salem R R, et al. Blood measurement of neuroendocrine gene transcripts defines the effectiveness of operative resection and ablation strategies. Surgery. 2016; 159: 336-347. doi: 310.1016/j.surg.2015.1006.1056. Epub 2015 October 1019.
22. Cwikla J B, Bodei L, Kolasinska-Cwikla A, Sankowski A, Modlin I M, Kidd M. Circulating transcript analysis (NETest) in GEP-NETs treated with Somatostatin Analogs defines Therapy. J Clin Endocrinol Metab. 2015; 100: E1437-1445.
23. Bodei L, Kidd M, Modlin I M, et al. Measurement of circulating transcripts and gene cluster analysis predicts and defines therapeutic efficacy of peptide receptor radionuclide therapy (PRRT) in neuroendocrine tumors. Eur J Nucl Med Mol Imaging. 2015; 23: 23.
24. Pavel M, Jann H, Prasad V, Drozdov I, Modlin I M, Kidd M. NET Blood Transcript Analysis defines the Crossing of the Clinical Rubicon: When Stable Disease becomes Progressive. Neuroendocrinology. 2017; 104: 170-182.
25. Merlino G, Herlyn M, Fisher D E, et al. The state of melanoma: challenges and opportunities. Pigment Cell Melanoma Res. 2016; 29: 404-416. doi: 410.1111/pcmr.12475. Epub 1216 April 12417.
26. Diaz-Uriarte R, Alvarez de Andres S. Gene selection and classification of microarray data using random forest. BMC Bioinformatics. 2006; 7: 3.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A kit comprising at least one of: 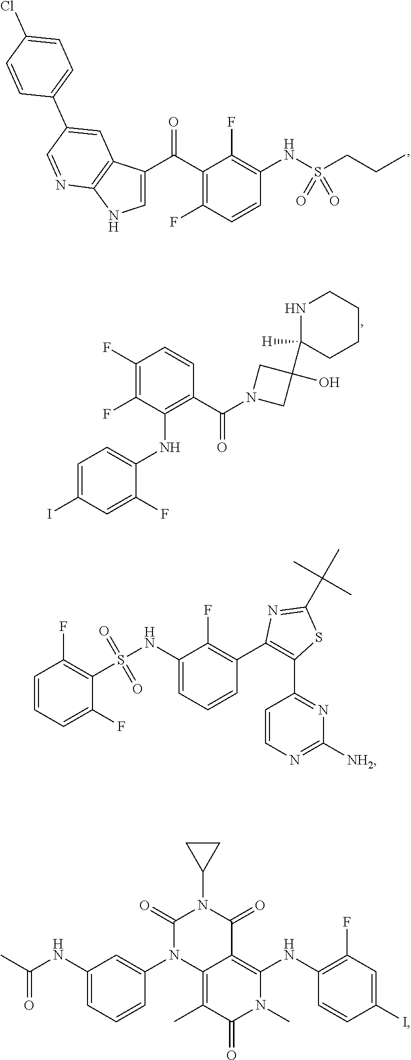
or any combination thereof, and
at least one species of probe that hybridizes to at least one of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2.

2. The kit of claim 1, wherein the kit comprises:

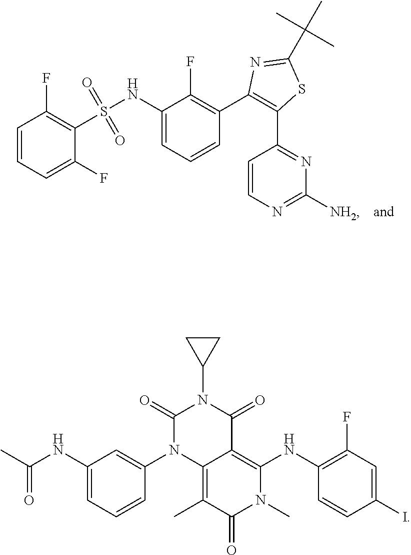 [SMILES: CC(C)(C)c1nc(-c2cccc(NS(=O)(=O)c3c(F)cccc3F)c2F)c(-c2ccnc(N)n2)s1 ; CC(=O)Nc1cccc(-n2c(=O)n(C3CC3)c(=O)c3c(Nc4ccc(I)cc4F)n(C)c(=O)c(C)c32)c1]

3. The kit of claim 1, wherein the kit comprises:

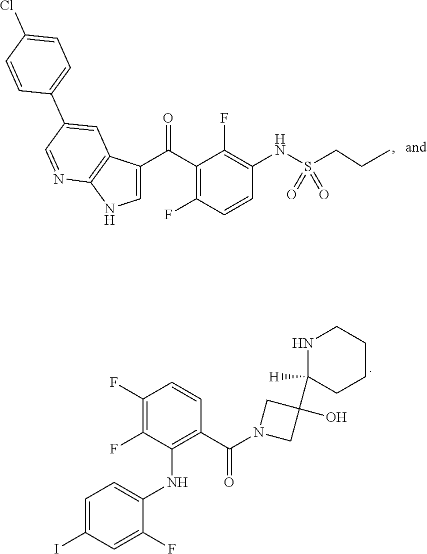 [SMILES: CCCS(=O)(=O)Nc1ccc(F)c(C(=O)c2c[nH]c3ncc(-c4ccc(Cl)cc4)cc23)c1F ; O=C(c1ccc(F)c(F)c1Nc1ccc(I)cc1F)N1CC(O)([C@@H]2CCCCN2)C1]

4. The kit of claim 1, wherein the kit comprises:

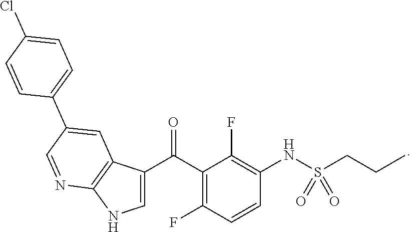 [SMILES: CCCS(=O)(=O)Nc1ccc(F)c(C(=O)c2c[nH]c3ncc(-c4ccc(Cl)cc4)cc23)c1F]

5. The kit of claim 1, wherein the kit comprises:

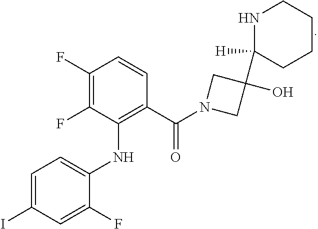 [SMILES: O=C(c1ccc(F)c(F)c1Nc1ccc(I)cc1F)N1CC(O)([C@@H]2CCCCN2)C1]

6. The kit of claim 1, wherein the kit comprises:

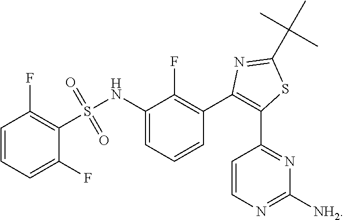 [SMILES: CC(C)(C)c1nc(-c2cccc(NS(=O)(=O)c3c(F)cccc3F)c2F)c(-c2ccnc(N)n2)s1]

7. The kit of claim 1, wherein the kit comprises:

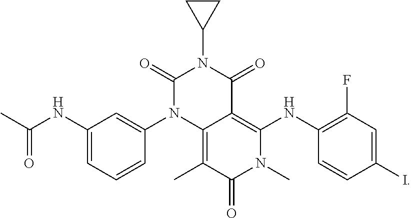 [SMILES: CC(=O)Nc1cccc(-n2c(=O)n(C3CC3)c(=O)c3c(Nc4ccc(I)cc4F)n(C)c(=O)c(C)c32)c1]

8. The kit of claim 1, wherein the at least one probe further comprises at least one detectable label.

9. The kit of claim 1, wherein the kit comprises at least 28 species of probe, wherein the at least 28 species of probe bind to each of ATL1, ATP6V0D, C1ORF21, CFLAR, CFLAR-AS1, CHP1, DDX55, DMD, DNAJC9, ENOSF1, FANCL, HJURP, HLA-DOA, HLA-DRA, HNRNPA3P1, IL23A, IQGAP1, LOC494127, LOC646471, LOH12CR, PBXIP1, RNF5, SERTAD2, SLC35G5, SPATS2L, TDRD7, TXK, YY2.

10. The kit of claim 1, wherein the kit further comprises at least one species of probe that binds to TOX4.

11. The kit of claim 1, wherein the kit further comprises at least one species of probe that binds to TPT1.

12. A method of treating a cancer in a subject in need thereof, the method comprising administering to the subject at least one therapeutically effective amount of at least one of: 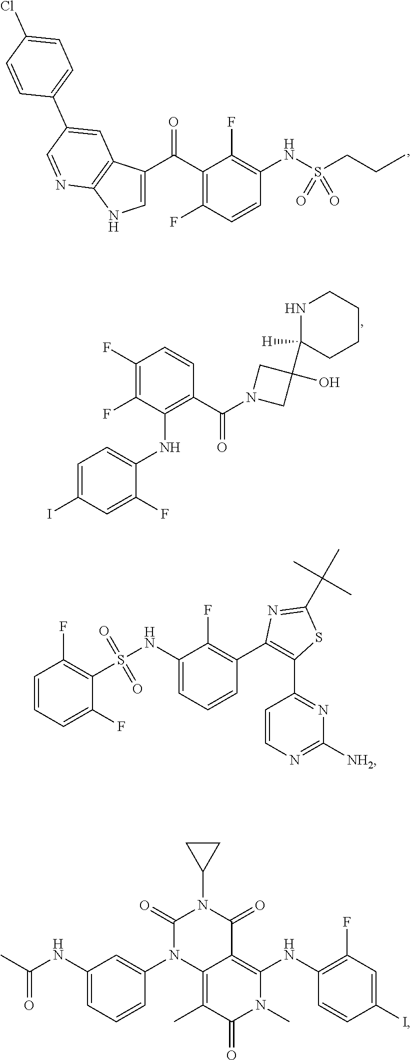
or any combination thereof.

13. The method of claim 12, wherein the method comprises administering at least one therapeutically effective amount of:

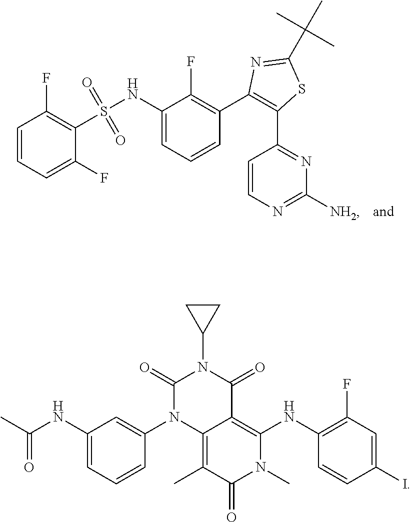 [SMILES: CC(C)(C)c1nc(-c2cccc(NS(=O)(=O)c3c(F)cccc3F)c2F)c(-c2ccnc(N)n2)s1 ; CC(=O)Nc1cccc(-n2c(=O)n(C3CC3)c(=O)c3c(Nc4ccc(I)cc4F)n(C)c(=O)c(C)c32)c1]

14. The method of claim 12, wherein the method comprises administering at least one therapeutically effective amount of:

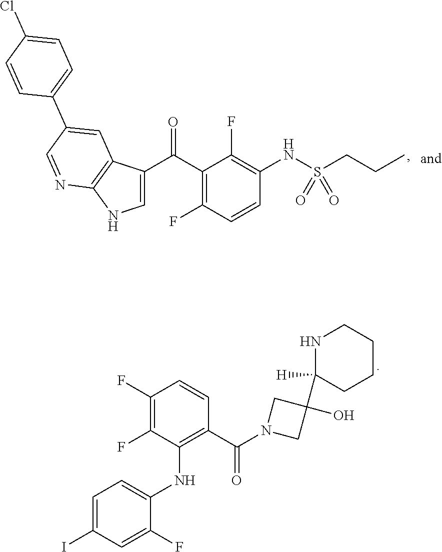 [SMILES: CCCS(=O)(=O)Nc1ccc(F)c(C(=O)c2c[nH]c3ncc(-c4ccc(Cl)cc4)cc23)c1F ; O=C(c1ccc(F)c(F)c1Nc1ccc(I)cc1F)N1CC(O)([C@@H]2CCCCN2)C1]

15. The method of claim 12, wherein the method comprises administering at least one therapeutically effective amount of:

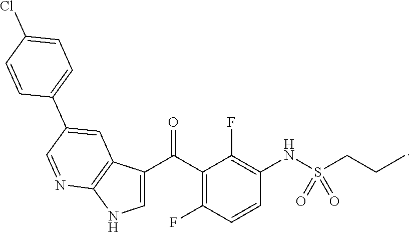 [SMILES: CCCS(=O)(=O)Nc1ccc(F)c(C(=O)c2c[nH]c3ncc(-c4ccc(Cl)cc4)cc23)c1F]

16. The method of claim 12, wherein the method comprises administering at least one therapeutically effective amount of: 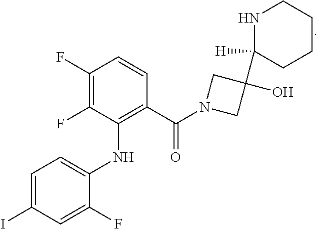 [SMILES: O=C(c1ccc(F)c(F)c1Nc1ccc(I)cc1F)N1CC(O)([C@@H]2CCCCN2)C1]
